SEQUENCE LISTING (225 sequences)

SEQ ID NO: 1 - DNA, 1962 bp, Homo sapiens
  misc_feature 1..1962: note = IDUA polynucleote sequence
  source 1..1962: mol_type = unassigned DNA
atgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatga

SEQ ID NO: 2 - protein, 653 aa, Homo sapiens
  REGION 1..653: note = IDUA polypeptide sequence
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 3 - DNA, 1383 bp, Homo sapiens
  misc_feature 1..1383: note = IDS polynucleotide sequence
  source 1..1383: mol_type = unassigned DNA
atgcctttgcgcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 4 - protein, 460 aa, Homo sapiens
  REGION 1..460: note = IDS polypeptide sequence
MPLRRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 5 - DNA, 1956 bp, Homo sapiens
  misc_feature 1..1956: note = ARSB polynucleotide sequence
  source 1..1956: mol_type = unassigned DNA
atggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcctgtttacttga

SEQ ID NO: 6 - protein, 533 aa, Homo sapiens
  REGION 1..533: note = ARSB polypeptide sequence
MGPRGAASLPRGPGPRRLLLPVVLPLLLLLLLAPPGSGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 7 - DNA, 1956 bp, Homo sapiens; identical to SEQ ID NO: 5
  misc_feature 1..1956: note = GUSB polynucleotide sequence
  source 1..1956: mol_type = unassigned DNA

SEQ ID NO: 8 - protein, 651 aa, Homo sapiens
  REGION 1..651: note = GUSB polypeptide sequence
MARGSAVAWAALGPLLWGCALGLQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSLFT

SEQ ID NO: 9 - DNA, 2238 bp, Homo sapiens
  misc_feature 1..2238: note = ABCD1 polynucleotide sequence
  source 1..2238: mol_type = unassigned DNA
atgccggtgctctccaggccccggccctggcgggggaacacgctgaagcgcacggccgtgctcctggccctcgcggcctatggagcccacaaagtctaccccttggtgcgccagtgcctggccccggccaggggtcttcaggcgcccgccggggagcccacgcaggaggcctccggggtcgcggcggccaaagctggcatgaaccgggtattcctgcagcggctcctgtggctcctgcggctgctgttcccccgggtcctgtgccgggagacggggctgctggccctgcactcggccgccttggtgagccgcaccttcctgtcggtgtatgtggcccgcctggacggaaggctggcccgctgcatcgtccgcaaggacccgcgggcttttggctggcagctgctgcagtggctcctcatcgccctccctgctaccttcgtcaacagtgccatccgttacctggagggccaactggccctgtcgttccgcagccgtctggtggcccacgcctaccgcctctacttctcccagcagacctactaccgggtcagcaacatggacgggcggcttcgcaaccctgaccagtctctgacggaggacgtggtggcctttgcggcctctgtggcccacctctactccaacctgaccaagccactcctggacgtggctgtgacttcctacaccctgcttcgggcggcccgctcccgtggagccggcacagcctggccctcggccatcgccggcctcgtggtgttcctcacggccaacgtgctgcgggccttctcgcccaagttcggggagctggtggcagaggaggcgcggcggaagggggagctgcgctacatgcactcgcgtgtggtggccaactcggaggagatcgccttctatgggggccatgaggtggagctggccctgctacagcgctcctaccaggacctggcctcgcagatcaacctcatccttctggaacgcctgtggtatgttatgctggagcagttcctcatgaagtatgtgtggagcgcctcgggcctgctcatggtggctgtccccatcatcactgccactggctactcagagtcagatgcagaggccgtgaagaaggcagccttggaaaagaaggaggaggagctggtgagcgagcgcacagaagccttcactattgcccgcaacctcctgacagcggctgcagatgccattgagcggatcatgtcgtcgtacaaggaggtgacggagctggctggctacacagcccgggtgcacgagatgttccaggtatttgaagatgttcagcgctgtcacttcaagaggcccagggagctagaggacgctcaggcggggtctgggaccataggccggtctggtgtccgtgtggagggccccctgaagatccgaggccaggtggtggatgtggaacaggggatcatctgcgagaacatccccatcgtcacgccctcaggagaggtggtggtggccagcctcaacatcagggtggaggaaggcatgcatctgctcatcacaggccccaatggctgcggcaagagctccctgttccggatcctgggtgggctctggcccacgtacggtggtgtgctctacaagcccccaccccagcgcatgttctacatcccgcagaggccctacatgtctgtgggctccctgcgtgaccaggtgatctacccggactcagtggaggacatgcaaaggaagggctactcggagcaggacctggaagccatcctggacgtcgtgcacctgcaccacatcctgcagcgggagggaggttgggaggctatgtgtgactggaaggacgtcctgtcgggtggcgagaagcagagaatcggcatggcccgcatgttctaccacaggcccaagtacgccctcctggatgaatgcaccagcgccgtgagcatcgacgtggaaggcaagatcttccaggcggccaaggacgcgggcattgccctgctctccatcacccaccggccctccctgtggaaataccacacacacttgctacagttcgatggggagggcggctggaagttcgagaagctggactcagctgcccgcctgagcctgacggaggagaagcagcggctggagcagcagctggcgggcattcccaagatgcagcggcgcctccaggagctctgccagatcctgggcgaggccgtggccccagcgcatgtgccggcacctagcccgcaaggccctggtggcctccagggtgcctccacctga

SEQ ID NO: 10 - protein, 745 aa, Homo sapiens [sequence omitted]

SEQ ID NO: 11 - DNA, 2058 bp, Homo sapiens
  misc_feature 1..2058: note = GALC polynucleotide sequence
  source 1..2058: mol_type = unassigned DNA
atggctgagtggctactctcggcttcctggcaacgccgagcgaaagctatgactgcggccgcgggttcggcgggccgcgccgcggtgcccttgctgctgtgtgcgctgctggcgcccggcggcgcgtacgtgctcgacgactccgacgggctgggccgggagttcgacggcatcggcgcggtcagcggcggcggggcaacctcccgacttctagtaaattacccagagccctatcgttctcagatattggattatctctttaagccgaattttggtgcctctttgcatattttaaaagtggaaataggtggtgatgggcagacaacagacggcactgagccctcccacatgcattatgcactagatgagaattatttccgaggatacgagtggtggttgatgaaagaagctaagaagaggaatcccaatattacactcattgggttgccatggtcattccctggatggctgggaaaaggtttcgactggccttatgtcaatcttcagctgactgcctattatgtcgtgacctggattgtgggcgccaagcgttaccatgatttggacattgattatattggaatttggaatgagaggtcatataatgccaattatattaagatattaagaaaaatgctgaattatcaaggtctccagcgagtgaaaatcatagcaagtgataatctctgggagtccatctctgcatccatgctccttgatgccgaactcttcaaggtggttgatgttataggggctcattatcctggaacccattcagcaaaagatgcaaagttgactgggaagaagctttggtcttctgaagactttagcactttaaatagtgacatgggtgcaggctgctggggtcgcattttaaatcagaattatatcaatggctatatgacttccacaatcgcatggaatttagtggctagttactatgaacagttgccttatgggagatgcgggttgatgacggcccaggagccatggagtgggcactacgtggtagaatctcctgtctgggtatcagctcataccactcagtttactcaacctggctggtattacctgaagacagttggccatttagagaaaggaggaagctacgtagctctgactgatggcttagggaacctcaccatcatcattgaaaccatgagtcataaacattctaagtgcatacggccatttcttccttatttcaatgtgtcacaacaatttgccacctttgttcttaagggatcttttagtgaaataccagagctacaggtatggtataccaaacttggaaaaacatccgaaagatttctttttaagcagctggattctctatggctccttgacagcgatggcagtttcacactgagcctgcatgaagatgagctgttcacactcaccactctcaccactggtcgcaaaggcagctacccgcttcctccaaaatcccagcccttcccaagtacctataaggatgatttcaatgttgattacccattttttagtgaagctccaaactttgctgatcaaactggtgtatttgaatattttacaaatattgaagaccctggcgagcatcacttcacgctacgccaagttctcaaccagagacccattacatgggctgccgatgcatccaacacaatcagtattataggagactacaactggaccaatctgactataaagtgtgatgtatacatagagacccctgacacaggaggtgtgttcattgcaggaagagtaaataaaggtggtattttgattagaagtgccagaggaattttcttctggatttttgcaaatggatcttacagggttacaggtgatttagctggatggattatatatgctttaggacgtgttgaagttacagcaaaaaaatggtatacactcacgttaactattaagggtcatttcacctctggcatgctgaatgacaagtctctgtggacagacatccctgtgaattttccaaagaatggctgggctgcaattggaactcactcctttgaatttgcacagtttgacaactttcttgtggaagccacacgctaa

SEQ ID NO: 12 - protein, 685 aa, Homo sapiens
  REGION 1..685: note = GALC polypeptide sequence
MAEWLLSASWQRRAKAMTAAAGSAGRAAVPLLLCALLAPGGAYVLDDSDGLGREFDGIGAVSGGGATSRLLVNYPEPYRSQILDYLFKPNFGASLHILKVEIGGDGQTTDGTEPSHMHYALDENYFRGYEWWLMKEAKKRNPNITLIGLPWSFPGWLGKGFDWPYVNLQLTAYYVVTWIVGAKRYHDLDIDYIGIWNERSYNANYIKILRKMLNYQGLQRVKIIASDNLWESISASMLLDAELFKVVDVIGAHYPGTHSAKDAKLTGKKLWSSEDFSTLNSDMGAGCWGRILNQNYINGYMTSTIAWNLVASYYEQLPYGRCGLMTAQEPWSGHYVVESPVWVSAHTTQFTQPGWYYLKTVGHLEKGGSYVALTDGLGNLTIIIETMSHKHSKCIRPFLPYFNVSQQFATFVLKGSFSEIPELQVWYTKLGKTSERFLFKQLDSLWLLDSDGSFTLSLHEDELFTLTTLTTGRKGSYPLPPKSQPFPSTYKDDFNVDYPFFSEAPNFADQTGVFEYFTNIEDPGEHHFTLRQVLNQRPITWAADASNTISIIGDYNWTNLTIKCDVYIETPDTGGVFIAGRVNKGGILIRSARGIFFWIFANGSYRVTGDLAGWIIYALGRVEVTAKKWYTLTLTIKGHFTSGMLNDKSLWTDIPVNFPKNGWAAIGTHSFEFAQFDNFLVEATR

SEQ ID NO: 13 - DNA, 1530 bp, Homo sapiens
  misc_feature 1..1530: note = ARSA polynucleotide sequence
  source 1..1530: mol_type = unassigned DNA
atgtccatgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcctga

SEQ ID NO: 14 - protein, 509 aa, Homo sapiens
  REGION 1..509: note = ARSA polypeptide sequence
MSMGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 15 - DNA, 1584 bp, Homo sapiens
  misc_feature 1..1584: note = PSAP polynucleotide sequence
  source 1..1584: mol_type = unassigned DNA
atgtacgccctcttcctcctggccagcctcctgggcgcggctctagccggcccggtccttggactgaaagaatgcaccaggggctcggcagtgtggtgccagaatgtgaagacggcgtccgactgcggggcagtgaagcactgcctgcagaccgtttggaacaagccaacagtgaaatcccttccctgcgacatatgcaaagacgttgtcaccgcagctggtgatatgctgaaggacaatgccactgaggaggagatccttgtttacttggagaagacctgtgactggcttccgaaaccgaacatgtctgcttcatgcaaggagatagtggactcctacctccctgtcatcctggacatcattaaaggagaaatgagccgtcctggggaggtgtgctctgctctcaacctctgcgagtctctccagaagcacctagcagagctgaatcaccagaagcagctggagtccaataagatcccagagctggacatgactgaggtggtggcccccttcatggccaacatccctctcctcctctaccctcaggacggcccccgcagcaagccccagccaaaggataatggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaggatcagcaacccaaggagatctgtgcgctggttgggttctgtgatgaggtgaaagagatgcccatgcagactctggtccccgccaaagtggcctccaagaatgtcatccctgccctggaactggtggagcccattaagaagcacgaggtcccagcaaagtctgatgtttactgtgaggtgtgtgaattcctggtgaaggaggtgaccaagctgattgacaacaacaagactgagaaagaaatactcgacgcttttgacaaaatgtgctcgaagctgccgaagtccctgtcggaagagtgccaggaggtggtggacacgtacggcagctccatcctgtccatcctgctggaggaggtcagccctgagctggtgtgcagcatgctgcacctctgctctggcacgcggctgcctgcactgaccgttcacgtgactcagccaaaggacggtggcttctgcgaagtgtgcaagaagctggtgggttatttggatcgcaacctggagaaaaacagcaccaagcaggagatcctggctgctcttgagaaaggctgcagcttcctgccagacccttaccagaagcagtgtgatcagtttgtggcagagtacgagcccgtgctgatcgagatcctggtggaggtgatggatccttccttcgtgtgcttgaaaattggagcctgcccctcggcccataagcccttgttgggaactgagaagtgtatatggggcccaagctactggtgccagaacacagagacagcagcccagtgcaatgctgtcgagcattgcaaacgccatgtgtggaactag

SEQ ID NO: 16 - protein, 527 aa, Homo sapiens
  REGION 1..527: note = PSAP polypeptide sequence
MYALFLLASLLGAALAGPVLGLKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQDQQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSILSILLEEVSPELVCSMLHLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCIWGPSYWCQNTETAAQCNAVEHCKRHVWN

SEQ ID NO: 17 - DNA, 1611 bp, Homo sapiens
  misc_feature 1..1611: note = GBA polynucleotide sequence
  source 1..1611: mol_type = unassigned DNA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 18 - protein, 536 aa, Homo sapiens
  REGION 1..536: note = GBA polypeptide sequence
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 19 - DNA, 1401 bp, Homo sapiens
  misc_feature 1..1401: note = FUCA1 polynucleotide sequence
  source 1..1401: mol_type = unassigned DNA
atgcgggctccggggatgaggtcgcggccggcgggtcccgcgctgttgctgctgctgctcttcctcggagcggccgagtcggtgcgtcgggcccagcctccgcgccgctacaccccagactggccgagcctggattctcggccgctgccggcctggttcgacgaagccaagttcggggtgttcatccactggggcgtgttctcggtgcccgcctggggcagcgagtggttctggtggcactggcagggcgaggggcggccgcagtaccagcgcttcatgcgcgacaactacccgcccggcttcagctacgccgacttcggaccgcagttcactgcgcgcttcttccacccggaggagtgggccgacctcttccaggccgcgggcgccaagtatgtagttttgacgacaaagcatcacgaaggcttcacaaactggccgagtcctgtgtcttggaactggaactccaaagacgtggggcctcatcgggatttggttggtgaattgggaacagctctccggaagaggaacatccgctatggactataccactcactcttagagtggttccatccactctatctacttgataagaaaaatggcttcaaaacacagcattttgtcagtgcaaaaacaatgccagagctgtacgaccttgttaacagctataaacctgatctgatctggtctgatggggagtgggaatgtcctgatacttactggaactccacaaattttctttcatggctctacaatgacagccctgtcaaggatgaggtggtagtaaatgaccgatggggtcagaactgttcctgtcaccatggaggatactataactgtgaagataaattcaagccacagagcttgccagatcacaagtgggagatgtgcaccagcattgacaagttttcctggggctatcgtcgtgacatggcattgtctgatgttacagaagaatctgaaatcatttcggaactggttcagacagtaagtttgggaggcaactatcttctgaacattggaccaactaaagatggactgattgttcccatcttccaagaaaggcttcttgctgttgggaaatggctgagcatcaatggggaggctatctatgcctccaaaccatggcgggtgcaatgggaaaagaacacaacatctgtatggtatacctcaaagggatcggctgtttatgccatttttctgcactggccagaaaatggagtcttaaaccttgaatcccccataactacctcaactacaaagataacaatgctgggaattcaaggagatctgaagtggtccacagatccagataaaggtctcttcatctctctaccccagttgccaccctctgctgtccccgcagagtttgcttggactataaagctgacaggagtgaagtaa

SEQ ID NO: 20 - protein, 466 aa, Homo sapiens
  REGION 1..466: note = FUCA1 polypeptide sequence
MRAPGMRSRPAGPALLLLLLFLGAAESVRRAQPPRRYTPDWPSLDSRPLPAWFDEAKFGVFIHWGVFSVPAWGSEWFWWHWQGEGRPQYQRFMRDNYPPGFSYADFGPQFTARFFHPEEWADLFQAAGAKYVVLTTKHHEGFTNWPSPVSWNWNSKDVGPHRDLVGELGTALRKRNIRYGLYHSLLEWFHPLYLLDKKNGFKTQHFVSAKTMPELYDLVNSYKPDLIWSDGEWECPDTYWNSTNFLSWLYNDSPVKDEVVVNDRWGQNCSCHHGGYYNCEDKFKPQSLPDHKWEMCTSIDKFSWGYRRDMALSDVTEESEIISELVQTVSLGGNYLLNIGPTKDGLIVPIFQERLLAVGKWLSINGEAIYASKPWRVQWEKNTTSVWYTSKGSAVYAIFLHWPENGVLNLESPITTSTTKITMLGIQGDLKWSTDPDKGLFISLPQLPPSAVPAEFAWTIKLTGVK

SEQ ID NO: 21 - DNA, 3036 bp, Homo sapiens
  misc_feature 1..3036: note = MAN2B1 polynucleotide sequence
  source 1..3036: mol_type = unassigned DNA
atgggcgcctacgcgcgggcttcgggggtctgcgctcgcggctgcctggactcagcaggcccctggaccatgtcccgcgccctgcggccaccgctcccgcctctctgctttttccttttgttgctggcggctgccggtgctcgggccgggggatacgagacatgccccacagtgcagccgaacatgctgaacgtgcacctgctgcctcacacacatgatgacgtgggctggctcaaaaccgtggaccagtacttttatggaatcaagaatgacatccagcacgccggtgtgcagtacatcctggactcggtcatctctgccttgctggcagatcccacccgtcgcttcatttacgtggagattgccttcttctcccgttggtggcaccagcagacaaatgccacacaggaagtcgtgcgagaccttgtgcgccaggggcgcctggagttcgccaatggtggctgggtgatgaacgatgaggcagccacccactacggtgccatcgtggaccagatgacacttgggctgcgctttctggaggacacatttggcaatgatgggcgaccccgtgtggcctggcacattgaccccttcggccactctcgggagcaggcctcgctgtttgcgcagatgggcttcgacggcttcttctttgggcgccttgattatcaagataagtgggtacggatgcagaagctggagatggagcaggtgtggcgggccagcaccagcctgaagcccccgaccgcggacctcttcactggtgtgcttcccaatggttacaacccgccaaggaatctgtgctgggatgtgctgtgtgtcgatcagccgctggtggaggaccctcgcagccccgagtacaacgccaaggagctggtcgattacttcctaaatgtggccactgcccagggccggtattaccgcaccaaccacactgtgatgaccatgggctcggacttccaatatgagaatgccaacatgtggttcaagaaccttgacaagctcatccggctggtaaatgcgcagcaggcaaaaggaagcagtgtccatgttctctactccacccccgcttgttacctctgggagctgaacaaggccaacctcacctggtcagtgaaacatgacgacttcttcccttacgcggatggcccccaccagttctggaccggttacttttccagtcggccggccctcaaacgctacgagcgcctcagctacaacttcctgcaggtgtgcaaccagctggaggcgctggtgggcctggcggccaacgtgggaccctatggctccggagacagtgcacccctcaatgaggcgatggctgtgctccagcatcacgacgccgtcagcggcacctcccgccagcacgtggccaacgactacgcgcgccagcttgcggcaggctgggggccttgcgaggttcttctgagcaacgcgctggcgcggctcagaggcttcaaagatcacttcaccttttgccaacagctaaacatcagcatctgcccgctcagccagacggcggcgcgcttccaggtcatcgtttataatcccctggggcggaaggtgaattggatggtacggctgccggtcagcgaaggcgttttcgttgtgaaggaccccaatggcaggacagtgcccagcgatgtggtaatatttcccagctcagacagccaggcgcaccctccggagctgctgttctcagcctcactgcccgccctgggcttcagcacctattcagtagcccaggtgcctcgctggaagccccaggcccgcgcaccacagcccatccccagaagatcctggtcccctgctttaaccatcgaaaatgagcacatccgggcaacgtttgatcctgacacagggctgttgatggagattatgaacatgaatcagcaactcctgctgcctgttcgccagaccttcttctggtacaacgccagtataggtgacaacgaaagtgaccaggcctcaggtgcctacatcttcagacccaaccaacagaaaccgctgcctgtgagccgctgggctcagatccacctggtgaagacacccttggtgcaggaggtgcaccagaacttctcagcttggtgttcccaggtggttcgcctgtacccaggacagcggcacctggagctagagtggtcggtggggccgatacctgtgggcgacacctgggggaaggaggtcatcagccgttttgacacaccgctggagacaaagggacgcttctacacagacagcaatggccgggagatcctggagaggaggcgggattatcgacccacctggaaactgaaccagacggagcccgtggcaggaaactactatccagtcaacacccggatttacatcacggatggaaacatgcagctgactgtgctgactgaccgctcccaggggggcagcagcctgagagatggctcgctggagctcatggtgcaccgaaggctgctgaaggacgatggacgcggagtatcggagccactaatggagaacgggtcgggggcgtgggtgcgagggcgccacctggtgctgctggacacagcccaggctgcagccgccggacaccggctcctggcggagcaggaggtcctggcccctcaggtggtgctggccccgggtggcggcgccgcctacaatctcggggctcctccgcgcacgcagttctcagggctgcgcagggacctgccgccctcggtgcacctgctcacgctggccagctggggccccgaaatggtgctgctgcgcttggagcaccagtttgccgtaggagaggattccggacgtaacctgagcgcccccgttaccttgaacttgagggacctgttctccaccttcaccatcacccgcctgcaggagaccacgctggtggccaaccagctccgcgaggcagcctccaggctcaagtggacaacaaacacaggccccacaccccaccaaactccgtaccagctggacccggccaacatcacgctggaacccatggaaatccgcactttcctggcctcagttcaatggaaggaggtggatggttag

SEQ ID NO: 22 - protein, 1011 aa, Homo sapiens
  REGION 1..1011: note = MAN2B1 polypeptide sequence
MGAYARASGVCARGCLDSAGPWTMSRALRPPLPPLCFFLLLLAAAGARAGGYETCPTVQPNMLNVHLLPHTHDDVGWLKTVDQYFYGIKNDIQHAGVQYILDSVISALLADPTRRFIYVEIAFFSRWWHQQTNATQEVVRDLVRQGRLEFANGGWVMNDEAATHYGAIVDQMTLGLRFLEDTFGNDGRPRVAWHIDPFGHSREQASLFAQMGFDGFFFGRLDYQDKWVRMQKLEMEQVWRASTSLKPPTADLFTGVLPNGYNPPRNLCWDVLCVDQPLVEDPRSPEYNAKELVDYFLNVATAQGRYYRTNHTVMTMGSDFQYENANMWFKNLDKLIRLVNAQQAKGSSVHVLYSTPACYLWELNKANLTWSVKHDDFFPYADGPHQFWTGYFSSRPALKRYERLSYNFLQVCNQLEALVGLAANVGPYGSGDSAPLNEAMAVLQHHDAVSGTSRQHVANDYARQLAAGWGPCEVLLSNALARLRGFKDHFTFCQQLNISICPLSQTAARFQVIVYNPLGRKVNWMVRLPVSEGVFVVKDPNGRTVPSDVVIFPSSDSQAHPPELLFSASLPALGFSTYSVAQVPRWKPQARAPQPIPRRSWSPALTIENEHIRATFDPDTGLLMEIMNMNQQLLLPVRQTFFWYNASIGDNESDQASGAYIFRPNQQKPLPVSRWAQIHLVKTPLVQEVHQNFSAWCSQVVRLYPGQRHLELEWSVGPIPVGDTWGKEVISRFDTPLETKGRFYTDSNGREILERRRDYRPTWKLNQTEPVAGNYYPVNTRIYITDGNMQLTVLTDRSQGGSSLRDGSLELMVHRRLLKDDGRGVSEPLMENGSGAWVRGRHLVLLDTAQAAAAGHRLLAEQEVLAPQVVLAPGGGAAYNLGAPPRTQFSGLRRDLPPSVHLLTLASWGPEMVLLRLEHQFAVGEDSGRNLSAPVTLNLRDLFSTFTITRLQETTLVANQLREAASRLKWTTNTGPTPHQTPYQLDPANITLEPMEIRTFLASVQWKEVDG

SEQ ID NO: 23 - DNA, 1041 bp, Homo sapiens
  misc_feature 1..1041: note = AGA polynucleotide sequence
  source 1..1041: mol_type = unassigned DNA
atggcgcggaagtcgaacttgcctgtgcttctcgtgccgtttctgctctgccaggccctagtgcgctgctccagccctctgcccctggtcgtcaacacttggccctttaagaatgcaaccgaagcagcgtggagggcattagcatctggaggctctgccctggatgcagtggagagcggctgtgccatgtgtgagagagagcagtgtgacggctctgtaggctttggaggaagtcctgatgaacttggagaaaccacactagatgccatgatcatggatggcactactatggatgtaggagcagtaggagatctcagacgaattaaaaatgctattggtgtggcacggaaagtactggaacatacaacacacacacttttagtaggagagtcagccaccacatttgctcaaagtatggggtttatcaatgaagacttatctaccactgcttctcaagctcttcattcagattggcttgctcggaattgccagccaaattattggaggaatgttataccagatccctcaaaatactgcggaccctacaaaccacctggtatcttaaagcaggatattcctatccataaagaaacagaagatgatcgtggtcatgacactattggcatggttgtaatccataagacaggacatattgctgctggtacatctacaaatggtataaaattcaaaatacatggccgtgtaggagactcaccaatacctggagctggagcctatgctgacgatactgcaggggcagccgcagccactgggaatggtgatatattgatgcgcttcctgccaagctaccaagctgtagaatacatgagaagaggagaagatccaaccatagcttgccaaaaagtgatttcaagaatccagaagcattttccagaattctttggggctgttatatgtgccaatgtgactggaagttacggtgctgcttgcaataaactttcaacatttactcagtttagtttcatggtttataattccgaaaaaaatcagccaactgaggaaaaagtggactgcatctaa

SEQ ID NO: 24 - protein, 346 aa, Homo sapiens
  REGION 1..346: note = AGA polypeptide sequence
MARKSNLPVLLVPFLLCQALVRCSSPLPLVVNTWPFKNATEAAWRALASGGSALDAVESGCAMCEREQCDGSVGFGGSPDELGETTLDAMIMDGTTMDVGAVGDLRRIKNAIGVARKVLEHTTHTLLVGESATTFAQSMGFINEDLSTTASQALHSDWLARNCQPNYWRNVIPDPSKYCGPYKPPGILKQDIPIHKETEDDRGHDTIGMVVIHKTGHIAAGTSTNGIKFKIHGRVGDSPIPGAGAYADDTAGAAAATGNGDILMRFLPSYQAVEYMRRGEDPTIACQKVISRIQKHFPEFFGAVICANVTGSYGAACNKLSTFTQFSFMVYNSEKNQPTEEKVDCI

SEQ ID NO: 25 - DNA, 1236 bp, Homo sapiens
  misc_feature 1..1236: note = ASAH1 polynucleotide sequence
  source 1..1236: mol_type = unassigned DNA
atgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 26 - protein, 411 aa, Homo sapiens
  REGION 1..411: note = ASAH1 polypeptide sequence
MNCCIGLGEKARGSHRASYPSLSALFTEASILGFGSFAVKAQWTEDCRKSTYPPSGPTYRGAVPWYTINLDLPPYKRWHELMLDKAPVLKVIVNSLKNMINTFVPSGKIMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDVMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 27 - DNA, 1590 bp, Homo sapiens
  misc_feature 1..1590: note = HEXA polynucleotide sequence
  source 1..1590: mol_type = unassigned DNA
atgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacatagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgaattgctgaggcgaggtgtccaggcccaacccctcaatgtaggcttctgtgagcaggagtttgaacagacctga

SEQ ID NO: 28 - protein, 529 aa, Homo sapiens
  REGION 1..529: note = HEXA polypeptide sequence
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 29 - DNA, 2859 bp, Homo sapiens
  misc_feature 1..2859: note = GAA polynucleotide sequence
  source 1..2859: mol_type = unassigned DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 30 - protein, 952 aa, Homo sapiens
  REGION 1..952: note = GAA polypeptide sequence
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 31 - DNA, 1896 bp, Homo sapiens
  misc_feature 1..1896: note = SMPD1 polynucleotide sequence
  source 1..1896: mol_type = unassigned DNA
atgccccgctacggagcgtcactccgccagagctgccccaggtccggccgggagcagggacaagacgggaccgccggagcccccggactcctttggatgggcctggtgctggcgctggcgctggcgctggcgctggcgctggctctgtctgactctcgggttctctgggctccggcagaggctcaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctag

SEQ ID NO: 32 - protein, 631 aa, Homo sapiens
  REGION 1..631: note = SMPD1 polypeptide sequence
MPRYGASLRQSCPRSGREQGQDGTAGAPGLLWMGLVLALALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 33 - DNA, 1200 bp, Homo sapiens
  misc_feature 1..1200: note = LIPA polynucleotide sequence
  source 1..1200: mol_type = unassigned DNA
atgaaaatgcggttcttggggttggtggtctgtttggttctctggaccctgcattctgaggggtctggagggaaactgacagctgtggatcctgaaacaaacatgaatgtgagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataaaactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaattaggacgattaccagatcatctcattaaggacttatttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtatatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataaaattattaatctaatgaggaaatatcagtga

SEQ ID NO: 34 - protein, 399 aa, Homo sapiens
  REGION 1..399: note = LIPA polypeptide sequence
MKMRFLGLVVCLVLWTLHSEGSGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 35 - DNA, 3093 bp, Homo sapiens
  misc_feature 1..3093: note = CDKL5 polynucleotide sequence
  source 1..3093: mol_type = unassigned DNA
atgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccagacggtggatgtgatggcagaagacagagacaccattctggaccccaagatagacgcttcatgttaaggacgacagaacaacaaggagaatacttctgctgtggtgacccaaagaagcctcacactccgtgcgtcccaaaccgagcccttcatcgtccaatctccagtcctgctccctatccagtactccaggtccgaggcacttccatgtgcccgacactccaggtccgaggcactgatgctttcagctgcccaacccagcaatccgggttctctttcttcgtgagacacgttatgagggaagccctgattcacagggcccaggtaaaccaagctgcgctcctgacataccatgagaatgcggcactgacgggcaagtga

SEQ ID NO: 36 - protein, 1030 aa, Homo sapiens
  REGION 1..1030: note = CDKL5 polypeptide sequence
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPDGGCDGRRQRHHSGPQDRRFMLRTTEQQGEYFCCGDPKKPHTPCVPNRALHRPISSPAPYPVLQVRGTSMCPTLQVRGTDAFSCPTQQSGFSFFVRHVMREALIHRAQVNQAALLTYHENAALTGK

SEQ ID NO: 37 - protein, 18 aa, Homo sapiens
  REGION 1..18: note = Albumin peptide
MKWVTFISLLFLFSSAYS

SEQ ID NO: 38 - protein, 18 aa, Homo sapiens
  REGION 1..18: note = chymotrypsinogen peptide
MAFLWLLSCWALLGTTFG

SEQ ID NO: 39 - protein, 14 aa, Homo sapiens
  REGION 1..14: note = interleukin-2 peptide
MQLLSCIALILALV

SEQ ID NO: 40 - protein, 15 aa, Homo sapiens
  REGION 1..15: note = trypsinogen-2 peptide
MNLLLILTFVAAAVA

SEQ ID NO: 41 - protein, 17 aa, Homo sapiens
  REGION 1..17: note = BM40 peptide
MRAWIFFLLCLAGRALA

SEQ ID NO: 42 - protein, 21 aa, synthetic construct
  REGION 1..21: note = Secrecon
MWWRLWWLLLLLLLLWPMVWA

SEQ ID NO: 43 - protein, 20 aa, synthetic construct
  REGION 1..20: note = Mouse IgKVIII
METDTLLLWVLLLWVPGSTG

SEQ ID NO: 44 - protein, 22 aa, synthetic construct
  REGION 1..22: note = Human IgKVIII
MDMRVPAQLLGLLLLWLRGARC

SEQ ID NO: 45 - protein, 16 aa, synthetic construct
  REGION 1..16: note = CD33
MPLLLLLPLLWAGALA

SEQ ID NO: 46 - protein, 23 aa, synthetic construct
  REGION 1..23: note = tPA
MDAMKRGLCCVLLLCGAVFVSPS

SEQ ID NO: 47 - protein, 16 aa, synthetic construct
  REGION 1..16: note = Consensus
MLLLLLLLLLLALALA

SEQ ID NO: 48 - protein, 17 aa, synthetic construct
  REGION 1..17: note = Native
MLLLLLLLGLRLQLSLG

SEQ ID NO: 49 - protein, 16 aa, synthetic construct
  REGION 1..16: note = Penetratin
RQIKIWFQNRRMKWKK

SEQ ID NO: 50 - protein, 11 aa, synthetic construct
  REGION 1..11: note = TAT
YGRKKRRQRRR

SEQ ID NO: 51 - protein, 18 aa, synthetic construct
  REGION 1..18: note = SynB1
RGGRLSYSRRRFSTSTGR

SEQ ID NO: 52 - protein, 10 aa, synthetic construct
  REGION 1..10: note = SynB3
RRLSYSRRRF

SEQ ID NO: 53 - protein, 12 aa, synthetic construct
  REGION 1..12: note = PTD-4
PIRRRKKLRRLK

SEQ ID NO: 54 - protein, 12 aa, synthetic construct
  REGION 1..12: note = PTD-5
RRQRRTSKLMKR

SEQ ID NO: 55 - protein, 15 aa, synthetic construct
  REGION 1..15: note = FHV Coat
RRRRNRTRRNRRRVR

SEQ ID NO: 56 - protein, 19 aa, synthetic construct
  REGION 1..19: note = BMV Gag
KMTRAQRRAAARRNRWTAR

SEQ ID NO: 57 - protein, 13 aa, synthetic construct
  REGION 1..13: note = HTLV-II Rex
TRRQRTRRARRNR

SEQ ID NO: 58 - protein, 13 aa, synthetic construct
  REGION 1..13: note = D-Tat
GRKKRRQRRRPPQ

SEQ ID NO: 59 - protein, 13 aa, synthetic construct
  REGION 1..13: note = R9-Tat
GRRRRRRRRRPPQ

SEQ ID NO: 60 - protein, 27 aa, synthetic construct
  REGION 1..27: note = Transportan
GWTLNSAGYLLGKINLKALAALAKKIL

SEQ ID NO: 61 - protein, 17 aa, synthetic construct
  REGION 1..17: note = MAP
KLALKLALKLALALKLA

SEQ ID NO: 62 - protein, 27 aa, synthetic construct
  REGION 1..27: note = SBP
MGLGLHLLVLAAALQGAWSQPKKKRKV

SEQ ID NO: 63 - protein, 27 aa, synthetic construct
  REGION 1..27: note = FBP
GALFLGWLGAAGSTMGAWSQPKKKRKV

SEQ ID NO: 64 - protein, 27 aa, synthetic construct
  REGION 1..27: note = MPG ac
GALFLGFLGAAGSTMGAWSQPKKKRKV

SEQ ID NO: 65 - protein, 27 aa, synthetic construct
  REGION 1..27: note = MPG(&#916;NLS)
GALFLGFLGAAGSTMGAWSQPKSKRKV

SEQ ID NO: 66 - protein, 21 aa, synthetic construct
  REGION 1..21: note = Pep-1
KETWWETWWTEWSQPKKKRKV

SEQ ID NO: 67 - protein, 21 aa, synthetic construct
  REGION 1..21: note = Pep-2
KETWFETWFTEWSQPKKKRKV

SEQ ID NO: 68 - protein, 18 aa, synthetic construct
  REGION 1..18: note = ApoE p1
LRKLRKRLLLRKLRKRLL

SEQ ID NO: 69 - protein, 30 aa, synthetic construct
  REGION 1..30: note = ApoE p2
LRKLRKRLLRDADDLLRKLRKRLLRDADDL

SEQ ID NO: 70 - protein, 17 aa, synthetic construct
  REGION 1..17: note = ApoE p3
LRVRLASHLRKLRKRLL

SEQ ID NO: 71 - protein, 20 aa, synthetic construct
  REGION 1..20: note = ApoE p4
TEELRVRLASHLRKLRKRLL

SEQ ID NO: 72 - protein, 34 aa, synthetic construct
  REGION 1..34: note = ApoE p5
LRVRLASHLRKLRKRLLLRVRLASHLRKLRKRLL

SEQ ID NO: 73 - protein, 40 aa, synthetic construct
  REGION 1..40: note = ApoE p6
TEELRVRLASHLRKLRKRLLTEELRVRLASHLRKLRKRLL

SEQ ID NO: 74 - protein, 10 aa, synthetic construct
  REGION 1..10: note = Myc Peptide
EQKLISEEDL

SEQ ID NO: 75 - protein, 39 aa, synthetic construct
  REGION 1..39: note = ApoB Peptide
SSVIDALQYKLEGTTRLTRKRGLKLATALSLSNKFVEGS

SEQ ID NO: 76 - DNA, 18242 bp, Homo sapiens
  misc_feature 1..18242: note = CX3CR1_loci
  source 1..18242: mol_type = unassigned DNA
ggggcgggccgtgcttaccaggccgtggacttaaaccaggatgagagaacccctggaggcgtttaagttggcagacttggatttcaggaagagctctctggcttctgggtggagaatggccagtggggtaagtggtgagaggaaagacagagaacggagaaggttagatgggcttgggaaattatccaggccctggatggaggtagagatgtgtgctcatgaacacggaggggattactgatgtggggtggatgagactgtcgtcaagagtgtgggacaggaagagagggagagtcttggccagatccaagaaaggagccctcagaagaggaggggagtcagaggcaaggaaggggctgaggcagccagcccagctgagtggaccccaggagaggtatcaaggggtggtgtggggtggggaggggccagtgtcagaaagtggatggggagcggcctgactctgcttttgtcctgtggccttctggccaaaggcagggaaaggtggccaaacactgagaccaagaacaaagaaagaaaactgctggtggacttcttccaccatgagcaggccaccaagcccgcagcactgcactgcagcccccagctctgtcctggggttgggggaggtgaggaggggcaaggtggggagcacacagagcacccgctgtcctcggaacaccacagcgactagaggtaagggagcaccggatgtggctgggatgtgggcagcaaggggccagaggggccttgaaggggtcacagaccatttaatgaaggtgtattgaaggccaccatgggccaggccctagttagggatggatcagaattatatagcatatgccaggggtcaggcaggtaatgaagtgatcggaaggtgatgaggcagtggcagttgagattcacgttgcagtcgccccaagctggccaggccagggagcagaagcatggctggatgccggagcccaccaggctccccactgcagggcaagagtggcagggggagagactgtgaaaggagcataggccaggtcctgggtgaaagctgtgtcctcagccttgactgatgggtatagggagccactaaatgccttggggcagagaggtgaggaaaaaaatatttaccgagcatctacaaggtgcaaggtactcactagatgccttcagtaccaaagcttctcaaacttagtatgcatatcactcttctaagaatttcattaaaatgcagattctaattcagcagatatagggcagggcttgaggtgctgtctttaataagctcccagtgcctgggactgcactttgaggagaagagctgtgtgtgccccagtgtggtccagtgagtactctgggctccctctcgtgggcagggaagctgagggccccatgagctctcccagcttcctgaaggctccccattaatgagagctgactgtgctgtgctttgctgactgcagggcctgctccctgccccccacctccaggttggggtaagtggcacctctctccctccagctccgcagtcttccctgaggtttagatcttccaggtttataaagtcaggccctcctgttggcagctggcctccaccctggagtatctgagcttgcctgtggcagcatctaaagatagtctcccttacaggaaacaagatactattggctaactctgcaaataaaatgctcttagagggaaggaaagggaaatactcgtctctggtaaagtctgagcaggacagggtggctgactggcagatccagaggttcccttggcagtccacgccaggtaggtgcacaggactagttgggtacctgtgggtggggtggagcagtggacagctaataggttaataatgcctgtttgcttacgtgcagacaatggaaaccattttcctggggatgttgtagcctaaatatgtccaaggggatggaagagtgggaggcaaggggtgatcagatcatttataatacactcaacctggtggaatagtattagaagcattagtaattacattttagagacatggagaaaagctcatgattttaaactaactgaaaaaagcatgaaaaattgcatctggatgctgttgaggaattattgctaattttttgagatgagaaattgtattaaagtctttttcaaaaaagagtccttaacttttagagatgcacacaagtgtttatgggtgaaattaaataattcagtagagacattaagtgggaaatagaggaaatattgaccatgggttgctaatagttgaagccaggtgttgggtataaggaggttctcgctgcttttatttgaaaagttgctttttatttgaaaatttaataataaagagtttttaaatttgtatctgtattttaatataaatataaatgcacttaatataaatataaaatatgtataacatttagatagagaaaagctaaaagattactgtggttgattctaggaaactgcattgcagatagtttcatgggtttttttttcctttttcttccactttttgtaccatctatgggtttttgtttgttttttgttttttgggttttctttcttgtttgtgttttgttttttgagacggagttttgctcttgttgcctaagctggagtgcaatggcacagtctcggctcactgcaacctctgcctcctgggttcaagtgattctcctgcctcagcctcccaagtagctgggattataggcatgtaccaccgcccggctaattttgtatttttagtagaggcggggtttctccattaataaattcctggcacaaatttagtgttcaattttgatatatgttgttataaccattgtgaggatactcaggctcaggtttgtgtgggtggaaaacatggtcttcagaaagaaattatgagtgcaagacaggaggaaatccatcagaggccccagctgaggactgaccacggcttgttatttctcttgccttgcctctggcaatcacagcctcacagagcctgcaatccttgctttgtgagtttatagctcagtccagagaatggctaagaaagtttaggattctttcaacacccactccacaaaaaaaaaaaaaaaaaagaaaagaaaaaaaaattaatttttgaaatacttgaggtagaaaacttgaggcagaaaaaaattgagccaaaaaaaaaggaaaattgaaccacgtgaaagcaggcaagaaagcttgcattgctcagggcatcccaggcccagagggcgctttggagggagctgggtttcctgagaggaggcagggtgggtgacggacctgtgctggagagccttgaggaccactgtgggttgggaatgggggcagtggattggggttcaaaacccctgggaatgagaaatgggctcaggaaggctagggtggattctttcatcttcctctttgcttggctttattttcacaaaggaaggcagggcaggaaatagtctcagcccaacttcagtgtggttcttcttagtgctcaggcttacctggcacttgccacacctctgggatgggagcacctactatccatcagccacgtgccagtctccacaaagtctgctcctgaaccctgctcctcagctggccccacttcacagatggggacataggcagcttggctttggaatgaaggaatgaagtcaggaatgaagtcctggctctgcacttggtgactgtgcactgggcttgctaagtctgtttcctgcttttaaaatggagattgtccatcagcctttgaagccatgtaatgggtatgtgtcaactttctgcaaggattaaaggcatggtataggaagtcccaaacacactgcctgacccatctttgatgctcaagaaacgatatatgttgttgtcatgaggaaactgagcctcagaaagtttggatacctgaaaaacactgactactattgaatgaggttgtgaagaatccagagctgtaggggcaggaaagcaaagaacgtattagagctgacccagtcaggacgatcgtctatccccttcctcaccccaccccatcccaggaggaagcctgcccggccctaggcagctatggcacagtggcaatgtcaggtatggttctccctagccagagaccctagcctcaaaaaacctccttcttgggatccaggcatccaactgctcctccccagccccagcctctgacccagtatcctgagtccagagacgtttggaaccagcacctgtaatggaggagctgaacaaggaggggaacttctgctgctccacagcaggtcacggtcataggagggagtggaaccagaatggcagaatccagatcttggctgcctttcccaaggacttgttctgattcctagcagcacagcccaggcattccgagaagttgggctctctggcatcactcactctgcccagaagagccaggggaaagttggggcttctagctgaaccttgatcccacctgccctcttgaggggctcagaatctgctggctgcttcacaggtgggattctcacggcacgctggccacagctgatgcttcgaccccctcatcttgtttggccaaagtgcagctttttagcttgtgagtaaggaagaaaagctgtatcatatgtctttaaacatcttcctagaccacctttgttttccccttaaagtgtgcttaaggagaaaatggaaagtctcttttcagtgtgttgtattttgtttattgcaaaatacaacacactgaaagaacaatgtctgggttagcaaagtattagatttaatttcccacattactaccatccaggctgagaaagagaaccccagaagcctctctcatgtcccttcctgaccacagcctcttcctctcccacaaagaaccacaagtctgacttctatggtgatcacttccttgcttttccctatagtttgactgaatctaaatatcatccctaaacaatatagcttagctttgcctatttttgatcttcatatttggattcatactttttggtttggctcaagactaaacttttaaaatgcatccacgttgtaggagatgtacatgcacaaaaattttcacggcaacattgtccaaactagcaacaaactgaaagccacctatgtgtgcattagcagtagaaatggcaataaatcatggactagtcacacagtggaatcctacgcagcaaggagaattagcagtctacagccaaaccaacagcatgggtgagatgcttccagaaatactgagatgaatttagagataaaggcacaattggtccatatctacagggcacttaatccagtgcatctccactagacaaaatttgtatttccatgagcaagagtcatttgcactgctatctgctataacctacggtgctgtaaaattagctcaggcaataaaagggggaggtagccccaaagagtatgccagaaaggactcccagtaatcttcagtgtgtgtctatttcaaagtctttcatcatttttccttgtagttacttagtgtggaatttcagacccctctctatgtccctcttctcccttttcagccttggctttctctgcatccttccccgaagccctgcactcagcctaaactgactttcctgaacactctgggagcatgtgggctcctcccaactccaccatcttcactgccttctgttcctatttccctctgcctggccttggccccaggaaaccttcctatgctcagaccctctgtgcctttgtcttcaaagcccacccactattcactgccacctccatcttggtcaacctggaagactttccccatccttaaaacatcagctcaaacggcagatttttttttttttttttgtgatatcctcttgatcaccctcccccagtttttaaggagacagatatagaggttcaatccttgggcttcccatgacttttcttatattctgttactgagcaataataaacacttctaggaactgtatcttaaacacttttgcctctacagaatctagcacagtgcctagtattggtgaaatataattaacaaagtcttctttcaacacagagattctctccacaaaaggagtagagaaagaacagttttattatggaataagcagtaaaccaaaatatgcagagcattataggccatctactaagaggttgcaagaacagaaagaaatctcccccttttgtatagccaagtagatacaacctgttacatacatgttatcaaggcaaacaataactgttcctcaagtaagaggtcttgccagcaccgtttgccgtacatggttcactctaaatttacctggtaattagggtaaccacttgtgttagctaactggctctacccagaggaaaatcaaatttatctttaagacaaggggtaattttgcagcactgagcaaggctcttcagttaggctcataccttcccacagaaactaagagatagaagcactatctcccttaggtttgtttacatttcaaagagatgacgcccaggtccttgggaaagactagcttagctcataaagctgacaaaaagcctatctagtttcaaaaggatttacacatgttcaaagagaggagaaagtatgtaaaagttttctaggtgggcacggtggctcacacctgtattcccagcactttgagaggctgaggcgggtggatcacctgaagtcaggaatttgagatcagcctggccaacatggtgaaaccttgtctctactaaaaatacaaaaattagctgggcatggtggtgggcgcctgtaatcccagctacttgggaggctgaggcaggagaatcacttgaacccaggaggtggaggtggcagtgagccaagatggcaccattgcactccagcctgggcgatgagagtgaaactccatgtcaaaaaaaaaaaaagttttctaaagtaaatgctctgagaaaaaaacagaaggtgaggagatctctgtccttatttttaacaagaataattaaatgtttttatttttaatttctacttagactagaatatggtacagatgctcaaaaatattgacttaattaataattaaacaattaacaagtctatctctttcctaattattaaaaaaattatgaagtatttcccttcaaaatttcacagtttactttgacataaataaataaataccataaagtattttgaagaggaaagggaagggtggggcttcaaacacaaaataatggcaggttttgtaaaggggcaattcactaaacccaataggattctcctgttgctattttttttttttttttttttttggtggtggttggagggaagggttgggtgtgcatttggcctgtgtgaaaaactggtgggtagacaatgccatgctgttctagatgggctcattgctataggaatagatagtcactgaatttacttgcataggggatggggtataaaatgtttccttatagctccttaacatggaattcagcacccctctccccatcagccttagctctctctgaatcccaagggatatgtgttataaaaatagctgccatcataaatgagaaaaccatcaggacctggtaaaacttggcttcccaaacagtcaggggtctgggcatggcagccgactgagaacctttctatctagtacaaggtaaacaagatttgccaagacttgatcaactgatccagctccctatctccaaacagaacctcatctgaatgctcacaggctgtgccccagaagtgtagaagtgtcatgttccctgggcaggctgctgggcagcctctctgttgacaatagaccacacttttgctgacctaggactcatgttgctctttaagactgcttccttggccaggcatgatggctcacacctgtaatcctggcactttgagaggcccaggcaggaggatctcttgaggccagatgttcaagaccagcctggtcaacatagtaagaccccatatctaccaaaaatagctgggcatggtggtgcacacctatactcccagctacttaggagactgaggtgggaggattgctacatcccaggagttcaaggctgtagtgagctatgatcatgccactgcactccagcctgggcaacagagcgagatcctgtctcaaacaaacagtttccttctgtttgattcttgctgaaaaattgagcatgccagagctagcaaggctcttagaggtggtctggtccaatgctttaatttcatacatcaagaaactgaagcagagcagagtgacctgcccagggtctctcagccattcatgctcagaaatgtatgggctcctgtgaaacatgtggctcttaaaagcactatcatatatttgaaggcagaaaataggctaaaccttcagccttcagacttttcctctccagagaaaatgaccccagtttcctcactatggttgctgggagctagattcctggggatctggcagtgtggaccacctagtggtggctagaggagcaaataatatcccgcattccattttccactcaccaatccctgaggggcagcctgctgggttatgagcccacagggggagaaccccaacgaattcagagatgcatcatggaccagtttctcttaaggggcctgggtctactattttcagttctacttcgagagaagtggcctgcaatatcctgcagatttcccctccagggagaaaagcattgtgcggtgcaaggagcacaggctttgtggaaagaggcatctggggttgagatcctggctcttttgcttccttgaacaagttaaccaaatctctgggcctctgctggttaatttataccatggggatcatcatttctccagtggggttgtgaagagaatgtggtgggatcttgtgtgtggagcatgacacttagcaggcatccgggaatggcagcctcctccctttctaaactggggctttctgagggtgacttcagattccacaatgtcaacagcacaatggcatcctcataaggaaagtttgggttggggctcctcaagcaattctctactctcatttggtacaaagaaaaaaattaagcctcacaattttcttggcaccagactgaacctcaaacccagtcttcacttttactaaaaagccataaacagagaccaggagggtaaaaactaccagaagatacactggatttagaaaacagtaagctggatgtgaagccaaaagagagggagaaagcaacataaaagaatgtcagattgaagaagaaatggattctggccacctaaagaagaagggaagtatagaaaagggaagctgattggaaacaatgggtaatgatgagtgtccctcccctaaaaagttaaaaaataaatgagcaggcatgagaaaaggaagtaggccagaggaagtagaccaggatcagccacggatgtgagtggggaattttacaaatttctacttgggtgtgaaacatgtgtacactcaaagctctgtctaggttcaccgaaactctcccagctcaaggccatttagtaaaaccttggcttaactgaatagtgggtaaggaggcctgaggatggcccagagaaggtttaaattcttactggctgctgcactaagttgtaaagtgcagccttagttgatgatgcctttattagtgcaacatcccagaatgcatgcgtcttacaacccttgtggatatgaaacccagctgggtagacactgcaaagtcttctcatttgattcttccacttggtttctagtgtccctcagggacaaaggaaagccatggtccagtctaagatgaaaaccaattagacctctggcaggcctttgaccaccctgagggccacccccaaggcacggccacactcgcatctgctgccaggaggccctatcttaggctcagctcccaaaccttgaacatcttggaggatccacaaataggaggtacactcagctcaggctcagtcttccttaaaaagcgccttgctaaagctatcaagttcactggaatacttctgcgaaggacacagcttcagcgatgtcagattttttatgtaaatggtgccttcacatgctctggggcatttggctaccaagggcggtttgaactagctccagcacacagaatacaagtctcttcagaaccaggcaaaaccctatgttgcccaatgactcctgctgtttcttgagatttgcacagaagacagagctgcaattacccacgctgatatctatttcatgaccacatatgttcaaaagccacatgtgagaggtatggttgaaatgagaggttgtgtttgccaagttgtcttctgactgtggagagctggtgagcctcttctcatctcctggggctccatattatagagctgacgaatctcttttcttgctccttgaagtctttcattcacttatttattaattcatttaacacatcaggcacctactaacttctcagagttcaaggcttcctagcctatgatcagaaaggacacctgtgtgctcatgagcacccctgggatcagggtgactgatagggtgctgtcatcactactagatgaaactctttggaacaaaggtctaggatataatatttatccttctgcaaatattcatatggcattcgctgtgtaccaggccttctgttgaacattggcctgcagagctgaagaggacgtgggtcttctcttaggaactcctagtcttgggaaaaaggaatggggaagggctgtaatgtgaagttaaaaaaaagtgctgaggccgaacacatctgggccctcactgcagccctattgcacacacactgcatcacctcgaacaagttattcaccctctctgaattcatctatttgcccataaaaatagagatgatccttttatatgttggctaccacttattatgtgcagcatgcttgttattgtactaagttactcattttttagtttttcatataactcatatttttgttgatttttattttaaaattccaaacgaattaattaaactattttccaaaatacgtagtatgtgtacatggtaaaaaacatttccaaaagtttaccataaaaactgtctcctttcccatatcctcaatccttcagaatccctctccagggataattaccaccaccagtgtattccttaagagatatttaaactttatacaaaatacgcacacatcattcttttccacaaatgacagcatactatgtacatggtacctcacctagcttttttcacttaccagtatatcttagagattgttgcatgacagaatatacagatctgcctgtttttgttgtttttcccaagtttccaagagctagaaactgtttggtttttaagcagctgcttggtattccattaattggacttatcgtgctttgatctgtccctagtgatggacattggggttgtttcccttcatttatatttgaagcttgttgcagtgacagtgtacacacttgcttgagcatgtgtgtgtctacgataaatcccaataagcaatttaaagtaatttaattcttatgctctcaacaaacctaagaggttatttttttagaggaggaagctaaggctgttgttttgatgcctttttaccactggacttgggactcaactgtttgatagtcatcgagtccttgactcttctcccctgccaggatttggcccctaagcccagggcccgagtctcctccatcttcaagggagagtgggaacagcacaggccttggcctcagtcctgctccccctgcttctagctgtgggatgggccaggtgcttcacccagctgtgcctcctgtgaggcacagtgtgtgcaaaatggaaatgtgaacatgaagatcaaaaatgtccctcaatgaccagtgctgttcctcagagtcacgtgggaactcatagaaagcgatattggtactgctctttcctctgtagcatggtccagatggctcatagcagggaccatgatatgctgggtgagcacccactgcatgcacccactgtgccagcactgagagactcctgtgggagccacagcaattctagggtcttcactggggactctgagacagcagggagctaggatgagggctgcagagtgttcgtctgccctcactgagcagaccccctggatggcagggagcagtcccaagccagatggatgcccataaccagccatttggctctcaatacataatatcaccacgtatcaggcaaaaccatcctgcccagagcattatctgaatttgcatcccatctgcagaagatacattcacccacttcttccattctgtcttaatcaaagtctttatgtgaattttccccattgagaagacaagccccttcctggcttagactgtacctgactgatcttttcatgagctccttgccaagccagaccacccccagcttatatggagacttggtgcaaattagagatgcccctgtgcacgtggcagccctgagcccaagcacccagtaaggcaaagggcctgatttgggacccctctgccactccaccaggcaatcagttgcttatttctaactttcccttccttctccacatttgtcccattccttcctctcatcatgaatatccccagaggcattcagcagtgcagtgaattaaatatagaacttttttttttcagaattgcagaacggattagatcaatattaatccaaacagagcaatgagcctgacagtttagtaaaagctcaataaagggtggcttacctcccccaaaataatctgaaaagaaagcatgtcttatttcagggggaaaaaaaaataaagtgacctttaaagaccaaattcccaggatacccagggtggaggtggaacatgggagtccacaggcagcctggatgtttccaaagatccaaagggcttttgcttcctcacataatgcaggaaacaaattgaacatgtattaagtgcttgctgtatgtgacacactgtgccaggtgctccccttaaaacagttctgtgggcaggcatgagaatgaatcccgatcttacagacaaggaatgttaggctcagaggtttcaagctcacccatcactcagccagagaggacagatgcaggattcaatctcgggagtgcccgagtccacagaagttcctgtgctgaaggaccgaccacaggcacataaagagatgcgagacaatttttactggatttggccacctctcgaggtcggctttgccagctcttctcactgggggaaggggagggagaaagtagctagctccagggtccctaacatagaaccaccaaggacttgactatttttactcatacagcagcttgtctgggaagatcatgctctgtgacaagctgcaggcactaagtagcaatttctgtttcccacatattagcttgagtcatataaaactgacatggatgtggctcaaaaatagctgtatgtcagccattttataccatttgacttaaatgttattaattaacgtcacagccagagattattctctgagaaaagggcattgtagcctgaagcagagaaagcatacacgttccctggggttgagaactcatcacagcctgagacagcttaggttgtaaagccccggcccacttatcccaggagagtctgggtgagatgcaggccccaaagcagaggctgggaagcgagaagtgacacaccctggctgggtgggccctcatcttggtgagacaccacctgggtaaaaccatcatggaaagggtgtagtggggcgtggaaactccctcggttaaagcgtgagctttgctgtaagttgtggtaaggagggaggcagtgacaaccaggaggcctgttttgagggtttctgagggacccatctgtggtatcacgaggagacgcccagaggagccgtgtgaaagggctgcctcccagccggctctggagtgaatgagcagcaagtcctggctgcgaaaagaaggggagtgcagcctgcagaagtgtcttcttttttcaattcctgctcagaaggaaacaggagataagaatagtggggaagtccaaaccaaagtgaactatagggctggtaatcgtagggggaattagtcacccggagactagcccagcagactaacggagccccatcctccatcttgaatcagtcagcccctctatgactgcagagtcctgaatgatggcaacaccttctcttcacttagcgttgtaggatgaccaacagtcctgatttgcctgggactgaggggttcccaatagatgggactttcagggctaaaaccaggaaagtcctgggcagcccaaaacaaggtagtcactctagagtgtatgactctgtctgatacctgctaagaaagagaaggacttgttgattataaggagaagaggaggtgaaatggttctcaaaaaacaaagatgagggcttccgggtgctgttctgcccaaggctctgggtctgaggcttctctctccaggcctagcttcatggaaaagtaaggggccagagggtggaaaaggtggaaacaaaggaagaggatggagaattgctttggggaagtttggactggaagtgtgaattacagctgcacccccaattcaccccatctcacccccctccccctcctgctcatggttctccctttctcatccacacattggtcaaactagctagcttttggagagattttgggcagtaaaagtaaaacagatctgtctcaagcttcaaaaagcctagagctggctgggcgctgtggctcacgcctgtaatcctagcattttgggaggctgaggcggaaggataatctgaggtcaggagtttgagaccagcctggctaacatgatgaaaccccatctctactaaaaatacaaaaattagccaggcgtggtagtgcacgcctataatcccagctatttgggaggctgaggcaggagaatcgcttgaaccccaggggacagaggttgcagtgagctgagatcgcaccactgcactccagcctgggtgacacagcgagactccatttaaaaaaaaaaaaatgcctagagccaaatgctcacagagccatttactgcatggctttgggcaagtcaaaggagtccgcctctcctgtcagaagagtctgttgcagtcttcatcacaagactgttgtggggattaaacaagatggcaagtgggaagttgggaaatgtagtgtgcacccaaccaatatttgtttcttcctgcctgcctacatatgaggccacacagaattccaactttgtttctctgataactaacacagttacttgtttttctttctgatccaggccttcaccatggatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtcctccaggaaatctggcccgtgctccgcaatgtggaaacaaattttcttggcttcctactccccctgctcattatgagttattgctacttcagaatcatccagacgctgttttcctgcaagaaccacaagaaagccaaagccattaaactgatccttctggtggtcatcgtgtttttcctcttctggacaccctacaacgttatgattttcctggagacgcttaagctctatgacttctttcccagttgtgacatgaggaaggatctgaggctggccctcagtgtgactgagacggttgcatttagccattgttgcctgaatcctctcatctatgcatttgctggggagaagttcagaagatacctttaccacctgtatgggaaatgcctggctgtcctgtgtgggcgctcagtccacgttgatttctcctcatctgaatcacaaaggagcaggcatggaagtgttctgagcagcaattttacttaccacacgagtgatggagatgcattgctccttctctgaagggaatcccaaagccttgtgtctacagagaacctggagttcctgaacctgatgctgactagtgaggaaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaagggtgtctgagccctcaaagtgaggggaaaccagggcctgagccaagctagaattccctctctctgactctcaaatcttttagtcattatagatcccccagactttacatgacacagctttatcaccagagagggactgacacccatgtttctctggccccaagggcaaaattcccagggaagtgctctgataggccaagtttgtatcaggtgcccatccctggaaggtgctgttatccatggggaagggatatataagatggaagcttccagtccaatctcatggagaagcagaaatacatatttccaagaagttggatgggtgggtactattctgattacacaaaacaaatgccacacatcacccttaccatgtgcctgatccagcctctcccctgattacaccagcctcgtcttcattaagccctcttccatcatgtccccaaacctgcaagggctccccactgcctactgcatcgagtcaaaactcaaatgcttggcttctcatacgtccaccatggggtcctaccaatagattccccattgcctcctccttcccaaaggactccacccatcctatcagcctgtctcttccatatgacctcatgcatctccacctgctcccaggccagtaagggaaatagaaaaaccctgcccccaaataagaagggatggattccaaccccaactccagtagcttgggacaaatcaagcttcagtttcctggtctgtagaagagggataaggtacctttcacatagagatcatcctttccagcatgaggaactagccaccaactcttgcaggtctcaacccttttgtctgcctcttagacttctgctttccacacctggcactgctgtgctgtgcccaagttgtggtgctgacaaagcttggaagagcctgcaggtgctgctgcgtggcatagcccagacacagaagaggctggttcttacgatggcacccagtgagcactcccaagtctacagagtgatagccttccgtaacccaactctcctggactgccttgaatatcccctcccagtcaccttgtggcaagcccctgcccatctgggaaaataccccatcattcatgctactgccaacctggggagccagggctatgggagcagcttttttttcccccctagaaacgtttggaacaatctaaaagtttaaagctcgaaaacaattgtaataatgctaaagaaaaagtcatccaatctaaccacatcaatattgtcattcctgtattcacccgtccagaccttgttcacactctcacatgtttagagttgcaatcgtaatgtacagatggttttataatctgatttgttttcctcttaacgttagaccacaaatagtgctcgctttctatgtagtttggtaattatcattttagaagactctaccagactgtgtattcattgaagtcagatgtggtaactgttaaattgctgtgtatctgatagctctttggcagtctatatgtttgtataatgaatgagagaataagtcatgttccttcaagatcatgtaccccaatttacttgccattactcaattgataaacatttaacttgtttccaatgtttagcaaatacatattttatagaacttcca

SEQ ID NO: 77 - DNA, 49 bp, synthetic construct
  misc_feature 1..49: note = CX3CR1_T01
  source 1..49: mol_type = other DNA
tctttcctctgtagcatggtccagatggctcatagcagggaccatgata

SEQ ID NO: 78 - DNA, 49 bp, synthetic construct
  misc_feature 1..49: note = CX3CR1_T02
  source 1..49: mol_type = other DNA
tatgctgggtgagcacccactgcatgcacccactgtgccagcactgaga

SEQ ID NO: 79 - DNA, 49 bp, synthetic construct
  misc_feature 1..49: note = CX3CR1_T03
  source 1..49: mol_type = other DNA
tgctgggtgagcacccactgcatgcacccactgtgccagcactgagaga

SEQ ID NO: 80 - DNA, 49 bp, synthetic construct
  misc_feature 1..49: note = CX3CR1_T04
  source 1..49: mol_type = other DNA
tgagagactcctgtgggagccacagcaattctagggtcttcactgggga

SEQ ID NO: 81 - DNA, 49 bp, synthetic construct
  misc_feature 1..49: note = CX3CR1_T05
  source 1..49: mol_type = other DNA
tcctgtgggagccacagcaattctagggtcttcactggggactctgaga

SEQ ID NO: 82 - DNA, 49 bp, synthetic construct
  misc_feature 1..49: note = CX3CR1_T06
  source 1..49: mol_type = other DNA
tgggagccacagcaattctagggtcttcactggggactctgagacagca

SEQ ID NO: 83 - DNA, 49 bp, synthetic construct
  misc_feature 1..49: note = CX3CR1_T07
  source 1..49: mol_type = other DNA
tcgtctgccctcactgagcagaccccctggatggcagggagcagtccca

SEQ ID NO: 84 - DNA, 49 bp, synthetic construct
  misc_feature 1..49: note = CX3CR1_T08
  source 1..49: mol_type = other DNA
tgccctcactgagcagaccccctggatggcagggagcagtcccaagcca

SEQ ID NO: 85 - DNA, 49 bp, synthetic construct
  misc_feature 1..49: note = CX3CR1_T09
  source 1..49: mol_type = other DNA
tggatggcagggagcagtcccaagccagatggatgcccataaccagcca

SEQ ID NO: 86 - DNA, 49 bp, synthetic construct
  misc_feature 1..49: note = CX3CR1_T10
  source 1..49: mol_type = other DNA
tctcaatacataatatcaccacgtatcaggcaaaaccatcctgcccaga

SEQ ID NO: 87 - DNA, 49 bp, synthetic construct
  misc_feature 1..49: note = CX3CR1_T11
  source 1..49: mol_type = other DNA
tatcaggcaaaaccatcctgcccagagcattatctgaatttgcatccca

SEQ ID NO: 88 - DNA, 49 bp, synthetic construct
  misc_feature 1..49: note = CX3CR1_T12
  source 1..49: mol_type = other DNA
tcctgcccagagcattatctgaatttgcatcccatctgcagaagataca

SEQ ID NO: 89 - DNA, 49 bp, synthetic construct
  misc_feature 1..49: note = CX3CR1_T13
  source 1..49: mol_type = other DNA
tctgaatttgcatcccatctgcagaagatacattcacccacttcttcca

SEQ ID NO: 90 - DNA, 49 bp, synthetic construct
  misc_feature 1..49: note = CX3CR1_T14
  source 1..49: mol_type = other DNA
ttccattctgtcttaatcaaagtctttatgtgaattttccccattgaga

SEQ ID NO: 91 - DNA, 49 bp, synthetic construct
  misc_feature 1..49: note = CX3CR1_T15
  source 1..49: mol_type = other DNA
tccattctgtcttaatcaaagtctttatgtgaattttccccattgagaa

SEQ ID NO: 92 - DNA, 49 bp, synthetic construct
  misc_feature 1..49: note = CX3CR1_T16
  source 1..49: mol_type = other DNA
ttctgtcttaatcaaagtctttatgtgaattttccccattgagaagaca

SEQ ID NO: 93 - DNA, 49 bp, synthetic construct
  misc_feature 1..49: note = CX3CR1_T17
  source 1..49: mol_type = other DNA
tctgtcttaatcaaagtctttatgtgaattttccccattgagaagacaa

SEQ ID NO: 94 - DNA, 49 bp, synthetic construct
  misc_feature 1..49: note = CX3CR1_T18
  source 1..49: mol_type = other DNA
tttatgtgaattttccccattgagaagacaagccccttcctggcttaga

SEQ ID NO: 95 - DNA, 49 bp, synthetic construct
  misc_feature 1..49: note = CX3CR1_T19
  source 1..49: mol_type = other DNA
ttcctggcttagactgtacctgactgatcttttcatgagctccttgcca

SEQ ID NO: 96 - DNA, 49 bp, synthetic construct
  misc_feature 1..49: note = CX3CR1_T20
  source 1..49: mol_type = other DNA
tcctggcttagactgtacctgactgatcttttcatgagctccttgccaa

SEQ ID NO: 97 - DNA, 23 bp, synthetic construct
  misc_feature 1..23: note = CX3CR1_gRNA1
  source 1..23: mol_type = other DNA
gtacagtctaagccaggaagggg

SEQ ID NO: 98 - DNA, 23 bp, synthetic construct
  misc_feature 1..23: note = CX3CR1_gRNA2
  source 1..23: mol_type = other DNA
gggagcagtcccaagccagatgg

SEQ ID NO: 99 - DNA, 23 bp, synthetic construct
  misc_feature 1..23: note = CX3CR1_gRNA3
  source 1..23: mol_type = other DNA
gactgctccctgccatccagggg

SEQ ID NO: 100 - DNA, 23 bp, synthetic construct
  misc_feature 1..23: note = CX3CR1_gRNA4
  source 1..23: mol_type = other DNA
gtgaatgtatcttctgcagatgg

SEQ ID NO: 101 - DNA, 23 bp, synthetic construct
  misc_feature 1..23: note = CX3CR1_gRNA5
  source 1..23: mol_type = other DNA
gtctctcagtgctggcacagtgg

SEQ ID NO: 102 - DNA, 23 bp, synthetic construct
  misc_feature 1..23: note = CX3CR1_gRNA6
  source 1..23: mol_type = other DNA
gacagcagggagctaggatgagg

SEQ ID NO: 103 - DNA, 23 bp, synthetic construct
  misc_feature 1..23: note = CX3CR1_gRNA7
  source 1..23: mol_type = other DNA
gaaggggcttgtcttctcaatgg

SEQ ID NO: 104 - DNA, 23 bp, synthetic construct
  misc_feature 1..23: note = CX3CR1_gRNA8
  source 1..23: mol_type = other DNA
gcaaattcagataatgctctggg

SEQ ID NO: 105 - DNA, 23 bp, synthetic construct
  misc_feature 1..23: note = CX3CR1_gRNA9
  source 1..23: mol_type = other DNA
gagcagaccccctggatggcagg

SEQ ID NO: 106 - DNA, 23 bp, synthetic construct
  misc_feature 1..23: note = CX3CR1_gRNA10
  source 1..23: mol_type = other DNA
gaactcatagaaagcgatattgg

SEQ ID NO: 107 - DNA, 1599 bp, Homo sapiens
  misc_feature 1..1599: note = CD11b_loci
  source 1..1599: mol_type = unassigned DNA
gtgcatgggggtggggtgggggactctgggtggggaggagggtaacttttgggtctgtcataaatagagggcccagaatatgtaggagtcagtctggggagaggcaaaggggatttggggaaggagaaagggttcaagaagaagcagggagaacagctagacccagacaggctggccagggaagcctggatgaatgaccacattcatggactgtgcaaggctgcttgccggtccccttgcttcacacatgaggagacggaggcccagggaggagaagtgacatggctcagggtgcgcagcaggtgtgagacccctttcctgagtgcttcctcctggatcccctctcaccatctccactttgcctccggttctattttccaaggtcccgggtgcaaatgtttgttgaatgactgatgaatgaaaatgatttgagtttgttaccttttatgcttatatgttgtggaaaatgaaattctcctcaaaagggaaggaaatacttgagagctgcataggaaggaaattatctaattaagaatgtatagaaacttcactgttgggcaaatcatcgttgtgacaccgggggaagaagccatttaggtgctcagaagggaggctggaattcagagcaggactggacgtgccccacgacggtggttcttaggtcaggagtcagcaaacagtggcctgggggcccgatatggcccacgacctgtttttgcacaacctgccagctagagattgaagatgaacactgataatcgatttgatgatagggagcaccacccccaaagaattctatttgtctcatttgtaaacccgtattacaaacaaattgtactcaatcattatgtttgaaatttccctaatgacaaatttgtggaaaagtattttctgtcttgttatataagtacttgtacaacatattctatcagcctcttggtctgcaaaacctaaaatttactatctggctgtttacagaataagtgtgctaatccccgccccaggctaacagagctggacctgggaggcagacatctggatgctgggttagttagggtgaccgaatggatgggaaagggaatggagcaggaagacatgctgctatcttttttttttttttttttttttgatacagggtctttctctgttgcccaggctgtagtgcagtggcatgatcatggttcactgcagccttgacctcctgggttcaagcaatcctcccacctcagcctcctgagtaccactacacccggctaattttttattttttgtagagatggggtctcactgtgttgcctaggctggtcttaaactcctgagcccaggtgatcctcccacgtcagcctcttaaattattgggataacaggcctgagccaccacacccagccattgctttcttttaaattattattagtttttgttttttttttgtatttaattttgagataggatctctctctgttgctcaggctggagtgcagtggcacaatcagggctcaagtgatcctcctgcctcagccctccaaagtgctaggactacagcccctaactggcatgctactttcctctgcttgatccttcccccattctccctttag

SEQ ID NO: 108 - DNA, 49 bp, synthetic construct
  misc_feature 1..49: note = CD11b_T01
  source 1..49: mol_type = other DNA
ttcagagcaggactggacgtgccccacgacggtggttcttaggtcagga

SEQ ID NO: 109 - DNA, 49 bp, synthetic construct
  misc_feature 1..49: note = CD11b_T02
  source 1..49: mol_type = other DNA
tatggcccacgacctgtttttgcacaacctgccagctagagattgaaga

SEQ ID NO: 110 - DNA, 49 bp, synthetic construct
  misc_feature 1..49: note = CD11b_T03
  source 1..49: mol_type = other DNA
tgatgatagggagcaccacccccaaagaattctatttgtctcatttgta

SEQ ID NO: 111 - DNA, 49 bp, synthetic construct
  misc_feature 1..49: note = CD11b_T04
  source 1..49: mol_type = other DNA
ttctatttgtctcatttgtaaacccgtattacaaacaaattgtactcaa

SEQ ID NO: 112 - DNA, 49 bp, synthetic construct
  misc_feature 1..49: note = CD11b_T05
  source 1..49: mol_type = other DNA
tatttgtctcatttgtaaacccgtattacaaacaaattgtactcaatca

SEQ ID NO: 113 - DNA, 49 bp, synthetic construct
  misc_feature 1..49: note = CD11b_T06
  source 1..49: mol_type = other DNA
tttgtaaacccgtattacaaacaaattgtactcaatcattatgtttgaa

SEQ ID NO: 114 - DNA, 49 bp, synthetic construct
  misc_feature 1..49: note = CD11b_T07
  source 1..49: mol_type = other DNA
ttgtaaacccgtattacaaacaaattgtactcaatcattatgtttgaaa

SEQ ID NO: 115 - DNA, 49 bp, synthetic construct
  misc_feature 1..49: note = CD11b_T08
  source 1..49: mol_type = other DNA
tacaaacaaattgtactcaatcattatgtttgaaatttccctaatgaca

SEQ ID NO: 116 - DNA, 49 bp, synthetic construct
  misc_feature 1..49: note = CD11b_T09
  source 1..49: mol_type = other DNA
ttgtactcaatcattatgtttgaaatttccctaatgacaaatttgtgga

SEQ ID NO: 117 - DNA, 49 bp, synthetic construct
  misc_feature 1..49: note = CD11b_T10
  source 1..49: mol_type = other DNA
tgtactcaatcattatgtttgaaatttccctaatgacaaatttgtggaa

SEQ ID NO: 118 - DNA, 49 bp, synthetic construct
  misc_feature 1..49: note = CD11b_T11
  source 1..49: mol_type = other DNA
tactcaatcattatgtttgaaatttccctaatgacaaatttgtggaaaa

SEQ ID NO: 119 - DNA, 49 bp, synthetic construct
  misc_feature 1..49: note = CD11b_T12
  source 1..49: mol_type = other DNA
tttccctaatgacaaatttgtggaaaagtattttctgtcttgttatata

SEQ ID NO: 120 - DNA, 49 bp, synthetic construct
  misc_feature 1..49: note = CD11b_T13
  source 1..49: mol_type = other DNA
ttccctaatgacaaatttgtggaaaagtattttctgtcttgttatataa

SEQ ID NO: 121 - DNA, 49 bp, synthetic construct
  misc_feature 1..49: note = CD11b_T14
  source 1..49: mol_type = other DNA
ttgtggaaaagtattttctgtcttgttatataagtacttgtacaacata

SEQ ID NO: 122 - DNA, 49 bp, synthetic construct
  misc_feature 1..49: note = CD11b_T15
  source 1..49: mol_type = other DNA
tgttatataagtacttgtacaacatattctatcagcctcttggtctgca

SEQ ID NO: 123 - DNA, 49 bp, synthetic construct
  misc_feature 1..49: note = CD11b_T16
  source 1..49: mol_type = other DNA
ttatataagtacttgtacaacatattctatcagcctcttggtctgcaaa

SEQ ID NO: 124 - DNA, 49 bp, synthetic construct
  misc_feature 1..49: note = CD11b_T17
  source 1..49: mol_type = other DNA
tatataagtacttgtacaacatattctatcagcctcttggtctgcaaaa

SEQ ID NO: 125 - DNA, 49 bp, synthetic construct
  misc_feature 1..49: note = CD11b_T18
  source 1..49: mol_type = other DNA
tacaacatattctatcagcctcttggtctgcaaaacctaaaatttacta

SEQ ID NO: 126 - DNA, 49 bp, synthetic construct
  misc_feature 1..49: note = CD11b_T19
  source 1..49: mol_type = other DNA
tcttggtctgcaaaacctaaaatttactatctggctgtttacagaataa

SEQ ID NO: 127 - DNA, 49 bp, synthetic construct
  misc_feature 1..49: note = CD11b_T20
  source 1..49: mol_type = other DNA
tgcaaaacctaaaatttactatctggctgtttacagaataagtgtgcta

SEQ ID NO: 128 - DNA, 49 bp, synthetic construct
  misc_feature 1..49: note = CD11b_T21
  source 1..49: mol_type = other DNA
tgaaaatgatttgagtttgttaccttttatgcttatatgttgtggaaaa

SEQ ID NO: 129 - DNA, 49 bp, synthetic construct
  misc_feature 1..49: note = CD11b_T22
  source 1..49: mol_type = other DNA
tttgttaccttttatgcttatatgttgtggaaaatgaaattctcctcaa

SEQ ID NO: 130 - DNA, 49 bp, synthetic construct
  misc_feature 1..49: note = CD11b_T23
  source 1..49: mol_type = other DNA
ttgttaccttttatgcttatatgttgtggaaaatgaaattctcctcaaa

SEQ ID NO: 131 - DNA, 49 bp, synthetic construct
  misc_feature 1..49: note = CD11b_T24
  source 1..49: mol_type = other DNA
tgttaccttttatgcttatatgttgtggaaaatgaaattctcctcaaaa

SEQ ID NO: 132 - DNA, 49 bp, synthetic construct
  misc_feature 1..49: note = CD11b_T25
  source 1..49: mol_type = other DNA
tttatgcttatatgttgtggaaaatgaaattctcctcaaaagggaagga

SEQ ID NO: 133 - DNA, 49 bp, synthetic construct
  misc_feature 1..49: note = CD11b_T26
  source 1..49: mol_type = other DNA
ttatgcttatatgttgtggaaaatgaaattctcctcaaaagggaaggaa

SEQ ID NO: 134 - DNA, 49 bp, synthetic construct
  misc_feature 1..49: note = CD11b_T27
  source 1..49: mol_type = other DNA
tatgcttatatgttgtggaaaatgaaattctcctcaaaagggaaggaaa

SEQ ID NO: 135 - DNA, 49 bp, synthetic construct
  misc_feature 1..49: note = CD11b_T28
  source 1..49: mol_type = other DNA
tgcttatatgttgtggaaaatgaaattctcctcaaaagggaaggaaata

SEQ ID NO: 136 - DNA, 49 bp, synthetic construct
  misc_feature 1..49: note = CD11b_T29
  source 1..49: mol_type = other DNA
tggaaaatgaaattctcctcaaaagggaaggaaatacttgagagctgca

SEQ ID NO: 137 - DNA, 49 bp, synthetic construct
  misc_feature 1..49: note = CD11b_T30
  source 1..49: mol_type = other DNA
tacttgagagctgcataggaaggaaattatctaattaagaatgtataga

SEQ ID NO: 138 - DNA, 23 bp, synthetic construct
  misc_feature 1..23: note = CD11b_gRNA1
  source 1..23: mol_type = other DNA
ggttgtgcaaaaacaggtcgtgg

SEQ ID NO: 139 - DNA, 23 bp, synthetic construct
  misc_feature 1..23: note = CD11b_gRNA2
  source 1..23: mol_type = other DNA
gggaggctggaattcagagcagg

SEQ ID NO: 140 - DNA, 23 bp, synthetic construct
  misc_feature 1..23: note = CD11b_gRNA3
  source 1..23: mol_type = other DNA
ggagtcagcaaacagtggcctgg

SEQ ID NO: 141 - DNA, 23 bp, synthetic construct
  misc_feature 1..23: note = CD11b_gRNA4
  source 1..23: mol_type = other DNA
gagtcagcaaacagtggcctggg

SEQ ID NO: 142 - DNA, 23 bp, synthetic construct
  misc_feature 1..23: note = CD11b_gRNA5
  source 1..23: mol_type = other DNA
gacctaagaaccaccgtcgtggg

SEQ ID NO: 143 - DNA, 23 bp, synthetic construct
  misc_feature 1..23: note = CD11b_gRNA6
  source 1..23: mol_type = other DNA
gcaaatcatcgttgtgacaccgg

SEQ ID NO: 144 - DNA, 23 bp, synthetic construct
  misc_feature 1..23: note = CD11b_gRNA7
  source 1..23: mol_type = other DNA
gagacaaatagaattctttgggg

SEQ ID NO: 145 - DNA, 23 bp, synthetic construct
  misc_feature 1..23: note = CD11b_gRNA8
  source 1..23: mol_type = other DNA
gccccacgacggtggttcttagg

SEQ ID NO: 146 - DNA, 23 bp, synthetic construct
  misc_feature 1..23: note = CD11b_gRNA9
  source 1..23: mol_type = other DNA
gaaatacttgagagctgcatagg

SEQ ID NO: 147 - DNA, 23 bp, synthetic construct
  misc_feature 1..23: note = CD11b_gRNA10
  source 1..23: mol_type = other DNA
ggtcaggagtcagcaaacagtgg

SEQ ID NO: 148 - DNA, 387 bp, Homo sapiens
  misc_feature 1..387: note = S100A9_loci
  source 1..387: mol_type = unassigned DNA
gtaagtgagctgccagcttccccaggcagaagcctgcctgccgattccttctttccttccctgacccaacttccttccaaatcctcctcctagaagccctccttggttggccctgcctactttaaagcttctttcacattttcttaggtcatgttcccctggggcctcctgccctcaaatgctttgctttttggcactctgtagatattctaaaaaatcattttgtacatgtgtgtgacaggccatctcccagttaagttgcagcctgtgctttctttttattttgcacttcccccactatttctgtgagtgcttagtaggaagtgtcaaagaagcttgacagcattttcttctaagtgtcccaactcttggttttccattacacag

SEQ ID NO: 149 - DNA, 49 bp, synthetic construct
  misc_feature 1..49: note = S100A9_T01
  source 1..49: mol_type = other DNA
tttccccgttgtattggttgaaataagtttcactaattggtaacctcca

SEQ ID NO: 150 - DNA, 49 bp, synthetic construct
  misc_feature 1..49: note = S100A9_T02
  source 1..49: mol_type = other DNA
tattggttgaaataagtttcactaattggtaacctccagagggaaggga

SEQ ID NO: 151 - DNA, 49 bp, synthetic construct
  misc_feature 1..49: note = S100A9_T03
  source 1..49: mol_type = other DNA
tttcactaattggtaacctccagagggaagggaagggagggcaggggaa

SEQ ID NO: 152 - DNA, 49 bp, synthetic construct
  misc_feature 1..49: note = S100A9_T04
  source 1..49: mol_type = other DNA
tggaactggcctctaagtcagatctgaatttgcatgccctcaatagtca

SEQ ID NO: 153 - DNA, 49 bp, synthetic construct
  misc_feature 1..49: note = S100A9_T05
  source 1..49: mol_type = other DNA
tctaagtcagatctgaatttgcatgccctcaatagtcaagctgtgaaaa

SEQ ID NO: 154 - DNA, 49 bp, synthetic construct
  misc_feature 1..49: note = S100A9_T06
  source 1..49: mol_type = other DNA
tgcatgccctcaatagtcaagctgtgaaaactaatgaccctctctagga

SEQ ID NO: 155 - DNA, 49 bp, synthetic construct
  misc_feature 1..49: note = S100A9_T07
  source 1..49: mol_type = other DNA
tgaaaactaatgaccctctctaggactggtttcaagtcttcctccagga

SEQ ID NO: 156 - DNA, 49 bp, synthetic construct
  misc_feature 1..49: note = S100A9_T08
  source 1..49: mol_type = other DNA
tcttcctccaggaagataccattcctagctgttaaagttgttataagga

SEQ ID NO: 157 - DNA, 49 bp, synthetic construct
  misc_feature 1..49: note = S100A9_T09
  source 1..49: mol_type = other DNA
tcctccaggaagataccattcctagctgttaaagttgttataaggacca

SEQ ID NO: 158 - DNA, 49 bp, synthetic construct
  misc_feature 1..49: note = S100A9_T10
  source 1..49: mol_type = other DNA
ttcctagctgttaaagttgttataaggaccaaatgaggtgacatttcca

SEQ ID NO: 159 - DNA, 49 bp, synthetic construct
  misc_feature 1..49: note = S100A9_T11
  source 1..49: mol_type = other DNA
ttaaagttgttataaggaccaaatgaggtgacatttccaggcttactca

SEQ ID NO: 160 - DNA, 49 bp, synthetic construct
  misc_feature 1..49: note = S100A9_T12
  source 1..49: mol_type = other DNA
tgaccagggcaagaccctggaactcagcttcctcttctataaatagaga

SEQ ID NO: 161 - DNA, 49 bp, synthetic construct
  misc_feature 1..49: note = S100A9_T13
  source 1..49: mol_type = other DNA
ttcctcttctataaatagagaatcagcacccaagtcacagggtcatgga

SEQ ID NO: 162 - DNA, 49 bp, synthetic construct
  misc_feature 1..49: note = S100A9_T14
  source 1..49: mol_type = other DNA
tcttctataaatagagaatcagcacccaagtcacagggtcatggaggga

SEQ ID NO: 163 - DNA, 49 bp, synthetic construct
  misc_feature 1..49: note = S100A9_T15
  source 1..49: mol_type = other DNA
tctataaatagagaatcagcacccaagtcacagggtcatggagggaata

SEQ ID NO: 164 - DNA, 49 bp, synthetic construct
  misc_feature 1..49: note = S100A9_T16
  source 1..49: mol_type = other DNA
tataaatagagaatcagcacccaagtcacagggtcatggagggaataaa

SEQ ID NO: 165 - DNA, 49 bp, synthetic construct
  misc_feature 1..49: note = S100A9_T17
  source 1..49: mol_type = other DNA
tggagagcgtttggtatgtgctcagtgtctgctccattgtgcgcactca

SEQ ID NO: 166 - DNA, 49 bp, synthetic construct
  misc_feature 1..49: note = S100A9_T18
  source 1..49: mol_type = other DNA
tggtatgtgctcagtgtctgctccattgtgcgcactcagcctatggtca

SEQ ID NO: 167 - DNA, 49 bp, synthetic construct
  misc_feature 1..49: note = S100A9_T19
  source 1..49: mol_type = other DNA
ttgtgcgcactcagcctatggtcatttttaatttttaaatccagcccca

SEQ ID NO: 168 - DNA, 49 bp, synthetic construct
  misc_feature 1..49: note = S100A9_T20
  source 1..49: mol_type = other DNA
ttcccttgtacatttgccagctggtcatttactgtgctcccagtcccca

SEQ ID NO: 169 - DNA, 49 bp, synthetic construct
  misc_feature 1..49: note = S100A9_T21
  source 1..49: mol_type = other DNA
ttttgttttcttttcaaatttggggaaagtcgggaaacagaggcctgca

SEQ ID NO: 170 - DNA, 49 bp, synthetic construct
  misc_feature 1..49: note = S100A9_T22
  source 1..49: mol_type = other DNA
tttcttttcaaatttggggaaagtcgggaaacagaggcctgcattaaga

SEQ ID NO: 171 - DNA, 49 bp, synthetic construct
  misc_feature 1..49: note = S100A9_T23
  source 1..49: mol_type = other DNA
ttcttttcaaatttggggaaagtcgggaaacagaggcctgcattaagaa

SEQ ID NO: 172 - DNA, 49 bp, synthetic construct
  misc_feature 1..49: note = S100A9_T24
  source 1..49: mol_type = other DNA
ttggggaaagtcgggaaacagaggcctgcattaagaagggtggaacaca

SEQ ID NO: 173 - DNA, 49 bp, synthetic construct
  misc_feature 1..49: note = S100A9_T25
  source 1..49: mol_type = other DNA
taggtccccagccctcccagtgcccctccctccgccttggtaaggtgga

SEQ ID NO: 174 - DNA, 49 bp, synthetic construct
  misc_feature 1..49: note = S100A9_T26
  source 1..49: mol_type = other DNA
ttcagagttaggggccctgacagctctccataggtggaggcctcaggca

SEQ ID NO: 175 - DNA, 49 bp, synthetic construct
  misc_feature 1..49: note = S100A9_T27
  source 1..49: mol_type = other DNA
ttaggggccctgacagctctccataggtggaggcctcaggcaggcagga

SEQ ID NO: 176 - DNA, 49 bp, synthetic construct
  misc_feature 1..49: note = S100A9_T28
  source 1..49: mol_type = other DNA
tccataggtggaggcctcaggcaggcaggatgctgggtggggtaggcaa

SEQ ID NO: 177 - DNA, 49 bp, synthetic construct
  misc_feature 1..49: note = S100A9_T29
  source 1..49: mol_type = other DNA
taggtggaggcctcaggcaggcaggatgctgggtggggtaggcaagaaa

SEQ ID NO: 178 - DNA, 49 bp, synthetic construct
  misc_feature 1..49: note = S100A9_T30
  source 1..49: mol_type = other DNA
tgggtggggtaggcaagaaagggcccagcagagaggccgcatggcaaaa

SEQ ID NO: 179 - DNA, 23 bp, synthetic construct
  misc_feature 1..23: note = S100A9_gRNA1
  source 1..23: mol_type = other DNA
gcacaggagagtgctcgcattgg

SEQ ID NO: 180 - DNA, 23 bp, synthetic construct
  misc_feature 1..23: note = S100A9_gRNA2
  source 1..23: mol_type = other DNA
ggtaccccacaggttctgggagg

SEQ ID NO: 181 - DNA, 23 bp, synthetic construct
  misc_feature 1..23: note = S100A9_gRNA3
  source 1..23: mol_type = other DNA
ggagccagacatcctggggtagg

SEQ ID NO: 182 - DNA, 23 bp, synthetic construct
  misc_feature 1..23: note = S100A9_gRNA4
  source 1..23: mol_type = other DNA
ggagagtgctcgcattggctggg

SEQ ID NO: 183 - DNA, 23 bp, synthetic construct
  misc_feature 1..23: note = S100A9_gRNA5
  source 1..23: mol_type = other DNA
ggaagcagagcctcatggatggg

SEQ ID NO: 184 - DNA, 23 bp, synthetic construct
  misc_feature 1..23: note = S100A9_gRNA6
  source 1..23: mol_type = other DNA
ggcttactcatgccatgaccagg

SEQ ID NO: 185 - DNA, 23 bp, synthetic construct
  misc_feature 1..23: note = S100A9_gRNA7
  source 1..23: mol_type = other DNA
gggaaacacctagaaaaactagg

SEQ ID NO: 186 - DNA, 23 bp, synthetic construct
  misc_feature 1..23: note = S100A9_gRNA8
  source 1..23: mol_type = other DNA
gtggggggtgaagcgggcatagg

SEQ ID NO: 187 - DNA, 23 bp, synthetic construct
  misc_feature 1..23: note = S100A9_gRNA9
  source 1..23: mol_type = other DNA
ggggggtgaagcgggcatagggg

SEQ ID NO: 188 - DNA, 23 bp, synthetic construct
  misc_feature 1..23: note = S100A9_gRNA10
  source 1..23: mol_type = other DNA
gagggctggggacctaccccagg

SEQ ID NO: 189 - DNA, 3424 bp, Homo sapiens
  misc_feature 1..3424: note = TMEM119
  source 1..3424: mol_type = unassigned DNA
gtgagtcctggggtcccctcctctgtcctgagagcctggagctatcttggagcttagggactggggactgttggagcactctggggggcctctctaagtgtgtgtgggctttgagtgtgtgtttggtgttgtgtgcatgagtgtggtggaatctgagtcccgtgtgcggtgctggggtgggccagggtgggccggagtgttgtgtgtgtgcggctggggccttggtgtagggggtgtgttgcttccacttttctgcaagtggatgccaggctgttgcttccaggatctgtgtgagggtgatgtggacggtattgtcctgtgtgcagggatcggctgtgtgtcagggggttgtgtatgtatatgtgttgtgtgtccgtgtgtgttctctgtgtattgtgtgcatgggccatgagagtgctgttgggtgtctcagtgctgtgtgagtcggtgtgcctgtgttgtgtgtctttatattgtgtgtccatgctgctctgtgtgtgtgtgtgtgtgtggagggggattgtgcccccaggacccccctccccatccagacctccagggcccaggtccctgtccttggctggccctggggtcgggggcatgtgcagtgctccaaccagaacacccctcccccacaaggccactgattgaagccaggtttccgtgggccgcccctcccccaaaggccaccagtctctgggtgggggtggggcagcccccggtcccctagcttcaagtcttggggcccccaggcctcaggggtgcatctgcactcctccacagactcaggaccatcttagggcatttgccacacttacacacggccagccctgcctgacatgtccccaggcagggagtcctgggcaatcacgtggtacctgctaattggcacccactgtgcaccaggcatggccgcatcttgtcctaacagcagtccagggaggtggctcttatccccattgtacagatatggaaaccgaggctcagagaggtgacatgcccagcccaaggacacatagccgggaagtgacctcctctgggatataccaagacaggcttctctgtccagggctctgctgggctcacccctgactttctgtgtgacctgggcaggctgtttccagtctctggacctgagttacctctaaggtgaagcaagaggcgttgggcctgaccccagctcctaatggcctgttagattctgagtccttgaaaaaattagggtccggggggaggtaggcagaataatgacttcagagctgcccagaacagtgcttcattccacagatgggaaactgaggcccagagagggcaggggccgtccagggccacacaaccatcttttttgaggctctgctgactgcaggtggggctgggtcctccatacgttgcccccacctttccctggaagccaggaggggttgtggggtgcaggggactctgagagtcagggtctcagccttaaagagccagtggcagtttgtgtgttctcctttcttgccctctgcctctgacttcgtgtgtgtgtgtgtgtgtgtgttgtatgtatatgtgtgtgtaggtgtatgtgtgcatgtgtgtgtatgtgtatgtgtgtgcatgtgtgtgtttggttctgcattccttggcggccagagctaaagaggtttcactgaacggggtgaaaaggttggtctttcactgggtggcggccggaccagacagtgagcgcatgttcccatccggcgatggtgctcatttgtcgactcagggtgggaaattgctggctgaacctggagttcggctagggaaggaattcagctaccccagaccctatctttcctcggggctcagctacttcgcaccaggcactctctgaaagttgtctcatttggccctaacagctctgagaggtgctacctgtggtcaccctgtcccaacttacggatggggaaagtgaggcccacaggagccagaccacagagctggatgcgatggagccgggattcttgacccagccagaacttgagtgggtctccagccacccatggtcccagctgccctctgcagtgagaagtggtagttgttgccacaaaagctctctcctaggcttgactgggggtgggaggcagggaggtgactggcaggtcacagaccccaaagctcaaggttgtctgtaggaacctttgctaacaggtgccagagaggtgagggggtcctttcctcatttagcatctgacaggtgatgtccaccagcgggaacaagccattcctggctcagagtgtgtggtgtgtgcatgtgtgtgtggttgtgtaggtgagtgtgtgtggtgtatgtgtatggttgtgtgtatgtggagtgtatgcatatatgtgtggtgtgtgcatgtgtgtatgattgtgtgtagtatgtgaatgtgtgtataggggtgtgtgtggggcatgtgtgtacgtggttgtgtgtggggtgtatatgggtatgtgtggcgtgtgtgcatgtttgtgtgtggggtgtgtgtgcatgtgtgtagatgattgtgtgtgtagtatgtgcatgtgtgtatatgggtgtgtgagtgtgtgtgtggtatgtgtgtgcatggttgtgtgtggtgtgtgtgcatgtgtgtatgattgtgtagtctgtgcatgtgtgtatatgggtgtgtgtgtgtgtggcgtgtgtgcatgattatgtgtggggtgtgtatggggatgtgtgtgtgagtgtgtggcgtgtatgaatggttgtgtgtgtggtgagtgtgcatgtgtatgtatggctgtgtatgagtgtgagtgtggtttgtgtgaatgtgcgtgtaaactcctgggatcagacagaggaaggttcagatcccagctcagctacttatcatctgtgtgtccttggggaagtcactgtctgtctctgaacctctgtttcctcatctgtaaaatggggataatgaagctgcctcagggctcctgtaggattaaaggcatgaaaagggcttagatgagtacccgcgaggaaagcacaggtgaccagctcatttaaaatgagcccctgagaagctttgtctcttttttaaaaaattgaaatattttaatatatttcatagagatggaggtttccttatgttgcccaggctggtcttgaactcctgggctcaagcgatcctcccaagtagctgggactacaggcatacaccaccaggcctggctacttttttatttttttgtagaggtggggtcttactatgttgctcaaactcctgccctcaatcaatcctccggcctcggcctcccaaagtgctgggattacaagtgtgagccaccacacctggccaattttgcctctttttatgtcctggaattatttttttattttttccatctaagccagtttgttcacatttaatttcttttttttcttttctttctttctttttttttttgacag

SEQ ID NO: 190 - DNA, 6273 bp, Homo sapiens
  misc_feature 1..6273: note = MERTK
  source 1..6273: mol_type = unassigned DNA
gtgagtgcgcccggctgggggccaggcgagggggtgggggctcccaggaggaagcaggggcctctggggagggagcgcgtccacaggggcgcgcctggctgctggacaagtttgcaaacaccctccacccactgcggtgccagaggagggggcgtaggcgaacctaccgtccactgaccgcggcgcctcaagcgtcccgagggcacccagcctggcttgcgggtgcgcatcgtggtgaagccgggtcggggtcggcgttgcaggctcccctcttcaccgcagaggaggaaatcgagctcggccgggcgcgctgcaagtcctgaagttccgacgagtggaacgtaggtagcaggtctggtctgggatgcgacgagaactttccactagagttgcatggtggggccccgctctggccggagtcccgggggccacagatccggtctcccgagggaccggcgcggggcgaacgaacgggctgcgggagtcggagccgcagtccgggagccgcgatagactgaggccgagcgacgggccagggggggacggcagtcctggctgctcccaatgggcccttacgggcattattagtatccctttttaggggcacccagagagtgtgggaaaaggggagcgttcttttcactcctgttatggcagaacagaacttacagtgctcggcagcgtttacgtttatctggattcagcgaagtgttgttacaggcttttttttttttaatttgagaccgagtctcgccctgtcgcccaggctggagtgccgcggcgcgatctcggctcactgcaggctccgcctcccgggttcaagcgattctcctgcctcagcctcccgagtagctgagactatagacgcgcgctaccacgtccggctaatttttgtatttttagtagagacggggtttcaccatgttggccaggatggtctcgatctcttgacctcgtgatctgctctccttggcctcccaaagttctgggattacagtcgtgagccaccgcgcccggcttgttccaggcattttaatacaagatgctggactgatacgtggaggcaaggaaggccggtttcattctgcttgaacagcttatttggggagtgagtgatgaacagatttcaagtttgatcgctgatgcacttcttgaggaggctggaataaacttaccaggctttacccaaaagaaaaagaaaggggaaaaatgctaaataaacacattccaagtattttctctcagctaaaaggtttctatttccattcagcatgtagattttctctcttttgatcgagaatgcacttgactagccttcacctattacagtcaaagatgttttttaggaatacctggaaagctcagtttatagtcaagtaagttacttaaaagtttttcctttgagattttcctttgaaagctcatttactcaaatgaaaaattcttaaacagtgcacccactttttgcacgttaaattacataagcaatttataatttggggcaacatagcaatttgaaacaaactttaagagccaggcaaagaactaaagcagttctgttctccactgcccctccctccaccaaaccttcaaaaattcctcagcatttctgccagccccatttttcataatctgctgcaaatctgttatttgttgctactagtgaggactggccgtgaatagtcttggaggtagtgaatgtggtgagaactatggagaaagtaatgggaacaatagttttgtgttaaatacttaaaaattggagtgcttttgtttttctaaaattttaaaaggctgtaataacaaatttttgtagctgacaatcaactttaatcagacaatcataaaaaggccttttaaaatccacccagctggaagaatgaaaagtttcaccaatcgttcttgcactttttgcttttggttttgaactttacctatgagatgtgtgacagttttacatctcaccttgtgaaaagcttaaagaatgggattctcactggcagaattacagcacaaaggagtgctgtatgaagaaattaaacatggaaattagactcttcatctaattttaagattttcctattacacattgtgggtctgagtgctttgctgaccacacaggggagacagagagagagacaatgagaatatgtgtgtagattctacactgttagcagtatagaagaatgggtaaataaaatttaagcatcactttaacccaggaaatcccctctgccccccaacacacatacacactacacgtctcacaaagacatgcacacacagaactggaactgccaaaccaggagcatttcgaggctttccagtctttcctttctgctcagtctctgatgggggtgccattgagcagcaaaatgaggcaagcggtggctctctgaagggccagggagaggatggagcaagataaatataaatgtggataaaaatgaatgcattatgaggacttagctcatgacgaggctgtgtgtgcagctaaactcaacccttagagtaaattaggcttttaagattctttaatgttaatatttcttttccaaactgatattgtaacatgtattccagtatattgtaaacatcttcccagagggaacttttaagttgttctgttgcttgtgggctttattccaaaggcttcaaatgctttttgaaagtacatcgtgcatattttaaaaatgaatacttttagaaaattattctgacccattaaagtgctgagtggaattcttttcttttctttctttcttttttttttttttttggcaggttctcattctgccacccaggctagagtgcagtggcaccatcacggctcactgcaacctcaaccttccgggctcaagtgatcctcccacctcagcctcccaagtagctatgaccacaggcacataccaccatgcctggctaattttttattttttgtagagacaggatctcactatgttgcccaggctggtcttgaactcctgggctcaagcgatcttcccaccttagcctctcagagtgtggggattataggcatgagccaccatgcacagctgaatacaatttttaaaaaattaacaataattatgtttttttgtttttgagatagagtttcactcttgctgtccaggctagagtgcaatggcacaatctcggctcaccgcaacttcccgcctcccgggttcaagcagttctcctgcctcagcctcctgagtagctgggattacaggcatgcaccaccacgcctggctaattttgtatttttggtagagacagggtttctccgtgttggttaggttggtctcgaactcccaacctcaggtgatctgcctgccttggcctcccaaagtgctgggattacaggcatgagccaccacacccagccaataattatatttttaaatcttaatttttgaaataatttccagataattgtagaagggccagtgagttactagtcagcattggtcaagaagaggttcaatctttttatgcctgtgaattgggaacatgctgctgggtagaaaaactgagctgggaccttgaacacacatgtgaaacagtcgctttccatataatagccagaatgagattttggaaacgaatctgattgtattttccatttgaaaatcttccagtggcttcctgttccctactagaatttgcagagactcacagcgaacctctttgacccatttccattcacttccctcttgttcctaccactccagccacctgtgtcttctttctagggcctgagcagcctttgcagtgactcttccctctggctggaacctttttcccaagatctcggcatgctggcttcttctcatcctttgggtttcagttcagaggttctccttgagaagccttccccaataaccaccccctgccaaagtatcatatgcatacaccccactatcccttaaatttaatttcctgataatgcttctcacaacctgatcagttccttccttccttccttccttcctccctccctccctccctctatttctttattgacagagtcttgcaatggcaaattctctgctcactgcaacctccgcctcccaggttcaagcaattctcctgcctcagccttcccagtagctgggattacaggcacctgccaccacagctggctaatttttgtatttttagtagagacagggtttcaccatgttggccaggctggtctcgaactcctgacctcaggtgatccgcctacctcagcctcccaaagtgcagagattacaggcgtgagctatggcacccagccttacctctttgtttactccttttcctccactgtccccccaccacaaggtaagctcctggtggcagggctgctgtctttttcactgctaacacagcatggagctcatacgggtgctcagtgagtatcagagcacagcatgaaggaaccaagcccctgcatgtcttaccaggtgctcttcacccttatcttttcctactggcttttccagaaacagaattgctggagctcccatgccctcacattcggggcttggttaatagaaaacaatcttttattttgctgtgatgccaatgtgatttcattgctggaggagctagtcaattaaaaccagacagatttttgggctgtattaaccccaaacatattttacattgtcctaactattccaactagaaaaaaaaatgtcagaaggtaatttttcatgttaaacacagttctaacaactccttgctcaggggcaggtagccctgggtgccttgactttggcccttgccgttagtttaatcttaacccaaagcacatggctctgaattggtatgtttctcagcaggagattaattattataaaaactgaaaggggtctcacactgtgtgaggcacactatgcctctggggactggaatgaaagtgttaaccgggactggcttctggggctttgggggtggtgattaactttcaggacctctgttcctttttgggtacagtgggaggctcttttcatagaattgttaggaggacgtaatgagctaatggcaggtgatatgtgcttaaaatatatttgccattattaccaatggttctcctccattcttctgaactcaagtatgggggaaatgtttttcctagacaaaaggtgttgggaggagggttcttttcttattgaagtgtatcctgaatggtccaccctccattgttcagaatgctttggtgaaggggccctccggtgtcgccctagagtgcccaagtcctttgctgcagctctttaggtttttgtttcctagtagtttgagcaaaacccacccaggaccaagcagttctccagagaagcagggcccgccctgactgccattcccctcctactcctgcccccataccttcccttatggagttcaaacttgacatagtacaggaatagcttcttgtcatgtgcctctcaggaaggtataaaaatgaagtccagttcctgttcaacttttcccacataactcttttccaaactgggtgtggtttaaatttttttatttttattttttgtgtttgttttgttttagggtttttgagtgtgaacatccagaaaattcttttttttttttttgagatggaatcttgctctgtcgcccaggctggagtgcatgtggcgcgatctcggctcactgcaacctccgcctcctggcttcaagcaattctctgcctcagcctcccaagtagctgggattacaggtgccctccaccagacctggctaatttttgtattttaagtagagacggggtttcaccgtgttgggcaggctggtcttgaactcctgacctcgcgatccacccacctcggcctctcaaagtgctgggattacaggagtgaaccacctcacccagctcagaaaattcttttcctttttaccacatcgagctaggttttcccaatgaggtcagttggctgcaacatagagaaaatgaggctttggttcttagagtagattccgtgtacaggcgtgagtatgtgtccgcaggtggtcttgtaggaaatgcaaagggcgccaggtgcactcctggcttctggtacttcctgcttaatggcatgggtgagccaaccctcaggtgcccgggagggagccctgattactctccctctggcactctgggcattggctaacccgtgaccggagttaaaccagttccctctcttttcattctatgtctcccactcctgcctccagccgaagtgctaaaaatgcagcctggcaggggcagagaattccattaggatcagcagg

SEQ ID NO: 191 - DNA, 2541 bp, Homo sapiens
  misc_feature 1..2541: note = CD164
  source 1..2541: mol_type = unassigned DNA
gtgggcgcgggccctgtgggcttcccgccctcccctgcccagccgagccgcactgcctcctcgccgtgcgggcggggcccgggttcgggccacggtcgggcgggcgggccgcgctcggggtgcggcggcgactccggcaacatggcggccagcctggagtccactcgtggccgggccgggtggggagcgcgctggggggaagcggacgcgcggttcccagtggctgcggggctggggcccccctcgaagtgttgcaagtcgaggtgccttttaaaagtgcatcgtaattcctagcctcacttggggtcggaaatggggacgtggcttccttccgcgggggcagcggtggggttgaggatggggccgggcgtcgcgcctctgaggttggcggtggggccgagctgcctctccctgccactggttgttcaccctcccggcgcctctctccggaggtagccttctccggaagtccgggaaagagaaacttaaagacggtaatgctcgttcgtgtctcagacttgagtctgagaaggtcgcttgagatttaattacattggttttgcagataggaatagaatgtagctgacgtttgagcacgtagttagtgaaaacttcacacttcaaagtatgcgaaaaggaagaaaagtacccatgtatgtaccgggtgttttgcgtacgtatcctaaggctaaactggaaggtaggtagtgaaacctaggaggtagatgcagagacggagatgagtaaactctcctgacattgtatgtttattcttttttcaattgtttttaaaattttcttaactatgtcctgtgctcagatagcgtatgtttagtaggtcgtggaacgggggtttgtgcccaggtctgtgggttttctaaaacccgatcaggagcgtgtcttttgttatgtgcagtcgcatagggctggttaaggcatacatacattttaaagcagaaattgccatataccgtgacaacacaaaagagagaaggaagataagtttgaggtaataagcgggagaaacactagaggtttaaaacctgcagtctcgtatgattggttattctagttaacattagttaaggagtaatgtttacgtagttaacattggcttcttgaccgaagaatgccgaagtgtaatgatttttatcacggcattcatattctacaaattctcctctagttttagtattttataaaagtttaataaagtatatagtaaacttaatgttttgtttttagtttaaggaagatgtaatggtaacatttcaaaacgagggaagtaaattgcagtataatactgggaattctaaccgcactattttaagatgatagagcagaatatgtttgtctcagtcttaatttgacagtttcctttcagaattgtagtgtgtgtgtaagagaaggttggaagtatcaaattaatttgtctttgtgtaattgaagtgcgtgcaaaggaggtatgtattttctggaaatcataagtggatgtagtgtggtctgaaaggtagcaagtttagttcattgtaaagaacatttcagacatttcactttgctacacccgtcaagcacaaaggtttttaaacagcatgttttatctatacaaacttcagaaaatttatttttactttatgaacccttccccaccccaccgtacatgtcccagtctttaataagtactaatgatgaagtattgaaagcagtgcctctgcagaactattttttaggcaggagcaaagcaaacgagaatgtttggaggcatctagtttctaggaaaatgaatggagcccaattcttttttttcttttattttgctgctttttctcactggaaaatagagcccaattttatagtccgttcttctccagaaattgaattatggtgatgagtacttgtttctggattgtttggcctaggatgaactattgacaaataccttgttaacctgaattgtatgcacttgaatatgggcttttagatgttgtctccagttgtggaggatcctgtcgagagtggatcatttatgaatcgagaatccctgggaaattatctttgtttgcagcagaatgggcatcatcagtgcttgggttcaacctgttaaactactactgttactactaaaaatctcagggttttatctgatgtttgggggtagtggttttcctatgtgtagtgttggtattatatcaaagcctttttctcgatatttttatgtattaaggatgctttgaaatgcctaaatccttagtggctgtgggtgtcttccaatagttgtcctgtgaaaaaggattggttagacagataccatcaggcactgtactgagagtgaccactagcttgtttttctaatgcgtttacttacagctttgtaatgattggaatttcttagtaaaccagtataaagttttatcatttatctgaattagtaattttgatgctgtatgtaatgcttattgttcaaaaagataagtaatacagattcaaataagatactaataaaatcattgatttcatgcatctctccccccaactccccag

SEQ ID NO: 192 - DNA, 357 bp, Homo sapiens
  misc_feature 1..357: note = TLR7
  source 1..357: mol_type = unassigned DNA
gtattttaaacattggaacacatatagataatttaagtaggtagatgtatgtgctgttataaggaagtggggaggagagaagagggaaccgaaatcatatgcacaaaaattttttttagaatataaataaaaaatgtggtagtctaaaatgtcaattcttcaaagataaagttaggctttcagtaacgttagaaatggttttctggaatatgtctccagtctacctaactttgaggaagtaaatactgtaaatagatgtttcaaacgcattttaaagcaatgatcctagcatgtctttaagctacagtattgtgctgtctttgaaatgtaaactttgatgtcttctctttctcttag

SEQ ID NO: 193 - DNA, 359 bp, Homo sapiens
  misc_feature 1..359: note = CD14
  source 1..359: mol_type = unassigned DNA
gtagattctctgggatataaggtagggggattggggggttggatagtgcagagtatggtactggcctaaggcactgaggatcatccttttcccacacccaccagagaaggcttaggctcccgagtcaacagggcattcaccgcctggggcgcctgagtcatcaggacactgccaggagacacagaaccctagatgccctgcagaatccttcctgttacggtccccctccctgaaacatccttcattgcaatatttccaggaaaggaagggggctggctcggaggaagagaggtggggaggtgatcagggttcacagaggagggaactgaatgacatcccaggattacataaactgtcag

SEQ ID NO: 194 - DNA, 855 bp, Homo sapiens
  misc_feature 1..855: note = FCGR3A (CD16)
  source 1..855: mol_type = unassigned DNA
gtaaggagccctggagccccggctcctaggctgacagaccagcccagatccagtggcccggaggggcctgagctaaatccgcaggacctgggtaacacgaggaaggtaaagagttcctgtcctcgcccctccccacccccaccttttctgtgatcttttcagcctttcgctggtgacttgttcttccagggcccatttctctaccctacctgggtttcttctaacctggaaatctaatgatcaaatcacactaaaaagtcagtagctcctgtggattacatatcccaggagcatatagattttgaattttgaattttgaaagaaattctgcgtggagataatattgaggcagagacactgctagtggtctgaagatttgaaaggaccactttctgtgtgcaggcagggcctcagctggagatagatgggtctgggcgaggcaggagagtgacaagttctgaggtgaaatgaaggaagccctcagagaatgctcctcccaccttgaatctcatccccagggtctcactgtcccattcttggtgctgggtggatccaaatccaggagatggggcaagcatcctgggatggctgagggcacactctggcagattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttag

SEQ ID NO: 195 - DNA, 2268 bp, Homo sapiens
  misc_feature 1..2268: note = TBXAS1
  source 1..2268: mol_type = unassigned DNA
gtaggagtggaatgaatgaatgaaggagtgggcgcgcgccgtgctccggaggagggcgggtgagtggaggagcttcggaaagccacacgaggtcagaggcaccgaagccctgccgtgcacgccgcggaaatgcagcccccggggtggtcgctgggttcctgccggagtctggcttccacgccaccctgatcttctcctcctcgcgtctacactgtcccgagggccgccactccatcacttgaatgggagctcacaactctctgggtcctcgcttttctcagcgctctctcctatcagggcttccgctaaacactctccagactctccccagccgggagcctctccaggtttgcttctccagctgcctcccggatgcttggataggatgccctgccatcccctccaacttaggaagacttgttgagtctcctcttaggcactgtttttaaaactgtgggtcgtgacacagtaatagatcataaaataaatgcagcggaaggcaaccagctttcaaaaagaacgaaatggaatccaatagaaaatattagagtccattgcccttagtaagggcaactattgtttcataaaccttttgtttcgattataggtactggacaagtgtactggaccatgatgtcaattttatttccgtctgtgaatcgtgggtttaaaaaagttgaaaagctatctgtgtaaaataatgatctctaacactgtataactttcttaaaaacaacctcgtgattttctcactacttcttatcctttccggtctccatcctccgttcccatccttcagtccttcccaaaaaaggcaagctctttcacacttttttagtggtacatcacgacaccttcctctgtgtgatcttgtactctgcggtcttccttgcccccataatcaatgctacacttcctatcccttctcttaacctaatgccattcacgtttgcagaatcctagactactgttgctaaaaggacctcagaggtcatctgggccagccctgcccttttagagttgaagaaatggagtccagagaagtaaaatctgttgcccaatgtcacacagtattgaagcttaggaataggtctcgtggctcccactctagtgttttttgctccacaccggtcatttatctccttcccttctatcctccctgcaatctttaactttagtcttggcccaggtcatcctaaatattgccatcttcataatacgtggttctggccatgccacgctcctggctaacatcctctaaggactcaacctctgcaggtggggctgagtgccttggcttttcatcaaggcaggcgagtcctgacttagctttctaaggacatgagttcagcaagtcactgcttctctagccaaactggtttacttgtagcccagtgccagcttcacccatctctgtcactgtgcctttgcccaagcggtcactttcccacccactttcccagaggggtaccttctccatcttctctctttaagcagattctacctgtgcctggctcacctcctccacaaaacagcccgagatagcgtgggagtaagtcagacctaggttcaaatcctagctcttccacttaactatgtgatcttggtcaaaatgcttaacctctctgagcctcagtgtctgtattttgattatgagaaggagaacaatagtaatgttctccttttgcatctgtaaatctctatcatagccttcaccacacaggtatgtaactccctgttaatacataccctacatgcatgtctctctgcccctttctagtgctggccattagaacttgctgccataatggcagtgtagctacttatgtctgtggagcacatgaaatgtggctagtatgactgaggaactgaatttttaattttaataaccatgggtggttagtggctaccattttggacaaggtgtctctagactgtaagctcagtaaggacagaaaccatgtttttcttgttttggattgtatgtccaataactgcattgcctaacgcatggtgagtactgcacacatatttgttgaatggatgatagctgcaatttgttgcatgctcccttgtgtccagcacaacactacaaactcgataaaaggcatttcatatagtcctcatcacagcctctgaggctgggttagattagagagagagttttatatagagggcagacacatagtaggggttcaataaaagttattattattctaactcctccctctgaccagccccactgacttttgtctcatctttccttag

SEQ ID NO: 196 - DNA, 602 bp, Homo sapiens
  misc_feature 1..602: note = DOK3
  source 1..602: mol_type = unassigned DNA
gtgagggggccctggaggggctggcctgggggagatgcaggccagaacacacaggtccctgagtgttgggctgaggagcctgggctgcaccccaggaaggggagagacaggttggtcttgggttgtggagagtgctgcatggaggcccagtggggcagtggcccctggggggatgccaaaggctggtgatgttcagtggctcgtgaggtggagaggagcaggtgaacacgagagagatttcagggagaaagaaaagcagcaagatggggtgaggcgctcaaaccaggacgccgagtgggaaagccaggagttggctagagaagggtcctgggtggaggcagggccagggagtgtcaggcccagggtgcgcagctgcccggggcgggacgggggaggggcgggaactcatgactcggggagccagactgcgatcagacgcgcgtgcccagctgaaccaggtgcgtgagaaggctgccttcaggtggccgggggctccctccaggtaggtgtgggcaccttgggcagccagaaccccagggaggaaatacatgctgcccccagcccctgagctgaagacaggctcccctccccccggcctgcag

SEQ ID NO: 197 - DNA, 14420 bp, Homo sapiens
  misc_feature 1..14420: note = ABCA1
  source 1..14420: mol_type = unassigned DNA
agtaagcttgggtttttcagcagcggggggttctctcattttttctttgtggttttgagttggggattggaggagggagggagggaaggaagctgtgttggttttcacacagggattgatggaatctggctcttatggacacaggactgtgtggtccggatatggcatgtggcttatcatagagggcagatttgcagccaggtagaaatagtagctttggtttgtgctactgcccaggcatgagttctgatccctaggacctggctccgaatcgcccctgagcaccccactttttccttttgctgcagccctgggagccacctggctctccaaaagcccctaatgggcccctgtatttctggaagctgtgggtgaagtgagttagtggccccactcttagagatcaatactgggtatcttggtgtcaatctggattctttccttcaggcctggaggaatataataactgagacttgttttatttctgcagagggttctaagccattcacttcccagatgggccaataatgctttgagtaatctggagatcatctttaatgcgcaggtgaatggaactcttccacagagggatgtgagggctgtagagcagagtgaactccctgaaactcagacgtcagctctttgtctctctatctctgaacacccttccttagagatcccatctctaggatgcatttctctgtagttagtttctaagtctcttgttcctgttctgcctttatttttttttcctggattctaagccagtatccccacttggctgtcttaatgtagcttaacatgtctgtaatcaaaatgatcatctttctgagattcaaagggctataagggactttggagagaatttcattcagttttcctcaaactagaataatgcttgcactgtctgtaaaagaacaaaagtgtcaaagcatccttttgttcactaaatttccttttttattatagtgttacttaaatattaggaagtaaaagtaggtataaacttcttataggctgttattatacaactatatgacccatacatatttacaaattaagtgcagccaaaattgcaaaatcaataccattcaaattaataccttaaatgtggtgaggcagctgttgttcaactgaaaccaaattataagttgcatggcagtaaatgctatcatgctgatcattttgagtttggccagtctatattatcatgtgctaatgattgaattctccacccatttttctacttgtatgaccttaatttgatggcacctgttccatcctcatgagtttgctacaattatactggtgccaacacaatcataaacacaaatataaacttgggctttgaaatcttgtgccagaacttggctttaaagtaagcatttaaaaaatccatatgtgtttattagactttgtttagatgactgttgaaatgaaaacaaagtgtttaaaatcctcttagagaacttaaatataatccctcagcaatatgtatacagatcttcctttgagaaaaactgattgtgttcagcctctcatgttacaaatggggaacctgaattctgaggtctctagtgagagaacagggactggaatctgtggatcctatctgttttaataataattgtaaagtataatagataatattatattaataaaataaaagcaaacacttagaatgagcttccatgtgtgaggcactaactgattaggcattattaactagatttattccttttaaggccccgcgatgtactgttatttccacatgttgtagctggggaacgtgctactcagagaggttaagtaacttgtctgaggtccacaccactaacaaggagcacaggtagggttcaaatccagataatctgactttggagctggcactctaactcaatgtgcctaatcgcttttcagtggtgtcattattttgcctattctccatctgagaatattgaagtttctgactccttccttgcctttctccctgcctcccgtggttatccccaggtcttggtgttccagtcctctatgtccgtccttactcttattcctttgctacagtgtgatccagggctcctgccccttcttatcctggtagagggggcccacttgctgggaaattgtctccgccatggtttatccatgttgtgtgtccattagtgagtagtgggaagaatcatatcatgttggcaatgaaaggggggctatggctctggggtagtctagtctgaacctcttattttacggatgagaaagctgaggtacaaagcagggaagggatttcttgaggtcacccagccagcaactgagctgcaaccagaagctgagatccccaggactagggccgagcctcattctgtcccatcacagtgacttttcttccctcctccaaactatttttattttttatttttttgcagctgcttagcagcttgaagttagaagaaagggcagggaaaaggttttccgtgcttagccagggaaggaatcctgcaacaggatgtggggttgggtcattcaaattgggccagactccactggtcttgttgcttcttgcttggtattgcagatgggtttaaaagtgttaggattagagagataggcaggtttagccaaaggcagtttgtagccttgtggcagagttctttttaaagaaggaagtgggatgcaacaccctgacacaaaggggcttaagttgttataccactgcctgctaacctgttttccttaactctcttcctgatttctaaaggaagtatattttgctgaatcagaaagaaaagtgatttatttcaggttgctgatgcttagattgttagagttggaaagatctggcttgcatcttgtacagctgacagaactggggctcaggggggcacaggtgcccagagttggtcagtcaggaaagtagcaccagaaccagtctcctggtggccctacagttgcagacccttttttgctttgctctctgtgtatactaaagcttctatgtctctgaatctcaagttctgactggtagctactttccaatccacctggcttagatttctagattatattgtttagacgtcagaacctcttaagggttttggggccacttgttagctcacatagtgagaaccagccctgcccattaggtaggggaagaagttagcagtccatgatagctgttgcctgcagcgtatggatgttcattgcacagttcctgtctcctgagatcctggagtgtatacgcttggcctcagagcccagcacagagcctggcccttgggacatgcttagtaagtatttactgaatgagtgggaaatgtcttaaggcccattagtttgcaggtcttgaggaggctcccttgcactaggaagaatagaaagcatacataaagcctgtgtgctgccgccaggaagactagaaacgctatgttcagcctggagctgaatggtataccccagagcaaccctgttgaaaggcagtgcttgccttttcattctgtgtcctggtttgctggtaactcctgggtcccctgcctctcctgtacccccattgtgcagactgaggggggaccatcagccagggttagttttccgctgtttctgttaggcaaagaataaattgaattgagttgtgaaagttgggtgcaaagctcagtttgggtccaaagtaacagttaacttgtgtgggtggcaggtattcagtacaaacagggctggggacaggaaggggaagagaacttcagagctttcacgatcctcatctggttttaggctgatccagaggccaaggtccccatggaacaaactggacaaagtgagggtggccacatggcctcttttcttttgcctttattattaattttctcaaatagatctgactagtcatgtggctgggaaaatagttaattgtgatttttttttttttaaactgagtctcactctattgcccaggctggagtgcagtggtatgatctcagctcgccgcaacctctgcctcccgggatcaagcaattgtcatgcctcagcctcccgggtagctgggattatgggcacacagcaccacgcctggctaatttttgtatttttagtagagacatggttttagcatgttggccaggctggtcttgaactcctgacctcaagtgatccacccacctcagccttccaatctgctgggattacaggcatgagccactgcacccagccagagtaccactatttgggcattctttaatgaaaaagaatgaactatccaaaaattaaaactcctcatttatgagcttttagagaattttacagagtagatggaaactctctgcatcctttccccacttctagtttcacctgacacatttcttccctgtccttactcctgggccggcagcagtggtcatgattccaatcccagcttggccaccatctgcctcagtggcctaggaaaactcctttctccagagctttagttttctcttctacggaatgaagaaagttaaaacaaatagacatttattgtttcatttggataaatatctattaagcatctattacttgtggtatggttagctgggtatatagtggtgaagcagctgggcatgagtactgctttcgtagagcttacagttcagtgaggccagcagatgtgaaacatatcatcacacaaataaaaatataactatcaactgtgatgaggattatgaaggaaaaaatccggcaaactatggtactggtgttagatactagcaggtgtgggtagggatttcatttagattgacaggttgtcacattaaagctgagagccctgaagttcaagcaatggttagccaggcaaagatcagaggcttagagatagggaaatccattccaggcagagagactgggggtgcctgtcccctaggtcagggaacagaagaaagccagtggcactggtggagtgaataagactggcgggggatgagttggtagtagacatgaccagatcatttagggccaattctcctggggaaggagaatttaatttaatatttatttatttatttatttatttatttatttatttatttatttatttttcaagacggagtctagttctgtcgcccaggctggagtgcagtggagcaatctcggctcactgcaacttttgcctcctggtttcaagcgattctcctgcctcagcctcctagtagctgggattacagacgcccaccaccatgcccagctaatttttgtatttttatagagatgcggtttcaccatattggccaggctggtctgaaactcctgaccttgtgatcctcctacctcggcttcccaaagtgctgggattacaggcgtgacccacagtgccccctgagaatttaattttattttatgtgcaagaggattccctgaggtagtcaggccacattgtctggtgactcttgggatagagggaacttgaatgacaaaggcccaagaaagcaattgtaatcattacatatacatggaccattttatgctgttttcttctttcatttaacattatttagtgtgcgtgttcacatatttctaaatcatcttctgatttagaataatgatttctgatgtgtaggctgtgttttatagttttgaaagtaatactttgatatccattactttcttgattctcacagcaattctgaggtgtatgcgttgcaatttctgtttcacagatgaagagagtattgttaataagttaatggccgggcatggtggctcacacctataattccagcactttgggagaccaaggtgggcggatcacttgaggccaggaatttgagaccagcctggtcaatgtggtgacacccatctctactaaaaatacaaaaattagccaggcgtggtagcacttgcctgtaatcccagctatttgggagggtgaggcaggagaatttgcttgaacctggcaggtggaagttgcagtgagccaagattgcaccactgtactcctgcctgggtgacagagcgagactctgtctcaaaaaataaaaagttgctaagaggagggctgggatcttttggctccaaatctactgtgggatgatgcctttgacattcctgatagctgtgcagtaatccattaacacagtttttataagttcaaaccctgttgccaacatttagattgttccatgtgtgctgttacaaataaattactataaagattctatacatttaatcttttattatttttgtattatttctgtaggccaaaatctgaggaacaggattactaggttgaagggaaatggcccttgaagtgtctgatcagatgtctttccagaggatccaaccaatttaaatagccaccatcaatgcatgagactttgtagttcagggaaggcaggcctggttttaaaaatcatttcccctctctagcatttttctgatgtgatccttaagatttcactttagttttcccaggtctcattggcatgtatgctgttagggatgggtctaaaattaatttttcttcacattcatatcatgtcatcccagtgattatttaataaataatcacttgattaaatagtgattccttttctagttatttttgggacatttattaaaacctggatatggtggctcatgcctgtattcccagcactttgggaggctgaggtggggggattgcttgagactaggagttcaacaccagcctgggcagcatagcaagactccatctctataaaaataaggaaattagtcaggcatggtggtacttgcctggagtcccagctacttggaaggctgaggtaggagaattgcttgagtccaggtggtcaaggctgcagtgagctatgaccatactactgtactccagcctgggcaacagagtgaaactctgtctgaaaaaaaaaaaaaaaaaaaaaaaaaaaaaaaaaaaaagatgtgtagggagcaattttggagttattcatttggtcatttgatatgtagttttagttttggtgctgatagagcccagaatgtaccctgaatttgatgaacattctgatatatgggggagctcattgtcccccacttacctttttgcctctcagaatatcttttgatatttttatctgttttttccccattgaatgttattaccttatcaagctcaaaaaagtaccctatcgctattttaagttcagttgtgttaaatctataaattagcttgggaaatttggatattaaatgaactcatgaagaagcagagtttagctctccttaattctcatcttcctttatttatcctactacagttctgtggttttcttttatgtaagaagcacatgtttggctaagttaatgcctaggtttttttgtttatgtgtccattctcactgtggatagtattctcttttccccacattatattaatttaactggttttcagagactaatagcaatgctattatttaggagaatttaccttggttctgattaacttacccatacttgcaaatcatttgcagctttttagttaactttgtgagttctcttagatttacgaccatgccagaaacagaaaggatattttcatctcttcctttctgatgtttattcttcttgtttcctttttttatcccccattatattctcaagaatctctcaatactaagaaatagcgacttcatttttcagcgcggagtgcattattttggctaccatgattcagaagcctcttgcctaaggcccaattttattctgctagttttctctgttctttgtacatggcccttgcgctgccctaaccttgaattaacgtggctaaatctcaagaatttaagagcaccgtgactgtgtcctcaggctagggagggaaatgggttcacagagtgactggattgtggtctatgaacttcggcagccagcagcaaaagtcaggcatgaataatcaagtggacagtgaacatctgtagtgtgggagatgttggcataactatgaatgatgattcaagagtggtttgatgcatattgaataacatgatgataagtactagactctgtgctaagccttctatgtgaaatacatttaattctcataataactctagagcagtggttctcgaccggggccggttatccccctaccccaccccaccctcacccttccaccagggacataacatctggagatatttttggttgtcacaatcctgggaatgtatgtgctgatatttagaggttgaggtcagggatgctgctgaacttcgtagaattcataggagagtctctcacaacacctatctggccccaaatgtcagtagggtcactatcaagaaaatctgctctagcagtgcctgctcatattatccccatgttgaaatagcaagatgggaagtgcaaagtggtgcttcggtactcttggagcagctttgactttggtgagaaacgccttttaaaaacaatgtttcttcccatcttcccaccccatggggaggtgtggggttgggtgggtaggcaccaaagcaagatttagaagagttttctgtaggaatttataatggtaaaggatcaacttcatttccaagctatttatgagggtttatgtttaggaaaagtgctaagcttagagaaggaggagaaatctgattttattaatgagtgtagccataatggcatatcctggcagaagtcaactttggtttctagagggaggctattatgaaaagaaatacctggaacattcccctgggtttggaaggtgagttctaggttcaatgatgggaagaattttagaggtccaagataaaagggcaaagattaaattttgtctctcatgagttctctggctcaggtggtgtgaactttgcagacagtctctttaattcactcatacatgctagtctcccagctcagcaagggctttgagagagcaggtgtctgtatgctctggtaagtgaaggcaaagtgcataaggaggttggggtccataatggcgaagagaaggagcccttcagtcagagtggctttgaatcttggctctgccatttgccaatcttggaccattgggcagtgtattaactctttgaatctcagcttcctcttctgtaaaatgtgtataacaagagtactaattggattgtttgatgattaaatgagttaatgtgtataaagcactcacaaccctggtacatagtaagacctttcattattattatcatcatcaattttttttaacctcttttcctgatctgcttacactcaccagcttcagctgctccaaatggcttgtaagattttttgtttgccctttgctgtcagttgccatggggaagatccattcatttttttcagtcaaccaacatattttgagcatctgctgccctacaggatcctagatatgggggctgcagagatatccaggaacataagccttgattaattgggtcagatcagtgctcagcagggctggcaagtgctaggtttcttttaagtggcatatcttaaaaggtatatgtcctaaacatagctttgtgatggcagcatgatgggtacaaaagcacacacttaagtgtcagtagatctgggttcaaacattggtgcagtttcttatggctcgtaacttgttcaaacctcagtttcttcacttctaaaacggtaatgatacaacctacctcacagggttattatgaattaaatactggagatgagatacacaaaacgtcttgagtacacagtagctgcccaatattggctgtaagtattataaatctacaagctgtgaattaattttacctctctggatcctgttgatatttctagaccattccacctagtggggccatttcctacctgagtcacccgtggtgtcaaatagaatgtcatgtggcctcctgagttgggtagaattggctgctcatctcaaccccgctactgactatctctgtgatttacccttcctccagccttagccttgctacatataaaatcaagacaataatgtttcctatctcacagggttgtcctgaggattaaattaagtaattaatataaaatgtgccttgtacatattgggccctaaataaacagtagctactatttatccttaaagtacaaatggtagtttcagagcttcaaggctgatggctatttatcttactcatactctttgtttagcttcatttttttcccctaatttcattagtattttcttttctttttttttttttttttttttttttttttttgaggtgaagtctcactctgttgcccaggctggagtgcaatggagcgatcttggctcaccccaacctctgtctcctgggttcaaacagttctcctgcctcagcctcccgagtagctgggattacaggctcccgccaccatgcccagctatttttttgtattttcagtagagatggggtttcacccttttgaccaggctggtcttgaactcctgacctcatgatcaacccacctcagcctcccaaagtgctgggattacaggtgtgagccaccacgcccggcctcataagtattttctaaatttatttacagtcatgccatttaaaaggaaagttgtattcctgtctttgttaatatttataagtgattttattcagctacaagcttggaatggcatataattttgtattctgcttttttcacttaatattacatggctaatgatttctgtgtttcataaacattattctgatgatggcatgatatattgttgagtacatgtaccataattgaatcatttccctattgctatgcaattaagttgtttccaatattttgcaattataatgtttcaatgaatgaataactttatgcatatagctttttgatatcttaagttcagtttcctaggatgaatttccaggaatagtaattgggcaaatgggataaacatgactcttgaatacgtattgttaacattgctttcccaaagggctcaactgatttatatttccgtgttcattatcttttaaaccagctcatttactcaccaaacatttttaaagccattatcatgtggtaggcttagtaagaagaaagtgaccctaagggagaagcttatatataaatagggtccctggtgtaccaagtgctgatacagacacaaagtacctggggaaattgagatgagggagtcctggctcagctgggagaaaagttcattttcatagagtcatggttttgttctttggcagaaagaaaattgctttcttccccacccccacccccagctttattgaggtataattgacaaataaaaattgtatatctttaagatatgcaatgtgatatatatgtatatctcaacttaaaaaataagctacagaataaaaaggtgtttgctattaaaaaaaaagaaaaggctgaatgtcattcccaagcttggaaatttgagtatgttgcctctttgggattatttacagaaatattagcaagaccagccccatctttggtcttgagtactccactgtcagcatgctttcttccagagagggatccatttgcctttatttttcattctgttgtgccgtctatgcaaactattcttgatagttttatggtaacagtgtttttttgttccatgagataatttatacatgctcattgtggaaaatttagaaaagacaggaaagtattaaaaacatcacttttttttttttttttttttttttttttttaagagacagagtcttgctctgtcgcccaggccggagtgcagtggcgtgatctcagctcacagcaacctccgcttcccaggtttaagtgattctcctgcctcagcctcccaagtagctgggagtacaggcatgcaccaccacgcccggctaattttgtatttttagtagagatggggtttcaccatgttggccaggctggtctcaaactcctgacctcaggtgatccgcctgccttggcctcgcaaagttctgggattataggcaggagccactgcgccagccacacctacgttcttatcatcctagtacatccactgtcattatcttgctgtatttccttctgcccagtctcactctgatcatgcagtggcgtgatcatgcagtgatctcggctcactgcaacctaggccttctgggttcgagtgattctcctgccttagcctcctgggttcaagtgattctcttgccttggcctcccaagtagctgggattacaggcatacacccccatgcccatctaatttttgtatttttagtagacacagcgtttcactaaaattttgtatttttagtagagatggggtttcaccatgttggccaggctggtctccaactcctgacctcaggtgatccgcctgccttggcctcacaaagtgattacaggcatgagccactgcatccatcgccaaaaagattttttaaaagagtttaatgtagaaccatatcaaaggtctttggaaataaaaaacagttttttaaaaatatcagaaataaaacaacaaataaataaataaataaaaacacccaaaacaatctgaagcacgagcacctagcagaaaggttcaattatgatctattcatagagtggaatatcaagtagacattacaggacatgttttaagattatattttatgtcatgggaaatgctctcccagtatgatgttaaatgaaaaaacagaatacaaaagtatatatgctgcatagtctcaatattgtagagaaaaaatattatttatgtatgcatgaaaaaagacaaaagatgttaacagagatccattgttacttcagtttactagggattgtctctgggaggtaggattaaggtgatttatatttacctttttaaacttttctgtatttttttattttcaaattttccataaaaatataaggacttgaagatcaagaaaaaatttctgctttggctcagtgcagtggctcacgcctgtaatcccagcagtttgggagccctaggggagaggatcacttgaacccaagagtttgacgttccagtgagctatgatctccggatcgtaccgcctggacgatggagcaagaccctgtctcaaaaaaaaaaatctttgctttttttttttgtttgtttttgagacggagtctctctctgttgccccagctggagtacagtggcacaatctcagctcaccgcaacctctgcctcctgggttcaagcgattctcttgcctcagcctcccaagtacctgggattccatgcacccaccactatgcccagctacttttttgtattttcagtagagacagggtttcaccatgttggccaggctggtctcgaattcctgacctcagctgatccaccggccttggcctcccaaagtgctgggattacaggcatgagccactgtgcccagcccaatcttttgctttttttaaaaaaagaagacaaaaagggattttataccagtattatcttggctgtgtgactctgaagccacagttgtaagttataattactctgaaacacaaggccctgtgactcttttgggctctttggtgtttatcttgattacaacgttggaatatagaaatgaaaggaatgggagaggtgatagacttcaggcagtgtaactagttgtctgaacactactggctcaattatattgtgtctagtgatttccatcttgtccgtctgctaatttatcgcctggtaactcactgaggcagggttttcctttggagaaacctcattgttttaaccagtgtatcatgcttgtttagaagttcaatgatctttttaactcatcggagaagatgatgaccagacctggacagatggggaaggactttgcactctctctttacagtcctgagtgcacacaggtcaatatggaactatgtgtgaattttcattgtctttgagagccctcttctctgccccatagggagcagctttgtgtgcaattagaggagcaagggttgtgtgtatttagcacagcaggttggcctggtcctctcctctcaacatagtcaccacatacctggcactatgctaaggctgggaatgcagacagatgggtgcctgctttcagagtgctcaatgtgctgaggaagccagcaacagaaacagatgatttcaggagctccaggaaaatgctacaggaggagtgtgcctgggttactggagtagcacaggaggagggcttctagctcaggctgagattttagtaaaggaaattatgccacgatgaatcctgaagaatgaatagaagtgaaccagataaagcacgataggaagcatcttcccttacctaagggaagacacagaggtatatggaatggtatgttaaaaggttgggactccaaacagttctgttaaagcttagagagtggtgggagagactggagaagttgattaattagtaaatgaagttgtctgtggatttcccagatcccagtggcattggatatccatattatttttaaatttacagtgttctatcttatttcccactcagt

SEQ ID NO: 198 - DNA, 1448 bp, Homo sapiens
  misc_feature 1..1448: note = TMEM195
  source 1..1448: mol_type = unassigned DNA
gtgagactttattggaagaggctactcttattcttgctgctttcttaataaatggtaaggtcaatctccttagggttttgttcactttcattattcaaatgattacattctaataataataattcagtttcttaaaggcgtgaagcaaaagtagaatttccacttgaattttcttacaatccatagccatcttcacctgtaaattccctcttttatatgtcactccagaatatgacaaaaacaaaagtttaaagttttcctggactccatggcattgtaaatgtagctattggaagctcaatgacaaatagttacaccacatcagtaaactttgtaacaatttcatttttaaatctttcacttgtccaccccttccagggaaactatttcctctttcatttggtagagtagttcagcagaatgaaggatatgcagcttgaccaggtccagctctgacttcctaagtttgaatttgacccaagttttctttgaaactctgaagtgctcagaggcacatttcacttctggttcctggctatgattggaagtggaaatatccagaattagaacttgaatactgaagttccccaccctatcaaagttattcctacaacactcactagttcatggcagaaatagaaaaaaaatgacatcatagaatatgttattgagagttcagtttagaaagcttcaaaactcatgattagcttccaaatgagtaaaatccgtatctttttcaagattagatattacactttctttctgagtttttaagttaaaaaaagtggcactgatatcttcataaattattattgtttatactgcaatatcaaaccaaattcaatgctccatcaccaatagtggagctttcaaaacatctcttattatgtgaaacaaaaatcaaaaccaaatgaataaaatctgaatcatgttttaagcaaacatgtaaaattaccatattctctaaaattctaagcagaaaattgtctctactctttgaatttcatcattagaaatagttctggtaagtctaatatgaaaaattagaattatgtgcatctttaaggaaagcctcttgaagtgggtctaaattatacatgtagagacaatagacctctaattatctaccttattctaaatgtattttttaaactaatttggaagatgttcctcttaatcttataatattataattatcgtggttatatctatgattacatttcatattcaaattggatgatagaatttgagcacatgtttagtgtatgtctaaaataattagacttctttaccactgtgtaggaagtaatcctctaaaatgagtgtctaccatctcctgcagggcttcccaaatctgggcctttcaattctgaaatgactaatcaggaaatgtaaaataatttcatatggaacataatagcatctcttttctgcaacttcctttccag

SEQ ID NO: 199 - DNA, 3633 bp, Homo sapiens
  misc_feature 1..3633: note = TLR4
  source 1..3633: mol_type = unassigned DNA
gtatgtggctggagtcagctcctctgaactttccctcacttctgcccagaacttctcactgtgtgccctggtttgtttatttttgcaaaaaaaaaaaagagttaaattaccttaaagactcaagaagccacagagatcaaataattcattgttacagggcactagaggcagccattgggggtttgttccatttggaaattttgagtgctaacaggggcatgagataacatagatctgcttaaggtccctgctctgctaccttgtggctctgtgaagaaattatcaaacctgtctgagactagttttcgcatctgtaagagaattataataccttcttcactagagagtaagcagactgcttcagtgtcatttcttcccactggtggtctttacactcagcttcaagcagtcaccctgctcctttcaatctcaggaaaaagatggcttttgtgtgtgtgtctctagagaaagaactttctaagtgggtgtcagacttctgtatgcagtaatatagtttagtccagaggatgaaaaaaataagagaatgaaaaaggaaaagagagagagagagaagaaaaaagcaagagggaaatatgtataatgtcagctaatgcaacagtttctttcttagtgaaataccaatcagctggttggtaatcttattcatgatggatctcttttgtttttcccctgcgcagacttcacagttgctttagaaacccatagtagagccgaacagctaagaaaatgatttacagtgaggcagggtcagaaactcaagagagaaaaagccagctgcagtcctgaagttgaggatataggagaaaatcaagtaatatttagcaaagactaattcattatcttgaagccatcccttccctcaattccctgcccatagtcctcctccttgtcctcttctctgtatccctctgctgttaggttaatggagatagattttctaattaggctcactgcgagataaaaccacagccaaacttgacttcttttccccatgtaccttttcctgtcagtccctgaagcctgtccatccctgcccatccccttagttccactgtaaggcaggccctcatttcccctggcattgactcttacacactaactgctttcctgattccagtcttcttcctttaattcattctgcacgttcttgtttgttatgtacttgcatttgttgttattatttttccttaggcttcaatctaacaaattactctccttaaaaacttttaataactctccattgccattagaacagctttctaccacagggcctttgcactggctatttcttctacctagaatgctagatcagtgctatccattggcaatattatgtgagccacatatgtacttttaaagtttttagtagcctcattaaaaaaagaaacaagtgaatttaatttcgataatagttttatttaacttagcgtatttaaaataatgtttaaaattttaatatatatttacctattattgatatttttacattccttgtttggtactaagtctggaatttagtatatgttttacatttaccacacttctcaatttacactattcacatttcttgtgtttgataactgtgtatggctagtgactaccgtattggtcagtgcagcccaagtccttttcatgctttaatcactccattcagatctctgattaaatgtcccctcctcagggcagtcttccttgattgccccatgtagagctctccagcctcacttatttgcctcaaatccccttatactgcttaatatttttttttctagagcacaacattttatatttttgtttgtttattttctctctctccctttgtaatggaatcggtaaggaggcaggatcattgctggttttatttaccactatatttccagtggccagcacacagtagccgctagatgtgtaagtgataaatgattgaaataattgctgcaggacaaagtctgaggccctcctgatctggcttgccctcttacttagatttcaccactcccaccactcaccagctaatctgagtttgttttccactctttacgtgctcacgttgtcctctccttaggacatgtttttcttcccctttccacatatctaaaccttactcatcttccaagacccactttaaaatcttccttttctgggaagcctttcctgaatccagacttgatctctgctttctctgaaccacagggcatattttctaagcctattttatggccccttgagatagtgttagctttgctcctatctaaactcttactctagactgtgagtccattgaagtctggagctgcatcatatttttctttgtaatgcccacagcacttggcaggaaatgcctacaatttggacttaagtaaaccttcatttaatcagttattcaatcagttagtgattcagcaaatatttattgagcaccaaccatttgccagacaccattctgagtgctggagacaaagcagtgggcaaacccatcaaacttgcaatggaatacaggagatgaacaatacgatgagaacaatcagatagacaacataatgttagatggttgtgcttcccgtgaaagggaataaaagagggcaaagaaagagtgcctggcactgtttctattagacaatattgtctttgaggctccatggcttgcaacatttaagcagacatacgaatgaagatctgcatgtttgaactctgactttgcgcatattacttcatttctttgaatttccattttcctcatctttaaatgcttatttgaagattaagtgaaagtatataacaaacaagaactatgcaggcatatggtaagggattaatgatagatgataataattaatgttgacatctattgatcacttatactgtagcgggcttttaaataaactctttaaacaccttatctcatttaatccttcaaacattctattggtttcaaacaacagaaaactacaattagctggcttctgcaaggaattttgttggaggaaatgagagcattcagaaattagatgggagcgttagagaattaggcttacaaagaatgtgggaaagtaggctagaaagcagtgtaaaaacaaagacagcataaagcacttgaccttatttactaggttccaccatgggaatccatgcactctaaagatttccccctatttctacatcactttgctcaagggtcaatgagccaagaaaaagaatgcagttgtcaaaatctgggccatgactaaggaaggtctggacatcttgactgccagacagtctccccaatgatatggagtatttaaaatgatactggatattttatttattttttgtattttcaacttttaagttcagaggcacatgtgcagagcatgcaggtttattacataagtaaatgtgtgccatggtgatttgctgcatagatcatgaaaatatggaacgcatcatggatttgtgtgtcatccttgtgcaggggccatgctcatcttctctgtatccttccaattttagtgtatgtgctactgcagcaagcacgatattggatattttattacctacattttacatatgataaaatgaggctcactgaggtttttcttttgttcgttttattttgttttgtttttaaag

SEQ ID NO: 200 - DNA, 422 bp, Homo sapiens
  misc_feature 1..422: note = MR1
  source 1..422: mol_type = unassigned DNA
gtaagtgttttgagcttattgtgctgggtgctagagtagcagggaggacagacctaagttgaagattacgggacaatctaagtgacacctgctatctcagacagagacttcagcatcatagggagccagagcagggagttccaagccgtgggaactgtgactctttcttactgacccttaagcaaaattctaagctccagggaagcagaggcagcagcttgagctccctgattgaccagatatgattaatctttgtttctctgtgctttgaacatgactatgctccataaatatttaaaaattgggaatagggtgaagatagatacaccaaaaggagcaagcagtgtctgaaaccagcaaagacttaaaagttaccgaagaaggtgttcatgctttcattttatcttttttttttaacctag

SEQ ID NO: 201 - DNA, 393 bp, Homo sapiens
  misc_feature 1..393: note = FCGR1A (CD64)
  source 1..393: mol_type = unassigned DNA
gtaagttggactcagaggggacagttagaagggtacaggctgtggctgttgtgagtcaagagttttgtcttcctgtggtaactctgggtagaactcatgagtatgaagcaacttgtatctgtgcttccatggtttattagagcttattttatgaaaaggatgggaagggcaaccctgaggtagcattaagcctggacgcaccgcagtgaagtttccttgataaccacctgtagcttgttcagttctgttagtactggattttgagaaagagaaatagaaactcaagagatctgagttgatccctcagagtctacattaattctgtctccccaattctctcttcctcattattttccttggaccaactgatatctttattctctgatctcttgc

SEQ ID NO: 202 - DNA, 1273 bp, Homo sapiens
  misc_feature 1..1273: note = CSF3R
  source 1..1273: mol_type = unassigned DNA
gtgagtaggggatccctagagaggggctgagccttgggcttgatggagagctggaatcctggggccactccccacagctctgccaagtgttcatgtccggggtcctaggcctggggcccgggattcgtcctttggaaccccagccctggacactgagtctcttttcacaggatgcccctacttttgggggtattgcaccagggaagtcactcacagccaagtatctccttggggacactgagctgggctggggacaggcataatcattctcccctccccatgcctctgtctctcaggacgtgtgtgtctgtgagttccagccgcgcaacccctccccgtctgggtctctatttcactctctgtttccatctttcctcctcagcttctctctggcttgctatctctctgagtttctctgtctcgcatatttcctgctatctcttgaatcttaaactctcggtaacagacgcttcccgggctccaggcctccgagtgccccccccccgcccactctctgggtcggcgtacattgggccctttttctctgtctctcgatatctctctggcctcaatcgtcctcttggcgagtctctctgtcgtttcagtctgtgtggatttcagtcaccgcctcactctgtcactcttcctgttgctctctctttttctttatctgcagcatatctggaaatgcctctcccctctgtttattcccagccccctcctccctccccacccttcccacagaaagaatctcgaggtgagagtagaaacagcaagggagcgaggggtgtgaagtggggaagagagtgtgaggggagagggctgagcgtgatgagggagaaacagaaagagggaatgtgaaggcagccagcccaggagacatcgagctgtgctgggccacaggagtgagcacctgcagagcccagcagagatggccgcagtgacaggagggccagggggcccaggagttaggaggagaggatccagaaagggggctttggaactgcgcagtgtggcacagacaaaagcaaggtggcagtcctgggaggtgtgaccacacaggctgtgagggaggcaggggtgaggggaaaactgaactgtgctggattctgtgagctgctgacctgctatttatggctcgtgaaaataacctatggttgctgagtgccaactccatgcaaggcacagggctaaatgcctttaccaaaataatctcatttcatcctcacaacaacgctgaggagtgagtgctaatgttatttctttcccacag

SEQ ID NO: 203 - DNA, 29639 bp, Homo sapiens
  misc_feature 1..29639: note = FGD4
  source 1..29639: mol_type = unassigned DNA
gtaagtggtttcattttctagtagatatatttttttcttccccagcgtgggtcactttataacactgcatgccgagggatgtaaaagtggcagagtttggttttcttttaaagttaataaagctcacattggttttcaagttcaagattaaaatatgtgctattgatgggttttggctctccaggaggcttcttctgggctaaccagatacaaatgagtttgatagttaagtttctctggcttatttttattatcataagtaaagtttggaaatttggtcattgaggggagaaatatagagaaagaaaggaacagttttctctttttcctcccatttctggtgtgaacaaatgttaagaaatatttaccactaatcatttttctagtgctactgcacggagcattgttctgttaaaacaaacaaaaattcccaccatcccagtcttgttttggtgaagttcaggggagtaagtattatttgtttttctgtaaaagacaacattgtagagataggcatgagtttgttttttaaaaattgttagtggtagtagtattaagccactgtggtctctttgagtgttgtagagagcatattaatgagaaattgagtctttgaaatagaaatgtaattcaactgtgtgatagaaatgtaatcatgtgacgggaaatgtctgctaaaaatatttttaatcactgaaatgggttcattttgtttattttcccattcgtttttgactggatataagcctcactgaaaaatgtccacactgctgaacaagataaactacaacttagaaccggcagccttcacagttaccagtattctttctgatggtggtgtggctctttccttccttaagtgtcaggggcatacgctgagtgtggaagcccaaggttggggttggtgacagcactgcctcaggagagctgaggcgccactcctgcagctgcacagctggctccgttcccaggttctagtagctgcgggagggttaagcagcccagtccatgcctgcccctgctccctccatcactcaggcagaagcctgaattcttttgcaggcgttccaccactcttgcataaagtttggaggtcttgtgatctctccaaaatttaccatcatactctaaatatgaaattaaaaaaaaaaaaagaaatgcagcaaaagggaagtgttttctcaccccaagcagtgaagttttaggttctctttttcctgaaatagcttctgtccattctcatttatcttcaaacagttagagatgattggagagacagtattccctcctggaaggcagctcttaattagagaggaaaatatttctagaacaggaagatgaagcttgagaggtgaatgttgataaacatagagaacttgttggctcttgttttctgctttaatggacagcaccctttagagagtggccactacaacaatatggactttataagttcctttttaggtttttaaactgagaagacagtaatatagcacttcctgcctacactactggataattttgttaaatcatctaagaaaaattgtaaatatgcatatttagtgcacatgttattttataacagttttttttttttaatgacctgaaaaaacttctctttctaaagcttacgctttcaggcaaactcctttcacatgaaagaaatgaaatatgatagagtatgatgaatgactcaataggaaatattatgcagttctttggaagaatgataacttggtgttcctttaataaacatccaaatgtacatgtaacctaaactgcaaactaggattaagatttaaatttaatcagttcttcttagatactagaatagacttgaggaggtcaagagtagataggagactcctggaataatccagggaaaagttgaagtcatttatttcaaaaataaaaaggtattttcttagatgttttgtggaccatccctgcccacttcctgagccaccttctgataacctttactgtaggcacctgttttctttataaaataacaagctaatttttattggctaaagtacatttgtaaatgtcttcaccctggtttgtgtgtgttctctgatttatctcccaccacagcacctgctaagctctggctagaacagatccttccatttttattccctggcgaattgacacaagttagttggttatacgttgaagcaggcagaaggaaaacacttaggagaagaaaaatagtgttaatcgtagaatttggtcattctacatggtggttattataataaaacttggcctggggaaaatgagtctttcaccctcttagagaataagtacacacaaatacatttttaaaacatttaaacaaacaaacaaaaaagactgtgtcactgtggtcttgtcttttccatgccattataaagcagttatttcttttttttctctctctcttttcttttttttttttgagacagagtctcgctctgtcacccaggctggagtgcagtggcgctaagctccacctccggggttcacaccattctcctgcctcagcctcccgagtagctgggactacaggcacccgccaccatgcccggctaattttttgcatttttagtagagacagggtttcaccgtgttagccaggatggtctcaatctcctgatctcattatccgcccacctcggcctcccaaagtgctgggattacaggcgtgagccaccacgcctggccataaagcagttattatttcttatgcttagtcccaaaaacctagtcttccttgagtcatttttctcttatgtacaatctggcaacaaatcttgcttcaagatatatcaaactgactatgtcttattgcctctactgttacttgccctgctttaggttacacacagctttcccctggattgcttctactagcctcctatctacccttgacctcctggagtccattgtctacctaacagcctgtgtgcacctctaaaaacggagggcagataattgccacttctctgctcgcagtccttcagtgaattcccatctctttcaaaggaaatcccctgttctttcaatgatgtgcaaggccctaccttctctagcccctatccctatccctaccccttaactttctgacctcagcatcaaatatgctccctagtctatccatgttgtcttccttatttctctttgaacatatcaactgtgcttctaccttaggatttttgtagttttacactttgcttggaatgctaatcctcaaaatatgttatgccttattcccttcatgtctactcagctagcagagtattagtgagacctccttgtcctccctgtatataggatataataatagcctctcacctaatagcacgcctgttccttcacccagctttactttcgttatagcagtttttttgtttgtttgttttgttttgttttgagacagagtctttctgtcatccaggctggagtgcagtggcgcagtcttggctcactgcaacctctgcctctcaggctgaaacgatcctcccacctcagcctccacagtagccaggacttacaggcacacaccaccatgcctggctactttttatatttttagtagagacatgggttttgtcatgttgcccaggcaggtctcgaactcctgagctcaagcaattcacctgccttggtttcccaaaatgctgggattacaggtgtgagccactgtgcctggcccatcatagcacttttaccatttagcatgttatacaggttatttttttgttgtggtgttcactgctatgttcctagggtctagaaaggtaactaatagatactgggtgctttgttaatatttgttgattgaatacatttctaatattcagtgcctttttctgttgggaaatgtaaaatgagcaggaatataagaagtttaataaattataaatatttgtagtgttctccatttcttcccttcagttctcttttaagaataaatatccttgcttgctaacctctggtgtcccttgttgagcagcatgtcaataatgagttcctgctattggaagcctcacaggccaagttgatataaagaagtatgaagaaagtatggaggggttagtgggttagtgagattggaatctgtggctgagcccgtacatgattttggatgattaattaatgggctgtgtcgtaagacagaaaagagatgagggagaagctttggggtgtgaaaccaagaaatctggagcgatactgtctgtgtggagaaaattgtggccaagattttttgctattaaagatgtacttaggccaggcgcagtggctcacacctgtaatcccagcactttgggaggccaaggagggaagatcatgaggtcagaagatccgagaccatcctggctaacaccgtgaaactccgtctctagtaaagatacaaaaaatcagctgggcgtggtggcacgcacctgtagtcccatctactcagtaggttgaggcaggagaattgcttgaacccaggaggtggaggttgcagtgagctgagatcgtgctattgcacttcagcctaggtgacagagggagactctgtctcaataaataaaaagataaaaataaataagaaaaagaggtccttaatattttatattaatgatatacttgccccactttttgttttttctcttcctacatttaagtcatttcactctttgaagcactcttgtaggagggtgataggaaaagtaaaacgattgaataaaaaataatctagaaatatttccttagtagttcctgtaaggcttgatgtggaatagaaaaccaatacacgggccaggtgcggtggctcacatccgtaatcccagcacattgggaggctgaggtgggtggatcatctgaggttgggagttcaagaccagcctgaccaacatggagaaaccgcatctctactaaaaatatcaaattagctgggcgtagtggcacatgcctataatcccagctacttgggaggctgaggcaggacaatcgcttgaacctgagaggcggaggttgcagtgagctgagatcgcgccattgcactccagcctgggcaacaagagcgaaactctgtctcaggggggaaaaaaaaaaaagccagtacatgaattaaagtgtaagaattatttgtgtttacacttcatgtaatttatttccaaatttatctagataattggaaattgacctgcattttaaaatctgtagccaacagttaatgttaaaaaaaaaagttcccttccccaaatgcagttaaaatctatgaactgctgtttgctatttgataatatacttgatctgaatagtaattatacattattagattacatgacttcttagaaatatggaatcaattttgactatagcaaaaagaaattatacaaagcttgaaaataattttgttattttataaatgtgtaagccccttaatgtctatcagtagttactgatttattagaaatatcatgtttttctctaatattgcccagtaaattttttttaaatttatttccatcagagttactaatttagagtgtaaaatagcaaaataagaaatatcaaggaaaaaaggtatttctggtaactttttatctaaaattgaccattttgatacagaatggtaacaagtcatgcttagggttaggaaatgtaactgtattcactatactcaatcctaaaaattgtgtaatacctctatgttcaaggaggtttaagttcgcctccctcatagacctctaaatgcagtgactatgagaaatgtcataggagaggtacaaaatatgtttggagaatagaaaaggaggaacaactgtttcattgattcaaggaaatttcctcaaaaaaggtaacattggagctagatattgataaatttgtttaaacaaagactttctgttttttaagctaaaagaaccaacgtatgcaaaagcagagagaaacatatgaaaagagaactgtgttttccaggaatggagaagatgagaatgtggctagagtgttaggggtgcatagttctgagtagatggatattaggcccttcaggagattgacgcctttttttttctttctttcttttcttttttttttttttgtgagacagagtttcactcttgtcgctcaagctggagtgtgatggtgtgatctcagctcaccgcaacctccacctcccaggttcaagcaattctcctgcctcagcctcccgagtagctgggattacaggcatgtgccaccacccccagctaattttgtatttttagtagagatgggatttctccatgtcggtcaggctggtctcgaactcctgacttcaggtgattcgcccgcctctgcctcccaaagcgctgggattacaggcaagattgatgtcttaatgtgaagggtcctttatgcaagctagaggttttgaggttccccctccccatcgagccttgcagttccattaaaggattttaagcaagggagtaataaaattcgttatatcattgaatgaataaaatagtcagactttttatttgtaggagaaaaattgtattagatgtagccaaatatggcttaagtaatgcagttttaaaattcagaattggaaaattggtttatctttgattaactggttcattttttttcttcctaacttctttcttctgtcagccaaaaaaaaataggaaataatggataagctagaagcagtatttgctttttaagttttgcatgactgaaacaatcccctacactgttagaagcctaacacacaatgctattgtgctctgggtctagaaaaaatgtgaggcctgctagtctgattttgcagcagggagtaaggaaagccaatacttaactttgtaagctgctaaatattaaatgtttagtaaaatggggtttatagtaatgttttttttgagacagagtctcactctgttgccaggctggagtacagtggctcagtctcggctcactgcaacctctgcctccgggttcaagcggttctccttcctcagcctcctgagtagctgggattagaggcgcctgccaccacacccagctaatttttgtatttttagtagagacggggtttcaccacgttggccaggctggtgtcaatctcctgacctggtgatccgcccgcctcagcctcccaaagtgctgggaatacaggcgttagccaccgtgcccggccagtaatattttttcttaaaaagagaatatcaaaataaagaggagagtcaggagtagagggtatcaccaagccttttttagagtcacctaatttgttacatgtttttcttaaaattaccaaccatcaaaaagaagaattttgatctatatctatgcctaagagatgtcagtagagcagtaaaaatatttctctaatttataaggaagttaagtaactttcccacagtcaaaataatcatgtcaaaaatttgacctttcaaccactagttttagtaatccaccctgcttttttcctcagggaaaaaaaaaaatgtacttcccagataaacatcaccttgcgtttcaccgcaggtctgggtgatccttaccttggaacaaatgatagtgttggtgcatttaaagtagcagcttttgggtgactggctggtcctgaggagtttttcctacttagcagtgttcattttatctattaatgccgtgaagcaaaatacaggttgaatgagcagtgttggtaactgcaaaacaaagtgttgcaacacagtaggctgaggtagttttaagcagataaagtctacagaaattagtttacagaaatagtgttaggctgggatgttttagctgctagatttcagatttagagaaatcaagtttagaaaaataaaccagcaaagtagtacaaaattaatagcaacaatgtcaggaagaatttgcttatgaaaaggatctattttcccttttgtgaagagaaacatttgttcacccttggttacttgtgattgagataattaaaaacaattttatttggttttagcagcctccttgtgaaaacttatgaaatattaggcagtttgattttcttctcctctctggacctgcagataaatttgttgtgaataaatggaaaatagtttggcagtgggcagaagcaagggaaaagccaagaatctggtcgatttataaaccagacattgtgaataaatgagttggctacagctcttagaaaaaggcttttgcctttcgagttcttttcctacatctgcttttatttctttcacactttatgaagtcaataaaaattttgatttcattgttacagaagtttctccttgtataggagcttttcatgccctctgttggccataaataatttacttttgatttctcaaggctcctaactggtgacttttcatataaggggatgtctaggttgtttttggtagtgatattatctctttgtacccagagaacacttctgacaccaaatggggggttttcttcacagcaacaaccagttcttcagctctacctggagttagcgtcagatcccataggtgaaacgctgagtcccacaagactatccccactttagatgccagtcacaagtcccaggcatcccgtagttctgactgactcaccgtaaatcagaggttcccatgaactcctcatcagatttaataatttgctagattgggtcacagaactcaggaaagcactttactgacatttgcttgtttattataaaggatactatgaagaatatagatgaacagccagataaagagagattaatacataaggtgaggtccagaagattcctgaacatgggtgctcctgtcccatggggttagggtaggccaccctccctgcacatggatgtattcaccaacttggaagctctctgatccttgtcacttagaggtttcatcatgtagacgtgatcaattattaactcagtctccaggcactctccctctccagaagttgggaggaataggtctgaaagttccatgcttctgatcattacttggtctttctggcagccacctccatcctgaggctacctagggttacgccaagagtcatttcatcaaaagaggcttctaccacccaggaagtgtcaagagatttaagaactctccgttaggaacaaaagatgctcctattatccccaccacttaggaaattacaagagttttgtttttgagacagggtctcactgtgttgcccaggctggagtgcagtggtgcaatctcagctcactgcaagctccgcctcctgggttcatgccattctcctgcctcagcctcccaagtagctgggactacagatgcccgccaccacgcccagctaatttttttgtatttttagtagagacggggtttcaccatcttagccaggatggtcttgatctcctgacctcatgatccgcccgcctcggcctcccaaagtgctgggattacaggtgtgagccaccgcacccagccggaaattacaagagatttaagtgctgtcctttgtcagaaaccagagatcaagtatatatatttttattatgtcacaaatggattctaaaatcagataatcttactattacttatctgtgaagaggacttcatactttgcacagaagtatttgctgtgacttaaacaagagtcacatggtgctatgagcttaaataattactagtaaaatttggccagagccagcagccatggcttatgtctgtaatcccagcactttgggagtccgagatgggtggatctcctgaggtcaggatttcgagaccagcctggccaacatggtgaaacaaaaaatgtctctacaaaaaatgcaaaaatgagctgggcgtggtggtgggtacctgtaatcccagctacttgggagcctgaggcaggagaatcacttgaacctgggagggagaggttgcagtgagccgagatggtgccactgcactccagcctgggtgacaaagcaagactctctcaaaacaaagaaaattgtttttggctgggtgcagtggctcatgcctgtaatcccagcactctgggaggctgaggcaggtggatcactttgaactcagtcgtttgagaccagcctggtcagtatggtgaaatcccgtctctacaaaaaaatacaaaaatgagccgggcgtggtggctcacacctgtggttccagctactcaggaggctgaggctagaggactgcttgagccaggaagtggaggttgcagtgagttgagatcgcaccattgcactccagcctggtgacagaaggatactctgtctccaaaaaaaaaaaattactattaaaattagtcttcatggtgttttggtgaactttagcttaccagctgaaaattatggaattcatttcatatagcttccactataaatactattttgatccaagcctgttaataacactaagcatggtgactctcaaccatgactgcacagtgggattgcctgggaagctttaacatacactgatacctgggtctcatatcccagagattgacttaagtggtttggggtgcaacccaggtattgcagttttctttcttaaagttccacaggtgattctaatgtagtcatggttgagagctactgaccttgagaagcatgattttttagaggtggctaacccgttctggtctactggtttacatgcagtagagatagttggggaagataatttttgcttaaaataggaaagcaactgacacaatcgcatggtaggcatgcagggtttagaacagtaagaaactgaagattataccatataacactgtgaatttattaaatacattccagagtgggttatcagccaagatgtgctagatcaactgtgtatggtgctgctaatgagttcacagaaacctagaggtttttacttgcccaaggtcacaccactagctggggacacaaccaaaactggaaaattatagtggtaattcatagtttgttgccttgattatttaggtcaagttctgtaaactttttctgtgaagggccagattgtaaacattttaggctttgcaggccatatggtccctgttgcaactactcaactctactaacatagtaaaaagcatcatacacattatgtaaatgaatgaatgtgtattctagtaaaactttattgacagaagctggatttggcctgtgacctgtaggctgcagatccctgattgaggattgttttaatcttcccattggaaagttggctccttaagagtaggctgaagatgttactggtcccacgtggtgccttacttcctggtactgttacagtggcctctgttgcaaggtgttagatgagtgcttgttgaattcatatctatgcagctcttaacgtttttctgttgcttctaacccttccagtaggggaaacagttggcacatattccagctttggagaggaaatgcttgaagtctgtagcagggtataattgctgaattcttcaagcgaattaaagatctgaatttgctctagggcattcataattattttaatttcctggaacagattctaagaattcattttaaatctgaaagtttttcttaaaatctgttttatgatacactgtctttatttttgtgaatattgttagtttctcacaacaatatttatttcagttcatttggatctaagatctttgttgtacttatgtgtggccagtagactctttagcaaaattaaacaaatgctaatgggttcccacagccacccaaatattagaagtacagctccagattatgagcctgtttctctttttcttttcttcttctcattattttaaagagatggagtctcagtatgttgcttaggctggtctcaaactcctgggctgaagccattctcccgcctcagccttccaagtagctgggattacaggcaggagccaccaccccagacatgaacccacttctaatgcaaaacacatattgacctgacaacagggccctcagttgtggtggaaagagcagtgaccttggaattaaaagccctgttgtgactcgtggctttgccacttttaaccaaccctacttttatttacctataatgtgcaggtatctgtctcactctttagggatgccaaaggtgaaatgaggtgtgctgtctatgtgacagtgccttgtgagcacttttctgacttctccctgaggaggtttgggtcaggcctagcagaaactgcagccatagagcatggagggaagtaggacttgactagcataaagtgagtctatgagagtgtgcccactggtttccaaagagaatgtcgaagagtctacaatggtgaggcctcatcagcctcgatttgagaagcaaatgtaggtcagaaatggggagcagaacaagaggaaaagaagaaggtcaggagtgtgcagagctgaagaggtggtgagaagtggttcaaagctggaaacaggcaaggtaatctggaattctgtcttgatgtctattgagttatcctaaatcaagactcatgttcctcttaccttatatttcttaaattgatttgtttgatgccctgggtattttgaagctcctagctgcataatcttgcctaataaaggcacagaattagggcaaatagctgcagtgacttgtgtacctgctcagtgaaagccaaagaatcaatagctagaatctgtgtccacaaattgcttacaaaccacatgtagatatgacattcaacatcaggcaaggctgggcacggtggctcagatctgtaattccagcactttgggaggccaaggcaggaggatcactagagttcaggagtttgagaccagcctgggcaagatgatgagactcagtctctacaaaaatgaaaaaaaaaatagctgagtgtggtggcatacacctgtagtcccacctccttgggaggctgaggtgagaggatcacttgagcccaggagtttgaggttgcagtgagatatggtcatgccactatattccagcctgtctgcatgtaaaaacaaaaaatacacacacatacacacacaccccagcctaaccacttaccacctcttaatcatctgtataatggacataaaaatgcctgctatactttacatcatagtattcttgagtggagtaaatgaaataatgcatataaggtgctttgcacaatgtgtgcttgcctcacaataaacactcaatattattagatatgatttctaccattattactaacagtagtaagaatagcagtagtatttaaaagcaagtttgcattagttttaaagcactatgaaatcccaagtcttttttaagacggagtctcactttgttgcccaggctggagtgcagtggtgcagtctctgctcactacaatctccgcctcccgggttcaagcgattctcctgcttcagcctcctgagtagctctgattataggtacccgccaccacacccagctaatttttattttttagtagagacggggttttaccatgttggccagactggtctcaaactgctgacctcaagtgatccacctgcctcggcctcccaaagtgctaggattacagggatgagccactgcacccggcctcaagttatgattgttaatatttttgtgacattcaggataaatattttttagtatgctactgaaggacatgtctcagcatcggtgtatcccttcctgaagctatatgttaccttgcatttacagagttgacttggtgtccagagaacaaatcagttgagaaaaggccattttgaaaacgattttttccagggagcatacgctagtaccctgttgcggcatccatgtgtgagggggtggttggattatagatcttcaatgtgaagtcaagtttcttatcaaatgaagtgttttgtccgatcttagagtttaaacccacaaggaggtaacaaagcaaagggctaatatatcttttcgctacttagaactacaggccagaaatttgcttttgtattctgagatcttatgatacttgtctcaatgaaataaaactgcatgcattattctgcatttccatattactattctctgtattggtaattattatgatacttgcttcctgtacttaaagcagcatgcagatttattttataatcacttcaaggaaagaaccagttcctttggagatttcctgtggttccaaagttgcatgcctccaagtctgttaggttgtgggattttgcatttgagagaaatgagggaggtggagagaatgaagagaaagtggactggcagaattacagaatgtggtttcatctgatcttttaggctgagggctactccatttgtagactccatgcataaaactcaggcatcctgggtcctgccctactgctgctttgtggtcagaacatcgtgagatcatgcagcctcaaggctgggaacatacgggatcaggtccaacagcaggttggagtttgggttcatgatttcaaagtcagggtcgtgagggcacccaagatgattaccgagaagtcaaaagcacgtgccagtaatggcaatgtgaaggtctgggaaacagccaaaacgtagctgtcaggaatgtgggacagagtccctagggcagcttttcccaaaacatgttccaagatatactagctctatgggatgttgacttacaataatatgtggtgaaaaggttttccagacaaacactgagttcagagttaagcaggtttttttctattgcagagcttgtcagagcctctgatatgcccactagtattgtaagtgtacaagatgaagctttaaaattgggcaaataacagatatttcatacagaatcctggatgtggccaggagtgatgggagatgaaggatcctcttgtcagagtagcactccacagaacacacttggggaaaagttgtcccagggcctgtggcatgtgctctgaattcattttcgcctggctcttaattctggggttcggaaggaactggcaaacacccacagaagttatctaggatcttattgagtgcttccccctcgtccttccatccgaatcacttggctgatatttgttgctgctattaacagaattcaggaaatcagcccttcttagaggaagaaggacaagtgagaccacaaacaacaaaacataatgaatggatcagagcctgtgtataattggccctgaattaggcaaataggctttccccactcccttaaaatgaaaacaaaatggtagtttaaagaaagtaccaaagggactatatctaataataacaatgagattgaaaatgagggcacatggtgtgtttagggacggctgaggccagtgaatgtcaaggcagactgcatctttgacagcctcgcctcactggctcctgaaatcctcacacacaaaacagcattcttcttgtgttcttaaagtgaaaggcactgtttccctgtcacactagcaatgttgcgaagtaaatgtcatcctttcctcctccttggccctgccttctacagctagtgcccctgccagttgtttcctcagcacgtctccagcgttcctcttctcttccccctcggcacggccttacttctggtctcatcatcctatgcctgggctgatgcctttgcttcccagctgcagtcggctggggatgaatgtggggttaaggcaggcattccagatatttatagaattgttcatggttcttttcccattagccatgagttacttgagggtgaagatagtgtcttatttgtctctctgtctctagtaattagccaagtacctggcacttttaattaaggagttatcactcagtacatttaaatagggagaaaaagaaaaaaacaaagaatggaataaagtaaaagcatgataggttttgttactctttttgcagttgatcattggctattcttaattcttatagaccagttagtactttagcagcatacagataagatatattaaaatcatacactgagtttttaaaaagtagatgggcatgagtggtggctcatgcctgtaatcccatcactttgggagggcgaggcagtaggatcgcttgagcccaagagttcgagaccagcctgggcaaaatagtgggaccccgtctctacaaaatattttttaaaaattagccgggtgtgatggtgcacctgccgtcctagctgcttgtgaggctgaagtgagaggatcacttgagcctgtaaggtcagagctgcagtgagccatgacagtgccactgcactccagcctgggcgacagagcaagacactgtctcaaaaaaaaaaaaaaaaaaaaagaaagagacaaaaagttcattagcacttaaacatgcttttcagaaaatgactaattatcaaaaactagcaattgtgatactagcaaatgaagaaacgtcaaatggagaagaaatgaaaaacgtttttagaatccaggacacacatttacaagaatgtaaaatattgtgaaaataatttggaggagaattatgattaaattatttgagacttgaattacctgcagtaaattcagctgcgtatcggttgttaggcattttatcattactaatcctgttaccatctttgttgaataagagattcatacttgatatacaaggtccatacatttgtaaataattcattattatattagtttgcaagagtcatgattttgccagaatttatccaaaatggtctattggtaatcccagagagtagcagaggcattgggcagtgaagctaacaggcggtgctaaagccagcatgagcaagtgctgggtggtaggaaaatagaactcaggcagtagtgagggcaaataggagcagacagggcagacctagaaggagacagcaagtaagaggcttaggaggctggaggagcagagactaaggaacgctcccctggcatcatgcagcattattacataagtgttagtaatttcaaatagttataactattttagatataccatgtgctagctactgttgtagtatgctcactagaatttaatacttgaatcctcacagcaacctaggtggatactattatgaatcctatgaattttcatttatagatgacaaaattgaagtacagggaaagtaagtcattcacccagctgatcatcagtaaatggaaaagccatgatttgacctcaggaaaatgagggttatttgacagggctgacttgaagaatgtttagattgctgtagaactgtatttagtgcttaacgatatataataactgatacaattggtatccaaacagccgaatgtcatgcttttagggatcaaggaaagttgcctaagtgtgcatgtgcctagcatttgagttttgatacggatgatcataccagaaaaatataccatcagttttcaggcctatctgcacagtaatatgcgtggagttcttcatgatgtattcactgatattaaagctcaaaactattatgaatcttaggctctggcgaaaaaatgtgatcgtttaaaaattcggcatttatcttatcctgtggaatgggcaacttctcaggcccatttgtcaccaccaacaaagcagtaacacgaggtgttttaataaatctgttgcccagtctcttccatattgctatgcttagctgtaaaagaagggaccaagaaatgggtgttaagctgaattggaaacaattttcagataaagcacagcagtctgcgaacctcctccagctcaagatggtgtcaactagaattgccttttgtaagcaggcagaaagagacacatgtacttgattgtggtttctctttactcccactttccactctggtcatccctgtagacacagagtaatttgtgtactgtaaatagctagatctttctgtaatctttatttgtttctttgtttgttttttgagatggagtctcacactgtcgcccaggctggagtgcagtggctcaatctcggctcactgcaacctccacctcctaggttcaagcgattctcctgcttcagcctcctgagtagctgggattacaggcgcccgccgccatgcccgactaatttttgcatttttttttttttttttttttttttgtagtagagacaatgtttcaccatgttggctaggcttctctcgaactcctgacctcaggtgatccgcctgccttggcctctcaaagtgctgggattacaggcgtgaaccaccactctcggctttaaccaacattcttagttgaacacagcagaaagggaaacctccattagtacatggaactcgggcagtagtgagggcaaatatgagggagagctgccttcctgttgcagtggtaagttcagtctctacctaagaaaacagcatgccaaggtacagcagagcacactggccagctccaatgcaagagtcatcatggagtgtaccttttttttttttgacagagtttcgctcttgttgcctaggctggagtgcaatggcgcgatcttggctcaccgcaacctccacctcctgggttcaagcgattctcctacctcagcctcttgagtagctgggattacaggcatgcgccaccacgcctggctaatttttttttttttttttttttgtatttttagtagagacagggtttctccatgttggtcaggctggtctcaaactccctacctcaggtgatccacccgcctcggcctcccaaagtgctgggattacaagcatgagccactgcacctggccggagtatgcacttttaacatgtctatgcagggacacatgtcctctaacaacaaatggccatcccgagagtggtggagcagagtgttggacctctggagcccaaatttttattgtagcaatacacagtcttcttataacaggcactgtggccatgggtcttatcagctcctattcaaaattggaattaggacaagtatagcaggttgtgttctaacagaattctaacttttaaggtgccacagacaataatattctttagaaatcttagtgttatcctttttatcctttcagggtagcaaacaaaaggcactcagtaaataattatggagtataatttggggaaaacaaaatatttctagtctgaaaattattgttttaaaaattatttttgtcttgagaggctgtacatttctgtcaactgcgtggccacattgttgatatgcttaagaaagataacagtcaaagttgcctttttacactttgtatttgtgctcatccaaccatgagacctttaaaaatattgaatatctgtagttatctcccttcaagtcttattttgagtttatattacctttgaactcaaaagatctcaagccatttgctttacctgacagtatgacattctttaattttaggtagcattgtatgatgagtctcaaatattggcagcctataatgcatataagttagtgatcctagtaaatgtaaataaaataataaacagaaacatgctgggcatggtggcttatgcctgtaattccgtaactttgggaggctgaggcaggaggattgcttcacaccaggagttagagaccagcttgggcaacagaaaccccaatttttaaaaaatcagctgaacgtggtggtgcatgcctgtagtcacagctactcaggaggctgaggtgggaggatcacttgagctcagattgaggctgcagtgagttgtgagtgcacctcagtctaggtggcagagtgaggtcctgtctcaaaaacataatgataaatgaaaataataacagaaacattcagtcaataactgggatttttttttactgttactattttttatcaagacaaacatctggagagttctacaattccagatgcttaccaatctgcttctgggttgtgaaaaaattatttgggtatgtatagcttttaactttttttttgtttgttttaactgaatattgaggattttggtagaataactttgaatgttttggattttagatttaaatggaaatatcaaattttatataggttgggctctagatatttccatattgtcagaatttagaaaacatcttttatgaaatagaaactaagaattttggcaaacagagaaagttgcccaaggtctccagaattcatttgtttgacaaatacatattgagcatctaccctgtgacaggtggtattcctgggatttgtcaggagcacaattgctgccttcctggagcttacattctaaccagggaaaactgacaataaacaataaaaaaagtaagtaaataatgtagtttgttagaaagccataggtgctacagaaaaaaataaactataaagtaggggtcggactagcaataaaactaaatagtgtggtcagggtagtccatgtggacaagatgcaatttgagccaagatttaaaaaagataaagcaattagcctcaaaataatcggggggaagaactttccagcaccaagtagtttgctcggcctgttggaggaatagcaaggagccagtgtgcctggaatggaatgcccaaggggcaatagtggccataaggtaaaagtggtaatggggctaaggccagatcctatagagcttacaaactactgcaaagaacttgagattttactctcagtagaatgtggatccattggagaattttaagcacaggatctgacttgggttttaaaagggtctctctggtcctggtttgacaatatggactgcactgggaacaagggaaggagcaggggtcttactgctgtaattcagttaaaaattatggtagtttaggctggaatagtagcaccagagatagtgctagatggtaaaatctggaaatattttgaaagtagagccaccaggatttcctggtgaataggatgtagagtatgagagaaaaagaggatttggggtgtttccaaggttttagcctgtgcataaatggagttattactattgattcagagaaaaccttaaggcagctacttttagataagagttgggtgatgaggggttcagatatgaatctgttgatagtgagatggctcttagacatactggggaagatgctggtaggcaactgcatgtgtaaatctggagacgggtgaggtctgcagtagagatttaagtgtaggataatctcaacatgtggaaggcatggaagtgtgggtgcaagtgggagacgagaaccaagcactaatccaagtgcacttcaatatttaattttaagagacaaggtctttttttttttttttttttcgagatggagtctcgctctgtcacccaggctgaagtgcagtggcgccatctcggctcactgcaagctccgcctcccggtttcacgccattctcctgcctcagcctcccgagtagctgggaccacaggcgcccaccaccacgcccggctaatttttgtatttttggtagagacggggtttcaccgtgttagccaggatggtctcgatctcctgacctcatgattcacctgcctcagcctcccaaagtgctaggattacaggcgtgagccactgcgcccagccgagacaaggtctttcactctgttacagctggagcacagtggtgcaatcacaggtcactgcaatctcaaactcctgggctcaagtaatcttcccaagtagctaagactataggtgtgcaccaccatgcccagctaatttttttttttactttttgtagagatagtgtcttgctgtgttgccgaagctagtctcaaactcttatcttaagcagtcctctctccgtggcgtccctaagtgctgggattacaggcatgagccatcatgcctggccaggcactttaattttaaagagcctttaatatggtttcttgccatgtcagagataagaacagcttaatatttcttgacttttccttggttaaacaaggggtatgctttaaaattttttttaattttaattgtggtaaaaggtacttaaaatttgccatcatagccatttttaaatgtacatttcactgtcattaaatacattcaccactatcatccacagaactcttcatcttgcaaaactgaattctgcacccactaaacaataactcccttttcttcttcctcccaggtcctcaaaaccttcattccattttctgtttctatgattttaaagtggaattatacagtatttctctttttgtaactggcttatttcacttagcattatgtatgtcctcaagattcatccatgctgtagcatatgatagaatttcctgctgggcgcagtggctcacacctgtaatcccagcactttgggaggccaaggtgggtggatcacctgaagtcaggagttcgagaccagcctggccgacgtggtgaaaccctgtctctactaaaaaatacaaaaattagctgcacatggtggtgggcgacacagcctcccaaagtgttgggattacaggtgtgagctgctgcgccaggcctgcttctactctttgactattgtgaattaatgctgctgtgaacatggatgtacaaatgtaagggatatgtttttgttttttgtttttaagatggagtctcactctgtcacctaggctggtgtgcagtggcacagtcttggctcactgcaacctccgcctcccggattcaagcaattctcccacctcagtctcctgagtagctgagattacaggtacccaccatcatgaccagctactttttgtatttttagtagaaactgggtttcaccatgtttcccaggctggtcttgaactcctgacctcaggtgatctgcccgcctcaccctcccaaagtgttgggattacaggcctgcgccacgatgcccagcagggatatgcttttttaaaagtctctgtgatatgtatacttggtgactttcatagatttttaaaaatataagtgatatataaatacatttttattataaaagatttaaatgatatagctgttaacagaccaaaatatgaaaattctcattcattctctcacctcacctccatctgcaattcccaaagttacatttggtgtgctatcttccctctctttcagtacatttctatacatttgtctataacttattctttttaaaaactgagatattatacatattctacagtttaataatgtcttggagacctctcaattatttatattttggaaatattcccagtctgctgcatatgttctatagtgttatatttacctttacatgtacagggctattactgtagattagagtcctagaagtgcatgcagttcctaggtccaaggaactttctttaactcttcatcagattttctttttgaagaacctttgtggcttgataattatgtttctagaaaatttattattcaggtttagaagtttggggagctgtttgaaataacactcaaataaatttcctggctatggatttgttccccatagcaagagagttgtaaactatgagttttataaccttaatgtcttttttttaaaaaaggcaattgatctcttaatctaggtgttttagctagcatatgggagatgtaatctgcaatctatgggacaaatgggggagttttgggagttatgatagaagactgttgaaaaatgttttataataatgttttacagtagagaaacctggtagatgccacctcatcagccgaccagggttaacatcacatgataatcatattgctatcatgcagtgatattaatataataaagtgagatgggtgcattgcctctgtgatattcttaccccaaacccataaccacagtctcataatgagaaaataacagaaacccaatttgagggacattccacaaaatacctgaccatgttcttcaaaagtgctatggtgccaaaagtcatgcaagacaaatctaagtaattctcacaggttgtagtacactaaggagacagtgaaaactaaatgcaaataaagtggtttttggttttttaaagtatgtttatccttatggacaatatgctcccataaagatcgtctaccctagaaaactgtgtcttccctcttaaatccaagagatgcttgtcccaaagggtggaccccattgagatctcctgggctagagacccagtgcatggatggatgagacagtataagcaagaccgtataaagctgtgatctgagctcatggacactgtggtaagaggtcaggctcgaagaatcagagaactgagtggattttgcaatggatagccaaagagagggtccaaacttaaaatgcgaaacaataattcaagaatgatttggtaaaatgtaatataacagcctataacagagtctagcaaatagtagccaaaagtcaccctaaccaatcgtagggcatatctaccacacaaaagtgtgacttagcgcatgactgtttaaatgccaaaaaagacagataaaggaaagttaagccatagtggcaacctctgcagttggaacgagggagctatatcagtcaacagtgaggctaattgagaatatttgaggttggcgtatgtctagggattcctataggagatacaacccagcaagcataggtcagcaacccaaggtcagagtacaaaatgggttaatactaaaaagtggttaaaaagatgaccagtcctcaagaggagatagtatttaaatcggatacctctccttccccatgctaactaacatactgtaatttgttatggctgtttctaaggtaactgtttataattataaatcagccctactggttcttcttgatattgtaatttgcaatcaaagaacctctagagttaatgtaaaacttcttgaatgagagaccatctggttacaattttggtttcaattccctgttaggcaactgacacccaaataaggactgtttccaaatatgagtaggctaagtcagtcataaacaaacaaaaagtttcagtttttgttgctattatccaagatgttagcagatgaagtgtttaatgaaagggtgcttcttttaggcaaacttcttttaccatgatttgtttaacctataccagtggtccgtacatttttttcctgtaaagggccaaataattattaatagtatttttagccttgcagccacgtatgatctctatgtatttgtcattgcctttttgtttttttaaacaaatgtttaaaaatgtaaatgttctctgtgtccttcaccatgttttcatgctttgtgatagtggctgaaatttagagacttgttttaataaacaatgttctgaaatgttgatctaattgcctctggcaaaagatgtgtaatcatgactttgatgaacttattttaaaaatagatgcacagttgatctcttattgcacgcattttggcacattcgtgggagatattctctgtaaacatgacacttgcgaaacctaccataccctgaaagtttgctgtctagaaattaagtgcctagttttcattcaaattaatatttaggtaagacatgttttatttctaaatagactgtcaaacatttttcatattttaatgtgtaatggtgatgacctttttaaaaagactcaacgtaatttgttttctcatttaacccattaactgccccctttgtgtagtattgttgctgctttctcttataaattgatgatgtctcagggtctcaatttacaattacattagtttcttcctctagtttgtagttgagctgtggctctgcactgttggggcctgtacagccatccttcactagaccccgggaggtggcctttagtaggctcaaaggtggcctttaagccattcttagtatcactgtgaaaaagcatccagattttccccctgaaaggccaagtggttgcatgatgtgttgtggattttagggatatactttgcctatttcacatacttttttccaagaagatagataagcttacacatgtaaaggtttcaactgtatttcttctgtttataattttatttattttgagatggtgtctcgctctgtcactcattttggagtgcagcgcaatggtgtgatcacggctcactgtggcctcaaactcccaggtttaagccatcctcgcacctcggccttctaagtagctgggactacagatgtatgccaccacacccagttaatgttttttgtatttttttgaagagacaggattttgccatggctgcctacctggtctcaaactcctgggcttaagtgatctgcctgcctcagcctcgcaaagtgttgagattacgtgagccactgcacccagcctttttataattttaataataatctgtcaatctttcattacttttgccttctaaattcttgttatattttactaaacaatgcttaatctcgctgataaaccattttagcagaagtatgtctgatgaggaccacccctgcctttttttttcttttctcagagttgcaaggtctccctctgttgaccaggctggagtgcagtggtgtgattatagctcactgcttcctcaacctcctgggctcaagagatcctctcacctcaacctccccagtagctgggaccacaagcgcgcaccaccatgcccagctaatttttattttttttaagtaaaggcagggtcttatgttgcccaggctggtctcaaattcctagcgtcatttaatttctgttttggtattggctctgaaagaactgttgagacaatacttggaactcttgatattttccaaaatctgttttaccaagataaattttaaggagagcaacagaaaataaagcagaaatgtggtttctttgttgggttttgttttttttttttttttttttttttttgagatggagtttcactcttgttgcccaggctggagtgcaatggtgcgatctcagctcactgcaacctccccccatcctgggttcaggtgattctcctgtctcaccctcctgagtagctaggattacaggcgcccaccaccacatctggctaatttttgtatttttagtagagacggggttttaccatgttggccaggctggtcttgaactcctgacctcaggtgatccacccgccttggcctcccagagtgctgggattacaggcatgagccaccgcacctggcccaaatgtggtttctaatcaatattatttaataatgaattggattaagaattttttgggttttttttactcaatagaaagttgattgggttttttaaaaatagcttgttttcaataattaaggcttaaaaaaatctctgagttctctttgagatatttggattctataaaaaatgaaactctttgtttcttaaccaaatgataattccttaaattgcattctttgattcaattatactgaatttcctggtagaaattttattttctctgaagcagagtcagtggtcttaatatcaccatgtcaaccagaagagaaggaaatctaatatgcataatagagaacactgacacatgacttctttaaaagtctctgaaattcaacatataactttttataccagaaagtgaacaaacatttaatccttcctcttcatctatgtctcatgatgtatttgtgtgattttaaagaacattttggagtttttgagttctatataaatgtgaccatgttatctgtataatcttgccatcctttctttggaaaacatgggtcaccgttagcgtatgtcatggatgttgatgtggagtctgagtaagctagtgggcgagagggtgtgtgtgtggacgtgggcgcatgtgtgtatttgttagtttcaaatcgagccattcttctagagtctttaggtcagctgtcacgttgggctttcctcctttgttcttttgagttgctctgtgactagccacagacccaacactaaaaaggaaggcttttaacggattaattccgtcctttccttcgcctacattgtagcagctgcgttctgccctttctgtggccctgcaaggagaggaggcctgtgtataacagtcttctgtcaacatccattataagattcctaaagcacatggaatgagatagttcccaggaaaactctttgtcaaggccagcccatgtgggaaaagtgtttctatgcttagtcttgtaactatgactgtttttgttttatggggtcaggggaaatttaacttattttatttttcttttttattgatcattcttgggtgtttctcgcagagggggatttggcagggtcataggacaatagtggagggaaggtcagcagataaacaagtgaacaaaggtctctggttttcctaggcagaggaccctgcggccttccgcagtgtttgtgtccctgggtacttgagattagggagtggtgatgattcttaacgagcatgctgccttcaagctctgtttaacaaagcacatcctgcaccgcccttaatccatttaaccctgagtggacacagcacatgtttcagagagcacagggttgggggtaaaaggtcaccgatcaacaggatcccaaggcagaagaatttttcttagtacagaacaaaatgaaaagtctcccatgtctacttctttctacacagacacggcaaccatccgacttctcaatcttttccccacctttcccccctttctattccacaaaaccgccattgtcatcccggcccgttctcaatgagctgttgggtacacctcccagacggggtggtggccgggcagaggggcttctcacatcccagtaggggcggccgggcagaggtgcccctcacctcccggacggggcggctggccgggcggggggctgacacccccacctccctcccggacggggcggctggccgggcggggggctgaccccccccacctccctcccggacgggacggctggcctggcgggggctgacccccacctccctcccggatggggtggctgccgggcggagacgctcctcacttcccagagggggtggctgccgggcagaggggctcctcacttctcagacggggcggttgccaggcggagggtctcctcacttctcagacggggcggccgggcagagacgctcctcacctcccagacggggtcgcggccgggcaggggcgctcctcacatcccagacggggtggcggggcagaggtgctccccacatctcagacgatgggcggccgggcagagacgctcctcagttcctagatgggatggcggccgggaagaggcactcctcacttcctagatgagatggcggccaggcagagacactcctcactttccagactgggcagccaggcagaggggctcctcacgtcccagacgatgggcagccgggcagagacgctcctcacttcccagacggggtggcggccgggcagaggctgcaatctcggcactttgggaggccaaggcaggcggctgggaggtggaggttgtagcgagccgcgatcacgccactgcactccagcctgggcaccattgagcactgagtgaaccagactccatctgcaatcccggcacctcgggaggccgaggctggcggatcactcgcggttaggagctggagaccagcccagccaacccagcgaaaccccgtctccaccaaaaaagtacgaaaaccaatcaggcatggcggcgcgggcatgtaatcgcaggcactcggcaggctgaggcaggagaatcaggcagggaggttgcagtgagccgagatggcagcagtacagtccagcttcggctcggcatcagagggagaccgtggaaagagagggagaccgtggaaaggggagagggagagggagggggagggggagggagagggagtgggagaggggaaatttaacttataaggcagtattgtgatatgcctccatacttacctgccctttctttctgaactcttgcagg

SEQ ID NO: 204 - DNA, 8716 bp, Homo sapiens
  misc_feature 1..8716: note = TSPAN14
  source 1..8716: mol_type = unassigned DNA
gtgagtagagcgcggagacctggctggggcgtcgggccctgcccctttctttgtctgaggccgacttccccgagcgttctccgggggcgagggctgctccttaggtccctgtttggcccctcagcctccctccccgcctgcccaggagcggccccgcgggcggcgctggctttgtctgccaggattgctcgctagaggttggaggcctccgccgggaccccgctgagccgaactccggcgccgcgcggacgagagccgcggcgggcccggggccggcactcctggttcggtagccctcgccccatcagcgcgccccggcgagtgcgcgcccgtgccgggcggggtgagggcgacgggagcctggacggccgctgctgctcggggctctgcttctcgtccccagcagcgagctcggggatgggccaccctttcggctcagtcacccaccctcggctcccaggagatcgggtccggggtgggggtgggcgtaggccggggcgggccgccccttcctcctgccccgccgtggttgtgctcagtctcccacgccgccgtgggtcccactctgcccggcttgggtttgggacggccggagtttgagtaggcggtgacccagcttctccgcgttagtcgtgtggggttgtgtttgggagcaaagcctgacccctgacctagccggagttctggtagagtgaggtttgtggctgcggactgtgcttgtcccgggccgctgctgcttctcctccacccccaggggtggggtgcgggtgggtaggaagtgcctgtgtgagggtgtgtggagggactttggccctgggaccgcaggagcacttgtcttggggacttaaagttgatctccggcgagaaacagtccgtgctagggggagtcgggggaagtcgtgggcccgggtactggcctggggctgggcgcagagcggtggtgagtgcggcggtgtcacacactcgttggctgtgctggcgctgctatccgggaggtcaggcggtgctgggcggctgtgcagagcattgtattggagcctctgcaggtgtgctcttggcagtggtctgctctccaggggatcttcctcccttccgagagggagttgctcttgggctagtgagagagtggcctgccaaatcctcaaaagtagccttccccttgccccttgaccttgaagtccagaagcagttagtgcctgctgagtgctccggccacccttctctatttgtgaagggaaacctggggacaggccatcggagcacccctatgtccttgccacatttcagctagccgagtggttggtgggggcggccgtctttccagggatcttcctgggccctggcacccccagaatgaagcccagactccaccataatggccctgtatgcccttctatccccttagctgctggtgtgatcatagcctcaaattgctgggctcaagtgatcctcccacctcagcctcccaaagcacagggattacaggtgtgagccaccacgcgcagtctgatcccactccgatccctaacgttatagtgacaggccctggaaacccagggccaccagatgtattttccctttccttctcattgtgttgcaccccaacttcagttggagactctggatatatccatccccacatctctgaactccactgcttgggccacctaatgtcatcctcagtttagttaacaaagcatggttgagatttctgtggggtgcgaggcattgttgcaggcactattccaagtctggaaagtccctcaccagctatgaacttggtgtggtcttagaacccaggctggacatggcataggtgctcagtagtgtttattgatctgagcttgatctcagcgtagcaggatcatttagggagacggggttgtggggggggtctgagcccatgggtgcttctgaaggtttgtggctgccccaggaagaaagggcatggagctgcagaatggttggctgcccagggaattgtccccttcaccagctgggcagctgtgctagtctctgaggactggtgaaggtccttctcatgtaggaagtggcagtatgaagtgggagaggagatggctctgccctgaaggttgtggcggcagtgaagtgaagttcccatggaggccggggagcaggaaagccctgggttctggaactatttcctgaggcctcactctatgccaccatgtgctcaggaatttgacccatttaacttcaactgaggtagaattccttttagcagtgcctgcaaagtggcattgtgccaggcctgggaaacctttgctggcttaaaatttagtgaaggacaagattatttcatagtgatactaatgtgagtttattgagacttacaccctagaagtttgacttgtaataactcatttaatccttgaaaacagctgtgatggctgggcgtgatggctcatgcctgtaatcccagcactttgggaggccgaggtgggtggatcacctgaggtcaggagttcaagaccagcctgaccaacacggtgaaaccctgtctctacgaaaaatacaaaattagctgggcgtggtggcacatgcctataatcccagctacttgggaggctgaggcaggagaattgcttcaatctgggaggtggaggttgcagtgagctgagatcgcgccgttgcactccagcctgggcaacaagaatgaaactccatctcaaaaaacaaaaaacaaaaagcaaaacaaccgtgatgtcaacagctccattttacagatgaggaaactgtggtcccgagaggtcacagagtaacttgcttgaagtctcaatatgagctgggttttctcatttctttttttttgatactgagtctcactctattgcccaggctggagtgcagtggcgtgatctcggctcactgcaacctccgcctcctgggttcaagtgattcttctcctgcctcagcctcccaagtagcttggactacaggtgtgcaccaccacacccagctaatttttgtatttttagtagagatggggtttcaccatgttggccaggctggtcttgaactcctgacctcaagtgatccgcccgcctcggcctcccaaagtgctgggattacaggcgtgagccactgtgccggaccaggttttctcatttctaatttagacaatatgttcctctcagaggtgattaaaatggattcatataccaaacaagcctggcccacagtggaactaatgaatgttttcagtgaaggtgcccttgtatccctgcctctgaacacatctcatctgtccagcacagcctgggcacatagcaggtaccccggaacccccgctgattggtgatggagcttctgtctccctaaggccagggaatgggaagaccagcactaccacatgcagaagattggggagtcactgcttgtgtctgtctggacagatcacggtttgctgtggtaacagcaatccccaggtctcagaggcttaaagcaacagaggtttatttcttgttcacactgcatgttcatcacttatctgctttatgttgtcctcactggggggacccatagtgattagtgggaacacccctggtggcttggacagagggacagagaacatgatgatccctgtgtcctgccactgagtcctggtggcctttaagatgcctatgggaaggggattaggttcctgctgcctgtctctgccctgccgtgtcagtccttggatatcttgcatgttccatccgctacctggatgtggcctgtaggggcagccacctttcctagaagcaggagcaggtagcgatacccaggcaggcctggtcctccccacttttgtgggactcggggattgagcttgcaggcataggggtgtagaaagcagtggtagcgaggtgactgaaggtggactcccactggggatgggattgagtgtggggctagatccctggtctgtagaagggagtaggaaccctgttagttaaattttccccctcaggccacgttagtaggattttgaagagtcagccacactgtggctcttgattttgtgcctggtcacctgcttggtctctggtgatttccctcggggccagcagacactgttggaggtggtgaggcctcaggttcttctgcttcatggttaactcactctgcttaggcatatttccaccttttccagggcctccgtttttctgccagtggattgagaggtggtggtggtctccagcagtggtctcactcttggcaggctatcagagtacccagggagctgcgtagaatgaagttttgagagtctttggtctaggtgaatgccagcgtttcagtttgcttatcttccattatagtagtgtcctctgtaactgtgaatcagggaggttattcataatatctccacatccattttctcccttaattgcgtatgcttttgatttagacagggcaagtagccattttggtggagcgcaggccctggcaggggggcagtgtccagtgccgctctcaattaaacgtgaagatgtgaagatgaataacactgtccagcttgcagagtggacggtggtgaggcaagctttcaagttgcacctactttctagttgagtgacctacacaagtctttgattttttttttttttcttcctggaaaaaaaaagttgtctacttttctcatctgtaaagtgagaagaatcaagtctgccttttctgtctcacggggttgcagtgaagcattagtaccctgagaagcaagatccaaagcccctgagttaggcctgacactggtgtgagacagctgccatctctggcggcactcagcaagtgttcaccagcagctgattctgggaacctcacttcctccgcccttgggcttggtggggttgggtagggttgggtggggctgtggttttcttttaggaggcagcaggccaggcctggagaccagagcttaagtgggcctgggcaggctggggttgaaactcttcaccccttgcggtctgtactgcctcccaactgagcagccagggagaaggcctagagcctgtgcctttcagctagatagctggaggaactggctcctccctccttaggctgtgctggcctgagctgggagcctgagagctggggcagttgtctctaaagtggcttctgggattctggtaagaggtgagctcctggtgctgcctcagagtctttgtgtttcctggcatttgggagagctggagttgggctgtcctgcatgggtaaggtttggggagggactggaacaaggggctagtgaaccttctctgggtttttcctgcctgactatgcgttgacagtcccagctgttgggcctgtgctcctgtacactgcacggccttgagaggagttcggagccctaacatccaggagagaggccccacagcagtggaaggaaatgggcctctcccgaatctcttgtttgtaccccgaggtctgagtggtgatcctggggatgctatgggactctcagcagtaggagtgtgtctgtccccagtctgggtgcccaccagctgtgctgagggtcctctcctgtgtccctgggccaggcagacagggtctttttctggggcttgttaggggaggtcagccacagccccagaccaagtaatttacacaccctgagtgaggggtgggagcagtgggtccaggaagacttagggagctggtgaaagaaaaacttaattctgacacttgttaaactggtaaggtagactgtatcaggactattacgatagatgtaggaattatcgcaatggattttgcagtagaggggagagagtgggccaactccaaatacaacaaagaaaagtgggaacttatagccaaggagcggagtgtgggggctcctgaaaatttctaagagggtagggaaatttttactaaacttagctaactgaattcttgctgcaggcaggccagggtgatcaggttttacctgggagatagtacaggatgagaaatacgttcagatatccagggtgattagatattgaggttgggggattctggttaacaggagttttgctaaaactaggctcttagagagaatggagttaggaaccctaggtcaggaccagatgagtagagggctcagaggaataaagtttggtgagggacagaatctctgtcagtatcccgtggttggcagtgtcctgtccattcttcttgcactggcctgtccctcaggagacaggtgtgagtggtcctgagcttgggccagtgtaagagtaggggacaggtttctctccttgtaggtgtcccttcttgtaagtgtcactctctttttttccagaaaccttggtccttttcagcatctgctggccccttgtcccttggctcccttgctcttggcctgctggatttcccctctgcacccaggaaatcgcatggacctgggtccttgtttccctacagcccaccaattttgctgcttttttctgccccctgaagctgagctcctgctgcaatttgtgttccctgctctccctgtcctgggcaaaccagtctgacaactttgtggttctcgctcccgcctccatcagcctggggattgactgtcccatttgtctgagctgggcagagggaggtgctgtgggggatctcttcctcttgcctggactgcacactcctgctgccttctagccggagctcctgggcattttgcctatgggagcttcaccagcttccttctgtctgaggcctacaagtccctgcctccagcctaccttgttcctcctccatgagtgaggctcctccttttctctctggccctcctgtctacttgatcagactctgcctctcttgagggccggctccctccagcctaccctgcacaggtgaccctgtttggcctgcctccttttcttgaggctgatcttgtctcaacagtcagcttttcaggaaccaggcccttgctgttgtaaggaaaacctgtcggctgtggatggggccttccctccttcctaaaggctctgtagccagcttccacccttgcagtggaacagtggtggtgccagaaccctgctctctgcagccatcctgcctaccacagtcattgtgttttgtaactctagtagcttcttgtgaaatacaagtgatggtataaacgtggataggtttttgaggggggcatgccaaaatcagagttggtggtagtggtgggggattcattcccaagggctctggggtgctaagtgtgtgagcaaagaggaacaagtggcatgtgcccaggatggggtggggcgggcaggtttagttgagggctcctctggtgtagggtagccctgacgctcccctccatggcatgactgatgaggtggcaaaggcaggtgccaggatttggtgtgttgaagattagtgcctgggttgggctctgctcactcctgcagaaagacggttggagaggggctggtcttggtttcacagaggattgtgggattacaggcaagacctgctgagggcttgcactgagccaccgagaggagcagaaagagatacgagggcttcaggtgccagtatggttctcactgttgtgagatctcatttgtgccttttttttttttccttcagacagggtctcactctgttgcccaggctggagtgcagtggccctatcacagctcactgaggcctccaccttcctggctcaagtgatcctcccgcctcagcctcctgagtagcagggaccacaggcatgcagcaccacacccagctaatttaaagaattttttgtagagattggatcttgctatgttgcctaggctggtgtcaaactcctgggcacttgatttgtgcttcttaattcaggggctacttattgaacatatttagcagtcttcagaaagcatcttattctgtaaggggaaccaaatatacccattccccctctctggccttctttccctgtggtctgggcggtctttgagcttgaggtcattaggaggtgtttatttctgtccgccctggtggcctagggcgtaggaatggggtgggatgggatgggatggggtggggtggggtggaggagttattctacatagatagagttctgtttatgcagcactccctgccatacacatctctcactcaaatggtctggccagtcctggttctaatggtcattgtgtcattcccctcagcagcaggaaaggctggaatggtttttgtattgttccttagggggaaaaatcacttggaggaagtatatcagtgcttctgagtgcagagtatttattgacacgtcagttaaaggaagcttttaaacaacatatatgtatctctttagtgaggaataaatctacaagtgcatctcacagctccattttgcagcttccatttcctggaaggtacatggagtttagtggttagggaatggggttttgagctgggattttggagaaactgtttttagcctgtccttggaattgggccaggcctagaaatctgttgaaatagctttctttagttgctgaaactgggatgtgttcatagcactaggacctcgagctccactgatgactgagtggggagggctcagagaagggtccctgtggatgcccgggatgcctgggtttcagtcccgtctcctgcatcgcctacattgcctgaatcttagggtcttacagatttatcatgaagtgggcttggcagcagatggtctggtttcttttgtgcctttgtcagaatctgttcttgctcttcttaggaaaactttctatttcag

SEQ ID NO: 205 - DNA, 3812 bp, Homo sapiens
  misc_feature 1..3812: note = B2M
  source 1..3812: mol_type = unassigned DNA
gcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcatttttttttaagaaaaacgcctgccttctgcgtgagattctccagagcaaactgggcggcatgggccctgtggtcttttcgtacagagggcttcctctttggctctttgcctggttgtttccaagatgtactgtgcctcttactttcggttttgaaaacatgagggggttgggcgtggtagcttacgcctgtaatcccagcacttagggaggccgaggcgggaggatggcttgaggtccgtagttgagaccagcctggccaacatggtgaagcctggtctctacaaaaaataataacaaaaattagccgggtgtggtggctcgtgcctgtggtcccagctgctccggtggctgaggcgggaggatctcttgagcttaggcttttgagctatcatggcgccagtgcactccagcgtgggcaacagagcgagaccctgtctctcaaaaaagaaaaaaaaaaaaaaagaaagagaaaagaaaagaaagaaagaagtgaaggtttgtcagtcaggggagctgtaaaaccattaataaagataatccaagatggttaccaagactgttgaggacgccagagatcttgagcactttctaagtacctggcaatacactaagcgcgctcaccttttcctctggcaaaacatgatcgaaagcagaatgttttgatcatgagaaaattgcatttaatttgaatacaatttatttacaacataaaggataatgtatatatcaccaccattactggtatttgctggttatgttagatgtcattttaaaaaataacaatctgatatttaaaaaaaaatcttattttgaaaatttccaaagtaatacatgccatgcatagaccatttctggaagataccacaagaaacatgtaatgatgattgcctctgaaggtctattttcctcctctgacctgtgtgtgggttttgtttttgttttactgtgggcataaattaatttttcagttaagttttggaagcttaaataactctccaaaagtcataaagccagtaactggttgagcccaaattcaaacccagcctgtctgatacttgtcctcttcttagaaaagattacagtgatgctctcacaaaatcttgccgccttccctcaaacagagagttccaggcaggatgaatctgtgctctgatccctgaggcatttaatatgttcttattattagaagctcagatgcaaagagctctcttagcttttaatgttatgaaaaaaatcaggtcttcattagattccccaatccacctcttgatggggctagtagcctttccttaatgatagggtgtttctagagagatatatctggtcaaggtggcctggtactcctccttctccccacagcctcccagacaaggaggagtagctgccttttagtgatcatgtaccctgaatataagtgtatttaaaagaattttatacacatatatttagtgtcaatctgtatatttagtagcactaacacttctcttcattttcaatgaaaaatatagagtttataatattttcttcccacttccccatggatggtctagtcatgcctctcattttggaaagtactgtttctgaaacattaggcaatatattcccaacctggctagtttacagcaatcacctgtggatgctaattaaaacgcaaatcccactgtcacatgcattactccatttgatcataatggaaagtatgttctgtcccatttgccatagtcctcacctatccctgttgtattttatcgggtccaactcaaccatttaaggtatttgccagctcttgtatgcatttaggttttgtttctttgttttttagctcatgaaattaggtacaaagtcagagaggggtctggcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcagg

SEQ ID NO: 206 - DNA, 23 bp, synthetic construct
  misc_feature 1..23: note = SS 3' albumin
  source 1..23: mol_type = other DNA
attggcgattttctttttagggc

SEQ ID NO: 207 - DNA, 20 bp, synthetic construct
  misc_feature 1..20: note = SS 3' consensus
  source 1..20: mol_type = other DNA
tttttttttttttttttcag

SEQ ID NO: 208 - skipped

SEQ ID NO: 209 - DNA, 888 bp, synthetic construct
  misc_feature 1..888: note = anHAL_S100A9
  source 1..888: mol_type = other DNA
ctgacccagcctcaccgcagtttgggttgacaagggaggatgggagtatgggctacagcaatcaaggggaagatttgagctcctggagcccagccccaagacgcagcgagtgtcctgttatacagggcaggtgctcacagttacacaggacgacagggtcaagaaattgctcaattgaacacctgctatttgtcgggccctgttctgggcagagggatgtagtggtaaatggggagcccactattccagtggagggagacacacagtaaagttgttggccaataaagagcacagataaagccaaatgccaataagtgcctggaagaaaatgagatagagtgcgctgtgggcaatggggctgggtggggtggaggtgaccagttagggtacatgagaagggcctctttgaggaggtaacatttgagctgagccccgaatgttggggagggaagcccctgaggatgacacttggcacaaagctgaggagaccctaagcctcaggcgggaacttggggtggaagacttgggggcttttctaatcctaagggtctgcggtggaaaatgaatgcataaagagcacatggagagcacctgcacagcactcagggaactgggaggtttttcccccgctccaaaaatgattaggcagttctaagaaaaaggctgagcacttccaacagcctttttgttttcttttcaaatttggggaaagtcgggaaacagaggcctgcattaagaagggtggaacacatgggtctcagtctcagttccagtcccggagccagacatcctggggtaggtccccagccctcccagtgcccctccctccgccttggtaaggtggagaattgcagccttcagagttaggggccctgacagctctccata

SEQ ID NO: 210 - DNA, 149 bp, synthetic construct
  misc_feature 1..149: note = Rewriten S100A9 ex1
  source 1..149: mol_type = other DNA
atgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaa

SEQ ID NO: 211 - DNA, 340 bp, synthetic construct
  misc_feature 1..340: note = anHAR_S100A9
  source 1..340: mol_type = other DNA
ggtggaggcctcaggcaggcaggatgctgggtggggtaggcaagaaagggcccagcagagaggccgcatggcaaaactatcctccatgtgaccccctatgcccgcttcaccccccacctgacatcccccaccagaagcaaagcgatgctgtgggaaaggaagcagagcctcatggatgggctgcacaggagagtgctcgcattggctgggtaccccacaggttctgggaggggacttagcgaggtgactcagccagagggtggcagagctggaaccaaccagtgctctcttggaccccgcctgggtctgggtcttccacctcccctgatgtctctccttt

SEQ ID NO: 212 - DNA, 895 bp, synthetic construct
  misc_feature 1..895: note = anHAL_CD11b
  source 1..895: mol_type = other DNA
aacttttgggtctgtcataaatagagggcccagaatatgtaggagtcagtctggggagaggcaaaggggatttggggaaggagaaagggttcaagaagaagcagggagaacagctagacccagacaggctggccagggaagcctggatgaatgaccacattcatggactgtgcaaggctgcttgccggtccccttgcttcacacatgaggagacggaggcccagggaggagaagtgacatggctcagggtgcgcagcaggtgtgagacccctttcctgagtgcttcctcctggatcccctctcaccatctccactttgcctccggttctattttccaaggtcccgggtgcaaatgtttgttgaatgactgatgaatgaaaatgatttgagtttgttaccttttatgcttatatgttgtggaaaatgaaattctcctcaaaagggaaggaaatacttgagagctgcataggaaggaaattatctaattaagaatgtatagaaacttcactgttgggcaaatcatcgttgtgacaccgggggaagaagccatttaggtgctcagaagggaggctggaattcagagcaggactggacgtgccccacgacggtggttcttaggtcaggagtcagcaaacagtggcctgggggcccgatatggcccacgacctgtttttgcacaacctgccagctagagattgaagatgaacactgataatcgatttgatgatagggagcaccacccccaaagaattctatttgtctcatttgtaaacccgtattacaaacaaattgtactcaatcattatgtttgaaatttccctaatgacaaatttgtggaaaagtattttctgtcttgttatataagtacttgtacaacatattctatcagcctctt

SEQ ID NO: 213 - DNA, 28 bp, synthetic construct
  misc_feature 1..28: note = Rewriten CD11b ex1
  source 1..28: mol_type = other DNA
atggctctcagagtccttctgttaacag

SEQ ID NO: 214 - DNA, 430 bp, synthetic construct
  misc_feature 1..430: note = anHAR_CD11b
  source 1..430: mol_type = other DNA
ggtctgcaaaacctaaaatttactatctggctgtttacagaataagtgtgctaatccccgccccaggctaacagagctggacctgggaggcagacatctggatgctgggttagttagggtgaccgaatggatgggaaagggaatggagcaggaagacatgctgctatcttttttttttttttttttttttgatacagggtctttctctgttgcccaggctgtagtgcagtggcatgatcatggttcactgcagccttgacctcctgggttcaagcaatcctcccacctcagcctcctgagtaccactacacccggctaattttttattttttgtagagatggggtctcactgtgttgcctaggctggtcttaaactcctgagcccaggtgatcctcccacgtcagcctcttaaattattgggataacagg

SEQ ID NO: 215 - skipped

SEQ ID NO: 216 - protein, 19 aa, synthetic construct
  REGION 1..19: note = aaP2A
ATNFSLLKQAGDVEENPGP

SEQ ID NO: 217 - protein, 18 aa, synthetic construct
  REGION 1..18: note = aaT2A
EGRGSLLTCGDVEENPGP

SEQ ID NO: 218 - protein, 239 aa, synthetic construct
  REGION 1..239: note = aaEGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 219 - protein, 925 aa, synthetic construct
  REGION 1..925: note = TALEN CD11b Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 220 - protein, 925 aa, synthetic construct
  REGION 1..925: note = TALEN CD11b Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 221 - protein, 925 aa, synthetic construct
  REGION 1..925: note = TALEN S100A9 Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 222 - protein, 925 aa, synthetic construct
  REGION 1..925: note = TALEN S100A9 Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 223 - DNA, 717 bp, synthetic construct
  misc_feature 1..717: note = polynucleotide EGFP
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 224 - DNA, 57 bp, synthetic construct
  misc_feature 1..57: note = polynucleotide P2A
  source 1..57: mol_type = other DNA
gctactaacttcagcctgctgaagcaggctggagacgtggaggagaaccctggacct

SEQ ID NO: 225 - DNA, 54 bp, synthetic construct
  misc_feature 1..54: note = polynucleotide T2A
  source 1..54: mol_type = other DNA
gagggcagaggcagcctgctgacctgcggcgacgtcgaggagaaccccgggccc

(19) United States
(12) Patent Application Publication
(10) Pub. No.: US 2026/0357297 A1
(43) Pub. Date: Nov. 12, 2026

(54) METHODS TO GENETICALLY MODIFY CELLS FOR DELIVERY OF THERAPEUTIC PROTEINS

(71) Applicant: CELLECTIS S.A., Paris (FR)

(72) Inventors: Alexandre JUILLERAT, New York, NY (US); Philippe DUCHATEAU, Draveil (FR); Patrick HONG, New York, NY (US); Laurent POIROT, Paris (FR); Brian BUSSER, New York, NY (US); Alex BOYNE, Jersey City, NJ (US)

(73) Assignee: CELLECTIS S.A., Paris (FR)

(21) Appl. No.: 19/427,517

(22) Filed: Dec. 19, 2025

Related U.S. Application Data
(63) Continuation of application No. 17/996,701, filed on May 6, 2021, now Pat. No. 12,534,744.
(60) Provisional application No. 63/020,894, filed on May 6, 2020.

(51) Int. Cl. C12N 15/90; C12N 15/86

(52) U.S. Cl. CPC C12N 15/907; C12N 15/86; C12N 2750/14143; C12N 2830/42

(57) ABSTRACT

The present disclosure provides methods to genetically modify cells by insertion of an artificial exon (ArtEx) for delivery of therapeutic proteins in specific cell types and more particularly engineered cells for expression of a transgene into the brain of a patient.

### CROSS-REFERENCE TO RELATED APPLICATIONS

This application is a continuation of U.S. application Ser. No. 17/996,701, filed Oct. 20, 2022, which is a U.S. Natl. Stage of International Appln. PCT/EP2021/062054, filed May 6, 2021, which claims the benefit of U.S. Provisional Appln. 63/020,894, filed May 6, 2020.

### SEQUENCE LISTING

The instant application contains a Sequence Listing, which has been submitted electronically in ASCII format and is hereby incorporated by reference in its entirety. Said ASCII copy, created on Apr. 18, 2023, is named 17996701_ST25_2.txt and is 338,526 bytes in size.

### FIELD OF THE INVENTION

The present invention generally relates to the field of gene therapy, and more specifically to the treatment and prevention of inherited genetic diseases. In particular, the present disclosure provides methods to genetically modify cells by insertion of an artificial exon (ArtEx) for delivery of therapeutic proteins in specific cell types and more particularly engineered cells for expression of a transgene into the brain of a patient

### BACKGROUND

Inborn errors of metabolism are a large class of monogenic disorders due to defects in genes that often code for enzymes. This genetic deficiency leads to the accumulation of un-degraded substrates in various tissues leading to variable clinical manifestations which can often affect the central nervous system. Standard of care for these diseases (if available) often involves intravenous delivery of the therapeutic protein. This improves the condition as the therapeutic protein is taken up by the affected cells which corrects the deficiency. This strategy in which gene products made in one cell (or delivered therapeutically) are taken up by affected cells to correct the deficiency is referred to as cross-correction. However, therapeutic delivery of the protein to the plasma does not resolve the neurological defects seen in these patients as the protein fails to cross the blood-brain barrier (or an inadequate amount of the protein crosses the blood-brain barrier). Thus, any therapeutic strategy for these diseases that delivers the therapeutic protein to the plasma, such as intravenous transfer or converting the liver or any other organ with gene therapy into a therapeutic protein production facility will fail to resolve the neurological symptoms of the disease.

Thus, there is a significant need for methods and therapeutic compositions for delivery to the brain to treat genetic diseases.

This background information is provided for informational purposes only. No admission is necessarily intended, nor should it be construed, that any of the preceding information constitutes prior art against the present invention.

### SUMMARY

It is to be understood that both the foregoing general description of the embodiments and the following detailed description are exemplary, and thus do not restrict the scope of the embodiments.

In one aspect, the invention provides genetically modified hematopoietic stem cells (HSC) comprising a therapeutic gene product that is co-expressed at an appropriate locus that is active in multiple hematopoietic lineages, such as macrophages, and more particularly tissue-resident microglial cells that populate the brain. The methods described herein can treat a patient through therapeutic gene product exogenous expression in myeloid lineages and tissues deriving therefrom. The invention is particularly advantageous for cross-expressing healthy alleles into the brain through by the microglial cells that populate the brain derivating from the hematopoietic lineages to obtain cross correction of deleterious alleles.

In another aspect, the invention relies on the ex vivo modification of hematopoietic stem cells (HSC) or iPS cells using programmable nucleases such as transcription activator-like effector nuclease (TALEN), zinc finger nuclease (ZFN), clustered regularly interspaced short palindromic repeats (CRISPR)-Cas, meganucleases and megaTAL (transcription activator-like (TAL) fused to a meganuclease) plus delivery of a repair template for that locus provided with recombinant adeno-associated virus (rAAV) to promote homology directed repair (HDR) of the locus. Any genetic modifications encoded in the repair template DNA can be incorporated at the target locus using this strategy including the incorporation of a therapeutic gene product such as a complementary DNA (cDNA). In some embodiments, the therapeutic gene product will be under the regulatory control of the target locus and promote expression in hematopoietic cells and in particular the microglial cells. The modified cells can subsequently be returned to the patient through adoptive cell transfer or autologous HSC transplantation. This process will deliver the therapeutic gene product systemically to treat the body but also locally in the brain to treat the totality of the symptoms of the disease.

In another aspect, the invention provides a method for expressing a transgene into the brain of a patient comprising:
i) obtaining genetically modified hematopoietic stem cells (HSC), wherein the HSC were isolated from the patient or were obtained from induced pluripotent stem (iPS) cells derived from the patient and differentiated into HSC, wherein the genetically modified HSC have been engineered to comprise a transgene integrated at a locus expressed in microglial cells; and
ii) engrafting the genetically modified HSC into the patient in order to have them differentiate into microglial cells expressing the transgene into the patient's brain.

In another aspect, the invention provides a method for expressing a transgene into the brain of a patient comprising:
i) obtaining genetically modified hematopoietic stem cells (HSC), wherein the HSC were isolated from a compatible donor or were obtained from induced pluripotent stem (iPS) cells derived from a compatible donor and differentiated into HSC, wherein the genetically modified HSC have been engineered to comprise a transgene integrated at a locus expressed in microglial cells; and
ii) engrafting the genetically modified HSC into the patient in order to have them differentiate into microglial cells expressing the transgene into the patient's brain.

In another aspect, the invention provides an isolated HSC or iPS cell which has a transgene integrated at a locus selected from TMEM119, CD11B, B2m, CX3CR1 or S100A9, said transgene being under the transcriptional control of the endogenous promoter of said genes. In some embodiments, the HSC or iPS cell is for use as a medicament. In some embodiments, the HSC or iPS cell is for use in the treatment a patient who has a deficiency in the expression of an endogenous gene homologous to the transgene (cross correction). In some embodiments, the HSC or iPS cell is for use in the treatment of a lysosomal storage disease.

In some embodiments, the locus expressed in microglial cells is selected from the group consisting of TMEM119, S100A9, CD11B, B2m, Cx3cr1, MERTK, CD164, Tlr4, Tlr7, Cd14, Fcgr1a, Fcgr3a, TBXAS1, DOK3, ABCA1, TMEM195, MR1, CSF3R, FGD4, TSPAN14, TGFBRI, CCR5, GPR34, SERPINE2, SLCO2B1, P2ry12, Olfml3, P2ry13, Hexb, Rhob, Jun, Rab3il1, Ccl2, Fcrls, Scoc, Siglech, Slc2a5, Lrrc3, Plxdc2, Usp2, Ctsf, Cttnbp2n1, Atp8a2, Lgmn, Mafb, Egr1, Bhlhe41, Hpgds, Ctsd, Hspa1a, Lag3, Csf1r, Adamts1, F11r, Golm1, Nuak1, Crybb1, Ltc4s, Sgce, Pla2g15, Ccl3l1, Abhd12, Ang, Ophn1, Sparc, Pros1, P2ry6, Lair1, Il1a, Epb41l2, Adora3, Rilpl1, Pmepa1, Ccl13, Pde3b, Scamp5, Ppp1r9a, Tjp1, Ak1, B4galt4, Gtf2 h2, Trem2, Ckb, Acp2, Pon3, Agmo, Tnfrsf17, Fscn1, St3gal6, Adap2, Ccl4, Entpd1, Tmem86a, Kctd12, Dst, Cts12, Abcc3, Pdgfb, Pald1, Tubgcp5, Rapgef5, Stab1, Lacc1, Tmc7, Nrip1, Kcnd1, Tmem206, Hps4, Dagla, Extl3, Mlph, Arhgap22, Cxxc5, P4ha1, Cysltr1, Fgd2, Kcnk13, Gbgt1, C18orf1, Cadm1, Bco2, Adrb1, C3ar1, Large, Leprel1, Liph, Upk1b, P2rx7, Slc46a1, Ebf3, Ppp1r15a, Il10ra, Rasgrp3, Fos, Tppp, Slc24a3, Havcr2, Nav2, Apbb2, Clstn1, Blnk, Gnaq, Ptprm, Frmd4a, Cd86, Tnfrsf11a, Spint1, Ppm1l, Tgfbr2, Cmklr1, Tlr6, Gas6, Hist1h2ab, Atf3, Acvr1, Abi3, Lrp12, Ttc28, Plxna4, Adamts16, Rgs1, Icam1, Snx24, Ly96, Dnajb4, and Ppfia4. In some embodiments, multiples copies of the transgene are integrated at the same locus separated by 2A self-cleaving peptide sequences.

As an independent embodiment, the present patent application presents a method for integrating an exogenous coding sequence into an endogenous intronic genomic region, which allows integration of said exogenous coding sequence preferably between the first and second endogenous exons of said genomic region. In some embodiments, this method, illustrated in FIG. 2, has the advantage to preserve stemness of HSCs and their ability to differentiate into various myeloid cells. In some embodiments, the transgene has been inserted into the HSC or iPS cell using a rare cutting endonuclease and/or a viral vector. In some embodiments, the viral vector is an AAV vector. The method, also referred to as “ArtEx” allows the insertion of an artificial Exon encoding transgenes that are placed under transcriptional control of an endogenous locus, preferably into an intronic sequence without having to inactivate the expression of the endogenous exons present at said locus.

The method more particularly comprises one or several of the steps of:
providing cell(s) comprising an endogenous intronic genomic region,
introducing into said cell(s) a polynucleotide template comprising an exogenous coding sequence, wherein said polynucleotide template comprises:
   a) a first homologous polynucleotide sequence, which is homologous to the intronic sequence upstream of the insertion site,
   b) a first strong splice site sequence, comprising a branch point and a splice acceptor;
   c) a first sequence encoding 2A self-cleaving peptide;
   d) an exogenous sequence coding for a protein of interest;
   e) a second sequence encoding 2A self-cleaving peptide;
   f) a copy of the coding sequence of the first exon;
   g) a second strong splice site sequence comprising a splice donor; and
   h) a second homologous polynucleotide sequence, which is homologous to the intronic sequence downstream of the insertion site; and optionally
inducing the integration of said exogenous polynucleotide into said intronic sequence, preferably by homologous recombination, to have said exogenous coding sequence being transcribed at said endogenous locus along with the first and preferably second exon, or a copy thereof.

This method is particularly usesful for cross correction of the expression of deficient protein in a large array of genetic disease, especially inherited diseases.

In some embodiments, the transgene is IDUA for treating Mucopolysaccharidosis Type I (Scheie, Hurler-Scheie or Hurler syndrome).

In some embodiments, the transgene is IDS for treating Mucopolysaccharidosis Type II (Hunter).

In some embodiments, the transgene is ARSB for treating Mucopolysaccharidosis Type VI (Maroteaux-Lamy).

In some embodiments, the transgene is GUSB for treating Mucopolysaccharidosis Type VII (Sly).

In some embodiments, the transgene is ABCD1 for treating X-linked Adrenoleukodystrophy.

In some embodiments, the transgene is GALC for treating Globoid Cell Leukodystrophy (Krabbe).

In some embodiments, the transgene is ARSA for treating Metachromatic Leukodystrophy.

In some embodiments, the transgene is GBA for treating Gaucher Disease.

In some embodiments, the transgene is FUCA1 for treating Fucosidosis.

In some embodiments, the transgene is MAN2B1 for treating Alpha-mannosidosis.

In some embodiments, the transgene is AGA for treating Aspartylglucosaminuria.

In some embodiments, the transgene is ASAH1 for treating Farber.

In some embodiments, the transgene is HEXA for treating Tay-Sachs.

In some embodiments, the transgene is GAA for treating Pompe.

In some embodiments, the transgene is SMPD1 for treating Niemann Pick.

In some embodiments, the transgene is LIPA for treating Wolman Syndrome.

In some embodiments, the transgene is CDKL5 for CDKL5-deficiency related disease.

Other objects, in particular vectors, cells and resulting populations of cells, as well as other features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. Schematic of ex vivo gene therapy platform to target therapeutic gene expression to the myeloid lineage including the microglia.

FIG. 2. Schematic representation of one gene editing strategy to obtain therapeutic gene expression to tissue-resident myeloid cells, including microglial cells, as per the present invention. The proposed strategy targets the intronic sequences of selected endogenous loci, among which TMEM119, MERTK, CD164, TLR7, CD14, FCGR3A (CD16), TBXAS1, DOK3, ABCA1, TMEM195, TLR4, MR1, FCGR1A (CD64), CSF3R, FGD4, TSPAN14, CXCR3, CD11B, S100A9 and B2M are preferred loci. This strategy specified in this application enables exogenous therapeutic gene insertion into the endogenous intronic sequences for transcription and expression of the therapeutic protein in the myeloid cells under the transcriptional control of the endogenous promoters present at said loci.

FIG. 3. Expression pattern of the targeted loci (TMEM119) in microglia. Human primary HSCs, were transplanted to NBSGW mice in order to differentiate to human microglial cells. CD11b marker is used as established microglial cell specific differentiation marker. A: 3 replicates of engineered cells (site directed insertion using AAV as polynucleotide template and TALE-nucleases as a rare-cutting endonuclease at the CCR5 locus). B: non-engineered primary HSCs. These experiments show that human HSCs can contribute to microglial cell turnover in the brain to serve as a vehicle for therapeutic molecular delivery to the brain as per the present invention.

FIG. 4. PCR Experiments performed on engineered HSC cells. BFP reporter gene was inserted at the CCR5 locus. A: PCR positive results showing integration of the transgene at the expected locus. B: BFP flow cytometry measurements show that genomic modification rate did not affect expression and correlated with myeloid specific differentiation (CD14 marker).

FIG. 5. Expression patterns in microglia from mice brain homogenate. CD11b marker was used as established microglial cell specific differentiation marker. A: CD11b/CCR5. B: CD11B/F4-80 was used as a control. C: CD11b/CX3CR1. D: CD11b/TMEM119. Results show that CX3CR1 and TMEM119 are either more uniformly expressed (TMEM119) or at least comparable (CX3CR1) to F4-80 expression levels and thus appear as more appropriate loci than CCR5 to express transgenes in microglia for therapeutic polypeptide delivery to the brain as per the methods of the present invention.

FIG. 6. Schematic representation of gene editing strategy to obtain specific expression of the therapeutic gene in myeloid cells by targeting intronic sequence. This strategy has a major advantage that it will avoid collateral effect (NHEJ events).

FIG. 7. Schematic representation of the experimental set up depicted in examples.

FIG. 8. A. Percentage of GFP+ cells among CD14hi HSCs after targeted integration of GFP at CD11b or S100A9 locus. B. Flow Cytometry results of CD14hi HSCs for CD11b and GFP after GFP targeted integration at CD11b locus. C. Flow Cytometry results of CD14hi HSCs for S110A9 and GFP after GFP targeted integration at S100A9 locus.

FIG. 9. IDUA quantification results after targeted integration of IDUA gene at CD11b or S100A9 locus.

FIG. 10. Percentage of chimerism characterized by the % age of human cells detected in blood (A) or in bone marrow (B) after targeted integration of GFP at S100A9 locus compared to untreated (UT) cells. Example of flow cytometry showing chimerism in the spleen by quantification of mouse and human CD45 positive cells.

FIG. 11. Percentage of targeted integration of GFP at S100A9 locus in hCD45+ or hCD45 and hCD33+ cells in Blood (A) or in Bone Marrow (B).

FIG. 12. A. Percentage of chimerism (percentage of human cells) in the brain. B. Percentage of microglial cells (P2RY12/TMEM119+ cells) within human cells detected in the brain.

FIG. 13. A. Increased expression of IDUA by HSCs edited lentivirus vector or by targeted artificial exon insertion in CD11b or S100A9 loci compared to untreated HSC (controls). B. Chimerism percentage of human cells detected in Blood, Spleen, Bone Marow after transplantation of edited HSCs. C. Detection in the brain of human cells and of human microglial cells (TMEM119+ and P2RY12+).

FIG. 14. Schematic representation of the methods of the present invention for treating diseases by ArtEx insertion in HSC or iPS in view of expression of transgenes into different hematopoietic lineages to obtain cross correction of deficient proteins in pathologic cell types. The HSCs are engineered ex-vivo and infused into the patient. By ArtEx is meant that an artificial exon comprising the sequence encoding the protein for cross correction is introduced by gene targeted insertion into an intron at an endogenous locus without altering the expression of the other exons at said locus. The locus is selected for its expression in the respective selected cell lineage or cell type (progenitor cells, blood cells, T-cells, B-cells, platelets, neutrophils, monocytes . . . ).

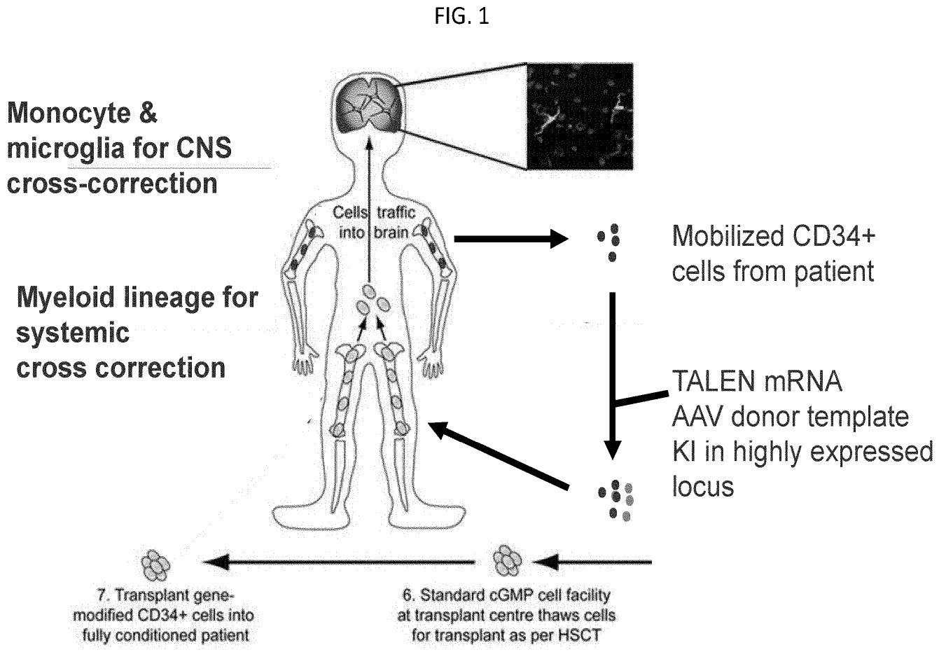

### DETAILED DESCRIPTION

Disclosed herein are methods for expressing a transgene into the brain of a patient.

The methods comprise obtaining genetically modified hematopoietic stem cells (HSC) or iPS cells (capable of differentiating into HSC) wherein the cells comprise a transgene encoding a therapeutic protein integrated into the cells at a locus expressed at least in microglial cells. The methods further comprise engrafting genetically modified HSC into a patient whereby the cells differentiate into microglial cells and express the therapeutic protein in the patient's brain. The hematopoietic stem cells or iPS cells can be derived from the patient (autologous approach) or from a donor (allogeneic approach). The invention can be regarded as a method for delivery into a patient of a therapeutic protein to correct a genetic disease or disorder, for example, a metabolic disease or lysosomal storage disease (LSD), wherein the patient expresses a defective copy of the protein. The cell is modified by targeted insertion of a transgene encoding the functional protein into a locus that is expressed at least in microglial cells, thereby treating the disease. The methods of the present invention also permits to address Central Nervous System disease, of genetic origin or not, by delivering therapeutic proteins or enzymes to the brain expressed by microglia originating from genetically engineered HSCs.

The therapeutic protein may be excreted from the microglial cell such that it is able to affect or be taken up by other cells in brain that do not harbor a functional protein corresponding to the transgene. The invention also provides methods for the production of an engineered HSC cell that produces high levels of the therapeutic where the introduction of a population of these engineered cells into a patient will supply the needed protein to treat a disease or condition.

Thus, the methods and compositions of the invention can be used to express from a transgene a therapeutically beneficial protein from a locus that is expressed in microglial cells to replace a protein that is defective in inherited metabolic disease or to deliver a therapeutic protein or enzyme to the brain. For instance, dopa-decarboxylase [EC 4.1.1.28] can be expressed the brain by microglia in order to convert L-Dopa to dopamine, which alleviates Parkinson disease's symptoms.

Additionally, the invention provides methods and compositions for treatment of these diseases by insertion of the sequences into a locus expressed in microglial cells.

In some embodiments, the transgene is introduced into HSC cells isolated from the patient or from a compatible donor. In some embodiments, the transgene is introduced into HSC derived from iPS cells or it is introduced into the iPS cells prior to their differentiation to HSC. As the HSC differentiate into the microglial cells, they will express therapeutically effective amounts of the replacement protein for delivery to cells in the brain.

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Unless specifically defined herein, all technical and scientific terms used have the same meaning as commonly understood by a skilled artisan in the fields of gene therapy, biochemistry, genetics, immunology, cancer and molecular biology. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

As used herein, the term “hematopoietic stem cells” (or “HSC”) refer to immature blood cells having the capacity to self-renew and to differentiate into mature blood cells comprising diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSC are CD34−. In addition, HSC also refer to long term repopulating HSC (LT-HSC) and short term repopulating HSC (ST-HSC). LT-HSC and ST-HSC are differentiated, based on functional potential and on cell surface marker expression. For example, in some embodiments, human HSC are a CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSC are CD34−, SCA-1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSC are CD34+, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSC are less quiescent (i.e., more active) and more proliferative than LT-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSC can be used in any of the methods described herein. In some embodiments, ST-HSC are useful because they are highly proliferative and thus, can more quickly give rise to differentiated progeny.

As used herein, a “recipient” is a patient that receives a transplant, such as a transplant containing a population of hematopoietic stem cells or a population of differentiated cells. The transplanted cells administered to a recipient may be, e.g., autologous, syngeneic, or allogeneic cells.

As used herein, a “donor” is a human or animal from which one or more cells are isolated prior to administration of the cells, or progeny thereof, into a recipient. The one or more cells may be, e.g., a population of hematopoietic stem cells to be expanded, enriched, or maintained according to the methods of the invention prior to administration of the cells or the progeny thereof into a recipient.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound, cell, or population of cells as disclosed herein into a subject by a method or route which results in at least partial delivery of the agent at a desired site. Pharmaceutical compositions comprising the compounds or cells disclosed herein can be administered by any appropriate route which results in an effective treatment in the subject.

As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

By “sequence specific reagent” is meant any active molecule that has the ability to specifically recognize a selected polynucleotide sequence from a genomic locus, preferably of at least 9 bp, more preferably of at least 10 bp and even more preferably of at least 12 pb in length, in view of modifying the expression of said genomic locus. In one embodiment, a sequence specific reagent that induces a stable mutation, is a reagent that has nickase or endonuclease activity.

The term “endonuclease” refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites.”

An “effective amount” or “therapeutically effective amount” refers to that amount of a composition described herein which, when administered to a subject (e.g., human), is sufficient to aid in treating a disease. The amount of a composition that constitutes a “therapeutically effective amount” will vary depending on the cell preparations, the condition and its severity, the manner of administration, and the age of the subject to be treated, but can be determined routinely by one of ordinary skill in the art having regard to his own knowledge and to this disclosure. When referring to an individual active ingredient or composition, administered alone, a therapeutically effective dose refers to that ingredient or composition alone. When referring to a combination, a therapeutically effective dose refers to combined amounts of the active ingredients, compositions or both that result in the therapeutic effect, whether administered serially, concurrently or simultaneously.

Endonucleases can be classified as rare-cutting endonucleases when having typically a polynucleotide recognition site greater than 10 base pairs (bp) in length. In some embodiments the rare-cutting endonuclease has a recognition site of from 14-55 bp. Rare-cutting endonucleases significantly increase homologous recombination by inducing DNA double-strand breaks (DSBs) at a defined locus thereby allowing gene repair or gene insertion therapies (Pingoud, A. and G. H. Silva (2007). Nat. Biotechnol. 25(7): 743-4).

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein.

Zinc finger and TALE binding domains can be “engineered” to bind to a predetermined nucleotide sequence, for example via engineering (altering one or more amino acids) of the recognition helix region of a naturally occurring zinc finger or TALE protein. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the endonuclease is engineered and is not found in nature. In some embodiments, the endonuclease is generated using a process such as phage display, interaction trap or hybrid selection. See e.g., U.S. Pat. Nos. 5,789,538; 5,925,523; 6,007,988; 6,013,453; 6,200,759; as well as WO 95/19431; WO 96/06166; WO 98/53057; WO 98/54311; WO 00/27878; WO 01/60970; WO 01/88197; WO 02/099084 and U.S. Patent Appl. Publication No. 2011/0301073.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This process requires nucleotide sequence homology and generally uses a “donor” molecule (also referred as “polynucleotide template”) to be integrated into the endogenous locus (“target” sequence) by homologous recombination or NHEJ repair. This leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to re-synthesize genetic information that will become part of the target, and/or related processes. Such specialized HR often results in an alteration of the sequence of the target molecule such that part or all of the sequence of the donor polynucleotide is incorporated into the target polynucleotide.

By “mutation” is intended the substitution, deletion, insertion of up to one, two, three, four, five, six, seven, eight, nine, ten, eleven, twelve, thirteen, fourteen, fifteen, twenty, twenty five, thirty, forty, fifty, or more nucleotides/amino acids in a polynucleotide (cDNA, gene) or a polypeptide sequence. In some embodiments, the mutation can affect the coding sequence of a gene or its regulatory sequence. It may also affect the structure of the genomic sequence or the structure/stability of the encoded mRNA.

By “vector” is meant a nucleic acid molecule capable of transporting another nucleic acid to which it has been linked. A “vector” in the present invention includes, but is not limited to, a viral vector, a plasmid, an oligonucleotide, a RNA vector or a linear or circular DNA or RNA molecule which may consists of a chromosomal, non-chromosomal, semisynthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e.g., adenoassociated viruses (AAV), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

As used herein, the term “locus” is the specific physical location of a DNA sequence (e.g. of a gene) into a genome. The term “locus” can refer to the specific physical location of a rare-cutting endonuclease target sequence on a chromosome or on an infection agent's genome sequence. Such a locus can comprise a target sequence that is recognized and/or cleaved by a sequence-specific endonuclease according to the invention. It is understood that the locus of interest of the present invention can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria as non-limiting examples.

The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA RNA hybrid cleavage can result in the production of either blunt ends or staggered ends.

“Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

As used herein, the terms “treat,” “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

“Expansion” in the context of cells refers to increase in the number of a characteristic cell type, or cell types, from an initial cell population of cells, which may or may not be identical. The initial cells used for expansion may not be the same as the cells generated from expansion.

“Cell population” refers to eukaryotic mammalian, preferably human, cells isolated from biological sources, for example, blood product or tissues and derived from more than one cell.

“Enriched” when used in the context of cell population refers to a cell population selected based on the presence of one or more markers, for example, CD34+.

The term “CD34+ cells” refers to cells that express at their surface CD34 marker. CD34+ cells can be detected and counted using for example flow cytometry and fluorescently labeled anti-CD34 antibodies.

“Enriched in CD34+ cells” means that a cell population has been selected based on the presence of CD34 marker. Accordingly, the percentage of CD34+ cells in the cell population after selection method is higher than the percentage of CD34+ cells in the initial cell population before selecting step based on CD34 markers. For example, CD34+ cells may represent at least 50%, 60%, 70%, 80% or at least 90% of the cells in a cell population enriched in CD34+ cells.

The term “subject” or “patient” as used herein includes all members of the animal kingdom including non-human primates and humans.

Where a numerical limit or range is stated herein, the endpoints are included. Also, all values and subranges within a numerical limit or range are specifically included as if explicitly written out.

### Therapeutic Methods

In one embodiment, the invention provides a method for expressing a transgene into the brain of a patient comprising:
i) obtaining genetically modified hematopoietic stem cells (HSC), wherein the HSC were isolated from the patient or were obtained from induced pluripotent stem (iPS) cells derived from the patient and differentiated into HSC, wherein the genetically modified HSC have been engineered to comprise a transgene integrated at a locus expressed in microglial cells; and
ii) engrafting the genetically modified HSC into the patient in order to have them differentiate into microglial cells expressing the transgene into the patient's brain.

In another embodiment, the invention provides a method for expressing a transgene into the brain of a patient comprising:
i) obtaining genetically modified hematopoietic stem cells (HSC), wherein the HSC were isolated from a compatible donor or were obtained from induced pluripotent stem (iPS) cells derived from a compatible donor and differentiated into HSC, wherein the genetically modified HSC have been engineered to comprise a transgene integrated at a locus expressed in microglial cells; and
ii) engrafting the genetically modified HSC into the patient in order to have them differentiate into microglial cells expressing the transgene into the patient's brain.

In another embodiment, the invention provides a method of treating a disease or condition in a patient comprising administering to the patient an effective amount of genetically modified HSC, wherein the genetically modified HSC have been engineered to comprise a transgene integrated at a locus expressed in microglial cells, wherein the genetically modified HSC differentiate into microglial cells in the patient and express the transgene into the patient's brain. In some embodiments, the HSC were isolated from a compatible donor or were obtained from induced pluripotent stem (iPS) cells derived from a compatible donor and differentiated into HSC. In some embodiments, the HSC were isolated from the patient or were obtained from induced pluripotent stem (iPS) cells derived from the patient and differentiated into HSC.

In some embodiments, the patient has a monogenic disease or condition. In some embodiments, the patient has a deficiency in the expression of an endogenous gene homologous to the transgene. In some embodiments, the patient has a lysosomal storage disease. In some embodiments, disease or condition is selected from Mucopolysaccharidosis Type I (Scheie, Hurler-Scheie or Hurler syndrome), Mucopolysaccharidosis Type II (Hunter syndrome), Mucopolysaccharidosis Type VI (Maroteaux-Lamy syndrome), Mucopolysaccharidosis Type VII (Sly disease), X-linked Adrenoleukodystrophy, Globoid Cell Leukodystrophy (Krabbe disease), Metachromatic Leukodystrophy, Gaucher disease, Fucosidosis, Alpha-mannosidosis, Aspartylglucosaminuria, Farber's disease, Tay-Sachs disease, Pompe disease, Niemann Pick disease and Wolman disease. In some embodiments, the patient has a Central Nervous System (CNS) disease. In some embodiments the CNS disease is selected from Alzheimer disease, Parkinson disease, Huntington's disease, multiple sclerosis disease. In some embodiments, the patient has a CDKL5-deficiency related disease. In some embodiments the CDKL5-deficiency disease is selected from Early infantile epileptic encephalopathy (EIEE), Atypical Rett syndrome, CDKL5-related epileptic encephalopathy, and West syndrome.

The methods can be part of an autologous or part of an allogenic treatment. By autologous, it is meant that cells used for treating patients are originating from said patient. By allogeneic is meant that the cells or population of cells used for treating patients are not originating from said patient but from a donor.

In some embodiments, the cells are administrated to patients undergoing an immunosuppressive treatment. In one embodiment, the administered cells have been made resistant to at least one immunosuppressive agent. In some embodiments, the immunosuppressive treatment helps the selection and expansion of the genetically modified HSC within the patient.

The administration of the cells may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions are administered by intravenous injection, where there are capable of migrating to the bone marrow.

While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, the administration of the cells or population of cells comprises administration of about 10₄-10⁹ cells per kg body weight. In some embodiments, about 10⁵ to 10⁶ cells/kg body weight are administered. All integer values of cell numbers within those ranges are contemplated.

The cells can be administrated in one or more doses. In another embodiment, am effective amount of cells are administrated as a single dose. In another embodiment, an effective amount of cells are administrated as more than one dose over a period of time. Timing of administration is within the judgment of managing physician and depends on the clinical condition of the patient.

In some embodiments, administering genetically modified HSC cells can include treating the patient with a myeloablative and/or immune suppressive regimen to deplete host bone marrow stem cells and prevent rejection. In some embodiments, the patient is administered chemotherapy and/or radiation therapy. In some embodiments, the patient is administered a reduced dose chemotherapy regimen. In some embodiments, reduced dose chemotherapy regimen with busulfan at 25% of standard dose can be sufficient to achieve significant engraftment of modified cells while reducing conditioning-related toxicity (Aiuti A. et al. (2013), Science 23; 341 (6148)). A stronger chemotherapy regimen can be based on administration of both busulfan and fludarabine as depleting agents for endogenous HSC. In some embodiments, the dose of busulfan and fludarabine are approximately 50% and 30% of the ones employed in standard allogeneic transplantation. In another embodiment, the cells are administered following B-cell ablative therapy such as agents that react with CD20, e.g., Rituxan. In some embodiments, the patient is administered chemotherapy agents such as fludarabine, external-beam radiation therapy (XRT), cyclophosphamide, or antibodies such as OKT3 or CAMPATH.

In certain embodiments, the genetically modified cells are administered to the subject as combination therapy comprising immunosuppressive agents. Exemplary immunosuppressive agents include sirolimus, tacrolimus, cyclosporine, mycophenolate, anti-thymocyte globulin, corticosteroids, calcineurin inhibitor, anti-metabolite, such as methotrexate, post-transplant cyclophosphamide or any combination thereof. In some embodiments, the subject is pretreated with only sirolimus or tacrolimus as prophylaxis against GVHD. In some embodiments, the cells are administered to the subject before an immunosuppressive agent. In some embodiments, the cells are administered to the subject after an immunosuppressive agent. In some embodiments, the cells are administered to the subject concurrently with an immunosuppressive agent. In some embodiments, the cells are administered to the subject without an immunosuppressive agent. In some embodiments, the patient receiving genetically modified cells receives immunosuppressive agent for less than 6 months, 5 months, 4 months, 3 months, 2 months, 1 month, 3 weeks, 2 weeks, or 1 week.

### Transgenes and Diseases

The transgene as used herein encodes a therapeutic protein of a disease associated gene. A disease associated gene is one that is defective in some manner in a disease. In some embodiments, the disease to be treated and transgene are shown below in Table 1.

TABLE 1
Monogenic diseases and transgenes for their treatment.

| | | Nucleotide | Amino acid |
|---|---|---|---|
| Disease | Transgene | sequence | sequence |
| Mucopolysaccharidosis | IDUA | SEQ ID NO: 1 | SEQ ID NO: 2 |
| Type I (Scheie, Hurler- | | | |
| Scheie or Hurler | | | |
| syndrome) | | | |
| Mucopolysaccharidosis | IDS | SEQ ID NO: 3 | SEQ ID NO: 4 |
| Type II (Hunter | | | |
| syndrome) | | | |
| Mucopolysaccharidosis | ARSB | SEQ ID NO: 5 | SEQ ID NO: 6 |
| Type VI (Maroteaux- | | | |
| Lamy syndrome) | | | |
| Mucopolysaccharidosis | GUSB | SEQ ID NO: 7 | SEQ ID NO: 8 |
| Type VII (Sly disease) | | | |
| X-linked | ABCD1 | SEQ ID NO: 9 | SEQ ID NO: 10 |
| Adrenoleukodystrophy | | | |
| Globoid Cell | GALC | SEQ ID NO: 11 | SEQ ID NO: 12 |
| Leukodystrophy | | | |
| (Krabbe disease) | | | |
| Metachromatic | ARSA | SEQ ID NO: 13 | SEQ ID NO: 14 |
| Leukodystrophy | | | |
| Metachromatic | PSAP | SEQ ID NO: 15 | SEQ ID NO: 16 |
| Leukodystrophy | | | |
| Gaucher disease | GBA | SEQ ID NO: 17 | SEQ ID NO: 18 |
| Fucosidosis | FUCA1 | SEQ ID NO: 19 | SEQ ID NO: 20 |
| Alpha-mannosidosis | MAN2B1 | SEQ ID NO: 21 | SEQ ID NO: 22 |
| Aspartylglucosaminuria | AGA | SEQ ID NO: 23 | SEQ ID NO: 24 |
| Farber's disease | ASAH1 | SEQ ID NO: 25 | SEQ ID NO: 26 |
| Tay-Sachs disease | HEXA | SEQ ID NO: 27 | SEQ ID NO: 28 |
| Pompe disease | GAA | SEQ ID NO: 29 | SEQ ID NO: 30 |
| Niemann Pick disease | SMPD1 | SEQ ID NO: 31 | SEQ ID NO: 32 |
| Wolman disease | LIPA | SEQ ID NO: 33 | SEQ ID NO: 34 |
| CDKL5-deficiency | CDKL5 | SEQ ID NO: 35 | SEQ ID NO: 36 |
| related diseases | | | |
| (e.g., Early infantile | | | |
| epileptic | | | |
| encephalopathy (EIEE) | | | |
| disease, Atypical Rett | | | |
| syndrome, CDKL5- | | | |
| related epileptic | | | |
| encephalopathy disease, | | | |
| or West syndrome | | | |
| disease) | | | |

In some embodiments, the transgene comprises a coding sequence of a gene selected from IDUA, IDS, ARSB, GUSB, ABCD1, GALC, ARSA, PSAP, GBA, FUCA1, MAN2B1, AGA, ASAH1, HEXA, GAA, SMPD1, LIPA and CDKL5.

In some embodiments, the nucleotide sequence of IDUA comprises SEQ ID NO:1 and the amino acid sequence comprises SEQ ID NO:2.

In some embodiments, the nucleotide sequence of IDS comprises SEQ ID NO:3 and the amino acid sequence comprises SEQ ID NO:4.

In some embodiments, the nucleotide sequence of ARSB comprises SEQ ID NO:5 and the amino acid sequence comprises SEQ ID NO:6.

In some embodiments, the nucleotide sequence of GUSB comprises SEQ ID NO:7 and the amino acid sequence comprises SEQ ID NO:8.

In some embodiments, the nucleotide sequence of ABCD1 comprises SEQ ID NO:9 and the amino acid sequence comprises SEQ ID NO:10.

In some embodiments, the nucleotide sequence of GALC comprises SEQ ID NO:11 and the amino acid sequence comprises SEQ ID NO: 12.

In some embodiments, the nucleotide sequence of ARSA comprises SEQ ID NO:13 and the amino acid sequence comprises SEQ ID NO: 14.

In some embodiments, the nucleotide sequence of PSAP comprises SEQ ID NO: 15 and the amino acid sequence comprises SEQ ID NO:16.

In some embodiments, the nucleotide sequence of GBA comprises SEQ ID NO: 17 and the amino acid sequence comprises SEQ ID NO:18.

In some embodiments, the nucleotide sequence of FUCA1 comprises SEQ ID NO: 19 and the amino acid sequence comprises SEQ ID NO:20.

In some embodiments, the nucleotide sequence of MAN2B1 comprises SEQ ID NO:21 and the amino acid sequence comprises SEQ ID NO:22.

In some embodiments, the nucleotide sequence of AGA comprises SEQ ID NO:23 and the amino acid sequence comprises SEQ ID NO:24.

In some embodiments, the nucleotide sequence of ASAH1 comprises SEQ ID NO:25 and the amino acid sequence comprises SEQ ID NO:26.

In some embodiments, the nucleotide sequence of HEXA comprises SEQ ID NO:27 and the amino acid sequence comprises SEQ ID NO:28.

In some embodiments, the nucleotide sequence of GAA comprises SEQ ID NO:29 and the amino acid sequence comprises SEQ ID NO:30.

In some embodiments, the nucleotide sequence of SMPD1 comprises SEQ ID NO:31 and the amino acid sequence comprises SEQ ID NO:32.

In some embodiments, the nucleotide sequence of LIPA comprises SEQ ID NO:33 and the amino acid sequence comprises SEQ ID NO:34.

In some embodiments, the nucleotide sequence of CDKL5 comprises SEQ ID NO:35 and the amino acid sequence comprises SEQ ID NO:36.

In some embodiments, the transgene comprises one or more copies of a nucleotide sequence selected from any one of SEQ ID NOS: 1, 3, 5, 7, 9, 11, 13, 15, 17, 19, 21, 23, 25, 27, 29, 31, 33 and 35.

In some embodiments, the transgene comprises one or more copies of a nucleotide sequence encoding an amino acid sequence selected from any one of SEQ ID NOS: 2, 4, 6, 8, 10, 12, 14, 16, 18, 20, 22, 24, 26, 28, 30, 32, 34 and 36.

In some embodiments, the transgene comprises a nucleotide sequence encoding a therapeutic protein that is a variant of any one of SEQ ID NOS: 2, 4, 6, 8, 10, 12, 14, 16, 18, 20, 22, 24, 26, 28, 30, 32, 34 and 36.

A particular nucleotide sequence encoding a therapeutic protein may be identical over its entire length to the coding sequence in SEQ ID NOS: 1, 3, 5, 7, 9, 11, 13, 15, 17, 19, 21, 23, 25, 27, 29, 31, 33 or 35. Alternatively, a particular nucleotide sequence encoding a therapeutic protein may be an alternate form of SEQ ID NOS: 1, 3, 5, 7, 9, 11, 13, 15, 17, 19, 21, 23, 25, 27, 29, 31, 33 or 35 due to degeneracy in the genetic code or variation in codon usage encoding the polypeptides of SEQ ID NOS: 2, 4, 6, 8, 10, 12, 14, 16, 18, 20, 22, 24, 26, 28, 30, 32, 34 and 36. In some embodiments, the transgene comprises a nucleotide sequence that is highly identical, at least 90% identical, with a nucleotide sequence encoding a therapeutic protein or at least 90% identical with the encoding nucleotide sequence set forth in SEQ ID NOS: 1, 3, 5, 7, 9, 11, 13, 15, 17, 19, 21, 23, 25, 27, 29, 31, 33 or 35. In some embodiments, the transgene comprises a nucleotide sequence that is at least 90%, 91%, 92%, 93%, 94%, 95%, 96%, 97%, 98%, or 99% identical to the nucleotide sequence set forth in SEQ ID NOS: 1, 3, 5, 7, 9, 11, 13, 15, 17, 19, 21, 23, 25, 27, 29, 31, 33 or 35.

When a transgene comprising a polynucleotide encoding the therapeutic proteins of the invention is used for the recombinant production of a therapeutic protein, the polynucleotide may include the coding sequence for the full-length polypeptide or a fragment thereof, by itself; the coding sequence for the full-length polypeptide or fragment in reading frame with other coding sequences, such as those encoding a leader or secretory sequence, a pre-, or pro or prepro-protein sequence, or other fusion peptide portions. The polynucleotide may also contain non-coding 5′ and 3′ sequences, such as transcribed, non-translated sequences, splicing and polyadenylation signals, ribosome binding sites and sequences that stabilize mRNA.

In some embodiments the therapeutic protein can further comprises secretory signal peptides allowing its secretion by the gene edited cells of the present invention. Some Examples of such signal peptides are listed in Table 2 below:

TABLE 2
Examples of useful signal peptides

| SEQ | | |
|---|---|---|
| ID | | |
| NO: # | Origin of the peptide | Polypeptide sequence |
| 37 | Human albumin peptide | MKWVTFISLLFLFSSAYS |
| 38 | Human | MAFLWLLSCWALLGTTFG |
| | chymotrypsinogen | |
| | peptide | |
| 39 | Human interleukin-2 | MQLLSCIALILALV |
| | peptide | |
| 40 | Human trypsinogen-2 | MNLLLILTFVAAAVA |
| | peptide | |
| 41 | Human BM40 peptide | MRAWIFFLLCLAGRALA |
| 42 | Secrecon | MWWRLWWLLLLLLLLWPMVWA |
| 43 | Mouse IgK VIII | METDTLLLWVLLLWVPGSTG |
| 44 | Human IgK VIII | MDMRVPAQLLGLLLLWLRGARC |
| 45 | CD33 | MPLLLLLPLLWAGALA |
| 46 | tPA | MDAMKRGLCCVLLLCGAVFVSPS |
| 47 | Consensus | MLLLLLLLLLLALALA |
| 48 | Native | MLLLLLLLGLRLQLSLG |

In some embodiments the therapeutic protein can further comprise peptide allowing cell uptake, such as cell penetrating peptides (CPP) and Apolipoproteins. Examples of cell penetrating peptides and Apolipoproteins are listed in Table 3 below.

TABLE 3
Examples of useful CPP and Apolipoproteins

| SEQ | | |
|---|---|---|
| ID | Origin of the | |
| NO: # | polypeptide | Polypeptide sequence |
| 49 | Penetratin | RQIKIWFQNRRMKWKK |
| 50 | TAT | YGRKKRRQRRR |
| 51 | SynB1 | RGGRLSYSRRRFSTSTGR |
| 52 | SynB3 | RRLSYSRRRF |
| 53 | PTD-4 | PIRRRKKLRRLK |
| 54 | PTD-5 | RRQRRTSKLMKR |
| 55 | FHV Coat | RRRRNRTRRNRRRVR |
| 56 | BMV Gag | KMTRAQRRAAARRNRWTAR |
| 57 | HTLV-II | TRRQRTRRARRNR |
| | Rex | |
| 58 | D-Tat | GRKKRRQRRRPPQ |
| 59 | R9-Tat | GRRRRRRRRRPPQ |
| 60 | Transportan | GWTLNSAGYLLGKINLKALAALAKKIL |
| 61 | MAP | KLALKLALKLALALKLA |
| 62 | SBP | MGLGLHLLVLAAALQGAWSQPKKKRKV |
| 63 | FBP | GALFLGWLGAAGSTMGAWSQPKKKRKV |
| 64 | MPG ac | GALFLGFLGAAGSTMGAWSQPKKKRKV |
| 65 | MPG(ΔNLS) | GALFLGFLGAAGSTMGAWSQPKSKRKV |
| 66 | Pep-1 | KETWWETWWTEWSQPKKKRKV |
| 67 | Pep-2 | KETWFETWFTEWSQPKKKRKV |
| 68 | ApoE p1 | LRKLRKRLLLRKLRKRLL |
| 69 | ApoE p2 | LRKLRKRLLRDADDLLRKLRKRLLRDADDL |
| 70 | ApoE p3 | LRVRLASHLRKLRKRLL |
| 71 | ApoE p4 | TEELRVRLASHLRKLRKRLL |
| 72 | ApoE p5 | LRVRLASHLRKLRKRLLLRVRLASHLRKLR |
| | | KRLL |
| 73 | ApoE p6 | TEELRVRLASHLRKLRKRLLTEELRVRLAS |
| | | HLRKLRKRLL |
| 74 | Myc Peptide | EQKLISEEDL |
| 75 | ApoB | SSVIDALQYKLEGTTRLTRKRGLKLATALS |
| | Peptide | LSNKFVEGS |

In some embodiments, the transgene comprises a polynucleotide having a nucleotide sequence at least 90% identical, and more preferably at least 91%, 92%, 93%, 94%, 95%, 96%, 97%, 98% or 99% identical to a nucleotide sequence encoding a therapeutic protein having the amino acid sequence in SEQ ID NOS: 2, 4, 6, 8, 10, 12, 14, 16, 18, 20, 22, 24, 26, 28, 30, 32, 34 and 36.

Conventional means utilizing known computer programs such as the BestFit program (Wisconsin Sequence Analysis Package, Version 10 for Unix, Genetics Computer Group. University Research Park, 575 Science Drive, Madison, Wis. 53711) may be utilized to determine if a particular nucleic acid molecule is at least 90%, 91%, 92%, 93%, 94%, 95%, 96%, 97%, 98% or 99% identical to any one of the nucleotide sequences shown in SEQ ID NOS: 1, 3, 5, 7, 9, 11, 13, 15, 17, 19, 21, 23, 25, 27, 29, 31, 33 or 35.

In some embodiments, the transgene comprises a polynucleotide encoding a therapeutic protein that has an amino acid sequence of the therapeutic protein of SEQ ID NOS: 2, 4, 6, 8, 10, 12, 14, 16, 18, 20, 22, 24, 26, 28, 30, 32, or 34, in which several, 1,1-2, 1-3, 1-5, 5-10, or 10-20 amino acid residues are substituted, deleted or added, in any combination.

In some embodiments, the transgene comprises a polynucleotide that is at least 90%, 91%, 92%, 93%, 94%, 95%, 96%, 97%, 98% or 99% identical over their entire length to a polynucleotide encoding a therapeutic protein having the amino acid sequence set out in SEQ ID NOS: 2, 4, 6, 8, 10, 12, 14, 16, 18, 20, 22, 24, 26, 28, 30, 32, 34 or 36.

In some embodiments, the therapeutic protein expressed by the transgene is identical to a wild-type amino acid sequence of the protein, e.g., any of SEQ ID NOS: 2, 4, 6, 8, 10, 12, 14, 16, 18, 20, 22, 24, 26, 28, 30, 32, 34 or 36.

In some embodiments, the therapeutic protein expressed by the transgene is a functional fragment or variant of any of SEQ ID NOS: 2, 4, 6, 8, 10, 12, 14, 16, 18, 20, 22, 24, 26, 28, 30, 32, 34 or 36.

In some embodiments, the therapeutic protein comprises the polypeptide of SEQ ID NOS: 2, 4, 6, 8, 10, 12, 14, 16, 18, 20, 22, 24, 26, 28, 30, 32, 34 or 36, as well as polypeptides and fragments which have activity and comprise at least 90% identity to the polypeptide of SEQ ID NOS: 2, 4, 6, 8, 10, 12, 14, 16, 18, 20, 22, 24, 26, 28, 30, 32, 34 or 36, or the relevant portion and more preferably at least 96%, 97% or 98% identity to the polypeptide of SEQ ID NOS: 2, 4, 6, 8, 10, 12, 14, 16, 18, 20, 22, 24, 26, 28, 30, 32, 34 or 36, and still more preferably at least 91%, 92%, 93%, 94%, 95%, 96%, 97%, 98%, 99%, or 100% identity to the polypeptide of SEQ ID NOS: 2, 4, 6, 8, 10, 12, 14, 16, 18, 20, 22, 24, 26, 28, 30, 32, 34 or 36.

The therapeutic protein may be a part of a larger protein such as a fusion protein. It is often advantageous to include additional amino acid sequence which contains secretory or leader sequences, pro-sequences, or other sequences which may aid in stability.

In some embodiments, the transgene encodes a biologically active fragment of any of SEQ ID NOS: 2, 4, 6, 8, 10, 12, 14, 16, 18, 20, 22, 24, 26, 28, 30, 32, or 34. A fragment is a polypeptide having an amino acid sequence that entirely is the same as part but not all of the amino acid sequence of one of the aforementioned therapeutic protein. As with the full length therapeutic proteins, fragments may be “free-standing,” or comprised within a larger polypeptide of which they form a part or region, most preferably as a single continuous region. In some embodiments, a fragment can constitute from about 10 contiguous amino acids identified in SEQ ID NOS: 2, 4, 6, 8, 10, 12, 14, 16, 18, 20, 22, 24, 26, 28, 30, 32, 34 or 36.

In some embodiments, fragments include, for example, truncation polypeptides having the amino acid sequence of the therapeutic protein, except for deletion of a continuous series of residues that includes the amino terminus, or a continuous series of residues that includes the carboxyl terminus or deletion of two continuous series of residues, one including the amino terminus and one including the carboxyl terminus. Also preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Functional fragments are those that mediate protein activity of the wild type protein, including those with a similar activity or an improved activity.

In some embodiments, the fragments can lack from 1-20 amino acids (i.e., 1, 2, 3, 4, 5, 6, 7, 8, 9, 10, 11, 12, 13, 14, 15, 16, 17, 18, 19, or 20 amino acids) of the N-terminus and/or C-terminus of any of SEQ ID NOS: 2, 4, 6, 8, 10, 12, 14, 16, 18, 20, 22, 24, 26, 28, 30, 32, 34 or 36.

In some embodiments, the transgene encodes a polypeptide having an amino acid sequence at least 90% identical to that of SEQ ID NOS: 2, 4, 6, 8, 10, 12, 14, 16, 18, 20, 22, 24, 26, 28, 30, 32, 34 or 36, or functional fragments thereof with at least 90% identity to the corresponding fragment of SEQ ID NOS: 2, 4, 6, 8, 10, 12, 14, 16, 18, 20, 22, 24, 26, 28, 30, 32, 34 or 36, all of which retain the biological activity of the therapeutic protein. Included in this group are variants of the defined sequence and fragment. In some embodiments, variants are those that vary from the reference sequence by conservative amino acid substitutions, i.e. those that substitute a residue with another of like characteristics. Typical substitutions are among Ala, Val, Leu and Ile; among Ser and Thr; among the acidic residues Asp and Glu; among Asn and Gln; and among the basic residues Lys and Arg, or aromatic residues Phe and Tyr. In some embodiments, the transgene encodes a polypeptide variants in which 1-20 amino acids are substituted, deleted, or added in any combination.

### CDKL5-Deficiency Related Diseases:

### Early Infantile Epileptic Encephalopathy (EIEE) Disease

Early Infantile Epileptic Encephalopathy (EIEE) is a neurological disorder characterized by seizures. The disorder affects newborns, usually within the first three months of life (most often within the first 10 days) in the form of epileptic seizures. Infants have primarily tonic seizures (which cause stiffening of muscles of the body, generally those in the back, legs, and arms), but may also experience partial seizures, and rarely, myoclonic seizures (which cause jerks or twitches of the upper body, arms, or legs). Episodes may occur more than a hundred times per day. Most infants with the disorder show underdevelopment of part or all of the cerebral hemispheres or structural anomalies. Some cases are caused by metabolic disorders or by mutations in several different genes. The cause for many cases can't be determined. There are several types of early infantile epileptic encephalopathy. The EEGs reveal a characteristic pattern of high voltage spike wave discharge followed by little activity. This pattern is known as “burst suppression.” The seizures associated with this disease are difficult to treat and the syndrome is severely progressive. Some children with this condition go on to develop other epileptic disorders such as West syndrome and Lennox-Gestaut syndrome.

EIEE may be the result of different etiologies. Many cases have been associated with structural brain abnormalities. Some cases are due to metabolic disorders (cytochrome C oxidase deficiency, carnitine palmitoyl transferase II deficiency) or brain malformations (such as porencephaly, or hemimegalencephaly) that may or not be genetic in origin. Genetic variants of EIEE have been associated with mutations in certain genes such as ARX (Xp22.13), CDKL5 (Xp22), SL25A22 (11p15.5) and STXBP1 (9q34.1), among others. The genetic abnormalities are thought to lead to EIEE as they are related to neuronal dysfunction or brain dysgenesis.

### Atypical Rett Syndrome

Atypical Rett syndrome is a neurodevelopmental disorder that is diagnosed when a child has some of the symptoms of Rett syndrome but does not meet all the diagnostic criteria. Like the classic form of Rett syndrome, atypical Rett syndrome mostly affects girls. Children with atypical Rett syndrome can have symptoms that are either milder or more severe than those seen in Rett syndrome. Several subtypes of atypical Rett syndrome have been defined. The early-onset seizure type is characterized by seizures in the first months of life with later development of Rett features (including developmental problems, loss of language skills, and repeated hand wringing or hand washing movements). It is frequently caused by mutations in the X-linked CDKL5 gene (Xp22).

### CDKL5-Related Epileptic Encephalopathy Disease

CDKL5-related epileptic encephalopathy is characterized by a 3-stage evolution consisting of early epilepsy (stage 1), then infantile spasms (stage 2) and, finally, multifocal and refractory myoclonic epilepsy (stage 3). See, e.g., Bahi-Buisson et al. Epilepsia. 49:1027-1037 (2008). Genetic abnormalities of cyclin-dependent kinase-like 5 (CDKL5) cause an early-onset epileptic encephalopathy.

### West Syndrome Disease

West syndrome is a type of epilepsy characterized by spasms, abnormal brain wave patterns called hypsarrhythmia and sometimes intellectual disability. The spasms that occur may range from violent jackknife or “salaam” movements where the whole body bends in half, or they may be no more than a mild twitching of the shoulder or eye changes. These spasms usually begin in the early months after birth and can sometimes be helped with medication. There are many different causes of West syndrome and if a specific cause can be identified, a diagnosis of symptomatic West syndrome can be made. If a cause cannot be determined, a diagnosis of cryptogenic West syndrome is made. A specific cause for West syndrome can be identified in approximately 70-75% of those affected. X-linked West syndrome (X-linked infantile spasm syndrome or ISSX) can be caused by a mutation in the CDKL5 gene or the ARX gene on the X chromosome.

### Mucopolysaccharidoses

Mucopolysaccharidoses (MPSs) are degenerative genetic diseases linked to an enzymatic defect. In particular, MPSs are caused by the deficiency or the inactivity of lysosomal enzymes which catalyze the gradual metabolism of complex sugar molecules called glycosaminoglycans (GAGs). These enzymatic deficiencies cause an accumulation of GAGs in the cells, the tissues and, in particular, the cell lysosomes of affected subjects, leading to permanent and progressive cell damage which affects the appearance, the physical capacities, the organ function and, in most cases, the mental development of affected subjects.

Eleven distinct enzymatic defects have been identified, corresponding to seven distinct clinical categories of MPS. Each MPS is characterized by a deficiency or inactivity of one or more enzymes which degrade mucopolysaccharides, namely heparan sulfate, dermatan sulfate, chondroitin sulfate and keratan sulfate.

MPS I is divided into three subtypes based on severity of symptoms. All three types result from an absence of, or insufficient levels of, the enzyme alpha-L-iduronidase (IDUA). Children born to an MPS I parent carry the defective gene.

MPS I H (also called Hurler syndrome or alpha-L-iduronidase deficiency), is the most severe of the MPS I subtypes. Developmental delay is evident by the end of the first year, and patients usually stop developing between ages 2 and 4. This is followed by progressive mental decline and loss of physical skills. Language may be limited due to hearing loss and an enlarged tongue. In time, the clear layers of the cornea become clouded and retinas may begin to degenerate. Carpal tunnel syndrome (or similar compression of nerves elsewhere in the body) and restricted joint movement are common. Affected children may be quite large at birth and appear normal but may have inguinal (in the groin) or umbilical (where the umbilical cord passes through the abdomen) hernias. Growth in height may be faster than normal but begins to slow before the end of the first year and often ends around age 3. Many children develop a short body trunk and a maximum stature of less than 4 feet. Distinct facial features (including flat face, depressed nasal bridge, and bulging forehead) become more evident in the second year. By age 2, the ribs have widened and are oar-shaped. The liver, spleen, and heart are often enlarged. Children may experience noisy breathing and recurring upper respiratory tract and ear infections. Feeding may be difficult for some children, and many experience periodic bowel problems. Children with Hurler syndrome often die before age 10 from obstructive airway disease, respiratory infections, and cardiac complications.

MPS I S, Scheie syndrome, is the mildest form of MPS 1. Symptoms generally begin to appear after age 5, with diagnosis most commonly made after age 10. Children with Scheie syndrome have normal intelligence or may have mild learning disabilities; some may have psychiatric problems. Glaucoma, retinal degeneration, and clouded corneas may significantly impair vision. Other problems include carpal tunnel syndrome or other nerve compression, stiff joints, claw hands and deformed feet, a short neck, and aortic valve disease. Some affected individuals also have obstructive airway disease and sleep apnea. Persons with Scheie syndrome can live into adulthood.

MPS I H-S, Hurler-Scheie syndrome, is less severe than Hurler syndrome alone. Symptoms generally begin between ages 3 and 8. Children may have moderate intellectual disability and learning difficulties. Skeletal and systemic irregularities include short stature, marked smallness in the jaws, progressive joint stiffness, compressed spinal cord, clouded corneas, hearing loss, heart disease, coarse facial features, and umbilical hernia. Respiratory problems, sleep apnea, and heart disease may develop in adolescence. Some persons with MPS I H-S need continuous positive airway pressure during sleep to ease breathing. Life expectancy is generally into the late teens or early twenties.

MPS II, also known as Hunter syndrome, is caused by lack of the enzyme iduronate sulfatase. Hunter syndrome has two clinical subtypes and (since it shows X-linked recessive inheritance) is the only one of the mucopolysaccharidoses in which the mother alone can pass the defective gene to a son. The incidence of Hunter syndrome is estimated to be 1 in 100,000 to 150,000 male births.

Mutations in the IDS gene cause MPS II. The IDS gene provides instructions for producing the I2S enzyme, which is involved in the breakdown of large sugar molecules called glycosaminoglycans (GAGs). Specifically, I2S removes a chemical group known as a sulfate from a molecule called sulfated alpha-L-iduronic acid, which is present in two GAGs called heparan sulfate and dermatan sulfate. I2S is located in lysosomes, compartments within cells that digest and recycle different types of molecules.

Mucopolysaccharidosis type VI (MPS VI) or Maroteaux-Lamy disease is a lysosomal storage disease, of the mucopolysaccharidosis group, characterized by severe somatic involvement and an absence of psycho-intellectual regression. The prevalence of this rare mucopolysaccharidosis is between 1/250,000 and 1/600,000 births. In the severe forms, the first clinical manifestations occur between 6 and 24 months and are gradually accentuated: facial dysmorphia (macroglossia, mouth constantly half open, thick features), joint limitations, very severe dysostosis multiplex (platyspondyly, kyphosis, scoliosis, pectus carinatum, genu valgum, long bone deformation), small size (less than 1.10 m), hepatomegaly, heart valve damage, cardiomyopathy, deafness, corneal opacities. Intellectual development is usually normal or virtually normal, but the auditory and ophthalmological damage can cause learning difficulties. The symptoms and the severity of the disease vary considerably from one patient to the other and intermediate forms, or even very moderate forms also exist (spondyloepiphyseal-metaphyseal dysplasia associated with cardiovascular involvement). Like the other mucopolysaccharidoses, Maroteaux-Lamy disease is linked to the defect of an enzyme of mucopolysaccharide metabolism, in the case in point N-acetylgalactosamine-4-sulfatase (also called arylsulfatase B) (ARSB). This enzyme metabolizes the sulfate group of dermatan sulfate (Neufeld et al.: “The mucopolysaccharidoses” The Metabolic Basis of Inherited Diseases, eds. Scriver et al, New York, McGraw-Hill, 1989, p. 1565-1587). This enzymatic defect blocks the gradual degradation of dermatan sulfate, thereby leading to an accumulation of dermatan sulfate in the lysosomes of the storage tissues.

Mucopolysaccharidosis type VII (MPS VII) or Sly disease is a very rare lysosomal storage disease of the mucopolysaccharidosis group. The symptomology is extremely heterogeneous: antenatal forms (nonimmune fetoplacental anasarca), severe neonatal forms (with dysmorphia, hernias, hepatosplenomegaly, club feet, dysostosis, significant hypotonia and neurological problems evolving to retarded growth and a profound intellectual deficiency in the event of survival) and very moderate forms discovered at adolescence or even at adult age (thoracic kyphosis). The disease is due to a defect in beta-D-glucuronidase (GUSB) responsible for accumulation, in the lysosomes, of various glycosaminoglycans: dermatan sulfate, heparan sulfate and chondroitin sulfate. There is at the current time no effective treatment for this disease.

### X-Linked Adrenoleukodystrophy

Adrenoleukodystrophy (ALD) is an X-linked disease affecting 1/20,000 males either as cerebral ALD in childhood or as adrenomyleneuropathy (AMN) in adults. Childhood ALD is the more severe form, with onset of neurological symptoms between 5-12 years of age. Central nervous system demyelination progresses rapidly and death occurs within a few years. AMN is a milder form of the disease with onset at 15-30 years of age and a more progressive course. Adrenal insufficiency (Addison's disease) may remain the only clinical manifestation of ALD. The principal biochemical abnormality of ALD is the accumulation of very long chain fatty acids (VLCFA) because of impaired β-oxidation in peroxisomes.

More than 650 mutations in the ABCD1 gene have been found to cause X-linked adrenoleukodystrophy. This condition is characterized by varying degrees of cognitive and movement problems as well as hormone imbalances. The mutations that cause X-linked adrenoleukodystrophy prevent the production of any ALDP in about 75 percent of people with this disorder. Other people with X-linked adrenoleukodystrophy can produce ALDP, but the protein is not able to perform its normal function. With little or no functional ALDP, VLCFAs are not broken down, and they build up in the body. The accumulation of these fats may be toxic to the adrenal glands (small glands on top of each kidney) and to the fatty layer of insulation (myelin) that surrounds many nerves in the body. Research suggests that the accumulation of VLCFAs triggers an inflammatory response in the brain, which could lead to the breakdown of myelin. The destruction of these tissues leads to the signs and symptoms of X-linked adrenoleukodystrophy.

### Globoid Cell Leukodystrophy

Infantile globoid cell leucodystrophy (GLD, galactosylceramide lipidosis or Krabbe's disease) is a rare, autosomal recessive hereditary degenerative disorder in the central and peripheral nervous systems. The incidence in the US is estimated to 1:100.000. It is characterized by the presence of globoid cells (cells with multiple nuclei), degeneration of the protective myelin layer of the nerves and loss of cells in the brain. GLD causes severe mental reduction and motoric delay. It is caused by a deficiency in galactocerebroside-β-galactosidase (GALC), which is an essential enzyme in the metabolism of myelin. The disease often affects infants prior to the age of 6 months, but it can also appear during youth or in adults. The symptoms include irritability, fever without any known cause, stiffness in the limbs (hypertony), seizures, problems associated with food intake, vomiting and delayed development of mental and motoric capabilities. Additional symptoms include muscular weakness, spasticity, deafness and blindness.

The galactosylceramidase gene (GALC) is about 60 kb in length and consists of 17 exons. Numerous mutations and polymorphisms have been identified in the murine and human GALC gene, causing GLD with different degrees of severity.

### Metachromatic Leukodystrophy

Metachromatic leukodystrophy is an inherited disorder characterized by the accumulation of fats called sulfatides in cells. This accumulation especially affects cells in the nervous system that produce myelin, the substance that insulates and protects nerves. Nerve cells covered by myelin make up a tissue called white matter. Sulfatide accumulation in myelin-producing cells causes progressive destruction of white matter (leukodystrophy) throughout the nervous system, including in the brain and spinal cord (the central nervous system) and the nerves connecting the brain and spinal cord to muscles and sensory cells that detect sensations such as touch, pain, heat, and sound (the peripheral nervous system).

In people with metachromatic leukodystrophy, white matter damage causes progressive deterioration of intellectual functions and motor skills, such as the ability to walk. Affected individuals also develop loss of sensation in the extremities (peripheral neuropathy), incontinence, seizures, paralysis, an inability to speak, blindness, and hearing loss. Eventually they lose awareness of their surroundings and become unresponsive. While neurological problems are the primary feature of metachromatic leukodystrophy, effects of sulfatide accumulation on other organs and tissues have been reported, most often involving the gallbladder.

The most common form of metachromatic leukodystrophy, affecting about 50 to 60 percent of all individuals with this disorder, is called the late infantile form. This form of the disorder usually appears in the second year of life. Affected children lose any speech they have developed, become weak, and develop problems with walking (gait disturbance). As the disorder worsens, muscle tone generally first decreases, and then increases to the point of rigidity. Individuals with the late infantile form of metachromatic leukodystrophy typically do not survive past childhood.

In 20 to 30 percent of individuals with metachromatic leukodystrophy, onset occurs between the age of 4 and adolescence. In this juvenile form, the first signs of the disorder may be behavioral problems and increasing difficulty with schoolwork. Progression of the disorder is slower than in the late infantile form, and affected individuals may survive for about 20 years after diagnosis.

Most individuals with metachromatic leukodystrophy have mutations in the ARSA gene, which provides instructions for making the enzyme arylsulfatase A. This enzyme is located in cellular structures called lysosomes, which are the cell's recycling centers. Within lysosomes, arylsulfatase A helps break down sulfatides. A few individuals with metachromatic leukodystrophy have mutations in the PSAP gene. This gene provides instructions for making a protein that is broken up (cleaved) into smaller proteins that assist enzymes in breaking down various fats. One of these smaller proteins is called saposin B; this protein works with arylsulfatase A to break down sulfatides.

Mutations in the ARSA or PSAP genes result in a decreased ability to break down sulfatides, resulting in the accumulation of these substances in cells. Excess sulfatides are toxic to the nervous system. The accumulation gradually destroys myelin-producing cells, leading to the impairment of nervous system function that occurs in metachromatic leukodystrophy.

In some cases, individuals with very low arylsulfatase A activity show no symptoms of metachromatic leukodystrophy. This condition is called pseudoarylsulfatase deficiency.

The adult form of metachromatic leukodystrophy affects approximately 15 to 20 percent of individuals with the disorder. In this form, the first symptoms appear during the teenage years or later. Often behavioral problems such as alcoholism, drug abuse, or difficulties at school or work are the first symptoms to appear. The affected individual may experience psychiatric symptoms such as delusions or hallucinations. People with the adult form of metachromatic leukodystrophy may survive for 20 to 30 years after diagnosis. During this time there may be some periods of relative stability and other periods of more rapid decline.

Metachromatic leukodystrophy gets its name from the way cells with an accumulation of sulfatides appear when viewed under a microscope. The sulfatides form granules that are described as metachromatic, which means they pick up color differently than surrounding cellular material when stained for examination.

### Gaucher Disease

Gaucher disease is an inherited disorder that affects many of the body's organs and tissues. The signs and symptoms of this condition vary widely among affected individuals. Researchers have described several types of Gaucher disease based on their characteristic features.

Type 1 Gaucher disease is the most common form of this condition. Type 1 is also called non-neuronopathic Gaucher disease because the brain and spinal cord (the central nervous system) are usually not affected. The features of this condition range from mild to severe and may appear anytime from childhood to adulthood. Major signs and symptoms include enlargement of the liver and spleen (hepatosplenomegaly), a low number of red blood cells (anemia), easy bruising caused by a decrease in blood platelets (thrombocytopenia), lung disease, and bone abnormalities such as bone pain, fractures, and arthritis.

Types 2 and 3 Gaucher disease are known as neuronopathic forms of the disorder because they are characterized by problems that affect the central nervous system. In addition to the signs and symptoms described above, these conditions can cause abnormal eye movements, seizures, and brain damage. Type 2 Gaucher disease usually causes life-threatening medical problems beginning in infancy. Type 3 Gaucher disease also affects the nervous system, but it tends to worsen more slowly than type 2.

The most severe type of Gaucher disease is called the perinatal lethal form. This condition causes severe or life-threatening complications starting before birth or in infancy. Features of the perinatal lethal form can include extensive swelling caused by fluid accumulation before birth (hydrops fetalis); dry, scaly skin (ichthyosis) or other skin abnormalities; hepatosplenomegaly; distinctive facial features; and serious neurological problems. As its name indicates, most infants with the perinatal lethal form of Gaucher disease survive for only a few days after birth.

Another form of Gaucher disease is known as the cardiovascular type because it primarily affects the heart, causing the heart valves to harden (calcify). People with the cardiovascular form of Gaucher disease may also have eye abnormalities, bone disease, and mild enlargement of the spleen (splenomegaly).

Mutations in the GBA gene cause Gaucher disease. The GBA gene provides instructions for making an enzyme called beta-glucocerebrosidase. This enzyme breaks down a fatty substance called glucocerebroside into a sugar (glucose) and a simpler fat molecule (ceramide). Mutations in the GBA gene greatly reduce or eliminate the activity of beta-glucocerebrosidase. Without enough of this enzyme, glucocerebroside and related substances can build up to toxic levels within cells. Tissues and organs are damaged by the abnormal accumulation and storage of these substances, causing the characteristic features of Gaucher disease.

### Fucosidosis

Fucosidosis is a condition that affects many areas of the body, especially the brain. Affected individuals have intellectual disability that worsens with age, and many develop dementia later in life. People with this condition often have delayed development of motor skills such as walking; the skills they do acquire deteriorate over time. Additional signs and symptoms of fucosidosis include impaired growth; abnormal bone development (dysostosis multiplex); seizures; abnormal muscle stiffness (spasticity); clusters of enlarged blood vessels forming small, dark red spots on the skin (angiokeratomas); distinctive facial features that are often described as “coarse”; recurrent respiratory infections; and abnormally large abdominal organs (visceromegaly).

In severe cases, symptoms typically appear in infancy, and affected individuals usually live into late childhood. In milder cases, symptoms begin at age 1 or 2, and affected individuals tend to survive into mid-adulthood.

In the past, researchers described two types of this condition based on symptoms and age of onset, but current opinion is that the two types are actually a single disorder with signs and symptoms that range in severity.

Mutations in the FUCA1 gene cause fucosidosis. The FUCA1 gene provides instructions for making an enzyme called alpha-L-fucosidase. This enzyme plays a role in the breakdown of complexes of sugar molecules (oligosaccharides) attached to certain proteins (glycoproteins) and fats (glycolipids). Alpha-L-fucosidase is responsible for cutting (cleaving) off a sugar molecule called fucose toward the end of the breakdown process.

FUCA1 gene mutations severely reduce or eliminate the activity of the alpha-L-fucosidase enzyme. A lack of enzyme activity results in an incomplete breakdown of glycolipids and glycoproteins. These partially broken down compounds gradually accumulate within various cells and tissues throughout the body and cause cells to malfunction. Brain cells are particularly sensitive to the buildup of glycolipids and glycoproteins, which can result in cell death. Loss of brain cells is thought to cause the neurological symptoms of fucosidosis. Accumulation of glycolipids and glycoproteins also occurs in other organs such as the liver, spleen, skin, heart, pancreas, and kidneys, contributing to the additional symptoms of fucosidosis.

### Alpha-Mannosidosis

Alpha-mannosidosis is an autosomal, recessively inherited lysosomal storage disorder that has been clinically well characterized (M. A. Chester et al., 1982, in Genetic Errors of Glycoprotein Metabolism pp 90-119, Springer Verlag, Berlin). Glycoproteins are normally degraded stepwise in the lysosome and one of the steps, namely the cleavage of .alpha.-linked mannose residues from the non-reducing end during the ordered degradation of N-linked glycoproteins is catalysed by the enzyme lysosomal α-mannosidase (EC 3.2.1.24). However, in alpha-mannosidosis, a deficiency of the enzyme α-mannosidase results in the accumulation of mannose rich oligosaccharides. As a result, the lysosomes increase in size and swell, which impairs cell functions.

The symptoms of α-mannosidosis include psychomotor retardation, ataxia, impaired hearing, vacuolized lymphocytes in the peripheral blood and skeletal changes.

Mutations in the MAN2B1 gene cause alpha-mannosidosis. This gene provides instructions for making the enzyme alpha-mannosidase. This enzyme works in the lysosomes, which are compartments that digest and recycle materials in the cell. Within lysosomes, the enzyme helps break down complexes of sugar molecules (oligosaccharides) attached to certain proteins (glycoproteins). In particular, alpha-mannosidase helps break down oligosaccharides containing a sugar molecule called mannose.

Mutations in the MAN2B1 gene interfere with the ability of the alpha-mannosidase enzyme to perform its role in breaking down mannose-containing oligosaccharides. These oligosaccharides accumulate in the lysosomes and cause cells to malfunction and eventually die. Tissues and organs are damaged by the abnormal accumulation of oligosaccharides and the resulting cell death, leading to the characteristic features of alpha-mannosidosis.

### Aspartylglucosaminuria

Aspartylglucosaminuria is a condition that causes a progressive decline in mental functioning. Infants with aspartylglucosaminuria appear healthy at birth, and development is typically normal throughout early childhood. The first sign of this condition, evident around the age of 2 or 3, is usually delayed speech. Mild intellectual disability then becomes apparent, and learning occurs at a slowed pace. Intellectual disability progressively worsens in adolescence. Most people with this disorder lose much of the speech they have learned, and affected adults usually have only a few words in their vocabulary. Adults with aspartylglucosaminuria may develop seizures or problems with movement.

People with this condition may also have bones that become progressively weak and prone to fracture (osteoporosis), an unusually large range of joint movement (hypermobility), and loose skin. Affected individuals tend to have a characteristic facial appearance that includes widely spaced eyes (ocular hypertelorism), small ears, and full lips. The nose is short and broad and the face is usually square-shaped. Children with this condition may be tall for their age, but lack of a growth spurt in puberty typically causes adults to be short. Affected children also tend to have frequent upper respiratory infections. Individuals with aspartylglucosaminuria usually survive into mid-adulthood.

Mutations in the AGA gene cause aspartylglucosaminuria. The AGA gene provides instructions for producing an enzyme called aspartylglucosaminidase. This enzyme is active in lysosomes, which are structures inside cells that act as recycling centers. Within lysosomes, the enzyme helps break down complexes of sugar molecules (oligosaccharides) attached to certain proteins (glycoproteins).

AGA gene mutations result in the absence or shortage of the aspartylglucosaminidase enzyme in lysosomes, preventing the normal breakdown of glycoproteins. As a result, glycoproteins can build up within the lysosomes. Excess glycoproteins disrupt the normal functions of the cell and can result in destruction of the cell. A buildup of glycoproteins seems to particularly affect nerve cells in the brain; loss of these cells causes many of the signs and symptoms of aspartylglucosaminuria.

### Farber's Disease

Farber's disease is an inherited condition involving the breakdown and use of fats in the body (lipid metabolism). People with this condition have an abnormal accumulation of lipids (fat) throughout the cells and tissues of the body, particularly around the joints. Farber's disease is characterized by three classic symptoms: a hoarse voice or weak cry, small lumps of fat under the skin and in other tissues (lipogranulomas), and swollen and painful joints. Other symptoms may include difficulty breathing, an enlarged liver and spleen (hepatosplenomegaly), and developmental delay. Researchers have described seven types of Farber's disease based on their characteristic features. This condition is caused by mutations in the ASAH1 gene and is inherited in an autosomal recessive manner.

### Tay-Sachs Disease

Tay-Sachs disease is a rare inherited disorder that progressively destroys nerve cells (neurons) in the brain and spinal cord.

The most common form of Tay-Sachs disease becomes apparent in infancy. Infants with this disorder typically appear normal until the age of 3 to 6 months, when their development slows and muscles used for movement weaken. Affected infants lose motor skills such as turning over, sitting, and crawling. They also develop an exaggerated startle reaction to loud noises. As the disease progresses, children with Tay-Sachs disease experience seizures, vision and hearing loss, intellectual disability, and paralysis. An eye abnormality called a cherry-red spot, which can be identified with an eye examination, is characteristic of this disorder. Children with this severe infantile form of Tay-Sachs disease usually live only into early childhood.

Other forms of Tay-Sachs disease are very rare. Signs and symptoms can appear in childhood, adolescence, or adulthood and are usually milder than those seen with the infantile form. Characteristic features include muscle weakness, loss of muscle coordination (ataxia) and other problems with movement, speech problems, and mental illness. These signs and symptoms vary widely among people with late-onset forms of Tay-Sachs disease.

Mutations in the HEXA gene cause Tay-Sachs disease. The HEXA gene provides instructions for making part of an enzyme called beta-hexosaminidase A, which plays a critical role in the brain and spinal cord. This enzyme is located in lysosomes, which are structures in cells that break down toxic substances and act as recycling centers. Within lysosomes, beta-hexosaminidase A helps break down a fatty substance called GM2 ganglioside.

Mutations in the HEXA gene disrupt the activity of beta-hexosaminidase A, which prevents the enzyme from breaking down GM2 ganglioside. As a result, this substance accumulates to toxic levels, particularly in neurons in the brain and spinal cord. Progressive damage caused by the buildup of GM2 ganglioside leads to the destruction of these neurons, which causes the signs and symptoms of Tay-Sachs disease.

Because Tay-Sachs disease impairs the function of a lysosomal enzyme and involves the buildup of GM2 ganglioside, this condition is sometimes referred to as a lysosomal storage disorder or a GM2-gangliosidosis.

### Pompe Disease

Pompe disease (also known as glycogen storage disease type II; acid alpha-glucosidase deficiency; acid maltase deficiency; GAA deficiency; GSD II; glycogenosis type II; glycogenosis, generalized, cardiac form; cardiomegalia glycogenica diffusa; acid maltase deficiency; AMD; or alpha-1,4-glucosidase deficiency) is an autosomal recessive metabolic genetic disorder characterized by mutations in the gene for the lysomsomal enzyme acid alpha-glucosidase (GAA) (also known as acid maltase). Mutations in the GAA gene eliminate or reduce the ability of the GAA enzyme to hydrolyze the α-1,4 and α-1,6 linkages in glycogen, maltose and isomaltose. As a result, glycogen accumulates in the lysosomes and cytoplasm of cells throughout the body leading to cell and tissue destruction. Tissues that are particularly affected include skeletal muscle and cardiac muscle. The accumulated glycogen causes progressive muscle weakness leading to cardiomegaly, ambulatory difficulties and respiratory insufficiency.

Three forms of Pompe disease have been identified, including the classic infantile-onset disease, non-classic infantile-onset disease and late onset disease. The classic infantile-onset form is characterized by muscle weakness, poor muscle tone, hepatomegaly and cardiac defects. The incidence of the disease is approximately 1 in 140,000 individuals. Patients with this form of the disease often die of heart failure in the first year of life. The non-classic infantile-onset form of the disease is characterized by delayed motor skills, progressive muscle weakness and in some instances cardiomegaly. Patients with this form of the disease often live only into early childhood due to respiratory failure. The late-onset form of the disease may present in late childhood, adolescence or adulthood and is characterized by progressive muscle weakness of the legs and trunk.

### Niemann Pick Disease

Niemann-Pick disease is a condition that affects many body systems. It has a wide range of symptoms that vary in severity. Niemann-Pick disease is divided into four main types: type A, type B, type C1, and type C2. These types are classified on the basis of genetic cause and the signs and symptoms of the condition.

Infants with Niemann-Pick disease type A usually develop an enlarged liver and spleen (hepatosplenomegaly) by age 3 months and fail to gain weight and grow at the expected rate (failure to thrive). The affected children develop normally until around age 1 year when they experience a progressive loss of mental abilities and movement (psychomotor regression). Children with Niemann-Pick disease type A also develop widespread lung damage (interstitial lung disease) that can cause recurrent lung infections and eventually lead to respiratory failure. All affected children have an eye abnormality called a cherry-red spot, which can be identified with an eye examination. Children with Niemann-Pick disease type A generally do not survive past early childhood.

Niemann-Pick disease type B usually presents in mid-childhood. The signs and symptoms of this type are similar to type A, but not as severe. People with Niemann-Pick disease type B often have hepatosplenomegaly, recurrent lung infections, and a low number of platelets in the blood (thrombocytopenia). They also have short stature and slowed mineralization of bone (delayed bone age). About one-third of affected individuals have the cherry-red spot eye abnormality or neurological impairment. People with Niemann-Pick disease type B usually survive into adulthood.

Niemann-Pick disease types A and B is caused by mutations in the SMPD1 gene. This gene provides instructions for producing an enzyme called acid sphingomyelinase. This enzyme is found in lysosomes, which are compartments within cells that break down and recycle different types of molecules. Acid sphingomyelinase is responsible for the conversion of a fat (lipid) called sphingomyelin into another type of lipid called ceramide. Mutations in SMPD1 lead to a shortage of acid sphingomyelinase, which results in reduced break down of sphingomyelin, causing this fat to accumulate in cells. This fat buildup causes cells to malfunction and eventually die. Over time, cell loss impairs function of tissues and organs including the brain, lungs, spleen, and liver in people with Niemann-Pick disease types A and B.

### Wolman Disease

Lysosomal acid lipase deficiency is an inherited condition characterized by problems with the breakdown and use of fats and cholesterol in the body (lipid metabolism). In affected individuals, harmful amounts of fats (lipids) accumulate in cells and tissues throughout the body, which typically causes liver disease. There are two forms of the condition. The most severe and rarest form begins in infancy. The less severe form can begin from childhood to late adulthood.

In the severe, early-onset form of lysosomal acid lipase deficiency, lipids accumulate throughout the body, particularly in the liver, within the first weeks of life. This accumulation of lipids leads to several health problems, including an enlarged liver and spleen (hepatosplenomegaly), poor weight gain, a yellow tint to the skin and the whites of the eyes (jaundice), vomiting, diarrhea, fatty stool (steatorrhea), and poor absorption of nutrients from food (malabsorption). In addition, affected infants often have calcium deposits in small hormone-producing glands on top of each kidney (adrenal glands), low amounts of iron in the blood (anemia), and developmental delay. Scar tissue quickly builds up in the liver, leading to liver disease (cirrhosis). Infants with this form of lysosomal acid lipase deficiency develop multi-organ failure and severe malnutrition and generally do not survive past 1 year.

In the later-onset form of lysosomal acid lipase deficiency, signs and symptoms vary and usually begin in mid-childhood, although they can appear anytime up to late adulthood. Nearly all affected individuals develop an enlarged liver (hepatomegaly); an enlarged spleen (splenomegaly) may also occur. About two-thirds of individuals have liver fibrosis, eventually leading to cirrhosis. Approximately one-third of individuals with the later-onset form have malabsorption, diarrhea, vomiting, and steatorrhea. Individuals with this form of lysosomal acid lipase deficiency may have increased liver enzymes and high cholesterol levels, which can be detected with blood tests.

Some people with this later-onset form of lysosomal acid lipase deficiency develop an accumulation of fatty deposits on the artery walls (atherosclerosis). Although these deposits are common in the general population, they usually begin at an earlier age in people with lysosomal acid lipase deficiency. The deposits narrow the arteries, increasing the chance of heart attack or stroke. The expected lifespan of individuals with later-onset lysosomal acid lipase deficiency depends on the severity of the associated health problems.

The two forms of lysosomal acid lipase deficiency were once thought to be separate disorders. The early-onset form was known as Wolman disease, and the later-onset form was known as cholesteryl ester storage disease. Although these two disorders have the same genetic cause and are now considered to be forms of a single condition, these names are still sometimes used to distinguish between the forms of lysosomal acid lipase deficiency.

Mutations in the LIPA gene cause lysosomal acid lipase deficiency. The LIPA gene provides instructions for producing an enzyme called lysosomal acid lipase. This enzyme is found in cell compartments called lysosomes, which digest and recycle materials the cell no longer needs. The lysosomal acid lipase enzyme breaks down lipids such as cholesteryl esters and triglycerides. The lipids produced through these processes, cholesterol and fatty acids, are used by the body or transported to the liver for removal.

Mutations in the LIPA gene lead to a shortage (deficiency) of functional lysosomal acid lipase. The severity of the condition depends on how much working enzyme is available. Individuals with the early-onset form of lysosomal acid lipase deficiency have no normal enzyme activity. Those with the later-onset form are thought to have some enzyme activity remaining, and the amount generally determines the severity of signs and symptoms.

Decreased lysosomal acid lipase activity results in the accumulation of cholesteryl esters, triglycerides, and other lipids within lysosomes, causing fat buildup in multiple tissues. The body's inability to produce cholesterol from the breakdown of these lipids leads to an increase in alternative methods of cholesterol production and higher-than-normal levels of cholesterol in the blood. The excess lipids are transported to the liver for removal. Because many of them are not broken down properly, they cannot be removed from the body; instead they accumulate in the liver, resulting in liver disease. The progressive accumulation of lipids in tissues results in organ dysfunction and the signs and symptoms of lysosomal acid lipase deficiency.

### Hematopoietic Stem Cells

As used herein, hematopoietic stem cells (HSC) refers to animal, preferably mammalian, more preferably human cells that have the ability to differentiate into any of several types of blood cells, including red blood cells, white blood cells, including lymphoid cells and myeloid cells. HSC can include hematopoietic cells having long-term engrafting potential in vivo. Long term engrafting potential (e.g., long term hematopoietic stem cells) can be determined using animal models or in vitro models. Animal models for long-term engrafting potential of candidate human hematopoietic stem cell populations include the SCID-hu bone model (Kyoizumi et al. (1992) Blood 79:1704; Murray et al. (1995) Blood 85 (2) 368-378) and the in utero sheep model (Zanjani et al. (1992) J. Clin. Invest. 89:1179). For a review of animal models of human hematopoiesis, see Srour et al. (1992) J. Hematother. 1:143-153 and the references cited therein. An in vitro model for stem cells is the long-term culture-initiating cell (LTCIC) assay, based on a limiting dilution analysis of the number of clonogenic cells produced in a stromal co-culture after 5-8 weeks (Sutherland et al. (1990) Proc. Nat'l Acad. Sci. 87:3584-3588). The LTCIC assay has been shown to correlate with another commonly used stem cell assay, the cobblestone area forming cell (CAFC) assay, and with long-term engrafting potential in vivo (Breems et al. (1994) Leukemia 8:1095).

Hematopoietic stem cells (HSC) reside in the bone marrow and have the unique ability to give rise to all of the different mature blood cell types and tissues. HSC are self-renewing cells: when they proliferate, at least some of their daughter cells remain as HSC, so that the pool of stem cells is not depleted. The other cells differentiate into common lymphoid progenitor cells that produce lymphocytes and into common myeloid progenitor cells that produce monocytes.

In some embodiments, the hematopoietic stem cells for use in genetic modification herein are isolated from bone marrow. In some embodiments, HSC can be taken from the pelvis, at the iliac crest, using a needle or syringe.

In some embodiments, the hematopoietic stem cells can be derived from human cord blood or mobilized peripheral blood. Hematopoietic stem cells obtained from human peripheral blood may be mobilized by one of a variety of strategies. Exemplary agents that can be used to induce mobilization of hematopoietic stem cells from the bone marrow into peripheral blood include chemokine (C—X—C motif) receptor 4 (CXCR4) antagonists, such as AMD3100 (also known as Plerixafor and MOZOBIL (Genzyme, Boston, Mass.)) and granulocyte colony-stimulating factor (GCSF), the combination of which has been shown to rapidly mobilize CD34+ cells in clinical experiments. Additionally, chemokine (C—X—C motif) ligand 2 (CXCL2, also referred to as GROβ) represents another agent capable of inducing hematopoietic stem cell mobilization to from bone marrow to peripheral blood. Agents capable of inducing mobilization of hematopoietic stem cells for use with the compositions and methods of the invention may be used in combination with one another. For instance, CXCR4 antagonists (e.g., AMD3100), CXCL2, and/or GCSF may be administered to a subject sequentially or simultaneously in a single mixture in order to induce mobilization of hematopoietic stem cells from bone marrow into peripheral blood. The use of these agents as inducers of hematopoietic stem cell mobilization is described, e.g., in Pelus, Current Opinion in Hematology 15:285 (2008), the disclosure of which is incorporated herein by reference.

In some embodiments, HSC are harvested from the circulating peripheral blood, while the blood donor is injected with an agent that mobilizes the HSC from the bone marrow. In some embodiments, the agent that mobilizes the HSC from the bone marrow to the peripheral blood is a cytokine, such as granulocyte-colony stimulating factor (G-CSF). In some embodiments, populations of HSC isolated from the peripheral blood are enriched in CD34+ cells, and comprise at least 50%, at least 70%, or at least 90% of CD34+ cells.

In some embodiments, for mobilized peripheral blood (MPB) leukapheresis, CD34+ cells can generally be processed and enriched using immunomagnetic beads such as CliniMACS, Purified CD34+ cells can be seeded on culture bags at 1×10⁶ cells/ml in serum-free medium in the presence of cells culture grade Stem Cell Factor (SCF), preferably 300 ng/ml (Amgen Inc., Thousand Oaks, CA, USA), preferably with FMS-like tyrosine kinase 3 ligand (FLT3L) 300 ng/ml, and Thrombopoietin (TPO), preferably around 100 ng/ml and further interleukline IL-3, preferably more than 60 ng/ml (all from Cell Genix Technologies) during between preferably 12 and 24 hours before being transferred to an electroporation buffer comprising the sequence specific reagent (e.g., mRNA). Upon electroporation, the cells are transferred back to the culture medium prior to being resuspended in saline and transferred in a syringe for infusion.

Methods for enriching or depleting specific cell populations in a mixture of cells are well known in the art. For example, cell populations can be enriched or depleted by density separation, rosetting tetrameric antibody complex mediated enrichment/depletion, magnetic activated cell sorting (MACS), multi-parameter fluorescence based molecular phenotypes such as fluorescence-activated cell sorting (FACS), or any combination thereof. Collectively, these methods of enriching or depleting cell populations may be referred to generally herein as “sorting” the cell populations or contacting the cells “under conditions” to form or produce an enriched (+) or depleted (−) cell population.

Upon collection of the mobilized cells, the withdrawn hematopoietic stem cells can be genetically modified as described herein and then infused into a patient in need thereof, which may be the donor or another subject, such as a subject that is at least partially HLA-matched to the donor, for the treatment of disease as described herein.

In some embodiments, these cells form a population of cells, which preferably originate from a single donor or patient. These populations of cells can be expanded under closed culture recipients to comply with highest manufacturing practices requirements and can be frozen prior to infusion into a patient, thereby providing “off the shelf” or “ready to use” therapeutic compositions.

In some embodiments, the HSC are CD34+. In some embodiments, the HSC can further be described as CD133+, CD90+, CD38−, CD45RA−, Lin−, or any combination thereof.

In some embodiments, the HSC capable of differentiating into microglial cells are derived from pluripotent stem cells, such as induced pluripotent stem cells (iPS). See, e.g., Abud et al., Neuron 94, 278-293 (2017). In some embodiments, the iPS cells are genetically modified as described herein and then differentiated into HSC cells. In some embodiments, the iPS cells are differentiated into HSC and then the HSC are genetically modified as described herein. In further embodiments, cells can be gene edited before being reprogrammed into iPS cells and HSCs as described for instance in Int. Appl. No. PCT/EP2018/083180. In some embodiments, the hematopoietic stem cells can be isolated from the patient to be treated or isolated from a compatible donor.

In some embodiments, hematopoietic stem cells are obtained from induced pluripotent stem (iPS) cells derived from cells of the patient to be treated or from a compatible donor.

In some embodiments, the HSC can be expanded ex vivo prior to genetic modification and/or infusion of these cells into the patient. See, e.g., U.S. Pat. Nos. 9,580,426; 9,956,249; 9,527,828; 9,428,748; 9,394,520; 9,328,085; 9,226,942; 9,115,341; 8,927,281.

In some embodiments, the cells are isolated from a donor that is an HLA matched sibling donor, an HLA matched unrelated donor, a partially matched unrelated donor, a haploidentical related donor, autologous donor, an HLA unmatched donor, a pool of donors or any combination thereof. In some embodiments, the population of therapeutic cells is allogeneic. In some embodiments, the population of therapeutic cells is autologous. In some embodiments, the population of therapeutic cells is haploidentical.

### Genetically Modified Cells

In some embodiments, the invention provides genetically modified HSC or iPS cells obtainable according to any one of the embodiments of the methods described herein.

In some embodiments, the invention provides genetically modified HSC or iPS cells comprising a transgene integrated at a locus that is at least transcriptionally active in microglial cells, wherein the transgene is under the transcriptional control of the endogenous promoter of the locus. In some embodiments, the transgene comprises a coding sequence of a gene selected from the group consisting of IDUA, IDS, ARSB, GUSB, ABCD1, GALC, ARSA, PSAP, GBA, FUCA1, MAN2B1, AGA, ASAH1, HEXA, GAA, SMPD1, LIPA and CDKL5.

In some embodiments, multiple copies of the transgene are integrated in the HSC or iPS cell. In some embodiments, the multiple copies are integrated at different loci. In some embodiments, the multiple copies are integrated at the same locus. In some embodiments, the multiple copies integrated at the same locus are separated by 2A self-cleaving peptide sequences.

In some embodiments, the introduced transgene is under the control of the endogenous promoter in the microglial cells. In some embodiments, the locus that is active in microglial cells is selected from the group consisting of TMEM119, S100A9, CD11B, B2m, Cx3cr1, MERTK, CD164, Tlr4, Tlr7, Cd14, Fcgr1a, Fcgr3a, TBXAS1, DOK3, ABCA1, TMEM195, MR1, CSF3R, FGD4, TSPAN14, TGFBRI, CCR5, GPR34, SERPINE2, SLCO2B1, P2ry12, Olfml3, P2ry13, Hexb, Rhob, Jun, Rab3il1, Ccl2, Fcrls, Scoc, Siglech, Slc2a5, Lrrc3, Plxdc2, Usp2, Ctsf, Cttnbp2nl, Atp8a2, Lgmn, Mafb, Egr1, Bhlhe41, Hpgds, Ctsd, Hspa1a, Lag3, Csflr, Adamts1, F11r, Golm1, Nuak1, Crybb1, Ltc4s, Sgce, Pla2g15, Ccl3l1, Abhd12, Ang, Ophn1, Sparc, Pros1, P2ry6, Lair1, Il1a, Epb41l2, Adora3, Rilpl1, Pmepa1, Ccl13, Pde3b, Scamp5, Ppp1r9a, Tjp1, Ak1, B4galt4, Gtf2 h2, Trem2, Ckb, Acp2, Pon3, Agmo, Tnfrsf17, Fscn1, St3gal6, Adap2, Ccl4, Entpd1, Tmem86a, Kctd12, Dst, Cts12, Abcc3, Pdgfb, Pald1, Tubgcp5, Rapgef5, Stab1, Lacc1, Tmc7, Nrip1, Kcnd1, Tmem206, Hps4, Dagla, Extl3, Mlph, Arhgap22, Cxxc5, P4ha1, Cysltr1, Fgd2, Kcnk13, Gbgt1, C18orf1, Cadm1, Bco2, Adrb1, C3ar1, Large, Leprel1, Liph, Upk1b, P2rx7, Slc46a1, Ebf3, Ppp1r15a, Il10ra, Rasgrp3, Fos, Tppp, Slc24a3, Havcr2, Nav2, Apbb2, Clstn1, Blnk, Gnaq, Ptprm, Frmd4a, Cd86, Tnfrsf11a, Spint1, Ppm1l, Tgfbr2, Cmklr1, Tlr6, Gas6, Hist1h2ab, Atf3, Acvr1, Abi3, Lrp12, Ttc28, Plxna4, Adamts16, Rgs1, Icam1, Snx24, Ly96, Dnajb4, and Ppfia4.

In some embodiments, the genetically modified HSC or iPS cells comprise a transgene integrated at a locus that is transcriptionally active in microglial cells selected from TMEM119, CD11B, B2m, CX3CR1 or S100A9, wherein the transgene is under the transcriptional control of the endogenous promoter of the locus.

In some embodiments, the genetically modified hematopoietic stem cells are obtained by genetically modifying the hematopoietic stem cells directly. In some embodiments, the genetically modified hematopoietic stem cells are obtained by genetically modifying induced pluripotent stem (iPS) cells and differentiating the iPS cells to become hematopoietic stem cells.

In some embodiments, the hematopoietic stem cells (HSC) or iPS cells are genetically modified such that the cells are capable of expressing the transgene upon their differentiation to microglial cells. In some embodiments, the locus that is genetically modified in the cells is transcriptionally active in microglial cells.

In some embodiments, a population of enriched HSC are subjected to a method to genetically modify the cells. In some embodiments, the enriched population comprises at least 50%, 55%, 60%, 65%, 70%, 75%, 80%, 85%, 90%, 91%, 92%, 93%, 94%, 95%, 96%, 97%, 98%, or 99% or more CD34+ HSC.

In some embodiments, the HSC or iPS cells are genetically modified using a sequence specific reagent. In some embodiments, the sequence specific reagent recognizes one or more sequences that are present in a locus that is expressed in microglial cells. In some embodiments, the sequence specific reagent cleaves a nucleic acid in the cells.

In some embodiments, the invention provides a method for making the genetically modified HSC or iPS cells comprising integrating a transgene into HSC or iPS cells. In some embodiments, the method comprises contacting the cells with a sequence specific reagent that cleaves a nucleic acid sequence at a locus expressed in microglial cells. In some embodiments, the method further comprises contacting the cells with a donor nucleic acid comprising the transgene.

In some embodiments, the sequence-specific reagent used to gene edit the cells of the present invention are rare-cutting endonucleases, such as TALE-nucleases (commercially available under Cellectis trademark TALEN®). Preferred reagents cleave one or several of the target sequences reported in Table 4 of the present specification.

In some embodiments, the sequence specific reagent targets intron of CX3CR1 preferably the first intron of CX3CR1 located between the first coding exon and second coding exon (SEQ ID NO: 76). The invention also provides with specific TALE nucleases that preferentially target endogenous polynucleotide sequences of CX3CR1 similar to SEQ ID NO:77 to 87. In some embodiments, the sequence specific reagents are CRISPR-Cas or CRISPR-Cpf using gRNA targeting endogenous sequences similar to SEQ ID NO:97 to 106.

In some embodiments, the sequence specific reagent targets intron of CD11B preferably the first intron of CD11B. The invention also provides with specific TALE nucleases that preferentially target endogenous polynucleotide sequences of CD11B similar to SEQ ID NO: 108 to 137. In some embodiment, the sequence specific reagents are CRISPR-Cas or CRISPR-Cpf using gRNA targeting endogenous sequences similar to SEQ ID NO:138 to 147.

In some embodiments, the sequence specific reagent targets intron of S100A9 preferably the first intron of S100A9. The invention also provides with specific TALE nucleases that preferentially target endogenous polynucleotide sequences of S100A9 similar to SEQ ID NO: 149 to 178. In some embodiment, the sequence specific reagents are CRISPR-Cas or CRISPR-Cpf using gRNA targeting endogenous sequences similar to SEQ ID NO:179 to 188.

In some embodiments, the polynucleotide template comprises a coding sequence of a transgene as described herein. In some embodiments, the polynucleotide template comprises a coding region of a gene selected from the group consisting of IDUA, IDS, ARSB, GUSB, ABCD1, GALC, ARSA, PSAP, GBA, FUCA1, MAN2B1, AGA, ASAH1, HEXA, GAA, SMPD1, LIPA and CDKL5.

In some embodiments, the donor nucleic acid comprises a nucleotide sequence selected from the group consisting of SEQ ID NOS: 1, 3, 5, 7, 9, 11, 13, 15, 17, 19, 21, 23, 25, 27, 29, 31, 33, 35 and variants thereof as described herein.

In some embodiments, the donor nucleic acid encodes a therapeutic protein comprising an amino acid sequence selected from any one of SEQ ID NOS: 2, 4, 6, 8, 10, 12, 14, 16, 18, 20, 22, 24, 26, 28, 30, 32, 34, 36 and variants thereof as described herein.

In some embodiments, the sequence specific reagent can be a chimeric polypeptide comprising a DNA binding domain and another domain displaying catalytic activity. Such catalytic activity can be nickase or double nickase to preferentially perform gene insertion by creating cohesive ends to facilitate gene integration by homologous recombination.

In some embodiments, the nuclease reagent induces NHEJ or homologous recombination mechanisms, which has the advantage of introducing stable and inheritable mutations into the genomic locus expressed in microglial cells.

By “nuclease reagent” is meant a nucleic acid molecule that contributes to a nuclease catalytic reaction in the target cell, preferably an endonuclease reaction, by itself or as a subunit of a complex such as a guide RNA Cas9, preferably leading to the cleavage of a nucleic acid sequence target.

The nuclease reagents of the invention are generally “sequence-specific nuclease reagents”, meaning that they can induce DNA cleavage in the cells at predetermined loci, referred to by extension as “targeted gene.” The nucleic acid sequence which is recognized by the sequence specific reagents is referred to as “target sequence.” Said target sequence is usually selected to be rare or unique in the cell's genome, and more extensively in the human genome, as can be determined using software and data available from human genome databases, such as http://www.ensembl.org/index.html.

In some embodiments, the sequence specific nuclease reagent used according to the invention, which specifically cleaves a sequence within the locus can also be used to induce the integration of an exogenous template at the locus. “Exogenous sequence” refers to any nucleotide or nucleic acid sequence that was not initially present at the selected locus. The exogenous sequence preferably comprises a sequence that codes for a therapeutic polypeptide as described herein for treating a disease herein. An endogenous sequence that is genetically modified by the insertion of a polynucleotide according to the method of the present invention, in order to express the polypeptide encoded thereby is broadly referred to as an exogenous coding sequence. In some embodiments, the targeted gene insertion comprises an exogenous sequence encoding a therapeutic polypeptide as described herein.

Exemplary selection methods applicable to DNA-binding domains, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237.

Selection of target sites; nucleases and methods for design and construction of fusion proteins (and polynucleotides encoding same) are known to those of skill in the art and described in detail in U.S. Patent Application Publication Nos. 20050064474 and 20060188987, incorporated by reference in their entireties herein.

DNA domains can be engineered to bind to any sequence of choice in a targeted locus. In some embodiments, the cells are genetically modified with a sequence specific reagent that has been engineered to bind a locus that is transcriptionally active in microglial cells. An engineered DNA-binding domain can have a novel binding specificity, compared to a naturally-occurring DNA-binding domain. Engineering methods include, but are not limited to, rational design and various types of selection. Rational design includes, for example, using databases comprising triplet (or quadruplet) nucleotide sequences and individual (e.g., zinc finger) amino acid sequences, in which each triplet or quadruplet nucleotide sequence is associated with one or more amino acid sequences of DNA binding domain which bind the particular triplet or quadruplet sequence. See, for example, U.S. Pat. Nos. 6,453,242 and 6,534,261, incorporated by reference herein in their entireties. Rational design of TAL-effector domains can also be performed. See, e.g., U.S. Patent Appl. Publication No. 2011/0301073.

In addition, as disclosed in these and other references, DNA-binding domains (e.g., multi-fingered zinc finger proteins) may be linked together using any suitable linker sequences, including for example, linkers of 5 or more amino acids. See, e.g., U.S. Pat. Nos. 6,479,626; 6,903,185; and 7,153,949 for exemplary linker sequences 6 or more amino acids in length. The proteins described herein may include any combination of suitable linkers between the individual DNA-binding domains of the protein. See, also, U.S. Patent Appl. Publication No. 2011/0301073.

The exogenous/donor sequence comprising the transgene is not identical over its entire length to sequences within the locus that is expressed in microglial cells. A donor sequence can contain a non-homologous sequence flanked by two regions of homology to allow for efficient HDR at the location of interest. Alternatively, a donor may have no regions of homology to the targeted location in the DNA and may be integrated by NHEJ-dependent end joining following cleavage at the target site. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

In some embodiments, the sequence specific reagent is a nucleic acid encoding an “engineered” or “programmable” rare-cutting endonuclease, such as a homing endonuclease as described for instance in WO 2004067736, a zing finger nuclease (ZFN) as described, for instance, by Urnov F., et al. (Nature 435:646-651 (2005)), a TALE-Nuclease as described, for instance, by Mussolino et al. (Nucl. Acids Res. 39(21):9283-9293 (2011)), or a MegaTAL nuclease as described, for instance by Boissel et al. (Nucleic Acids Research 42 (4):2591-2601 (2013)).

In some embodiments, the endonuclease reagent is transiently expressed into the cells, meaning that the reagent is not supposed to integrate into the genome or persist over a long period of time, such as the case of RNA, more particularly mRNA, proteins or complexes mixing proteins and nucleic acids (e.g.: Ribonucleoproteins).

In some embodiments, the sequence specific reagent is a nuclease that introduces DNA double strand break at a targeted locus, whose subsequent repair is exploited to achieve different outcomes. In some embodiments, a repair pathway based on homologous recombination can be used to copy information from an introduced DNA homology template. Such homology directed repair (HDR) can promote a specific addition of exogenous polynucleotide sequence (See, e.g., U.S. Pat. No. 8,921,332), e.g., a transgene as described herein, that can be expressed under the control of a promoter present on the exogenous polynucleotide sequence. In some embodiments, the transgene as described herein, can be expressed under the control of an endogenous promoter and at the same time that gene disruption is achieved. In some embodiments where gene disruption is achieved, the transgene is inserted at the stop codon of the endogenous gene and comprises a self-cleaving 2A peptide or IRES sequence. In a more preferred embodiments, the transgene is expressed under the control of an endogenous promoter without gene disruption. In some embodiments, the non-homologous end joining (NHEJ) repair pathway can be utilized (See, e.g., U.S. Pat. No. 9,458,439; He et al., Nucleic Acids Research, 44 e85, https://doi.org/10.1093/nar/gkw064).

In some embodiments, one or more targeted nucleases (e.g., CRISPR/Cas, ZFNS or TALENs) creates a double-stranded break in the target sequence (e.g., cellular chromatin) at a locus that is expressed in microglial cells. In some embodiments, a donor polynucleotide that comprises the transgene encoding the therapeutic protein and homology to the nucleotide sequence flanking the region of the break is introduced into the cell. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a sequence in cellular chromatin at a locus expressed in microglial cells can be altered and, in certain embodiments, can be modified to comprise a sequence present in a donor polynucleotide.

In some embodiments, the exogenous nucleotide sequence (the “donor sequence” that comprises the transgene) can contain sequences that are homologous, but not identical, to genomic sequences in the locus of interest expressed in microglial cells, thereby stimulating homologous recombination to insert a non-identical sequence in the locus of interest. In some embodiments, portions of the donor sequence that are homologous to sequences in the locus of interest exhibit between about 70 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. A non-homologous portion of the donor sequence contains sequences not present in the locus of interest, such that new sequence, viz., sequence encoding the transgene, are introduced into the locus of interest. In some embodiments, the non-homologous sequence is generally flanked by sequences of 50-1,000 base pairs (or any integral value therebetween) or any number of base pairs greater than 1,000, that are homologous or identical to sequences in the locus of interest. In some embodiments, the donor sequence is non-homologous to the first sequence, and is inserted into the genome by non-homologous recombination mechanisms.

The nucleases can target a gene that is active in microglial cells for the insertion of the transgene. In some embodiments, the nuclease is non-naturally occurring, i.e., engineered in the DNA-binding domain and/or cleavage domain. For example, the DNA-binding domain of a naturally-occurring nuclease or nuclease system may be altered to bind to a selected target site (e.g., a meganuclease that has been engineered to bind to site different than the cognate binding site or a CRISPR/Cas system utilizing an engineered single guide RNA). In other embodiments, the nuclease comprises heterologous DNA-binding and cleavage domains (e.g., zinc finger nucleases; TAL-effector nucleases; meganuclease DNA-binding domains with heterologous cleavage domains).

In some embodiments, the nuclease is a meganuclease (homing endonuclease). Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-Scell, I-PpoI, I-SceIII, I-CreI, I-TevI, I-TevII and I-TevIII. Their recognition sequences are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

In some embodiments, the nuclease comprises an engineered (non-naturally occurring) homing endonuclease (meganuclease). In some embodiments, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain.

In some embodiments, the DNA-binding domain comprises a naturally occurring or engineered (non-naturally occurring) TAL effector DNA binding domain. See, e.g., U.S. Patent Application Publication No. 2011/0301073, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like effectors (TALE) which mimic plant transcriptional activators and manipulate the plant transcriptome (Kay et al. (2007) Science 318:648-651). These proteins contain a DNA binding domain and a transcriptional activation domain. One of the most well characterized TALEs is AvrBs3 from Xanthomonas campestgris pv. vesicatoria (see Bonas et al. (1989) Mol Gen Genet 218:127-136 and WO 2010/079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al. (2006) J Plant Physiol 163 (3): 256-272). In addition, in the phytopathogenic bacteria Ralstonia solancearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al. (2007) Appl and Envir Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

In some embodiments, the DNA binding domain that binds to a target site in a target locus is an engineered domain from a TAL effector similar to those derived from the plant pathogens Xanthomonas (see Boch et al. (2009) Science 326:1509-1512 and Moscou and Bogdanove (2009) Science 326: 1501) and Ralstonia (see Heuer et al. (2007) Applied and Environmental Microbiology 73(13): 4379-4384); U.S. Pat. Nos. 8,420,782 and 8,440,431 and U.S. Patent Appl. Publication No. 2011/0301073.

In some embodiments, the DNA binding domain comprises a zinc finger protein. In some embodiments, the zinc finger protein is non-naturally occurring in that it is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Appl. Publication Nos. 2005/0064474; 2007/0218528; 2005/0267061, all incorporated herein by reference in their entireties.

An engineered zinc finger binding or TALE domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

In some embodiments, DNA domains (e.g., multi-fingered zinc finger proteins or TALE domains) may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The DNA binding proteins described herein may include any combination of suitable linkers between the individual zinc fingers of the protein. In addition, enhancement of binding specificity for zinc finger binding domains has been described, for example, in co-owned WO 02/077227.

DNA-binding domains and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496 and U.S. Patent Appl. Publication No. 2011/0301073.

Any suitable cleavage domain can be operatively linked to a DNA-binding domain to form a nuclease. For example, ZFP DNA-binding domains have been fused to nuclease domains to create ZFNs-a functional entity that is able to recognize its intended nucleic acid target through its engineered (ZFP) DNA binding domain and cause the DNA to be cut near the ZFP binding site via the nuclease activity. See, e.g., Kim et al. (1996) Proc Nat'l Acad Sci USA 93(3):1156-1160. More recently, ZFNs have been used for genome modification in a variety of organisms. See, for example, United States Patent Appl. Pub. Nos.: 2003/0232410; 2005/0208489; 2005/0026157; 2005/0064474; 2006/0188987; 2006/0063231; and International Publication WO 07/014275. Likewise, TALE DNA-binding domains have been fused to nuclease domains to create TALENs. See, e.g., U.S. Patent Appl. Publication No. 2011/0301073.

As noted above, the cleavage domain may be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides or nucleotide pairs can intervene between two target sites (e.g., from 2 to 50 nucleotide pairs or more). In general, the site of cleavage lies between the target sites.

In some embodiments, the dimerized cleavage half domains comprise one inactive cleavage domain and one active cleavage domain such that the targeted DNA is nicked on one strand rather than being completely cleaved (a “nickase”, see U.S. Patent Appl. Publication No. 2010/0047805). In other embodiments, two pairs of such nickases are used to cleave a target that is nicked on both DNA strands.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994) Proc. Natl. Acad. Sci. USA 91:883-887; Kim et al. (1994) J. Biol. Chem. 269:31,978-31,982. In one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95:10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a Fok I cleavage half-domain, can be used to reconstitute a catalytically active cleavage domain. Alternatively, a single polypeptide molecule containing a DNA binding domain and two Fok I cleavage half-domains can also be used.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Publication WO 07/014275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domains (also referred to as dimerization domain mutants) that minimize or prevent homodimerization, as described, for example, in U.S. Patent Appl Publication Nos. 2005/0064474; 2006/0188987 and 2008/0131962, the disclosures of all of which are incorporated by reference in their entireties herein. Amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains.

Exemplary engineered cleavage half-domains of Fok I that form obligate heterodimers include a pair in which a first cleavage half-domain includes mutations at amino acid residues at positions 490 and 538 of Fok I and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:1538K” and by mutating positions 486 (Q→E) and 499 (I→L) in another cleavage half-domain to produce an engineered cleavage half-domain designated “Q486E: 1499L”. The engineered cleavage half-domains described herein are obligate heterodimer mutants in which aberrant cleavage is minimized or abolished. See, e.g., U.S. Patent Publication No. 2008/0131962, the disclosure of which is incorporated by reference in its entirety for all purposes.

In some embodiments, the engineered cleavage half-domain comprises mutations at positions 486, 499 and 496 (numbered relative to wild-type FokI), for instance mutations that replace the wild type Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). (See US Patent Appl. Publication No. 2011/0201055, incorporated by reference herein). Engineered cleavage half-domains described herein can be prepared using any suitable method, for example, by site-directed mutagenesis of wild-type cleavage half-domains (Fok I) as described in U.S. Patent Appl. Publication Nos. 2005/0064474; 2008/0131962; and 2011/0201055.

In some embodiments, nucleases may be assembled in vivo at the nucleic acid target site using so-called “split-enzyme” technology (see, e.g. U.S. Patent Application Publication No. 2009/0068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in WO 2009/042163 and 2009/0068164. Nuclease expression constructs can be readily designed using methods known in the art. See, e.g., United States Patent Appl. Publication Nos.: 2003/0232410; 2005/0208489; 2005/0026157; 2005/0064474; 2006/0188987; 2006/0063231; and International Publication WO 07/014275. Expression of the nuclease may be under the control of a constitutive promoter or an inducible promoter, for example the galactokinase promoter which is activated (de-repressed) in the presence of raffinose and/or galactose and repressed in presence of glucose.

In some embodiments, the endonuclease reagent is a RNA-guide to be used in conjunction with a RNA guided endonuclease, such as Cas9 or Cpf1, as per, inter alia, the teaching by Doudna, J. et al., (Science 346 (6213): 1077) (2014)) and Zetsche, B. et al. (Cell 163(3): 759-771 (2015)) the teaching of which is incorporated herein by reference.

In some embodiments, the cells are genetically modified using the CRISPR (Clustered Regularly Interspaced Short Palindromic Repeats)/Cas (CRISPR Associated) nuclease system. The CRISPR/Cas is an engineered nuclease system based on a bacterial system that can be used for genome engineering. It is based on part of the adaptive immune response of many bacteria and archea. When a virus or plasmid invades a bacterium, segments of the invader's DNA are converted into CRISPR RNAs (crRNA) by the ‘immune’ response. This crRNA then associates, through a region of partial complementarity, with another type of RNA called tracrRNA to guide the Cas9 nuclease to a region homologous to the crRNA in the target DNA called a “protospacer”. Cas9 cleaves the DNA to generate blunt ends at the DSB at sites specified by a 20-nucleotide guide sequence contained within the crRNA transcript. Cas9 requires both the crRNA and the tracrRNA for site specific DNA recognition and cleavage. This system has now been engineered such that the crRNA and tracrRNA can be combined into one molecule (the “single guide RNA”), and the crRNA equivalent portion of the single guide RNA can be engineered to guide the Cas9 nuclease to target any desired sequence (see Jinek et al. (2012) Science 337, p. 816-821, Jinek et al., (2013), eLife 2:e00471, and David Segal, (2013) eLife 2:e00563).

The CRISPR (clustered regularly interspaced short palindromic repeats) locus, which encodes RNA components of the system, and the cas (CRISPR-associated) locus, which encodes proteins (Jansen et al., 2002 . Mol. Microbiol. 43:1565-1575; Makarova et al., 2002 . Nucleic Acids Res. 30: 482-496; Makarova et al., 2006 . Biol. Direct 1: 7; Haft et al., 2005 . PLOS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Wastson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called Cas' proteins are involved with the natural function of the CRISPR/Cas system and serve roles in functions such as insertion of the alien DNA etc.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein. Is also encompassed in RNA-guided endonucleases in the meaning of the present invention, the endonuclease Cpf1 as taught by Zetsche, B. et al. (Cell 163(3): 759-771 (2015)).

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong et al., (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA).

### Delivery Methods

The nucleases, polynucleotides encoding these nucleases, donor polynucleotides and compositions comprising the proteins and/or polynucleotides described herein for genetically modifying the cells may be delivered in vivo or ex vivo by any suitable means.

In some embodiments, polypeptides may be synthesized in situ in the cell as a result of the introduction of polynucleotides encoding the polypeptides into the cell. In some embodiments, the polypeptides can be produced outside the cell and then introduced into the cell. Methods for introducing a polynucleotide construct into cells are known in the art and include, as non-limiting examples, stable transformation methods wherein the polynucleotide construct is integrated into the genome of the cell, transient transformation methods wherein the polynucleotide construct is not integrated into the genome of the cell and virus mediated methods. In some embodiments, the polynucleotides may be introduced into a cell by recombinant viral vectors (e.g. retroviruses, adenoviruses), liposomes and the like. For example, transient transformation methods include, for example microinjection, electroporation or particle bombardment. The polynucleotides can be included in vectors, more particularly plasmids or virus, in view of being expressed in cells.

In some embodiments, the cells are transfected with a nucleic acid encoding an endonuclease reagent. In some embodiments, 80% of the endonuclease reagent is degraded by 30 hours, preferably by 24, more preferably by 20 hours after transfection.

In some embodiments, an endonuclease encoded by mRNA can be synthetized with a cap to enhance its stability according to techniques well known in the art, as described, for instance, by Kore A. L., et al. (Locked nucleic acid (LNA)-modified dinucleotide mRNA cap analogue: synthesis, enzymatic incorporation, and utilization (2009) J Am Chem Soc. 131 (18):6364-5).

In some embodiments, nucleases and/or donor constructs as described herein may also be delivered using vectors containing sequences encoding one or more of the CRISPR/Cas system(s), zinc finger or TALEN protein(s). Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more of the sequences needed for treatment. Thus, when one or more nucleases and a donor construct are introduced into the cell, the nucleases and/or donor polynucleotide may be carried on the same vector or on different vectors. When multiple vectors are used, each vector may comprise a sequence encoding one or multiple nucleases and/or donor constructs.

Conventional viral and non-viral based gene transfer methods can be used to introduce nucleic acids encoding nucleases and donor constructs in cells (e.g., mammalian cells) and target tissues.

Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology, Doerfler and Bohm (eds.) (1995); and Yu et al., Gene Therapy 1:13-26 (1994).

In some embodiments, methods of non-viral delivery of nucleic acids include electroporation, lipofection, microinjection, biolistics, virosomes, liposomes, immunoliposomes, polycation or lipid: nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

In some embodiments, electroporation steps can be used to transfect cells. In some embodiments, these steps are typically performed in closed chambers comprising parallel plate electrodes producing a pulse electric field between said parallel plate electrodes greater than 100 volts/cm and less than 5,000 volts/cm, substantially uniform throughout the treatment volume such as described in WO 2004/083379, which is incorporated by reference, especially from page 23, line 25 to page 29, line 11. One such electroporation chamber preferably has a geometric factor (cm⁻¹) defined by the quotient of the electrode gap squared (cm²) divided by the chamber volume (cm³), wherein the geometric factor is less than or equal to 0.1 cm⁻¹, wherein the suspension of the cells and the sequence specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

In some embodiments, different transgenes or multiple copies of the transgene can be included in one vector. The vector can comprise a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see Donnelly et al., J. of General Virology 82: 1013-1025 (2001); Donnelly et al., J. of Gen. Virology 78: 13-21 (1997); Doronina et al., Mol. And. Cell. Biology 28(13): 4227-4239 (2008); Atkins et al., RNA 13: 803-810 (2007)).

By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

In one embodiment, a polynucleotide encoding a sequence specific reagent according to the present invention can be mRNA which is introduced directly into the cells, for example by electroporation. In some embodiments, the cells can be electroporated using cytoPulse technology which allows, by the use of pulsed electric fields, to transiently permeabilize living cells for delivery of material into the cells. The technology, based on the use of PulseAgile (BTX Havard Apparatus, 84 October Hill Road, Holliston, Mass. 01746, USA) electroporation waveforms grants the precise control of pulse duration, intensity as well as the interval between pulses (see U.S. Pat. No. 6,010,613 and published International Application WO 2004/083379). All these parameters can be modified in order to reach the best conditions for high transfection efficiency with minimal mortality. The first high electric field pulses allow pore formation, while subsequent lower electric field pulses allow moving the polynucleotide into the cell.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam and Lipofectin). Cationic and neutral lipids that are suitable for efficient receptor-recognition lipofection of polynucleotides include those of Felgner, WO 91/17424, WO 91/16024.

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In some embodiments, the donor sequence and/or sequence specific reagent is encoded by a viral vector. In some embodiments, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). Other AAV serotypes, including by non-limiting example, AAV1, AAV3, AAV4, AAV5, AAV6, AAV8, AAV 8.2, AAV9, and AAV rh 10 and pseudotyped AAV such as AAV2/8, AAV2/5 and AAV2/6 can also be used in accordance with the present invention.

In some embodiments, the cells are administered an effective amount of one or more caspase inhibitors in combination with an AAV vector.

The nuclease-encoding sequences and donor constructs can be delivered using the same or different systems. For example, a donor polynucleotide can be carried by a viral vector, while the one or more nucleases can be delivered as mRNA compositions.

In some embodiments, one or more reagents can be delivered to cells using nanoparticles. In some embodiments, nanoparticles are coated with ligands, such as antibodies, having a specific affinity towards HSC surface proteins, such as CD105 (Uniprot #P17813). In some embodiments, the nanoparticles are biodegradable polymeric nanoparticles in which the sequence specific reagents under polynucleotide form are complexed with a polymer of polybeta amino ester and coated with polyglutamic acid (PGA).

Strategy for Exon Integration into Endogenous Intronic Genomic Loci

As a particular embodiment, the present patent application presents a method for integrating an exogenous coding sequence into an endogenous intronic genomic region, which allows to integrate said exogenous coding sequence preferably between the first and second endogenous coding exons of said genomic region.

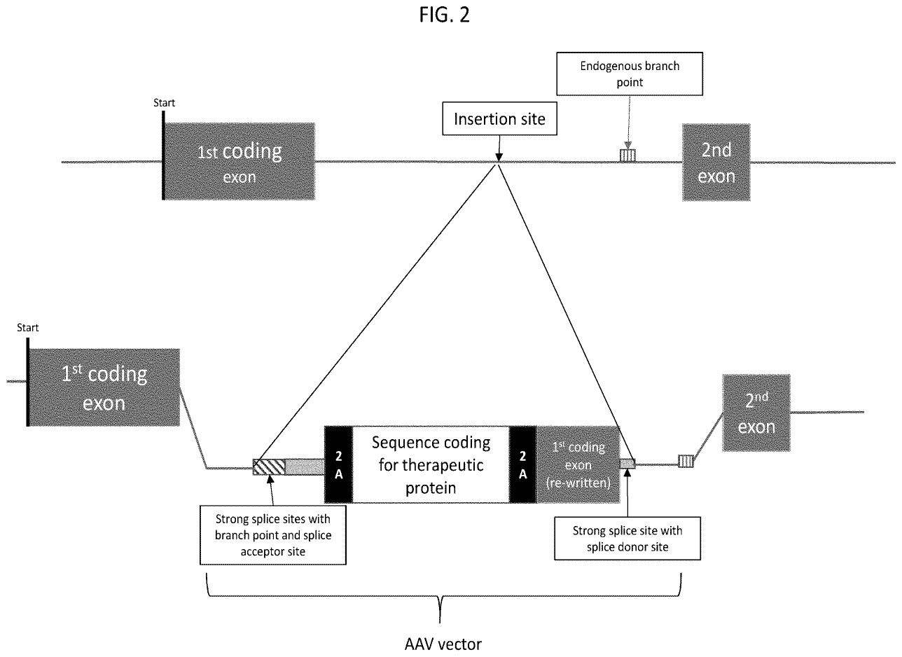

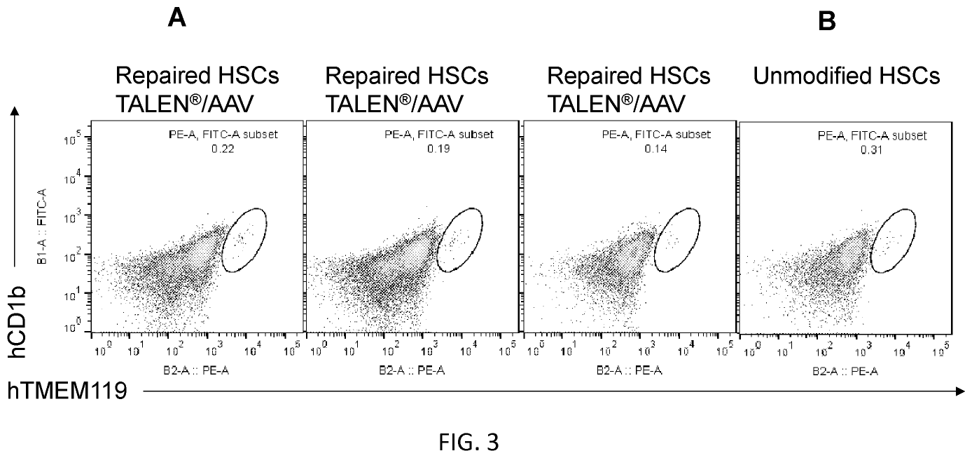

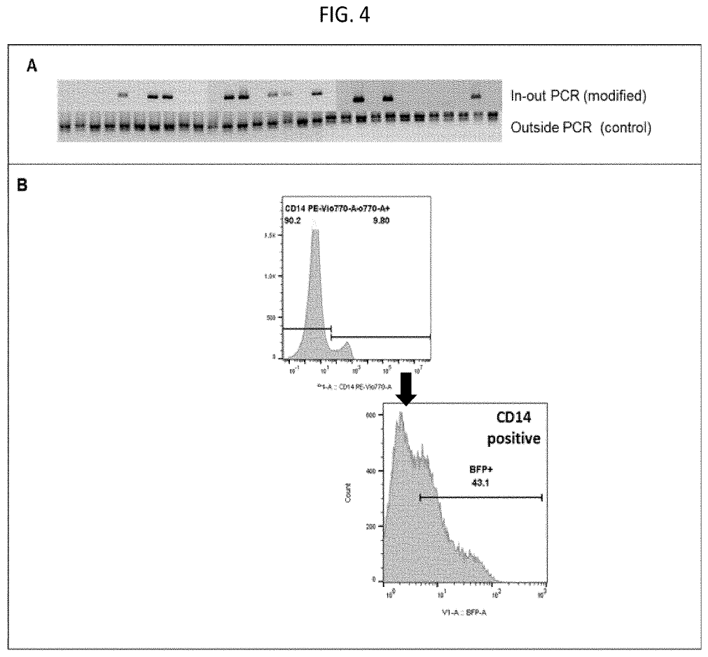

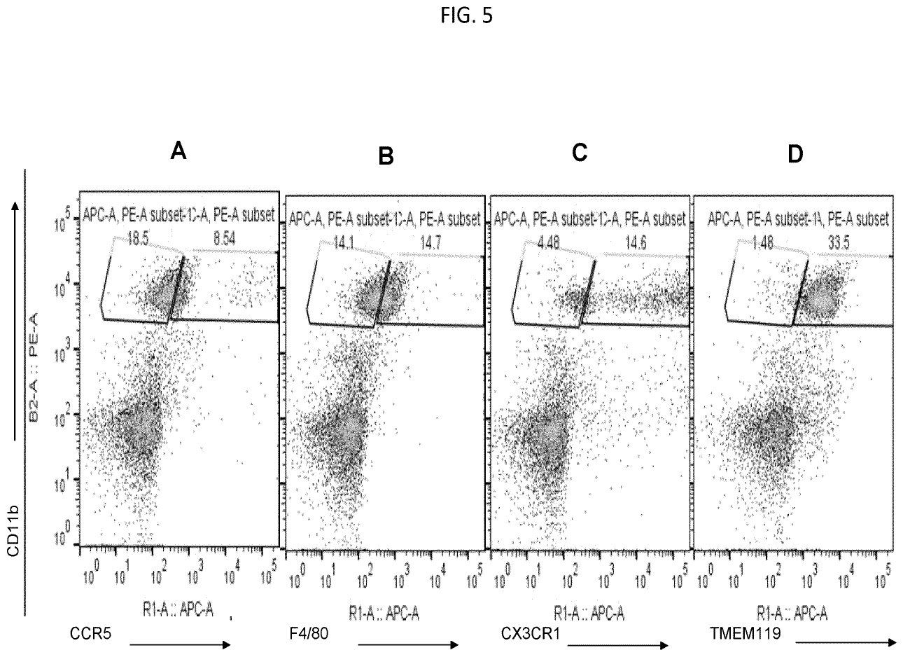

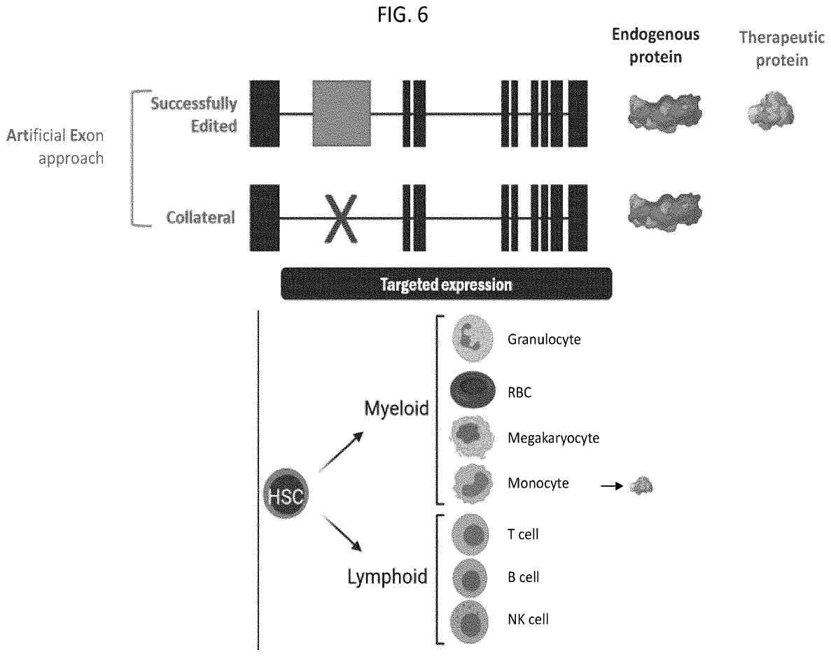

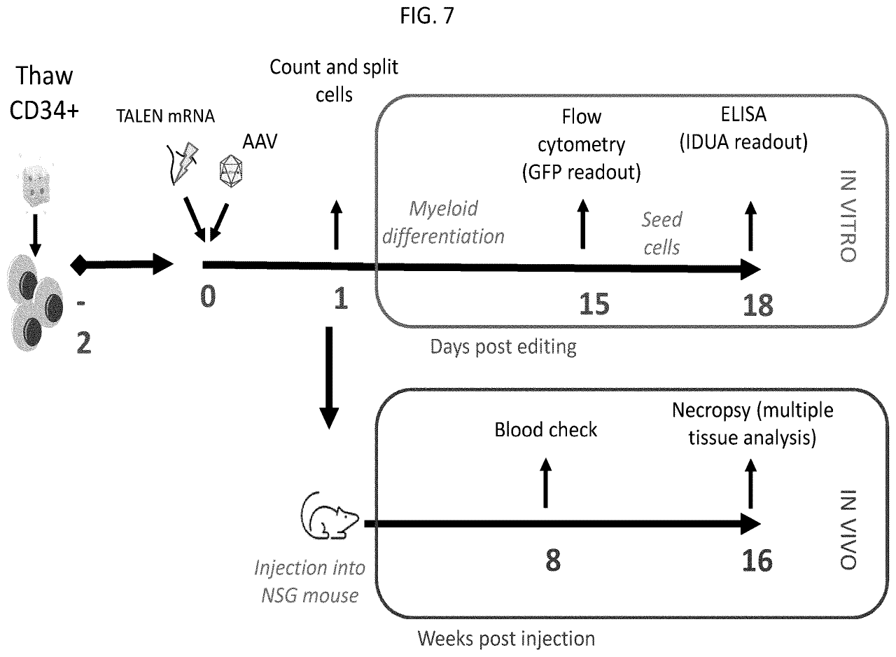

Said method is particularly useful, for instance, when said exons into said genomic regions are generally actively transcribed from a common endogenous promoter located upstream the first exon, such as illustrated in FIG. 2.

Said method has the advantage of preventing disruption of the transcript encoding the endogenous exonic regions, while allowing their transcription together with the exogenous coding sequence.

In general, said method comprises one or several of the following steps:
providing cell(s) comprising an endogenous intronic genomic region,
introducing into said cell(s) a polynucleotide template comprising an exogenous coding sequence,
   said polynucleotide template comprising or consisting of, in the 5′ to 3′ orientation:
      a first homologous polynucleotide sequence, which is homologous to the intronic sequence upstream of the insertion site,
      while said first polynucleotide sequence does not preferably comprise a branch point;
      a first strong splice site sequence, preferably comprising a branch point and a splice acceptor;
      a first sequence encoding 2A self-cleaving peptide;
      an exogenous sequence coding for a protein of interest;
      a second sequence encoding 2A self-cleaving peptide;
      a copy of the coding sequence of the first exon, optionally rewritten;
      a second strong splice site sequence preferably comprising a splice donor; and
      a second homologous polynucleotide sequence, which is homologous to the intronic sequence downstream of the insertion site;
inducing the integration of said exogenous polynucleotide into said intronic sequence, preferably by homologous recombination, to have said exogenous coding sequence being transcribed at said endogenous locus along with the first exon and preferably second (endogenous) exon, or a copy thereof.

In general, the second homologous polynucleotide, downstream of the copy of the first exon comprises a branch point, preferably that initially present in the endogenous sequence, in order to allow proper RNA splicing and expression of the second exon.

As a preferred embodiment, the copy of the sequence of said first exon can be rewritten at the polynucleotide level for codon optimization and/or reducing nucleotide sequence homologies with the endogenous locus sequences.

Each of the above steps can be performed by using methods well known in the art. According to a preferred embodiment, also in accordance with the delivery of therapeutic protein by HSCs or cells differentiated therefrom, the cells may originate from the patient himself, donors or from iPS cells, for instance as per the methods described in WO2018/189360, which are incorporated by reference.

The steps of the present method are generally performed ex-vivo, which means that the cells are cultivated and manufactured outside the human body. In general, the cells are not germinal cells or cells originating from human embryos and the methods are not intended to modify the germline or the genetic identity of human beings.

Integration of the polynucleotide template into said intronic sequence by homologous recombination can be facilitated by cleavage of a rare-cutting endonuclease at the insertion site. Accordingly, said method for exon integration may thus comprise the step of introducing or expressing into the cell a rare-cutting endonuclease, in particular TALE-nuclease, Zinc-Finger nuclease, meganuclease, CRISPR such as already described in this application to cleave said intronic sequence at the insertion site.

Insertion sites are thus generally determined by the target sequences of said rare-cutting endonucleases. Insertion sites are thus comprised within the rare-cutting endonuclease target sequences, which are themselves comprised into the intronic sequences of the selected loci, and more particularly into the loci contemplated by the present invention for the engineering of the HSCs as disclosed herein.

Preferred loci are those selected for the expression and delivery of the therapeutic proteins which comprise at least two endogenous exon sequences, in particular one of the intron sequences selected from CXCR3 (SEQ ID NO:76), CD11B (SEQ ID NO:107), S100A9 (SEQ ID NO:148) TMEM119 (SEQ ID NO:189), MERTK (SEQ ID NO:190), CD164 (SEQ ID NO:191), TLR7 (SEQ ID NO: 192), CD14 (SEQ ID NO:193), FCGR3A (CD16) (SEQ ID NO:194), TBXAS1 (SEQ ID NO: 195), DOK3 (SEQ ID NO: 196), ABCA1 (SEQ ID NO: 197), TMEM195 (SEQ ID NO: 198), TLR4 (SEQ ID NO:199), MR1 (SEQ ID NO:200), FCGR1A (CD64) (SEQ ID NO:201), CSF3R (SEQ ID NO:202), FGD4 (SEQ ID NO: 203) and TSPAN14 (SEQ ID NO:204), and B2M (SEQ ID NO: 205).

The polynucleotide template used for integrating the exogenous coding sequence usually includes a first and second polynucleotide sequence homologous to the intronic sequences referred to above, or at least 80%, preferably at least 75%, at least 80%, at least 90 or even preferably at least 95% identical to said polynucleotide sequences. The first and second homologous sequences are generally respectively homologous to the endogenous sequences upstream and downstream of the insertion site preferably over more than 50 base pairs (bp), more preferably over more than 100 bp, 200 bp, 500 bp and even more preferably between 50 et 500 bp.

As per the proposed method, the polynucleotide template to be inserted into the endogenous intronic genomic sequence comprises strong splice site sequences upstream and downstream of the exogenous coding sequence. Splice sites are particular motifs by which spliceosome identifies exons and removes intervening introns. Exogenous splice sites sequences can be introduced by cloning or alternatively by introducing mutations into the homologous sequences. Criteria to identify or design strong splice sites and examples of such sequences are provided in the literature, for instance in Shepard, P. J et al. [Efficient internal exon recognition depends on near equal contributions from the 3′ and 5′ splice sites (2011) Nucleic acids research, 39(20), 8928-37].

The first homologous sequence (i.e. upstream homologous sequence or left homology arm) is generally selected to exclude a branch point, which is generally located between 10 and 100 bp, preferably between 20 to 50 bp upstream of the second exon sequence. Human consensus for branch point sequences is generally yUnAy, where A is the branch point and the lowercase pyrimidines (‘y’) are not as well conserved as the uppercase U and A. Branch points are usually located 21-34 nucleotides upstream of the 3′ end of an intron, whereas so-called polypyrimidine tract spans 4-24 nucleotides downstream of the branch point [Gao, K., et al. (2008). Human branch point consensus sequence is yUnAy. Nucleic acids research, 36(7), 2257-67].

2A self-cleaving peptides, or 2A peptides, is a class of 18-22 aa-long peptides, which can induce the cleaving of the recombinant protein in cell. 2A peptides are derived from the 2A region in the genome of virus. Four members of 2A peptides family are frequently used in life science research: P2A, E2A, F2A and T2A. F2A, which is more commonly used, is derived from foot-and-mouth disease virus 18; E2A is derived from equine rhinitis A virus; P2A is derived from Porcine teschovirus-1 2A; T2A is derived from Thosea asigna virus 2A [Liu et al. (2017). “Systematic comparison of 2A peptides for cloning multi-genes in a polycistronic vector”. Scientific Reports. 7 (1)].

The present method for integrating an exogenous coding sequence into an endogenous intronic genomic region can be regarded as an invention in itself, as it is broadly applicable to any types of cells not being restricted to HSCs and irrespective the type of exogenous coding sequence that can inserted at the endogenous locus.

However, said method has been found to be particularly adapted to HSCs to produce therapeutic cells as described herein. There are significant advantages to use the above method for integrating corrected copy(ies) or additional copy(ies) of a gene, for their subsequent expression in differentiated HSCs to obtain cross correction of genetic deficiencies. Indeed, this preserves as much as possible the expression of the endogenous gene locus targeted by the insertion and thus is less susceptible to disturb cell differentiation and cell function of the resulting cells. This is especially sought with engineered HSCs that are intended to differentiate into macrophages to fulfill microglial function in the brain.

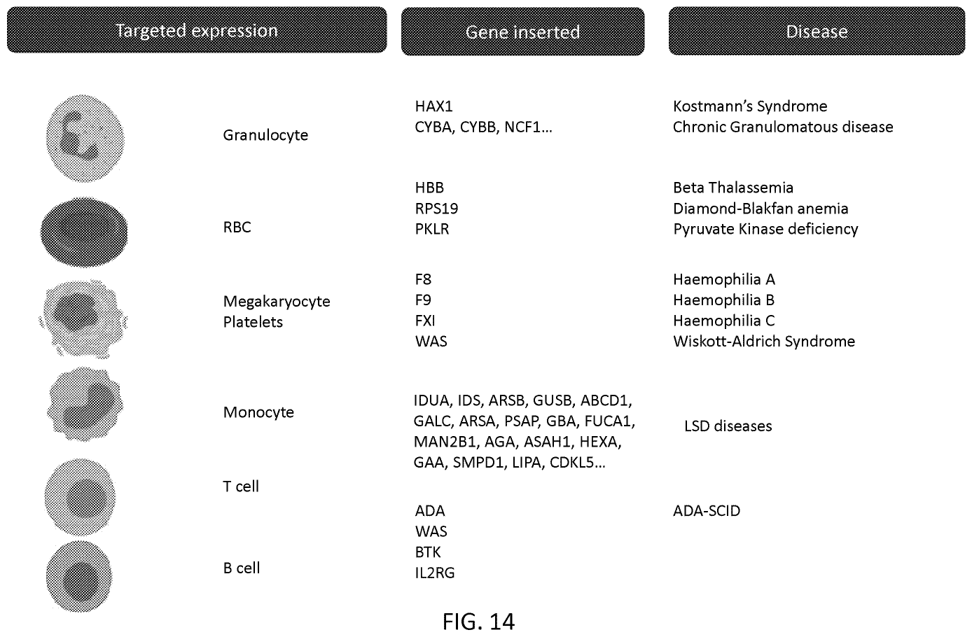

The present invention thus encompasses cells obtainable by the method described above and illustrated in FIG. 2 for integrating an exogenous coding sequence into an endogenous intronic genomic region, especially cells dedicated to gene therapy, and especially for cross correction of deficient alleles. This genetic insertion can be performed in HSCs to obtain expression in more differentiated stages, such as those cells used for the delivery of therapeutic proteins to the brain, in particular macrophages and microglial cells, for the treatment of disease, as more particularly described in FIG. 14.

As mentioned before, the present invention is drawn to a general method for integrating an exogenous coding sequence into an endogenous intronic genomic region or locus, without inactivating the expression of the endogenous exons present at this locus, especially sequences downstream the insertion site. This method thus prevents so-called “polar effects” generally observed in transgene genomic integration.

Said method comprises the following steps:
providing cell(s) comprising an endogenous intronic genomic region,
introducing into said cell(s) a polynucleotide template comprising an exogenous coding sequence, wherein said polynucleotide template comprises:
   a) a first homologous polynucleotide sequence, which is homologous to the intronic sequence upstream of the insertion site,
   b) a first strong splice site sequence, comprising a branch point and a splice acceptor;
   c) a first sequence encoding 2A self-cleaving peptide;
   d) an exogenous sequence coding for a protein of interest;
   e) a second sequence encoding 2A self-cleaving peptide;
   f) a copy of the coding sequence of the first exon(s);
   g) a second strong splice site sequence comprising a splice donor; and
   h) a second homologous polynucleotide sequence, which is homologous to the intronic sequence downstream of the insertion site; and optionally
inducing the integration of said exogenous polynucleotide into said intronic sequence, preferably by homologous recombination, to have said exogenous coding sequence being transcribed at said endogenous locus along with the first exon(s) or a copy thereof.

By the method of the invention, the above integration forms an artificial exon (Artex) that can be introduced into a hematopoietic stem cell (HSC) in order to obtain for instance expression of an exogenous coding sequence into at least one hematopoietic cell lineage.

In some preferred embodiments, said exogenous coding sequence encodes a protein of interest for treating a genetic disease for its expression in progenitor cells, red blood cells, granulocytes, megacaryocytes, monocytes, B-cells and/or T-cells as shown in FIG. 14.

According to some embodiments, this method is used for expression of a protein selected from FANCA, FANCC or FANCG in progenitor cells.

According to some embodiments, this method is used for expression of a protein selected from HBB, PKLR or RPS19 in red blood cells.

According to some embodiments, this method is used for expression of a protein selected from HAX1, CYBA, CYBB, NCF1, NCF2 or NCF4 in granulocytes.

According to some embodiments, this method is used for expression of a protein selected from Factor 8, Factor 9, Factor 11 or WAS in megakaryocytes.

According to some embodiments, this method is used for expression of a protein selected from IDUA, IDS, ARSB, GUSB, ABCD1, GALC, ARSA, PSAP, GBA, FUCA1, MAN2B1, AGA, ASAH1, HEXA, GAA, SMPD1, LIPA and CDKL5 in Monocytes.

According to some embodiments, this method is used for expression of a protein selected from from ADA, IL2RG, WAS or BTK in B-cells.

According to some embodiments, this method is used for expression of a protein selected from from ADA, IL2RG, WAS, BTK or CCR5 in T-cells.

Accordingly the expression of said exogenous coding sequence results into a protein of interest allowing the cross correction of an endogenous deficient protein. This method can be performed ex-vivo to produce engineered therapeutic cells for the treatment of at least one of the disease listed in FIG. 14, especially the multiple forms of lysosomal storage disease (LSD) identified so far.

The present invention is also drawn to insertion vectors that can be used to carry out the above methods, such as an AAV vector, preferably AAV6, characterized in that it comprises an exogenous polynucleotide sequence for insertion at an endogenous locus comprising the following sequences:
a) a first homologous polynucleotide sequence, which is homologous to the intronic sequence upstream of the insertion site,
b) a first strong splice site sequence, comprising a branch point and a splice acceptor;
c) a first sequence encoding 2A self-cleaving peptide;
d) an exogenous sequence coding for a protein of interest;
e) a second sequence encoding 2A self-cleaving peptide;
f) a copy of the coding sequence of the first exon;
g) a second strong splice site sequence comprising a splice donor; and
h) a second homologous polynucleotide sequence, which is homologous to the intronic sequence downstream of the insertion site.

In preferred embodiments, said first and second homologous sequences are homologous to an endogenous locus selected from: tmem119, s100a9, cd11b, b2m, cx3cr1, mertk, cd164, tlr4, tlr7, cd14, fcgr1a, fcgr3a, tbxas1, dok3, abca1, tmem195, mr1, csf3r, fgd4, tspan14, tgfbri, ccr5, gpr34, serpine2, slco2b1, p2ry12, olfml3, p2ry13, hexb, rhob, jun, rab3il1, cc12, fcrls, scoc, siglech, slc2a5, lrrc3, plxdc2, usp2, ctsf, cttnbp2nl, atp8a2, lgmn, mafb, egr1, bhlhe41, hpgds, ctsd, hspa1a, lag3, csf1r, adamts1, fl1r, golm1, nuak1, crybb1, ltc4s, sgce, pla2g15, ccl3l1, abhd12, ang, ophn1, sparc, pros1, p2ry6, lair1, il1a, epb41l2, adora3, rilpl1, pmepa1, ccl13, pde3b, scamp5, ppp1r9a, tjp1, ak1, b4galt4, gtf2 h2, trem2, ckb, acp2, pon3, agmo, tnfrsf17, fscn1, st3gal6, adap2, ccl4, entpd1, tmem86a, kctd12, dst, cts12, abcc3, pdgfb, pald1, tubgcp5, rapgef5, stab1, lacc1, tmc7, nrip1, kcnd1, tmem206, hps4, dagla, extl3, mlph, arhgap22, cxxc5, p4ha1, cysltr1, fgd2, kcnk13, gbgt1, c18orf1, cadm1, bco2, adrb1, c3ar1, large, leprel1, liph, upk1b, p2rx7, slc46a1, ebf3, ppp1r15a, il10ra, rasgrp3, fos, tppp, slc24a3, havcr2, nav2, apbb2, clstn1, blnk, gnaq, ptprm, frmd4a, cd86, tnfrsf11a, spint1, ppm1l, tgfbr2, cmklr1, tlr6, gas6, hist1h2ab, atf3, acvr1, abi3, lrp12, ttc28, plxna4, adamts16, rgs1, icam1, snx24, ly96, dnajb4, and ppfia4.

The present invention also encompasses an engineered cell obtainable by any of the previous methods, and more particularly one characterized in that an exogenous polynucleotide sequence has been inserted into an intron at an endogenous locus, wherein said polynucleotide sequence preferably comprises:
a first strong splice site sequence comprising a branch point and an acceptor site;
a first sequence encoding 2A self-cleaving peptide;
an exogenous sequence coding for a protein of interest, such as a therapeutic protein;
a second sequence encoding 2A self-cleaving peptide;
a copy of the coding sequence of the preceding exon endogenous to said locus;
a second strong splice site sequence comprising a splice donor site;
2. Still according to some preferred embodiments, said exogenous polynucleotide sequence in said engineered cell can inserted at an endogenous locus selected from: tmem119, s100a9, cd11b, B2m, Cx3cr1, mertk, cd164, tlr4, tlr7, cd14, fcgr1a, fcgr3a, tbxas1, dok3, abca1, tmem195, mr1, csf3r, fgd4, tspan14, tgfbri, ccr5, gpr34, serpine2, slco2b1, P2ry12, Olfml3, P2ry13, Hexb, Rhob, Jun, Rab3il1, Ccl2, Fcrls, Scoc, Siglech, Slc2a5, Lrrc3, Plxdc2, Usp2, Ctsf, Cttnbp2nl, Atp8a2, Lgmn, Mafb, Egr1, Bhlhe41, Hpgds, Ctsd, Hspa1a, Lag3, Csflr, Adamts1, F11r, Golm1, Nuak1, Crybb1, Ltc4s, Sgce, Pla2g15, Ccl3l1, Abhd12, Ang, Ophn1, Sparc, Pros1, P2ry6, Lair1, Il1a, Epb41l2, Adora3, Rilpl1, Pmepa1, Ccl13, Pde3b, Scamp5, Ppp1r9a, Tjp1, Ak1, B4galt4, Gtf2 h2, Trem2, Ckb, Acp2, Pon3, Agmo, Tnfrsf17, Fscn1, St3gal6, Adap2, Ccl4, Entpd1, Tmem86a, Kctd12, Dst, Cts12, Abcc3, Pdgfb, Pald1, Tubgcp5, Rapgef5, Stab1, Lacc1, Tmc7, Nrip1, Kcnd1, Tmem206, Hps4, Dagla, Ext13, Mlph, Arhgap22, Cxxc5, P4ha1, Cysltr1, Fgd2, Kcnk13, Gbgt1, C18orf1, Cadm1, Bco2, Adrb1, C3ar1, Large, Leprel1, Liph, Upk1b, P2rx7, Slc46a1, Ebf3, Ppp1r15a, Il10ra, Rasgrp3, Fos, Tppp, Slc24a3, Havcr2, Nav2, Apbb2, Clstn1, Blnk, Gnaq, Ptprm, Frmd4a, Cd86, Tnfrsf11a, Spint1, Ppm1l, Tgfbr2, Cmklr1, Tlr6, Gas6, Hist1h2ab, Atf3, Acvr1, Abi3, Lrp12, Ttc28, Plxna4, Adamts16, Rgs1, Icam1, Snx24, Ly96, Dnajb4, and Ppfia4. Preferred endogenous gene loci are S100A9 or CD11b

According to some preferred embodiments of the present invention, said exogenous polynucleotide sequence is inserted into an intron located between the first and second endogenous coding exons as illustrated in FIG. 2. The first and second encoding 2 self-cleaving peptide are generally different to avoid undesirable rare recombination events, which can be selected from SEQ ID NO:216 and SEQ ID NO:217.

According to some preferred embodiments, the first splice site referred to above comprises SEQ ID NO:206 or SEQ ID NO:207 as shown in the examples.

The coding sequences, in particular that of the first endogenous exon which can be replaced by homologous recombination as a result of the present method of integration as illustrated in FIG. 2, can be codon optimized (i.e. rewritten) to add polynucleotide sequence diversity and prevent undesired recombination events at the endogenous locus. The present invention thus more particularly provides engineered cells in which one exogenous sequence encoding a therapeutic protein selected from IDUA, IDS, ARSB, GUSB, ABCD1, GALC, ARSA, PSAP, GBA, FUCA1, MAN2B1, AGA, ASAH1, HEXA, GAA, SMPD1, LIPA and CDKL5 (SEQ ID NO:1 to SEQ ID NO: 35—see Table 1) is integrated at one locus selected from TMEM119, MERTK, CD164, TLR7, CD14, FCGR3A (CD16), TBXAS1, DOK3, ABCA1, TMEM195, TLR4, MR1, FCGR1A (CD64), CSF3R, FGD4, TSPAN14, CXCR3, CD11B, S100A9 and B2M, more particularly into their intronic polynucleotide sequences or any intronic sequences, at least 80%, preferably at least 75%, at least 80%, at least 90 or at least 95% identical to said polynucleotide sequences (to take into account the variability of these sequences throughout the animal kingdom and more particularly the human species).

The invention is thus more specifically directed to one of the following types of engineered cells, wherein:
IDUA is introduced at the CXCR3 locus, preferably into SEQ ID NO:76;
IDS is introduced at the CXCR3 locus, preferably into SEQ ID NO:76;
ARSB is introduced at the CXCR3 locus, preferably into SEQ ID NO:76;
GUSB is introduced at the CXCR3 locus, preferably into SEQ ID NO:76;
ABCD1 is introduced at the CXCR3 locus, preferably into SEQ ID NO:76;
GALC is introduced at the CXCR3 locus, preferably into SEQ ID NO:76;
ARSA is introduced at the CXCR3 locus, preferably into SEQ ID NO:76;
PSAP is introduced at the CXCR3 locus, preferably into SEQ ID NO:76;
GBA is introduced at the CXCR3 locus, preferably into SEQ ID NO:76;
FUCA1 is introduced at the CXCR3 locus, preferably into SEQ ID NO:76;
MAN2B1 is introduced at the CXCR3 locus, preferably into SEQ ID NO:76;
AGA is introduced at the CXCR3 locus, preferably into SEQ ID NO:76;
ASAH1 is introduced at the CXCR3 locus, preferably into SEQ ID NO:76;
HEXA is introduced at the CXCR3 locus, preferably into SEQ ID NO:76;
GAA is introduced at the CXCR3 locus, preferably into SEQ ID NO:76;
SMPD1 is introduced at the CXCR3 locus, preferably into SEQ ID NO:76;
LIPA is introduced at the CXCR3 locus, preferably into SEQ ID NO:76;
CDKL5 is introduced at the CXCR3 locus, preferably into SEQ ID NO:76;
IDUA is introduced at the CD11B locus, preferably into SEQ ID NO:107;
IDS is introduced at the CD11B locus, preferably into SEQ ID NO: 107;
ARSB is introduced at the CD11B locus, preferably into SEQ ID NO: 107;
GUSB is introduced at the CD11B locus, preferably into SEQ ID NO: 107;
ABCD1 is introduced at the CD11B locus, preferably into SEQ ID NO:107;
GALC is introduced at the CD11B locus, preferably into SEQ ID NO:107;
ARSA is introduced at the CD11B locus, preferably into SEQ ID NO:107;
PSAP is introduced at the CD11B locus, preferably into SEQ ID NO: 107;
GBA is introduced at the CD11B locus, preferably into SEQ ID NO: 107;
FUCA1 is introduced at the CD11B locus, preferably into SEQ ID NO:107;
MAN2B1 is introduced at the CD11B locus, preferably into SEQ ID NO: 107;
AGA is introduced at the CD11B locus, preferably into SEQ ID NO: 107;
ASAH1 is introduced at the CD11B locus, preferably into SEQ ID NO: 107;
HEXA is introduced at the CD11B locus, preferably into SEQ ID NO:107;
GAA is introduced at the CD11B locus, preferably into SEQ ID NO: 107;
SMPD1 is introduced at the CD11B locus, preferably into SEQ ID NO:107;
LIPA is introduced at the CD11B locus, preferably into SEQ ID NO: 107;
CDKL5 is introduced at the CD11B locus, preferably into SEQ ID NO: 107;
IDUA is introduced at the S100A9 locus, preferably into SEQ ID NO:148;
IDS is introduced at the S100A9 locus, preferably into SEQ ID NO:148;
ARSB is introduced at the S100A9 locus, preferably into SEQ ID NO: 148;
GUSB is introduced at the S100A9 locus, preferably into SEQ ID NO: 148;
ABCD1 is introduced at the S100A9 locus, preferably into SEQ ID NO:148;
GALC is introduced at the S100A9 locus, preferably into SEQ ID NO: 148;
ARSA is introduced at the S100A9 locus, preferably into SEQ ID NO: 148;
PSAP is introduced at the S100A9 locus, preferably into SEQ ID NO: 148;
GBA is introduced at the S100A9 locus, preferably into SEQ ID NO:148;
FUCA1 is introduced at the S100A9 locus, preferably into SEQ ID NO:148;
MAN2B1 is introduced at the S100A9 locus, preferably into SEQ ID NO:148;
AGA is introduced at the S100A9 locus, preferably into SEQ ID NO:148;
ASAH1 is introduced at the S100A9 locus, preferably into SEQ ID NO:148;
HEXA is introduced at the S100A9 locus, preferably into SEQ ID NO: 148;
GAA is introduced at the S100A9 locus, preferably into SEQ ID NO: 148;
SMPD1 is introduced at the S100A9 locus, preferably into SEQ ID NO:148;
LIPA is introduced at the S100A9 locus, preferably into SEQ ID NO:148;
CDKL5 is introduced at the S100A9 locus, preferably into SEQ ID NO:148;
IDUA is introduced at the TMEM119 locus, preferably into SEQ ID NO:189;
IDS is introduced at the TMEM119 locus, preferably into SEQ ID NO:189;
ARSB is introduced at the TMEM119 locus, preferably into SEQ ID NO:189;
GUSB is introduced at the TMEM119 locus, preferably into SEQ ID NO:189;
ABCD1 is introduced at the TMEM119 locus, preferably into SEQ ID NO:189;
GALC is introduced at the TMEM119 locus, preferably into SEQ ID NO:189;
ARSA is introduced at the TMEM119 locus, preferably into SEQ ID NO:189;
PSAP is introduced at the TMEM119 locus, preferably into SEQ ID NO:189;
GBA is introduced at the TMEM119 locus, preferably into SEQ ID NO: 189;
FUCA1 is introduced at the TMEM119 locus, preferably into SEQ ID NO:189;
MAN2B1 is introduced at the TMEM119 locus, preferably into SEQ ID NO: 189;
AGA is introduced at the TMEM119 locus, preferably into SEQ ID NO:189;
ASAH1 is introduced at the TMEM119 locus, preferably into SEQ ID NO:189;
HEXA is introduced at the TMEM119 locus, preferably into SEQ ID NO:189;
GAA is introduced at the TMEM119 locus, preferably into SEQ ID NO: 189;
SMPD1 is introduced at the TMEM119 locus, preferably into SEQ ID NO:189;
LIPA is introduced at the TMEM119 locus, preferably into SEQ ID NO:189;
CDKL5 is introduced at the TMEM119 locus, preferably into SEQ ID NO:189;
IDUA is introduced at the MERTK locus, preferably into SEQ ID NO: 190;
IDS is introduced at the MERTK locus, preferably into SEQ ID NO: 190;
ARSB is introduced at the MERTK locus, preferably into SEQ ID NO: 190;
GUSB is introduced at the MERTK locus, preferably into SEQ ID NO: 190;
ABCD1 is introduced at the MERTK locus, preferably into SEQ ID NO: 190;
GALC is introduced at the MERTK locus, preferably into SEQ ID NO: 190;
ARSA is introduced at the MERTK locus, preferably into SEQ ID NO: 190;
PSAP is introduced at the MERTK locus, preferably into SEQ ID NO: 190;
GBA is introduced at the MERTK locus, preferably into SEQ ID NO: 190;
FUCA1 is introduced at the MERTK locus, preferably into SEQ ID NO:190;
MAN2B1 is introduced at the MERTK locus, preferably into SEQ ID NO:190;
AGA is introduced at the MERTK locus, preferably into SEQ ID NO: 190;
ASAH1 is introduced at the MERTK locus, preferably into SEQ ID NO: 190;
HEXA is introduced at the MERTK locus, preferably into SEQ ID NO: 190;
GAA is introduced at the MERTK locus, preferably into SEQ ID NO:190;
SMPD1 is introduced at the MERTK locus, preferably into SEQ ID NO: 190;
LIPA is introduced at the MERTK locus, preferably into SEQ ID NO: 190;
CDKL5 is introduced at the MERTK locus, preferably into SEQ ID NO: 190;
IDUA is introduced at the CD164 locus, preferably into SEQ ID NO:191;
IDS is introduced at the CD164 locus, preferably into SEQ ID NO: 191;
ARSB is introduced at the CD164 locus, preferably into SEQ ID NO: 191;
GUSB is introduced at the CD164 locus, preferably into SEQ ID NO: 191;
ABCD1 is introduced at the CD164 locus, preferably into SEQ ID NO: 191;
GALC is introduced at the CD164 locus, preferably into SEQ ID NO: 191;
ARSA is introduced at the CD164 locus, preferably into SEQ ID NO: 191;
PSAP is introduced at the CD164 locus, preferably into SEQ ID NO: 191;
GBA is introduced at the CD164 locus, preferably into SEQ ID NO: 191;
FUCA1 is introduced at the CD164 locus, preferably into SEQ ID NO:191;
MAN2B1 is introduced at the CD164 locus, preferably into SEQ ID NO:191;
AGA is introduced at the CD164 locus, preferably into SEQ ID NO: 191;
ASAH1 is introduced at the CD164 locus, preferably into SEQ ID NO: 191;
HEXA is introduced at the CD164 locus, preferably into SEQ ID NO: 191;
GAA is introduced at the CD164 locus, preferably into SEQ ID NO:191;
SMPD1 is introduced at the CD164 locus, preferably into SEQ ID NO:191;
LIPA is introduced at the CD164 locus, preferably into SEQ ID NO:191;
CDKL5 is introduced at the CD164 locus, preferably into SEQ ID NO:191;
IDUA is introduced at the TLR7 locus, preferably into SEQ ID NO: 192;
IDS is introduced at the TLR7 locus, preferably into SEQ ID NO: 192;
ARSB is introduced at the TLR7 locus, preferably into SEQ ID NO: 192;
GUSB is introduced at the TLR7 locus, preferably into SEQ ID NO: 192;
ABCD1 is introduced at the TLR7 locus, preferably into SEQ ID NO: 192;
GALC is introduced at the TLR7 locus, preferably into SEQ ID NO: 192;
ARSA is introduced at the TLR7 locus, preferably into SEQ ID NO: 192;
PSAP is introduced at the locus TLR7, preferably into SEQ ID NO: 192;
GBA is introduced at the TLR7 locus, preferably into SEQ ID NO: 192;
FUCA1 is introduced at the TLR7 locus, preferably into SEQ ID NO: 192;
MAN2B1 is introduced at the TLR 7locus, preferably into SEQ ID NO: 192;
AGA is introduced at the TLR7 locus, preferably into SEQ ID NO: 192;
ASAH1 is introduced at the TLR7 locus, preferably into SEQ ID NO: 192;
HEXA is introduced at the TLR7 locus, preferably into SEQ ID NO: 192;
GAA is introduced at the TLR7 locus, preferably into SEQ ID NO: 192;
SMPD1 is introduced at the TLR7 locus, preferably into SEQ ID NO:192;
LIPA is introduced at the TLR7 locus, preferably into SEQ ID NO:192;
CDKL5 is introduced at the TLR7 locus, preferably into SEQ ID NO:192;
IDUA is introduced at the CD14 locus, preferably into SEQ ID NO:193;
IDS is introduced at the CD14 locus, preferably into SEQ ID NO: 193;
ARSB is introduced at the CD14 locus, preferably into SEQ ID NO: 193;
GUSB is introduced at the CD14 locus, preferably into SEQ ID NO: 193;
ABCD1 is introduced at the CD14 locus, preferably into SEQ ID NO: 193;
GALC is introduced at the CD14 locus, preferably into SEQ ID NO: 193;
ARSA is introduced at the CD14 locus, preferably into SEQ ID NO: 193;
PSAP is introduced at the locus CD14, preferably into SEQ ID NO: 193;
GBA is introduced at the CD14 locus, preferably into SEQ ID NO: 193;
FUCA1 is introduced at the CD14 locus, preferably into SEQ ID NO:193;
MAN2B1 is introduced at the CD14 locus, preferably into SEQ ID NO: 193;
AGA is introduced at the CD14 locus, preferably into SEQ ID NO: 193;
ASAH1 is introduced at the CD14 locus, preferably into SEQ ID NO: 193;
HEXA is introduced at the CD14 locus, preferably into SEQ ID NO: 193;
GAA is introduced at the CD14 locus, preferably into SEQ ID NO:193;
SMPD1 is introduced at the CD14 locus, preferably into SEQ ID NO:193;
LIPA is introduced at the CD14 locus, preferably into SEQ ID NO: 193;
CDKL5 is introduced at the CD14 locus, preferably into SEQ ID NO:193;
IDUA is introduced at the FCGR3A locus, preferably into SEQ ID NO: 194;
IDS is introduced at the FCGR3A locus, preferably into SEQ ID NO: 194;
ARSB is introduced at the FCGR3A locus, preferably into SEQ ID NO: 194;
GUSB is introduced at the FCGR3A locus, preferably into SEQ ID NO: 194;
ABCD1 is introduced at the FCGR3A locus, preferably into SEQ ID NO: 194;
GALC is introduced at the FCGR3A locus, preferably into SEQ ID NO: 194;
ARSA is introduced at the FCGR3A locus, preferably into SEQ ID NO: 194;
PSAP is introduced at the FCGR3A locus, preferably into SEQ ID NO: 194;
GBA is introduced at the FCGR3A locus, preferably into SEQ ID NO: 194;
FUCA1 is introduced at the FCGR3A locus, preferably into SEQ ID NO: 194;
MAN2B1 is introduced at the FCGR3A locus, preferably into SEQ ID NO:194;
AGA is introduced at the FCGR3A locus, preferably into SEQ ID NO: 194;
ASAH1 is introduced at the FCGR3A locus, preferably into SEQ ID NO: 194;
HEXA is introduced at the FCGR3A locus, preferably into SEQ ID NO: 194;
GAA is introduced at the FCGR3A locus, preferably into SEQ ID NO: 194;
SMPD1 is introduced at the FCGR3A locus, preferably into SEQ ID NO: 194;
LIPA is introduced at the FCGR3A locus, preferably into SEQ ID NO: 194;
CDKL5 is introduced at the FCGR3A locus, preferably into SEQ ID NO: 194;
IDUA is introduced at the TBXAS1 locus, preferably into SEQ ID NO: 195;
IDS is introduced at the TBXAS1 locus, preferably into SEQ ID NO: 195;
ARSB is introduced at the TBXAS1 locus, preferably into SEQ ID NO: 195;
GUSB is introduced at the TBXAS1 locus, preferably into SEQ ID NO: 195;
ABCD1 is introduced at the TBXAS1 locus, preferably into SEQ ID NO: 195;
GALC is introduced at the TBXAS1 locus, preferably into SEQ ID NO: 195;
ARSA is introduced at the TBXAS1 locus, preferably into SEQ ID NO: 195;
PSAP is introduced at the TBXAS1 locus, preferably into SEQ ID NO: 195;
GBA is introduced at the TBXAS1 locus, preferably into SEQ ID NO: 195;
FUCA1 is introduced at the TBXAS1 locus, preferably into SEQ ID NO: 195;
MAN2B1 is introduced at the TBXAS1 locus, preferably into SEQ ID NO:195;
AGA is introduced at the TBXAS1 locus, preferably into SEQ ID NO: 195;
ASAH1 is introduced at the TBXAS1 locus, preferably into SEQ ID NO: 195;
HEXA is introduced at the TBXAS1 locus, preferably into SEQ ID NO: 195;
GAA is introduced at the TBXAS1 locus, preferably into SEQ ID NO:195;
SMPD1 is introduced at the TBXAS1 locus, preferably into SEQ ID NO:195;
LIPA is introduced at the TBXAS1 locus, preferably into SEQ ID NO: 195;
CDKL5 is introduced at the TBXAS1 locus, preferably into SEQ ID NO:195;
IDUA is introduced at the DOK3 locus, preferably into SEQ ID NO: 196;
IDS is introduced at the DOK3 locus, preferably into SEQ ID NO: 196;
ARSB is introduced at the DOK3 locus, preferably into SEQ ID NO: 196;
GUSB is introduced at the DOK3 locus, preferably into SEQ ID NO: 196;
ABCD1 is introduced at the DOK3 locus, preferably into SEQ ID NO: 196;
GALC is introduced at the DOK3 locus, preferably into SEQ ID NO: 196;
ARSA is introduced at the DOK3 locus, preferably into SEQ ID NO: 196;
PSAP is introduced at the DOK3 locus, preferably into SEQ ID NO: 196;
GBA is introduced at the DOK3 locus, preferably into SEQ ID NO: 196;
FUCA1 is introduced at the DOK3 locus, preferably into SEQ ID NO:196;
MAN2B1 is introduced at the DOK3 locus, preferably into SEQ ID NO: 196;
AGA is introduced at the DOK3 locus, preferably into SEQ ID NO: 196;
ASAH1 is introduced at the DOK3 locus, preferably into SEQ ID NO: 196;
HEXA is introduced at the DOK3 locus, preferably into SEQ ID NO: 196;
GAA is introduced at the DOK3 locus, preferably into SEQ ID NO: 196;
SMPD1 is introduced at the DOK3 locus, preferably into SEQ ID NO:196;
LIPA is introduced at the DOK3 locus, preferably into SEQ ID NO: 196;
CDKL5 is introduced at the DOK3 locus, preferably into SEQ ID NO:196;
IDUA is introduced at the ABCA1 locus, preferably into SEQ ID NO:197;
IDS is introduced at the ABCA1 locus, preferably into SEQ ID NO: 197;
ARSB is introduced at the ABCA1 locus, preferably into SEQ ID NO: 197;
GUSB is introduced at the ABCA1 locus, preferably into SEQ ID NO: 197;
ABCD1 is introduced at the ABCA1 locus, preferably into SEQ ID NO: 197;
GALC is introduced at the ABCA1 locus, preferably into SEQ ID NO: 197;
ARSA is introduced at the ABCA1 locus, preferably into SEQ ID NO: 197;
PSAP is introduced at the ABCA1 locus, preferably into SEQ ID NO: 197;
GBA is introduced at the ABCA1 locus, preferably into SEQ ID NO: 197;
FUCA1 is introduced at the ABCA1 locus, preferably into SEQ ID NO: 197;
MAN2B1 is introduced at the ABCA1 locus, preferably into SEQ ID NO:197;
AGA is introduced at the ABCA1 locus, preferably into SEQ ID NO: 197;
ASAH1 is introduced at the ABCA1 locus, preferably into SEQ ID NO: 197;
HEXA is introduced at the ABCA1 locus, preferably into SEQ ID NO: 197;
GAA is introduced at the ABCA1 locus, preferably into SEQ ID NO:197;
SMPD1 is introduced at the ABCA1 locus, preferably into SEQ ID NO: 197;
LIPA is introduced at the ABCA1 locus, preferably into SEQ ID NO:197;
CDKL5 is introduced at the ABCA1 locus, preferably into SEQ ID NO: 197;
IDUA is introduced at the TMEM195 locus, preferably into SEQ ID NO: 198;
IDS is introduced at the TMEM195 locus, preferably into SEQ ID NO: 198;
ARSB is introduced at the TMEM195 locus, preferably into SEQ ID NO: 198;
GUSB is introduced at the TMEM195 locus, preferably into SEQ ID NO: 198;
ABCD1 is introduced at the TMEM195 locus, preferably into SEQ ID NO: 198;
GALC is introduced at the TMEM195 locus, preferably into SEQ ID NO: 198;
ARSA is introduced at the TMEM195 locus, preferably into SEQ ID NO: 198;
PSAP is introduced at the TMEM195 locus, preferably into SEQ ID NO: 198;
GBA is introduced at the TMEM195 locus, preferably into SEQ ID NO: 198;
FUCA1 is introduced at the TMEM195 locus, preferably into SEQ ID NO:198;
MAN2B1 is introduced at the TMEM195 locus, preferably into SEQ ID NO: 198;
AGA is introduced at the TMEM195 locus, preferably into SEQ ID NO: 198;
ASAH1 is introduced at the TMEM195 locus, preferably into SEQ ID NO: 198;
HEXA is introduced at the TMEM195 locus, preferably into SEQ ID NO: 198;
GAA is introduced at the TMEM195 locus, preferably into SEQ ID NO: 198;
SMPD1 is introduced at the TMEM195 locus, preferably into SEQ ID NO: 198;
LIPA is introduced at the TMEM195 locus, preferably into SEQ ID NO: 198;
CDKL5 is introduced at the TMEM195 locus, preferably into SEQ ID NO: 198;
IDUA is introduced at the TLR4 locus, preferably into SEQ ID NO: 199;
IDS is introduced at the TLR4 locus, preferably into SEQ ID NO: 199;
ARSB is introduced at the TLR4 locus, preferably into SEQ ID NO: 199;
GUSB is introduced at the TLR4 locus, preferably into SEQ ID NO: 199;
ABCD1 is introduced at the TLR4 locus, preferably into SEQ ID NO: 199;
GALC is introduced at the TLR4 locus, preferably into SEQ ID NO: 199;
ARSA is introduced at the TLR4 locus, preferably into SEQ ID NO: 199;
PSAP is introduced at the TLR4 locus, preferably into SEQ ID NO: 199;
GBA is introduced at the TLR4 locus, preferably into SEQ ID NO: 199;
FUCA1 is introduced at the TLR4 locus, preferably into SEQ ID NO: 199;
MAN2B1 is introduced at the TLR4 locus, preferably into SEQ ID NO:199;
AGA is introduced at the TLR4 locus, preferably into SEQ ID NO: 199;
ASAH1 is introduced at the TLR4 locus, preferably into SEQ ID NO: 199;
HEXA is introduced at the TLR4 locus, preferably into SEQ ID NO: 199;
GAA is introduced at the TLR4 locus, preferably into SEQ ID NO: 199;
SMPD1 is introduced at the TLR4 locus, preferably into SEQ ID NO: 199;
LIPA is introduced at the TLR4 locus, preferably into SEQ ID NO:199;
CDKL5 is introduced at the TLR4 locus, preferably into SEQ ID NO: 199;
IDUA is introduced at the MR1 locus, preferably into SEQ ID NO:200;
IDS is introduced at the MR1 locus, preferably into SEQ ID NO: 200;
ARSB is introduced at the MR1 locus, preferably into SEQ ID NO: 200;
GUSB is introduced at the MR1 locus, preferably into SEQ ID NO: 200;
ABCD1 is introduced at the MR1 locus, preferably into SEQ ID NO: 200;
GALC is introduced at the MR1 locus, preferably into SEQ ID NO: 200;
ARSA is introduced at the MR1 locus, preferably into SEQ ID NO: 200;
PSAP is introduced at the MR1 locus, preferably into SEQ ID NO: 200;
GBA is introduced at the MR1 locus, preferably into SEQ ID NO: 200;
FUCA1 is introduced at the MR1 locus, preferably into SEQ ID NO: 200;
MAN2B1 is introduced at the MR1 locus, preferably into SEQ ID NO: 200;
AGA is introduced at the MR1 locus, preferably into SEQ ID NO: 200;
ASAH1 is introduced at the MR1 locus, preferably into SEQ ID NO: 200;
HEXA is introduced at the MR1 locus, preferably into SEQ ID NO: 200;
GAA is introduced at the MR1 locus, preferably into SEQ ID NO: 200;
SMPD1 is introduced at the MR1 locus, preferably into SEQ ID NO: 200;
LIPA is introduced at the MR1 locus, preferably into SEQ ID NO: 200;
CDKL5 is introduced at the MR1 locus, preferably into SEQ ID NO: 200;
IDUA is introduced at the FCGR1A locus, preferably into SEQ ID NO:201;
IDS is introduced at the FCGR1A locus, preferably into SEQ ID NO: 201;
ARSB is introduced at the FCGR1A locus, preferably into SEQ ID NO: 201;
GUSB is introduced at the FCGR1A locus, preferably into SEQ ID NO: 201;
ABCD1 is introduced at the FCGR1A locus, preferably into SEQ ID NO: 201;
GALC is introduced at the FCGR1A locus, preferably into SEQ ID NO: 201;
ARSA is introduced at the FCGR1A locus, preferably into SEQ ID NO: 201;
PSAP is introduced at the FCGR1A locus, preferably into SEQ ID NO: 201;
GBA is introduced at the FCGR1A locus, preferably into SEQ ID NO: 201;
FUCA1 is introduced at the FCGR1A locus, preferably into SEQ ID NO: 201;
MAN2B1 is introduced at the FCGR1A locus, preferably into SEQ ID NO: 201;
AGA is introduced at the FCGR1A locus, preferably into SEQ ID NO: 201;
ASAH1 is introduced at the FCGR1A locus, preferably into SEQ ID NO: 201;
HEXA is introduced at the FCGR1A locus, preferably into SEQ ID NO: 201;
GAA is introduced at the FCGR1A locus, preferably into SEQ ID NO: 201;
SMPD1 is introduced at the FCGR1A locus, preferably into SEQ ID NO: 201;
LIPA is introduced at the FCGR1A locus, preferably into SEQ ID NO: 201;
CDKL5 is introduced at the FCGR1A locus, preferably into SEQ ID NO: 201;
IDUA is introduced at the CSF3R locus, preferably into SEQ ID NO:202;
IDS is introduced at the CSF3R locus, preferably into SEQ ID NO: 202;
ARSB is introduced at the CSF3R locus, preferably into SEQ ID NO: 202;
GUSB is introduced at the CSF3R locus, preferably into SEQ ID NO: 202;
ABCD1 is introduced at the CSF3R locus, preferably into SEQ ID NO: 202;
GALC is introduced at the CSF3R locus, preferably into SEQ ID NO: 202;
ARSA is introduced at the CSF3R locus, preferably into SEQ ID NO: 202;
PSAP is introduced at the CSF3R locus, preferably into SEQ ID NO: 202;
GBA is introduced at the CSF3R locus, preferably into SEQ ID NO: 202;
FUCA1 is introduced at the CSF3R locus, preferably into SEQ ID NO: 202;
MAN2B1 is introduced at the CSF3R locus, preferably into SEQ ID NO: 202;
AGA is introduced at the CSF3R locus, preferably into SEQ ID NO: 202;
ASAH1 is introduced at the CSF3R locus, preferably into SEQ ID NO: 202;
HEXA is introduced at the CSF3R locus, preferably into SEQ ID NO: 202;
GAA is introduced at the CSF3R locus, preferably into SEQ ID NO: 202;
SMPD1 is introduced at the CSF3R locus, preferably into SEQ ID NO: 202;
LIPA is introduced at the CSF3R locus, preferably into SEQ ID NO: 202;
CDKL5 is introduced at the CSF3R locus, preferably into SEQ ID NO: 202;
IDUA is introduced at the FGD4 locus, preferably into SEQ ID NO:203;
IDS is introduced at the FGD4 locus, preferably into SEQ ID NO: 203;
ARSB is introduced at the FGD4 locus, preferably into SEQ ID NO: 203;
GUSB is introduced at the FGD4 locus, preferably into SEQ ID NO: 203;
ABCD1 is introduced at the FGD4 locus, preferably into SEQ ID NO: 203;
GALC is introduced at the FGD4 locus, preferably into SEQ ID NO: 203;
ARSA is introduced at the FGD4 locus, preferably into SEQ ID NO: 203;
PSAP is introduced at the FGD4 locus, preferably into SEQ ID NO: 203;
GBA is introduced at the FGD4 locus, preferably into SEQ ID NO: 203;
FUCA1 is introduced at the FGD4 locus, preferably into SEQ ID NO: 203;
MAN2B1 is introduced at the FGD4 locus, preferably into SEQ ID NO: 203;
AGA is introduced at the FGD4 locus, preferably into SEQ ID NO: 203;
ASAH1 is introduced at the FGD4 locus, preferably into SEQ ID NO: 203;
HEXA is introduced at the FGD4 locus, preferably into SEQ ID NO: 203;
GAA is introduced at the FGD4 locus, preferably into SEQ ID NO: 203;
SMPD1 is introduced at the FGD4 locus, preferably into SEQ ID NO: 203;
LIPA is introduced at the FGD4 locus, preferably into SEQ ID NO: 203;
CDKL5 is introduced at the FGD4 locus, preferably into SEQ ID NO: 203;
IDUA is introduced at the TSPAN14 locus, preferably into SEQ ID NO:204;
IDS is introduced at the TSPAN14 locus, preferably into SEQ ID NO: 204;
ARSB is introduced at the TSPAN14 locus, preferably into SEQ ID NO: 204;
GUSB is introduced at the TSPAN14 locus, preferably into SEQ ID NO: 204;
ABCD1 is introduced at the TSPAN14 locus, preferably into SEQ ID NO: 204;
GALC is introduced at the TSPAN14 locus, preferably into SEQ ID NO: 204;
ARSA is introduced at the TSPAN14 locus, preferably into SEQ ID NO: 204;
PSAP is introduced at the TSPAN14 locus, preferably into SEQ ID NO: 204;
GBA is introduced at the TSPAN14 locus, preferably into SEQ ID NO: 204;
FUCA1 is introduced at the TSPAN14 locus, preferably into SEQ ID NO: 204;
MAN2B1 is introduced at the TSPAN14 locus, preferably into SEQ ID NO: 204;
AGA is introduced at the TSPAN14 locus, preferably into SEQ ID NO: 204;
ASAH1 is introduced at the TSPAN14 locus, preferably into SEQ ID NO: 204;
HEXA is introduced at the TSPAN14 locus, preferably into SEQ ID NO: 204;
GAA is introduced at the TSPAN14locus, preferably into SEQ ID NO: 204;
SMPD1 is introduced at the TSPAN14 locus, preferably into SEQ ID NO: 204;
LIPA is introduced at the TSPAN14 locus, preferably into SEQ ID NO: 204;
CDKL5 is introduced at the TSPAN14 locus, preferably into SEQ ID NO: 204;
IDUA is introduced at the B2M locus, preferably into SEQ ID NO:205;
IDS is introduced at the B2M locus, preferably into SEQ ID NO: 205;
ARSB is introduced at the B2M locus, preferably into SEQ ID NO: 205;
GUSB is introduced at the B2M locus, preferably into SEQ ID NO: 205;
ABCD1 is introduced at the B2M locus, preferably into SEQ ID NO: 205;
GALC is introduced at the B2M locus, preferably into SEQ ID NO: 205;
ARSA is introduced at the B2M locus, preferably into SEQ ID NO: 205;
PSAP is introduced at the B2M locus, preferably into SEQ ID NO: 205;
GBA is introduced at the B2M locus, preferably into SEQ ID NO: 205;
FUCA1 is introduced at the B2M locus, preferably into SEQ ID NO: 205;
MAN2B1 is introduced at the B2M locus, preferably into SEQ ID NO: 205;
AGA is introduced at the B2M locus, preferably into SEQ ID NO: 205;
ASAH1 is introduced at the B2M locus, preferably into SEQ ID NO: 205;
HEXA is introduced at the B2M locus, preferably into SEQ ID NO: 205;
GAA is introduced at the B2M locus, preferably into SEQ ID NO: 205;
SMPD1 is introduced at the B2M locus, preferably into SEQ ID NO: 205;
LIPA is introduced at the B2M locus, preferably into SEQ ID NO: 205; and
CDKL5 is introduced at the B2M locus, preferably into SEQ ID NO: 205.

Such engineered cells, preferably human cells, are more particularly characterized in that they include at one endogenous locus a polynucleotide sequence comprising the following:
a first strong splice site sequence preferably comprising a branch point and an acceptor site;
a first sequence encoding 2A self-cleaving peptide;
an exogenous sequence coding for a protein of interest, such as a therapeutic protein;
a second sequence encoding 2A self-cleaving peptide;
a copy of the coding sequence of the preceding exon endogenous to said locus, preferably rewritten;
optionally, a second strong splice site sequence, preferably comprising a splice donor site.

The invention also pertains to the DNA template or any polynucleotides, which can be used as insertion vectors useful to perform the engineering of the above cells, especially AAV vectors, more preferably AAV6 vectors as described by Ling, C. et al. [High-Efficiency Transduction of Primary Human Hematopoietic Stem/Progenitor Cells by AAV6 Vectors: Strategies for Overcoming Donor-Variation and Implications in Genome Editing (2016) Scientific Reports 6: 35495]. Such polynucleotides are characterized in that they comprise one or several of the following sequences:
a first strong splice site comprising a branch point and an acceptor site;
a first sequence encoding 2A self-cleaving peptide;
an exogenous sequence coding for a protein of interest;
a second sequence encoding 2A self-cleaving peptide;
a copy of the coding sequence of the preceding exon endogenous to said locus, preferably rewritten;
optionally, a second strong splice site comprising a splice donor site.
   According to a preferred embodiment, said polynucleotide also comprises upstream and downstream sequences, which are homologous to the endogenous locus as previously described. In general, at least one or both of these upstream and downstream sequences are homologous to intronic sequences, especially those intronic sequences referred to herein as SEQ ID NO:76, SEQ ID NO: 107, SEQ ID NO:148 and SEQ ID NO:189 to SEQ ID NO:205, and more preferably exclusively homologous to such intronic sequences (i.e. with spanning exon sequences present at the locus).

### Compositions

The invention is also drawn to a composition comprising an effective amount of genetically engineered HSC or iPS cells as described herein. In some embodiments, the invention provides a pharmaceutical composition comprising an effective amount of genetically engineered HSC or iPS cells as described herein.

In some embodiments, the composition can be used as a medicament. In some embodiments, the composition can be used for treating a monogenic disease as described herein.

In some embodiments, the composition comprises a population of cells, wherein at least 40% of the cells in the population have been modified according to any one the methods described herein. In some embodiments, at least 50%, 60%, 70%, 80%, 85%, 90%, 91%, 92%, 93%, 94%, 95%, 96%, 97%, 98%, 99% of the cells in the population have been modified according to any one the methods described herein. In some embodiments, the composition comprises a pure population of cells wherein 100% of the cells have been genetically modified as described herein.

The genetically modified cells can be administered either alone, or as a pharmaceutical composition in combination with diluents and/or with other components. In some embodiments, pharmaceutical compositions can comprise genetically modified HSC or iPS cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g. aluminum hydroxide); and preservatives. In some embodiments, compositions are formulated for intravenous administration.

The genetically modified HSC or iPS cells can be used as a medicament in the treatment of disease. In some embodiments, the genetically modified HSC or iPS cell is for use in the treatment a patient who has a deficiency in the expression of an endogenous gene homologous to the transgene (cross correction). In some embodiments, the genetically modified HSC or iPS cell is for use in the treatment of a lysosomal storage disease. In some embodiments, the genetically modified HSC or iPS cell is for use in the treatment of a disease selected from Mucopolysaccharidosis Type I (Scheie, Hurler-Scheie or Hurler syndrome), Mucopolysaccharidosis Type II (Hunter syndrome), Mucopolysaccharidosis Type VI (Maroteaux-Lamy syndrome), Mucopolysaccharidosis Type VII (Sly disease), X-linked Adrenoleukodystrophy, Globoid Cell Leukodystrophy (Krabbe disease), Metachromatic Leukodystrophy, Gaucher disease, Fucosidosis, Alpha-mannosidosis, Aspartylglucosaminuria, Farber's disease, Tay-Sachs disease, Pompe disease, Niemann Pick disease and Wolman disease.

In some embodiments, the HSC and iPS cells as described herein can be cryopreserved. In some embodiments, the cells can be cryopreserved after their isolation from subjects and prior to any genetic modification. In some embodiments, the genetically modified cells are cryopreserved after genetic modification and prior to infusion in subjects. In some embodiments, the genetically modified cells are cryopreserved after they have been expanded ex vivo.

In one embodiment, the invention provides a cryopreserved pharmaceutical composition comprising: (a) a viable composition of genetically modified HSC or iPS cells (b) an amount of cryopreservative sufficient for the cryopreservation of the HSC or iPS cells; and (c) a pharmaceutically acceptable carrier.

As used herein, “cryopreservation” refers to the preservation of cells by cooling to low sub-zero temperatures, such as (typically) 77 K or −196 °C. (the boiling point of liquid nitrogen). Cryopreservation also refers to storing the cells at a temperature between 0°-10 °C in the absence of any cryopreservative agents. At these low temperatures, any biological activity, including the biochemical reactions that would lead to cell death, is effectively stopped. Cryoprotective agents are often used at sub-zero temperatures to preserve the cells from damage due to freezing at low temperatures or warming to room temperature.

In some embodiments, the injurious effects associated with freezing can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO), glycerol, polyvinylpyrrolidine, polyethylene glycol, albumin, dextran, sucrose, ethylene glycol, i-erythritol, D-Sorbitol, D-mannitol, D-sorbitol, i-inositol, D-lactose, choline chloride, amino acids, methanol, acetamide, glycerol monoacetate, and inorganic salts. In a preferred embodiment, DMSO is used, a liquid which is nontoxic to cells in low concentration. Being a small molecule, DMSO freely permeates the cell and protects intracellular organelles by combining with water to modify its freezability and prevent damage from ice formation. Addition of plasma (e.g., to a concentration of 20-25%) can augment the protective effect of DMSO. After the addition of DMSO, cells should be kept at 0-4 °C until freezing, since DMSO concentrations of about 1% are toxic at temperatures above 4 °C.

Different cryoprotective agents (Rapatz, G., et al., 1968, Cryobiology 5(1):18-25) and different cell types have different optimal cooling rates (see e.g., Rowe, A. W. and Rinfret, A. P., 1962, Blood 20:636; Rowe, A. W., 1966, Cryobiology 3(1):12-18; Lewis, J. P., et al., 1967, Transfusion 7(1):17-32; and Mazur, P., 1970, Science 168:939-949 for effects of cooling velocity on survival of marrow-stem cells and on their transplantation potential). The heat of fusion phase where water turns to ice should be minimal. The cooling procedure can be carried out by use of, e.g., a programmable freezing device or a methanol bath procedure.

After thorough freezing, cells can be rapidly transferred to a long-term cryogenic storage vessel. In one embodiment, the expanded HSC or IPs cells can be cryogenically stored in liquid nitrogen (−196 °C) or its vapor (−165 °C). Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum

In a particular embodiment, the cryopreservation procedure described in Current Protocols in Stem Cell Biology, 2007, (Mick Bhatia, et. al., ed., John Wiley and Sons, Inc.) is used and is hereby incorporated by reference. Mainly when the HSC on a 10-cm tissue culture plate have reached approximately 50% confluency, the media within the plate is aspirated and the HSC s are rinsed with phosphate buffered saline. The adherent HSC are then detached by 3 ml of 0.025% trypsin/0.04% EDTA treatment. The trypsin/EDTA is neutralized by 7 ml of media and the detached HSC are collected by centrifugation at 200×g for 2 min. The supernatant is aspirated off and the pellet of HSC is resuspended in 1.5 ml of media. An aliquot of 1 ml of 100% DMSO is added to the suspension of HSC and gently mixed. Then 1 ml aliquots of this suspension of HSC in DMSO are dispensed into CRYULES in preparation for cryopreservation. The sterilized storage CRYULES preferably have their caps threaded inside, allowing easy handling without contamination. Suitable racking systems are commercially available and can be used for cataloguing, storage, and retrieval of individual specimens.

Considerations and procedures for the manipulation, cryopreservation, and long-term storage of HSC, particularly from bone marrow or peripheral blood can be found, for example, in the following references, incorporated by reference herein: Gorin, N.C., 1986, Clinics In Haematology 15(1):19-48; Bone-Marrow Conservation, Culture and Transplantation, Proceedings of a Panel, Moscow, Jul. 22-26, 1968, International Atomic Energy Agency, Vienna, pp. 107-186.

Other methods of cryopreservation of viable cells, or modifications thereof, are available and envisioned for use (e.g., cold metal-minor techniques; Livesey, S. A. and Linner, J. G., 1987, Nature 327:255; Linner, J. G., et al., 1986, J. Histochem. Cytochem. 34(9):1123-1135; U.S. Pat. Nos. 4,199,022, 3,753,357, and 4,559,298 and all of these are incorporated hereby reference in their entirety.

In some embodiments, the frozen HSC or iPS cells are thawed quickly (e.g., in a water bath maintained at 37°-41 °C) and chilled on ice immediately upon thawing. In particular, the cryogenic vial containing the frozen HSC or iPS cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

In one embodiment, the thawing procedure after cryopreservation is described in Current Protocols in Stem Cell Biology 2007 (Mick Bhatia, et al., ed., John Wiley and Sons, Inc.) and is hereby incorporated by reference. Immediately after removing the cryogenic vial from the cryo-freezer, the vial is rolled between the hands for 10 to 30 sec until the outside of the vial is frost free. The vial is then held upright in a 37 °C water-bath until the contents are visibly thawed. The vial is immersed in 95% ethanol or sprayed with 70% ethanol to kill microorganisms from the water-bath and air dry in a sterile hood. The contents of the vial are then transferred to a 10-cm sterile culture containing 9 ml of media using sterile techniques. The HSC can then be cultured and further expanded in an incubator at 37 °C with 5% humidified CO₂.

It may be desirable to treat the HSC or IPs cells in order to prevent cellular clumping upon thawing. To prevent clumping, various procedures can be used, including but not limited to, the addition before and/or after freezing of DNase (Spitzer, G., et al., 1980, Cancer 45:3075-3085), low molecular weight dextran and citrate, hydroxyethyl starch (Stiff, P. J., et al., 1983, Cryobiology 20:17-24).

The cryoprotective agent, if toxic in humans, should be removed prior to therapeutic use of the thawed HSC or iPS cells. In an embodiment employing DMSO as the cryopreservative, it is preferable to omit this step in order to avoid cell loss, since DMSO has no serious toxicity. However, where removal of the cryoprotective agent is desired, the removal is preferably accomplished upon thawing.

One way in which to remove the cryoprotective agent is by dilution to an insignificant concentration. This can be accomplished by addition of medium, followed by, if necessary, one or more cycles of centrifugation to pellet the cells, removal of the supernatant, and resuspension of the cells. For example, the intracellular DMSO in the thawed cells can be reduced to a level (less than 1%) that will not adversely affect the recovered cells. This is preferably done slowly to minimize potentially damaging osmotic gradients that occur during DMSO removal.

After removal of the cryoprotective agent, cell count (e.g., by use of a hemocytometer) and viability testing (e.g., by trypan blue exclusion; Kuchler, R. J. 1977, Biochemical Methods in Cell Culture and Virology, Dowden, Hutchinson & Ross, Stroudsburg, Pa., pp. 18-19; 1964, Methods in Medical Research, Eisen, H. N., et al., eds., Vol. 10, Year Book Medical Publishers, Inc., Chicago, pp. 39-47) can be done to confirm cell survival.

In one embodiment, thawed cells are tested by standard assays of viability (e.g., trypan blue exclusion) and of microbial sterility as described herein, and tested to confirm and/or determine their identity relative to the recipient.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

TABLE 4
Preferred target sequences defined to gene edit
the cells of the present invention

| | Name of | |
|---|---|---|
| SEQ ID | sequence- | |
| NO # | specific reagent | Target sequence |
| 77 | CX3CR1_T01 | TCTTTCCTCTGTAGCATGGTCCAGATGGCTCATAGCAGGGACCATGATA |
| 78 | CX3CR1_T02 | TATGCTGGGTGAGCACCCACTGCATGCACCCACTGTGCCAGCACTGAGA |
| 79 | CX3CR1_T03 | TGCTGGGTGAGCACCCACTGCATGCACCCACTGTGCCAGCACTGAGAGA |
| 80 | CX3CR1_T04 | TGAGAGACTCCTGTGGGAGCCACAGCAATTCTAGGGTCTTCACTGGGGA |
| 81 | CX3CR1_T05 | TCCTGTGGGAGCCACAGCAATTCTAGGGTCTTCACTGGGGACTCTGAGA |
| 82 | CX3CR1_T06 | TGGGAGCCACAGCAATTCTAGGGTCTTCACTGGGGACTCTGAGACAGCA |
| 83 | CX3CR1_T07 | TCGTCTGCCCTCACTGAGCAGACCCCCTGGATGGCAGGGAGCAGTCCCA |
| 84 | CX3CR1_T08 | TGCCCTCACTGAGCAGACCCCCTGGATGGCAGGGAGCAGTCCCAAGCCA |
| 85 | CX3CR1_T09 | TGGATGGCAGGGAGCAGTCCCAAGCCAGATGGATGCCCATAACCAGCCA |
| 86 | CX3CR1_T10 | TCTCAATACATAATATCACCACGTATCAGGCAAAACCATCCTGCCCAGA |
| 87 | CX3CR1_T11 | TATCAGGCAAAACCATCCTGCCCAGAGCATTATCTGAATTTGCATCCCA |
| 88 | CX3CR1_T12 | TCCTGCCCAGAGCATTATCTGAATTTGCATCCCATCTGCAGAAGATACA |
| 89 | CX3CR1_T13 | TCTGAATTTGCATCCCATCTGCAGAAGATACATTCACCCACTTCTTCCA |
| 90 | CX3CR1_T14 | TTCCATTCTGTCTTAATCAAAGTCTTTATGTGAATTTTCCCCATTGAGA |
| 91 | CX3CR1_T15 | TCCATTCTGTCTTAATCAAAGTCTTTATGTGAATTTTCCCCATTGAGAA |
| 92 | CX3CR1_T16 | TTCTGTCTTAATCAAAGTCTTTATGTGAATTTTCCCCATTGAGAAGACA |
| 93 | CX3CR1_T17 | TCTGTCTTAATCAAAGTCTTTATGTGAATTTTCCCCATTGAGAAGACAA |
| 94 | CX3CR1_T18 | TTTATGTGAATTTTCCCCATTGAGAAGACAAGCCCCTTCCTGGCTTAGA |
| 95 | CX3CR1_T19 | TTCCTGGCTTAGACTGTACCTGACTGATCTTTTCATGAGCTCCTTGCCA |
| 96 | CX3CR1_T20 | TCCTGGCTTAGACTGTACCTGACTGATCTTTTCATGAGCTCCTTGCCAA |
| 97 | CX3CR1_gRNA1 | GTACAGTCTAAGCCAGGAAGGGG |
| 98 | CX3CR1_gRNA2 | GGGAGCAGTCCCAAGCCAGATGG |
| 99 | CX3CR1_gRNA3 | GACTGCTCCCTGCCATCCAGGGG |
| 100 | CX3CR1_gRNA4 | GTGAATGTATCTTCTGCAGATGG |
| 101 | CX3CR1_gRNA5 | GTCTCTCAGTGCTGGCACAGTGG |
| 102 | CX3CR1_gRNA6 | GACAGCAGGGAGCTAGGATGAGG |
| 103 | CX3CR1_gRNA7 | GAAGGGGCTTGTCTTCTCAATGG |
| 104 | CX3CR1_gRNA8 | GCAAATTCAGATAATGCTCTGGG |
| 105 | CX3CR1_gRNA9 | GAGCAGACCCCCTGGATGGCAGG |
| 106 | CX3CR1_gRNA10 | GAACTCATAGAAAGCGATATTGG |
| 108 | CD11b_T01 | TTCAGAGCAGGACTGGACGTGCCCCACGACGGTGGTTCTTAGGTCAGGA |
| 109 | CD11b_T02 | TATGGCCCACGACCTGTTTTTGCACAACCTGCCAGCTAGAGATTGAAGA |
| 110 | CD11b_T03 | TGATGATAGGGAGCACCACCCCCAAAGAATTCTATTTGTCTCATTTGTA |
| 111 | CD11b_T04 | TTCTATTTGTCTCATTTGTAAACCCGTATTACAAACAAATTGTACTCAA |
| 112 | CD11b_T05 | TATTTGTCTCATTTGTAAACCCGTATTACAAACAAATTGTACTCAATCA |
| 113 | CD11b_T06 | TTTGTAAACCCGTATTACAAACAAATTGTACTCAATCATTATGTTTGAA |
| 114 | CD11b_T07 | TTGTAAACCCGTATTACAAACAAATTGTACTCAATCATTATGTTTGAAA |
| 115 | CD11b_T08 | TACAAACAAATTGTACTCAATCATTATGTTTGAAATTTCCCTAATGACA |
| 116 | CD11b_T09 | TTGTACTCAATCATTATGTTTGAAATTTCCCTAATGACAAATTTGTGGA |
| 117 | CD11b_T10 | TGTACTCAATCATTATGTTTGAAATTTCCCTAATGACAAATTTGTGGAA |
| 118 | CD11b_T11 | TACTCAATCATTATGTTTGAAATTTCCCTAATGACAAATTTGTGGAAAA |
| 119 | CD11b_T12 | TTTCCCTAATGACAAATTTGTGGAAAAGTATTTTCTGTCTTGTTATATA |
| 120 | CD11b_T13 | TTCCCTAATGACAAATTTGTGGAAAAGTATTTTCTGTCTTGTTATATAA |
| 121 | CD11b_T14 | TTGTGGAAAAGTATTTTCTGTCTTGTTATATAAGTACTTGTACAACATA |
| 122 | CD11b_T15 | TGTTATATAAGTACTTGTACAACATATTCTATCAGCCTCTTGGTCTGCA |
| 123 | CD11b_T16 | TTATATAAGTACTTGTACAACATATTCTATCAGCCTCTTGGTCTGCAAA |
| 124 | CD11b_T17 | TATATAAGTACTTGTACAACATATTCTATCAGCCTCTTGGTCTGCAAAA |
| 125 | CD11b_T18 | TACAACATATTCTATCAGCCTCTTGGTCTGCAAAACCTAAAATTTACTA |
| 126 | CD11b_T19 | TCTTGGTCTGCAAAACCTAAAATTTACTATCTGGCTGTTTACAGAATAA |
| 127 | CD11b_T20 | TGCAAAACCTAAAATTTACTATCTGGCTGTTTACAGAATAAGTGTGCTA |
| 128 | CD11b_T21 | TGAAAATGATTTGAGTTTGTTACCTTTTATGCTTATATGTTGTGGAAAA |
| 129 | CD11b_T22 | TTTGTTACCTTTTATGCTTATATGTTGTGGAAAATGAAATTCTCCTCAA |
| 130 | CD11b_T23 | TTGTTACCTTTTATGCTTATATGTTGTGGAAAATGAAATTCTCCTCAAA |
| 131 | CD11b_T24 | TGTTACCTTTTATGCTTATATGTTGTGGAAAATGAAATTCTCCTCAAAA |
| 132 | CD11b_T25 | TTTATGCTTATATGTTGTGGAAAATGAAATTCTCCTCAAAAGGGAAGGA |
| 133 | CD11b_T26 | TTATGCTTATATGTTGTGGAAAATGAAATTCTCCTCAAAAGGGAAGGAA |
| 134 | CD11b_T27 | TATGCTTATATGTTGTGGAAAATGAAATTCTCCTCAAAAGGGAAGGAAA |
| 135 | CD11b_T28 | TGCTTATATGTTGTGGAAAATGAAATTCTCCTCAAAAGGGAAGGAAATA |
| 136 | CD11b_T29 | TGGAAAATGAAATTCTCCTCAAAAGGGAAGGAAATACTTGAGAGCTGCA |
| 137 | CD11b_T30 | TACTTGAGAGCTGCATAGGAAGGAAATTATCTAATTAAGAATGTATAGA |
| 138 | CD11b_gRNA1 | GGTTGTGCAAAAACAGGTCGTGG |
| 139 | CD11b_gRNA2 | GGGAGGCTGGAATTCAGAGCAGG |
| 140 | CD11b_gRNA3 | GGAGTCAGCAAACAGTGGCCTGG |
| 141 | CD11b_gRNA4 | GAGTCAGCAAACAGTGGCCTGGG |
| 142 | CD11b_gRNA5 | GACCTAAGAACCACCGTCGTGGG |
| 143 | CD11b_gRNA6 | GCAAATCATCGTTGTGACACCGG |
| 144 | CD11b_gRNA7 | GAGACAAATAGAATTCTTTGGGG |
| 145 | CD11b_gRNA8 | GCCCCACGACGGTGGTTCTTAGG |
| 146 | CD11b_gRNA9 | GAAATACTTGAGAGCTGCATAGG |
| 147 | CD11b_gRNA10 | GGTCAGGAGTCAGCAAACAGTGG |
| 149 | S100A9_T01 | TTTCCCCGTTGTATTGGTTGAAATAAGTTTCACTAATTGGTAACCTCCA |
| 150 | S100A9_T02 | TATTGGTTGAAATAAGTTTCACTAATTGGTAACCTCCAGAGGGAAGGGA |
| 151 | S100A9_T03 | TTTCACTAATTGGTAACCTCCAGAGGGAAGGGAAGGGAGGGCAGGGGAA |
| 152 | S100A9_T04 | TGGAACTGGCCTCTAAGTCAGATCTGAATTTGCATGCCCTCAATAGTCA |
| 153 | S100A9_T05 | TCTAAGTCAGATCTGAATTTGCATGCCCTCAATAGTCAAGCTGTGAAAA |
| 154 | S100A9_T06 | TGCATGCCCTCAATAGTCAAGCTGTGAAAACTAATGACCCTCTCTAGGA |
| 155 | S100A9_T07 | TGAAAACTAATGACCCTCTCTAGGACTGGTTTCAAGTCTTCCTCCAGGA |
| 156 | S100A9_T08 | TCTTCCTCCAGGAAGATACCATTCCTAGCTGTTAAAGTTGTTATAAGGA |
| 157 | S100A9_T09 | TCCTCCAGGAAGATACCATTCCTAGCTGTTAAAGTTGTTATAAGGACCA |
| 158 | S100A9_T10 | TTCCTAGCTGTTAAAGTTGTTATAAGGACCAAATGAGGTGACATTTCCA |
| 159 | S100A9_T11 | TTAAAGTTGTTATAAGGACCAAATGAGGTGACATTTCCAGGCTTACTCA |
| 160 | S100A9_T12 | TGACCAGGGCAAGACCCTGGAACTCAGCTTCCTCTTCTATAAATAGAGA |
| 161 | S100A9_T13 | TTCCTCTTCTATAAATAGAGAATCAGCACCCAAGTCACAGGGTCATGGA |
| 162 | S100A9_T14 | TCTTCTATAAATAGAGAATCAGCACCCAAGTCACAGGGTCATGGAGGGA |
| 163 | S100A9_T15 | TCTATAAATAGAGAATCAGCACCCAAGTCACAGGGTCATGGAGGGAATA |
| 164 | S100A9_T16 | TATAAATAGAGAATCAGCACCCAAGTCACAGGGTCATGGAGGGAATAAA |
| 165 | S100A9_T17 | TGGAGAGCGTTTGGTATGTGCTCAGTGTCTGCTCCATTGTGCGCACTCA |
| 166 | S100A9_T18 | TGGTATGTGCTCAGTGTCTGCTCCATTGTGCGCACTCAGCCTATGGTCA |
| 167 | S100A9_T19 | TTGTGCGCACTCAGCCTATGGTCATTTTTAATTTTTAAATCCAGCCCCA |
| 168 | S100A9_T20 | TTCCCTTGTACATTTGCCAGCTGGTCATTTACTGTGCTCCCAGTCCCCA |
| 169 | S100A9_T21 | TTTTGTTTTCTTTTCAAATTTGGGGAAAGTCGGGAAACAGAGGCCTGCA |
| 170 | S100A9_T22 | TTTCTTTTCAAATTTGGGGAAAGTCGGGAAACAGAGGCCTGCATTAAGA |
| 171 | S100A9_T23 | TTCTTTTCAAATTTGGGGAAAGTCGGGAAACAGAGGCCTGCATTAAGAA |
| 172 | S100A9_T24 | TTGGGGAAAGTCGGGAAACAGAGGCCTGCATTAAGAAGGGTGGAACACA |
| 173 | S100A9_T25 | TAGGTCCCCAGCCCTCCCAGTGCCCCTCCCTCCGCCTTGGTAAGGTGGA |
| 174 | S100A9_T26 | TTCAGAGTTAGGGGCCCTGACAGCTCTCCATAGGTGGAGGCCTCAGGCA |
| 175 | S100A9_T27 | TTAGGGGCCCTGACAGCTCTCCATAGGTGGAGGCCTCAGGCAGGCAGGA |
| 176 | S100A9_T28 | TCCATAGGTGGAGGCCTCAGGCAGGCAGGATGCTGGGTGGGGTAGGCAA |
| 177 | S100A9_T29 | TAGGTGGAGGCCTCAGGCAGGCAGGATGCTGGGTGGGGTAGGCAAGAAA |
| 178 | S100A9_T30 | TGGGTGGGGTAGGCAAGAAAGGGCCCAGCAGAGAGGCCGCATGGCAAAA |
| 179 | S100A9_gRNA1 | GCACAGGAGAGTGCTCGCATTGG |
| 180 | S100A9_gRNA2 | GGTACCCCACAGGTTCTGGGAGG |
| 181 | S100A9_gRNA3 | GGAGCCAGACATCCTGGGGTAGG |
| 182 | S100A9_gRNA4 | GGAGAGTGCTCGCATTGGCTGGG |
| 183 | S100A9_gRNA5 | GGAAGCAGAGCCTCATGGATGGG |
| 184 | S100A9_gRNA6 | GGCTTACTCATGCCATGACCAGG |
| 185 | S100A9_gRNA7 | GGGAAACACCTAGAAAAACTAGG |
| 186 | S100A9_gRNA8 | GTGGGGGGTGAAGCGGGCATAGG |
| 187 | S100A9_gRNA9 | GGGGGGTGAAGCGGGCATAGGGG |
| 188 | S100A9_gRNA10 | GAGGGCTGGGGACCTACCCCAGG |

As per the present disclosure, the present invention more particularly encompass the following items:
1. A method for expressing a transgene into the brain of a patient comprising:
   i) obtaining genetically modified hematopoietic stem cells (HSC), wherein the HSC were isolated from the patient or were obtained from induced pluripotent stem (iPS) cells derived from the patient and differentiated into HSC, wherein the genetically modified HSC have been engineered to comprise a transgene integrated at a locus expressed in microglial cells; and
   ii) engrafting the genetically modified HSC into the patient in order to have them differentiate into microglial cells expressing the transgene into the patient's brain.
2. A method for expressing a transgene into the brain of a patient comprising:
   i) obtaining genetically modified hematopoietic stem cells (HSC), wherein the HSC were isolated from a compatible donor or were obtained from induced pluripotent stem (iPS) cells derived from a compatible donor and differentiated into HSC, wherein the genetically modified HSC have been engineered to comprise a transgene integrated at a locus expressed in microglial cells; and
   ii) engrafting the genetically modified HSC into the patient in order to have them differentiate into microglial cells expressing the transgene into the patient's brain.
3. The method according to item 1 or 2, wherein said locus is selected from the group consisting of TMEM119, S100A9, CD11B, B2m, Cx3cr1, MERTK, CD164, Tlr4, Tlr7, Cd14, Fcgr1a, Fcgr3a, TBXAS1, DOK3, ABCA1, TMEM195, MR1, CSF3R, FGD4, TSPAN14, TGFBRI, CCR5, GPR34, SERPINE2, SLCO2B1, P2ry12, Olfml3, P2ry13, Hexb, Rhob, Jun, Rab3il1, Ccl2, Fcrls, Scoc, Siglech, Slc2a5, Lrrc3, Plxdc2, Usp2, Ctsf, Cttnbp2nl, Atp8a2, Lgmn, Mafb, Egr1, Bhlhe41, Hpgds, Ctsd, Hspa1a, Lag3, Csf1r, Adamts1, F11r, Golm1, Nuak1, Crybb1, Ltc4s, Sgce, Pla2g15, Ccl3l1, Abhd12, Ang, Ophn1, Sparc, Pros1, P2ry6, Lair1, Il1a, Epb41l2, Adora3, Rilpl1, Pmepa1, Ccl13, Pde3b, Scamp5, Ppp1r9a, Tjp1, Ak1, B4galt4, Gtf2 h2, Trem2, Ckb, Acp2, Pon3, Agmo, Tnfrsf17, Fscn1, St3gal6, Adap2, Ccl4, Entpd1, Tmem86a, Kctd12, Dst, Cts12, Abcc3, Pdgfb, Pald1, Tubgcp5, Rapgef5, Stab1, Lacc1, Tmc7, Nrip1, Kcnd1, Tmem206, Hps4, Dagla, Extl3, Mlph, Arhgap22, Cxxc5, P4ha1, Cysltr1, Fgd2, Kcnk13, Gbgt1, C18orf1, Cadm1, Bco2, Adrb1, C3ar1, Large, Leprel1, Liph, Upk1b, P2rx7, Slc46a1, Ebf3, Ppp1r15a, Il10ra, Rasgrp3, Fos, Tppp, Slc24a3, Havcr2, Nav2, Apbb2, Clstn1, Blnk, Gnaq, Ptprm, Frmd4a, Cd86, Tnfrsf11a, Spint1, Ppm1l, Tgfbr2, Cmklr1, Tlr6, Gas6, Hist1h2ab, Atf3, Acvr1, Abi3, Lrp12, Ttc28, Plxna4, Adamts16, Rgs1, Icam1, Snx24, Ly96, Dnajb4, and Ppfia4.
4. The method according to any of items 1-3, wherein said locus is cx3cr1.
5. The method according to any of items 1-3, wherein said locus is cd11b.
6. The method according to any of items 1-3, wherein said locus is tmem119.
7. The method according to any of items 1-3, wherein said locus is s100a9.
8. The method of any of items 1-7, wherein the cells have been genetically modified using a sequence specific reagent and a donor sequence comprising the transgene.
9. The method of item 8, wherein the donor sequence comprising the transgene is provided to the cells in a viral vector.
10. The method of item 9, wherein the viral vector is an AAV vector.
11. The method of any of items 8-10, wherein the sequence specific reagent comprises an engineered rare-cutting endonuclease.
12. The method of item 11, wherein the engineered rare-cutting endonuclease is selected from the group consisting of transcription activator-like effector nuclease (TALEN), zinc finger nuclease (ZFN), clustered regularly interspaced short palindromic repeats (CRISPR)-Cas, meganuclease and megaTAL.
13. The method of item 12, wherein the sequence specific reagent is provided to cells as a nucleic acid.
14. The method of item 13, wherein the nucleic acid is mRNA.
15. The method according to any one items 1 to 14, wherein said transgene comprises IDUA for treating Mucopolysaccharidosis Type I (Scheie, Hurler-Scheie or Hurler syndrome).
16. The method according to any one items 1 to 14, wherein said transgene comprises IDS for treating Mucopolysaccharidosis Type II (Hunter).
17. The method according to any one items 1 to 14, wherein said transgene comprises ARSB for treating Mucopolysaccharidosis Type VI (Maroteaux-Lamy).
18. The method according to any one items 1 to 14, wherein said transgene comprises GUSB for treating Mucopolysaccharidosis Type VII (Sly).
19. The method according to any one items 1 to 14, wherein said transgene comprises ABCD1 for treating X-linked Adrenoleukodystrophy.
20. The method according to any one items 1 to 14, wherein said transgene comprises GALC for treating Globoid Cell Leukodystrophy (Krabbe).
21. The method according to any one items 1 to 14, wherein said transgene comprises ARSA for treating Metachromatic Leukodystrophy.
22. The method according to any one items 1 to 14, wherein said transgene comprises GBA for treating Gaucher Disease.
23. The method according to any one items 1 to 14, wherein said transgene comprises FUCA1 for treating Fucosidosis.
24. The method according to any one items 1 to 14, wherein said transgene comprises MAN2B1 for treating Alpha-mannosidosis.
25. The method according to any one items 1 to 14, wherein said transgene comprises AGA for treating Aspartylglucosaminuria.
26. The method according to any one items 1 to 14, wherein said transgene comprises ASAH1 for treating Farber.
27. The method according to any one items 1 to 14, wherein said transgene comprises HEXA for treating Tay-Sachs.
28. The method according to any one items 1 to 14, wherein said transgene comprises GAA for treating Pompe.
29. The method according to any one items 1 to 14, wherein said transgene comprises SMPD1 for treating Niemann Pick.
30. The method according to any one items 1 to 14, wherein said transgene comprises LIPA for treating Wolman Syndrome.
31. The method according to any one items 1 to 14, wherein said transgene comprises CDKL5 for treating CDKL5-deficiency related disease.
32. A genetically modified HSC or iPS cell which has a transgene integrated at a locus selected from tmem119, cd11b or cx3cr1, said transgene being under the transcriptional control of the endogenous promoter of said genes.
33. A HSC or iPS cell according to item 32 for use as a medicament.
34. A HSC or iPS cell according to item 32, for use in the treatment a patient who has a deficiency in the expression of an endogenous gene homologous to said transgene (cross correction).
35. A HSC or iPS cell according to item 33, for use in the treatment of a lysosomal storage disease.
36. A HSC or iPS cell according to item 32, wherein said transgene comprises IDUA for its use in the treatment of Mucopolysaccharidosis Type I (Scheie, Hurler-Scheie or Hurler syndrome).
37. A HSC or iPS cell according to item 32, wherein said transgene comprises IDS for its use in the treatment of Mucopolysaccharidosis Type II (Hunter).
38. A HSC or iPS cell according to item 32, wherein said transgene comprises ARSB for its use in the treatment of Mucopolysaccharidosis Type VI (Maroteaux-Lamy).
39. A HSC or iPS cell according to item 32, wherein said transgene comprises GUSB for its use in the treatment of Mucopolysaccharidosis Type VII (Sly).
40. A HSC or iPS cell according to item 32, wherein said transgene comprises ABCD1 for treating X-linked Adrenoleukodystrophy.
41. A HSC or iPS cell according to item 32, wherein said transgene comprises GALC for its use in the treatment of Globoid Cell Leukodystrophy (Krabbe).
42. A HSC or iPS cell according to item 32, wherein said transgene comprises ARSA for its use in the treatment of Metachromatic Leukodystrophy.
43. A HSC or iPS cell according to item 32, wherein said transgene comprises GBA for its use in the treatment of Gaucher Disease.
44. A HSC or iPS cell according to item 32, wherein said transgene comprises FUCA1 for its use in the treatment of Fucosidosis.
45. A HSC or iPS cell according to item 32, wherein said transgene comprises MAN2B1 for its use in the treatment of Alpha-mannosidosis.
46. A HSC or iPS cell according to item 32, wherein said transgene comprises AGA for its use in the treatment of Aspartylglucosaminuria.
47. A HSC or iPS cell according to item 32, wherein said transgene comprises ASAH1 for its use in the treatment of Farber.
48. A HSC or iPS cell according to item 32, wherein said transgene comprises HEXA for treating Tay-Sachs.
49. A HSC or iPS cell according to item 32, wherein said transgene comprises GAA for its use in the treatment of Pompe.
50. A HSC or iPS cell according to item 32, wherein said transgene comprises SMPD1 for its use in the treatment of Niemann Pick.
51. A HSC or iPS cell according to item 32, wherein said transgene comprises LIPA for its use in the treatment of Wolman Syndrome.
52. A HSC or iPS cell according to item 32, wherein said transgene comprises CDKL5 for its use in the treatment of CDKL5-deficiency related disease
53. A HSC or iPS cell according to any one of items 32-50, wherein multicopies of said transgene are integrated at the same locus separated by 2A self-cleaving peptide sequences.
54. A pharmaceutical composition comprising an HSC or iPS cell according to any of items 32-51.
55. A method for integrating an exogenous coding sequence into an endogenous intron genomic region at an insertion site comprising the following steps:
   providing cell(s) comprising an endogenous intronic genomic region,
   introducing into said cell(s) a polynucleotide template comprising an exogenous coding sequence, wherein said polynucleotide template comprises:
      a) a first homologous polynucleotide sequence, which is homologous to the intronic sequence upstream of the insertion site,
      b) a first strong splice site sequence, comprising a branch point and a splice acceptor;
      c) a first sequence encoding 2A self-cleaving peptide;
      d) an exogenous sequence coding for a protein of interest;
      e) a second sequence encoding 2A self-cleaving peptide;
      f) a copy of the coding sequence of the first exon;
      g) a second strong splice site sequence comprising a splice donor; and
      h) a second homologous polynucleotide sequence, which is homologous to the intronic sequence downstream of the insertion site;
   inducing the integration of said exogenous polynucleotide into said intronic sequence, preferably by homologous recombination, to have said exogenous coding sequence being transcribed at said endogenous locus along with the first and preferably second exon, or a copy thereof.
56. An insertion vector, such as an AAV vector, characterized in that it comprises an exogenous polynucleotide sequence for insertion at an endogenous locus comprising the following sequences:
   a) a first homologous polynucleotide sequence, which is homologous to the intronic sequence upstream of the insertion site,
   b) a first strong splice site sequence, comprising a branch point and a splice acceptor;
   c) a first sequence encoding 2A self-cleaving peptide;
   d) an exogenous sequence coding for a protein of interest;
   e) a second sequence encoding 2A self-cleaving peptide;
   f) a copy of the coding sequence of the first exon;
   g) a second strong splice site sequence comprising a splice donor; and
   h) a second homologous polynucleotide sequence, which is homologous to the intronic sequence downstream of the insertion site.
57. An insertion vector according to item 56, wherein said first and second homologous sequences are homologous to an endogenous locus selected from: tmem119, s100a9, cd11b, b2m, cx3cr1, mertk, cd164, tlr4, tlr7, cd14, fcgr1a, fcgr3a, tbxas1, dok3, abca1, tmem195, mr1, csf3r, fgd4, tspan14, tgfbri, ccr5, gpr34, serpine2, slco2b1, p2ry12, olfm13, p2ry13, hexb, rhob, jun, rab3il1, ccl2, fcrls, scoc, siglech, slc2a5, lrrc3, plxdc2, usp2, ctsf, cttnbp2nl, atp8a2, lgmn, mafb, egr1, bhlhe41, hpgds, ctsd, hspa1a, lag3, csflr, adamts1, fl1r, golm1, nuak1, crybb1, ltc4s, sgce, pla2g15, ccl3l1, abhd12, ang, ophn1, sparc, pros1, p2ry6, lair1, il1a, epb41l2, adora3, rilpl1, pmepa1, ccl13, pde3b, scamp5, ppp1r9a, tjp1, ak1, b4galt4, gtf2 h2, trem2, ckb, acp2, pon3, agmo, tnfrsf17, fscn1, st3gal6, adap2, ccl4, entpd1, tmem86a, kctd12, dst, cts12, abcc3, pdgfb, pald1, tubgcp5, rapgef5, stab1, lacc1, tmc7, nrip1, kcnd1, tmem206, hps4, dagla, extl3, mlph, arhgap22, cxxc5, p4ha1, cysltr1, fgd2, kcnk13, gbgt1, c18orf1, cadm1, bco2, adrb1, c3ar1, large, leprel1, liph, upk1b, p2rx7, slc46a1, ebf3, ppp1r15a, il10ra, rasgrp3, fos, tppp, slc24a3, havcr2, nav2, apbb2, clstn1, blnk, gnaq, ptprm, frmd4a, cd86, tnfrsf11a, spint1, ppm1l, tgfbr2, cmklr1, tlr6, gas6, hist1h2ab, atf3, acvr1, abi3, lrp12, ttc28, plxna4, adamts16, rgs1, icam1, snx24, ly96, dnajb4, and ppfia4.
58. An insertion according to item 56 or 57, wherein said therapeutic protein encoded by said exogenous coding sequence has at least 80% polypeptide sequence identity with IDUA, IDS, ARSB, GUSB, ABCD1, GALC, ARSA, PSAP, GBA, FUCA1, MAN2B1, AGA, ASAH1, HEXA, GAA, SMPD1, LIPA and CDKL5 (SEQ ID NO: 1 to SEQ ID NO:35-see Table 1)
59. An engineered cell, characterized in that an exogenous polynucleotide sequence has been inserted at an endogenous locus comprising the following:
   a first strong splice site sequence comprising a branch point and an acceptor site;
   a first sequence encoding 2A self-cleaving peptide;
   an exogenous sequence coding for a protein of interest, such as a therapeutic protein;
   a second sequence encoding 2A self-cleaving peptide;
   a copy of the coding sequence of the preceding exon endogenous to said locus;
   a second strong splice site sequence comprising a splice donor site;
60. An engineered cell according to item 59, wherein said exogenous polynucleotide sequence is inserted at an endogenous locus selected from: tmem119, s100a9, cd11b, B2m, Cx3cr1, mertk, cd164, tlr4, tlr7, cd14, fcgr1a, fcgr3a, tbxas1, dok3, abca1, tmem195, mr1, csf3r, fgd4, tspan14, tgfbri, ccr5, gpr34, serpine2, slco2b1, P2ry12, Olfml3, P2ry13, Hexb, Rhob, Jun, Rab3il1, Ccl2, Fcrls, Scoc, Siglech, Slc2a5, Lrrc3, Plxdc2, Usp2, Ctsf, Cttnbp2nl, Atp8a2, Lgmn, Mafb, Egr1, Bhlhe41, Hpgds, Ctsd, Hspa1a, Lag3, Csf1r, Adamts1, F11r, Golm1, Nuak1, Crybb1, Ltc4s, Sgce, Pla2g15, Ccl3l1, Abhd12, Ang, Ophn1, Sparc, Pros1, P2ry6, Lair1, Il1a, Epb41l2, Adora3, Rilpl1, Pmepa1, Ccl13, Pde3b, Scamp5, Ppp1r9a, Tjp1, Ak1, B4galt4, Gtf2 h2, Trem2, Ckb, Acp2, Pon3, Agmo, Tnfrsf17, Fscn1, St3gal6, Adap2, Ccl4, Entpd1, Tmem86a, Kctd12, Dst, Cts12, Abcc3, Pdgfb, Pald1, Tubgcp5, Rapgef5, Stab1, Lacc1, Tmc7, Nrip1, Kcnd1, Tmem206, Hps4, Dagla, Extl3, Mlph, Arhgap22, Cxxc5, P4ha1, Cysltr1, Fgd2, Kcnk13, Gbgt1, C18orf1, Cadm1, Bco2, Adrb1, C3ar1, Large, Leprel1, Liph, Upk1b, P2rx7, Slc46a1, Ebf3, Ppp1r15a, Il10ra, Rasgrp3, Fos, Tppp, Slc24a3, Havcr2, Nav2, Apbb2, Clstn1, Blnk, Gnaq, Ptprm, Frmd4a, Cd86, Tnfrsf11a, Spint1, Ppm1l, Tgfbr2, Cmklr1, Tlr6, Gas6, Hist1h2ab, Atf3, Acvr1, Abi3, Lrp12, Ttc28, Plxna4, Adamts16, Rgs1, Icam1, Snx24, Ly96, Dnajb4, and Ppfia4.
61. An engineered cell according to item 59 or 60, wherein said therapeutic protein encoded by said exogenous coding sequence has at least 80% polypeptide sequence identity with IDUA, IDS, ARSB, GUSB, ABCD1, GALC, ARSA, PSAP, GBA, FUCA1, MAN2B1, AGA, ASAH1, HEXA, GAA, SMPD1, LIPA and CDKL5 (SEQ ID NO: 1 to SEQ ID NO:35-see Table 1).
   Having generally described this invention, a further understanding can be obtained by reference to certain specific examples, which are provided herein for purposes of illustration only, and are not intended to limit the scope of the claimed invention.

### EXAMPLES

### Example 1: Materials and Method

### Cell Culture:

HSC culture: HSCs prepared from GCS-F mobilized Leukopak (Miltenyi), were thawed and seeded at 0.4×10⁶ cells/ml into HSC media composed of STEM Span II media (cat. #09655, Stemcell Technologies), with 1× final concentrations of CD34+ expansion cocktail (#02691, Stemcell Technologies) and Pen-Strep (#15140-122, Gibco Life Technologies). The cells were incubated at 37 °C and 5% CO₂ for 48 hrs for recovery after thawing before TALEN transfection and AAV transduction.

### Repair Template Constructs:

For S100A9, AAV6 particles obtained from Vigene were used to insert the synthetic exon sequence into the first intron of the S100A9 locus. This donor contains a left homology arm for this intronic region (SEQ ID NO: 209), followed by the 3′ albumin splicing signal sequence (SEQ ID NO: 206), followed by a sequence encoding a GSG linker (SEQ ID NO: 215), followed by a sequence encoding a P2A peptide (SEQ ID NO: 224), followed by a sequence encoding EGFP (SEQ ID NO: 218) or IDUA (SEQ ID NO: 2) without a stop codon, followed by the T2A self-cleaving peptide (SEQ ID NO: 225), followed by the first exon of S100A9 (SEQ ID NO: 210), followed by the 5′ albumin splicing signal sequence (SEQ ID NO: 208), followed by a right homology arm (SEQ ID NO: 211) for this intronic region.

For CD11b, AAV6 particles obtained from Vigene were used to insert the synthetic exon sequence into the first intron of the CD11b locus. This donor contains a left homology arm for this intronic region (SEQ ID NO: 212), followed by the 3′ albumin splicing signal sequence (SEQ ID NO: 206), followed by a sequence encoding a GSG linker (SEQ ID NO: 215), followed by a P2A self-cleaving peptide (SEQ ID NO: 224), followed by a sequence encoding EGFP (SEQ ID NO: 218) or IDUA (SEQ ID NO: 2) without a stop codon, followed by the T2A self-cleaving peptide (SEQ ID NO: 225), followed by the first exon of CD11b rewritten (SEQ ID NO: 213), followed by the 5′ albumin splicing signal sequence (SEQ ID NO: 208), followed by a right homology arm for this intronic region (SEQ ID NO: 214).

### TALE-Nucleases Reagents:

mRNA encoding TALE-Nucleases targeting CD11b (SEQ ID NO: TALEN_CD11B Left and SEQ ID NO: TALEN_CD11B Right), and S100A9 (SEQ ID NO: TALENS100A9 Left and SEQ ID NO: TALEN S100A9 Right) were produced according to previously described protocol (Poirot et al. 2015). The targeted sequences are TACAACATATTCTATCAgcctcttggtctgcaAAACCTAAAATTTACTA (SEQ ID NO: 125) for CD11b and TTAGGGGCCCTGACAGCtctccataggtggagGCCTCAGGCAGGCAGGA (SEQ ID NO: 175) for the S100A9 loci. (TALEN is a trademark designating TALE-nucleases heterodimers designed by Cellectis (8 rue de la Croix Jarry, Paris, France) comprising Fok-1 nuclease catalytic head as described in WO2011072246.

### Gene Editing Protocols: Transfection

Forty-eight hours after thawing, HSC were gene edited. For this, HSCs were harvested, washed once with PBS, and resuspended in High Performance electroporation buffer (#45-0802, BTX) at a concentration of 10×10⁶ cells/ml. TALEN mRNAs were mixed with cell suspension at 5 μg for each TALEN arm and per million cells. The cell and mRNA mixtures were electroporated on BTX PulseAgile, using the program shown in Table 5. The HSCs were transferred into the prewarmed expansion media in a final concentration of 2×10⁶ cells/ml.

TABLE 5
BTX PulseAgile settings for HSCs

| Settings | Group1 | Group2 | Group3 |
|---|---|---|---|
| Amplitude (V) | 1000 | 1000 | 130 |
| Duration (ms) | 0.1 | 0.1 | 0.2 |
| Interval (ms) | 0.2 | 100 | 2 |
| Number | 1 | 1 | 4 |

### Gene Editing: AAV Preparation and Transduction:

Immediately after electroporation, HSCs were transduced with AAV at various doses including 0.3e4, 1e4, or 3.2e4 viral genome per cell (vg/cell), incubated for 15 min at 37 C and transferred to 30 °C for 22 h for recovery. The following day cells were counted and diluted at 0.2-0.6×10⁶ cells/ml in expansion media and cultivated at 37 °C.

### Myeloid Differentiation

Myeoid genes like CD11b and S100A9 are not expressed in HSC. In order to see a phenotypic expression of these edited loci, HSC were incubated in myeloid differentiation media. Twenty-four hours after transfection/transduction, HSCs were counted and resuspended in myeloid expansion supplement (Stemcell Technologies, cat. no. 02694) diluted in STEM Span II (Stemcell Technologies, cat. no. 09655) and supplemented with Pen/strep at 2e5 cells/mL. Cells in myeloid expansion media were then plated in non-tissue culture treated plates and split every 3-4 days, seeded at approximately 2e5 cells/mL. After 14 days in culture, cells were recovered for flow cytometry staining, and/or seeded for IDUA secretion.

### IDUA Secretion Assay

Fourteen days after myeloid differentiation, cells were seeded in equal numbers (2e5 cells/well) in non-tissue culture 96-well culture plates and incubated in myeloid media at 37 °C. Four days after seeding, supernatant were collected and the amount of IDUA was characterized using a commercial ELISA (G-Biosciences, cat: IT2013).

### In Vivo Mouse Experiments

Six-week old female NSG mice were ordered from Jackson Laboratories and housed with ad libitum water and food. Mice were pre-conditioned for HSC transplant with busulfan (Sigma Aldrich, cat. no. B3625) reconstituted in DMSO and further diluted in PBS, freshly made on first day of injection. Busulfan was then sterilized using 0.2 um syringe filters. Mice were injected with 15 mg/kg busulfan i.p. once daily for 3 consecutive days prior to injection with HSCs. On the day of injection, animals were anesthetized with 3% isoflorine and injected with 1.5e6 HSC by retro-orbital injection. Engraftment was assessed 16 weeks after injection by analyzing for the presence of human cells in blood, bone marrow and brain.

### Brain Isolation

Mice were first anesthetized with isofluorane 4% and perfused with 50 mL cold PBS. Brain was extracted and placed in 5 mL DMEM with 5% FBS+Pen/Strep and kept on ice while all mice and tissue are being processed. Once all brains have been extracted, brain was cut into small pieces using sterile scissors and passed through 16 g needle 3 times to homogenize. To further digest brain tissue, 500 ug/mL papain and 20 U/mL DNase I was added to brain homogenate and incubated for 30 minutes at 37 °C. After incubation, remaining brain tissue was passed through 40 μM cell strainer and washed with fresh DMEM. Cells were then resuspended in 30% Percoll gradient, underlay with 70% Percoll gradient, and spun for 25 minutes at 600 g to remove myelin. Cells in the interphase of Percoll gradient were recovered, washed, and stained for flow cytometry or kept for genomic DNA extraction.

### Flow Cytometry

Cells were spun down at 400×g for 5 minutes. Supernatant was removed and cells were washed once in FACS buffer (1 mM EDTA+0.5% BSA in PBS). After wash, cells were resuspended in 50 μL Fc block (BD Bioscience, cat. no. 564220) diluted at 5 μg/mL in FACS buffer. After 5 minutes incubation at 4 °C, 50 μL of surface antibodies master mix diluted in FACS buffer was added to cells and incubated for an additional 30 minutes at 4 °C. Cells were then washed twice in FACS buffer, followed by fixation in 100 μuL Fixative/Perm solution (BD Bioscience, cat. no. 554722) for 20 minutes at 4 °C. Cells were washed once in Permeabilization buffer, followed by incubation in intracellular antibodies mix (diluted in Permeabilization buffer) for 30 minutes at 4 °C. Cells were washed once again in Permeabilization buffer and resuspended in 100-200 μL FACS buffer prior to analysis on BD Canto.

For in vitro myeloid differentiation, the following antibodies were used: CD11b APC (Miltenyi 130-110-554), CD14 VioBlue (Miltenyi 130-113-152), and S100A9 PE (Invitrogen MA5-28130). For staining of bone marrow cells, the following antibodies were used: hCD45 PE-Cy7 (BD 103114), mCD45 V450 (BD 560501), CD33 PE (Miltenyi 130-113-349), CD3 PerCP-Cy5.5 (BD 560835), CD34 APC-V770 (Miltenyi 130-113-180), CD19 FITC (BD 555412). For staining of isolated brain cells, the following antibodies were used: hCD45 FITC (Miltenyi 130-113-117), mCD45 APC-Cy7 (BD 557659), P2RY12 BV421 (Biolegend 392106), purified TMEM119 (Biolegend 853302), anti-mIgG2b AF647 (Biolegend 406716), CD11b PE (Biolegend 101208). For each antibody, fluorescence minus one (FMO) controls and single stain compensation beads were included.

### Example 2: An Artificial Exon (ArtEx) for the Expression of GFP can be Inserted Between Exon #1 and #2 and Adequately Processed for Expression—In Vitro Results

Insertion of an artificial exon in between the 2 first exons of the myeloid genes S100A9 or CD11b, should achieve expression of a therapeutic protein from the myeloid lineage, without compromising the endogenous expression of S100A9 or CD11b. For a proof of concept, we generated TALEN targeting the intronic region of each gene, as well AAV donors carrying a GFP cassettes allowing expression monitoring. As a control for lineage specific expression of our approach, we also generated reagents to insert a promoter containing GFP cassette into the AAVS1 locus as a more traditional safe harbor approach that will be expressed in all blood lineages.

Pre-stimulated HSCs were transfected with TALEN mRNA targeting the AAVS1, S100A9 and CD11b loci, and transduced with an increasing dose of the corresponding AAV-GFP repair template. Edited HSC were then differentiated in myeloid-like cells. Fourteen days after, differentiated cells were screened by flow cytometry to characterize GFP expression in different cellular subsets.

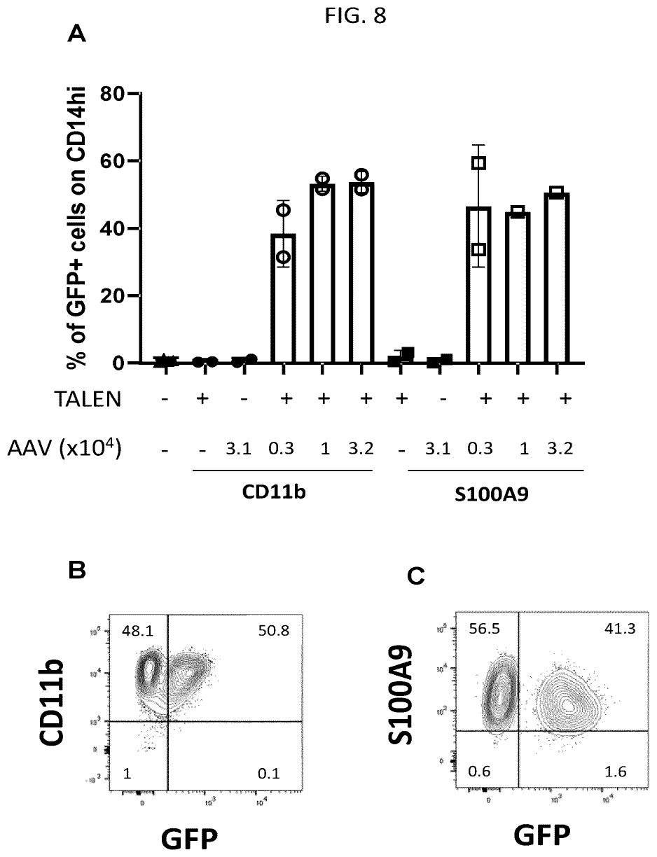

Fourteen days after differentiation between 40-60% of cells were CD14+. Among CD14high cells, gene editing rates defined by GFP expression ranged from 26% to 60% largely depending on the AAV dose used, achieving a maximum value of 56%, and 60% for CD11b and S100A9 locus, respectively (FIG. 8A). We also evaluated the expression of the endogenous S100A9 and CD11b in CD14 high cells. All CD14 high cells were positive for CD11b and S100A9, regardless of whether they were edited (GFP+) or not (GFP−) (FIGS. 8B and 8C).

The presence of a substantial amount of GFP cells in either loci, demonstrate that the GFP cassette was adequately inserted in between the first 2 exons, and that our added splicing signals were adequately processed by the splicing cellular machinery. This ArtEx strategy allows expression of a bi-functional mRNA molecule able to translated for both the inserted gene (i.e. GFP) protein and the endogenous CD11b or S100A9 proteins.

### Example 3: An Artificial Exon for the Expression of IDUA can be Inserted Between Exon #1 and #2 and Adequately Processed for Expression and Secretion—In Vitro Results

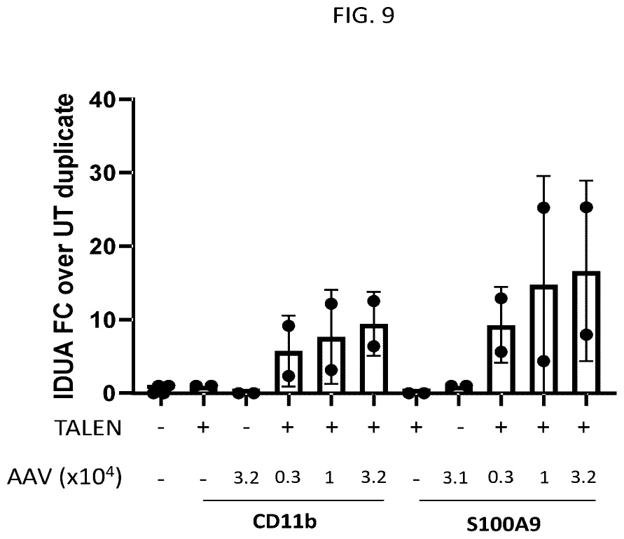

Pre-stimulated HSCs were transfected with TALEN mRNA targeting S100A9 and CD11b loci, and transduced with the increasing dose of the corresponding AAV-IDUA repair template. Edited HSC were placed into myeloid differentiation media. Fourteen days after, myeloid-like differentiated cells were seeded for IDUA production without any enrichment (% of myeloid between 40-60%). Cell supernatants were collected 3 days after and IDUA was quantified by ELISA. We observed that cells edited at CD11b and S100A9 loci secreted 10× and 15×fold more IDUA than unedited controls, respectively (FIG. 9).

These results confirm that ArtEx strategy allows a specific expression and secretion of therapeutic proteins.

### Example 4: Edited HSC Successfully Engrafted in Blood and Bone Marrow of an Animal Model: In Vivo Results

One of the more relevant characteristics of HSC, basis of our therapeutic approach, is their ability to provide a lifelong supply of edited cells after a single intervention. To do so, HSC need to engraft in the bone marrow, proliferate and produce blood cells that will later populate multiple tissues in the body.

To provide some insight of this HSC ability, an immunodeficient animal model was used. The edited HSC with the abovementioned GFP cassettes targeted at the S100A9 locus were injected 24 h post editing into conditioned NSG females. This animal model has shown to sustain the engraftment of human HSC in the animal bone marrow.

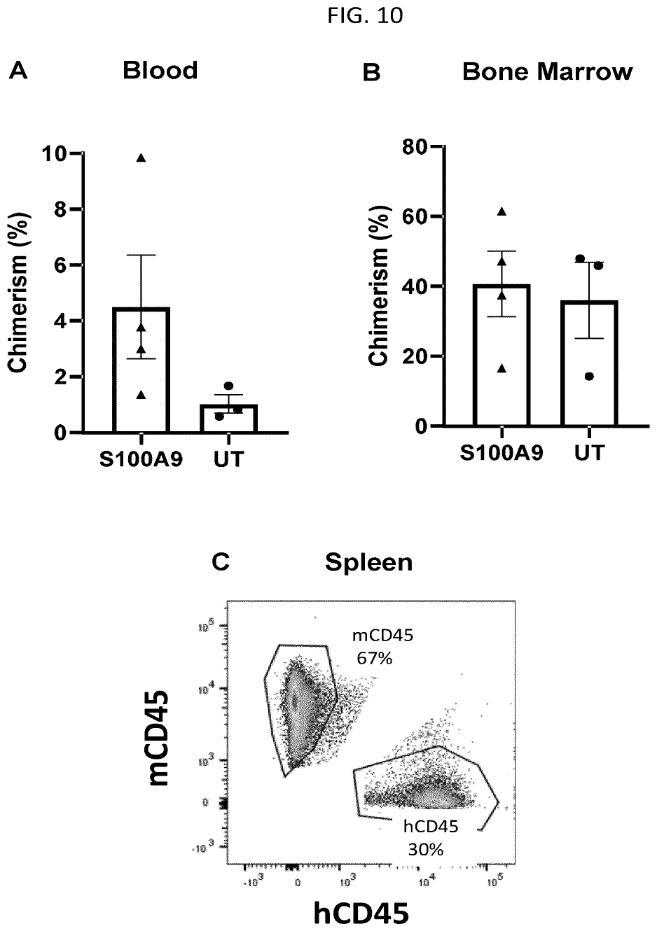

Sixteen weeks after the injection of edited HSC, engraftment in blood and bone marrow was detected in all animals at similar levels among study groups, averaging 3.3% in blood and 40.8% in bone marrow (FIGS. 10A and B). In addition, between 24 and 30% of human cells could be detected in the spleens of animals (FIG. 10C). More importantly, edited cells in all these compartments could be detected.

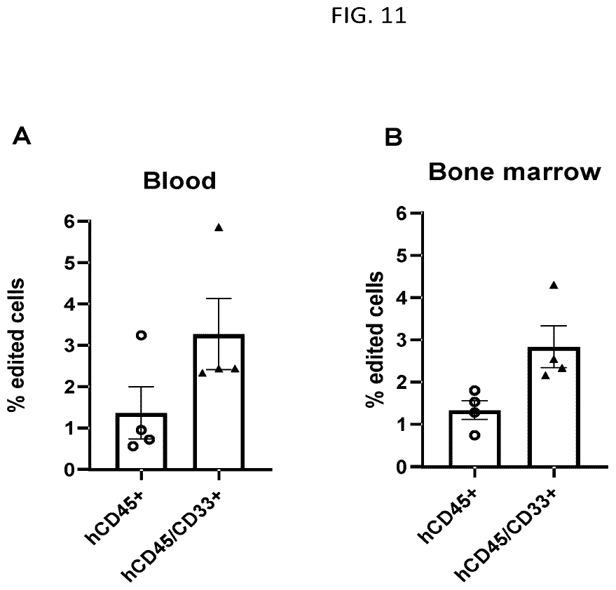

The presence of edited cells in blood of these animals was analyzed. On bulk human CD45 cells, we found an average of 1.4% of GFP+ cells in blood of animals injected with HSC edited at the S100A9 locus. However, when analyzing into the myeloid compartment, defined in this model as CD33+ cells, the editing rate increased up to 3.3%, twice as higher than in bulk population (FIG. 11A).

The percentage of edited cells was also analyzed in the bone marrow, and 1.3% of human cells were GFP+. In addition, 2.8% of hCD45+ and CD33+ human cells in the bone marrow were GFP+ (FIG. 10B)

### Example 5: Edited HSC Successfully Engrafted in the Brain of an Animal Model: In Vivo Results

Another potential advantage of this therapeutic approach is the ability that HSC derived microglia can secrete a deficient LSD enzyme in the brain compartment, enabling the treatment of the devastating neurological symptoms associated with these LSD diseases. To investigate about this potential feature, the presence of human cells in the brains of the aforementioned animals was analyzed.

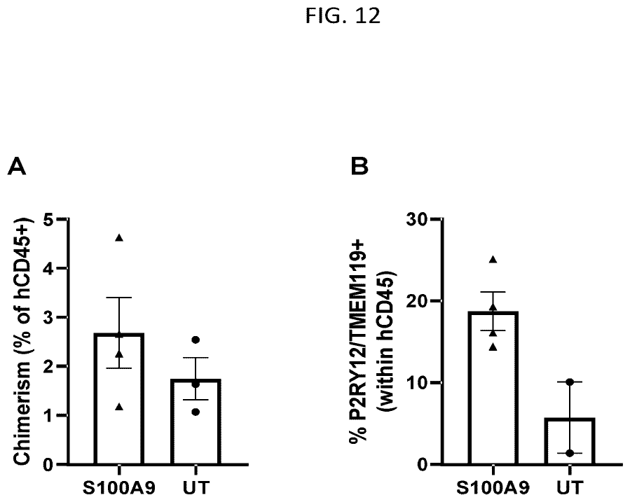

After isolation of mouse brains and cell processing, significant amount of human cells in mouse brains could be detected. On average 2.7% of cells in the brain had human origin (FIG. 12A). More importantly, 18.5% of these human cells contained derived microglia, using P2RY12 and TMEM119 microglial markers (FIG. 12B). Since these 2 markers were not found in any human cells present in the peripheral blood of these animals, it ruled out any potential contamination of brain isolate with peripheral blood cells during extraction.

In this brain compartment, GFP positive cells represented at least 1.2% and 1.6% of all human cells and of the human microglial cells, respectively.

### Example 6: ArtEx Edited HSCs have High Secretion Profile

ArtEx edited HSCs were compared to classical lentiviral edited HSCs for their ability to secrete the therapeutic protein.

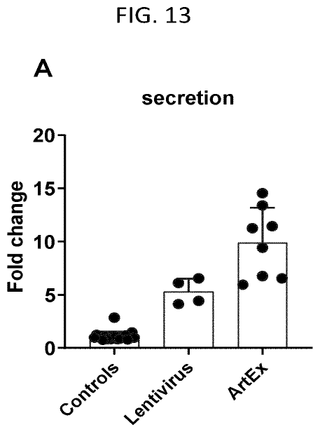

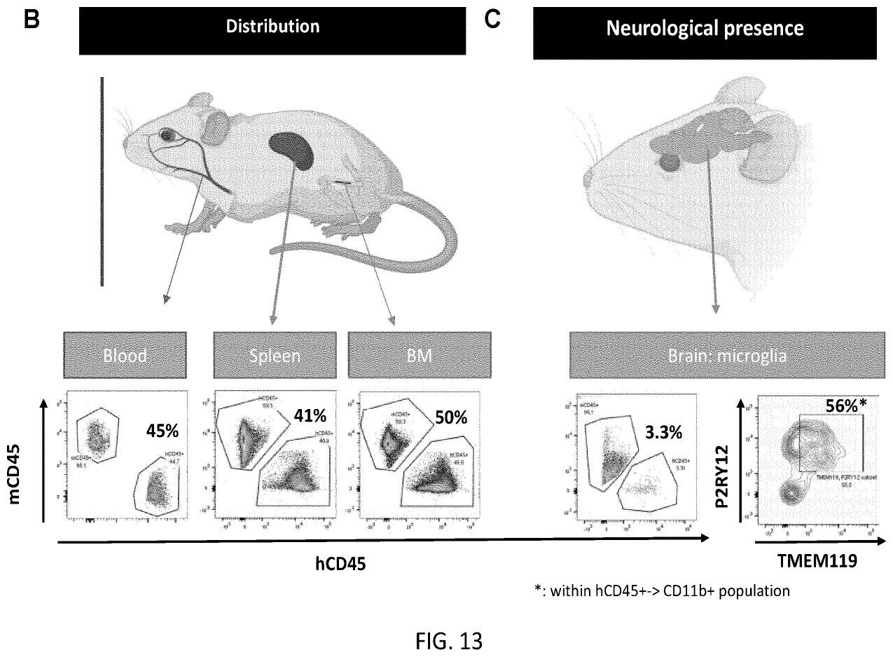

Untreated HSCs, HSCs transduced with a lentiviral vector allowing the expression of IDUA and HSCs with targeted integration of IDUA at the S100A9 or CD11b loci as described in preceding examples. Edited HSC were placed into myeloid differentiation media. Fourteen days after, myeloid-like differentiated cells were seeded for IDUA production. Cell supernatants were collected 3 days after and IDUA was quantified by ELISA. We observed that cells edited at CD11b and S100A9 loci secreted 10× and 15×fold more IDUA than unedited controls, respectively (FIG. 13A).

Results demonstrate that ArtEx edited HSCs were able to stimulate IDUA secretion by a 10fold factor whereas HSCs transduced with a lentivirus vector stimulate IDUA secretion by a 5 fold factor.

In addition the HSCs were tested for engraftment efficacy in mice and as previously observed the edited HSCs could engraft efficiently into the bone marrow (50%), the spleen (41%) and the blood (45%) and most importantly into the brain up to 3,3% with a significant amount of microglial cells (FIG. 13B ad C).

All together these results reveal the potential of ArtEx strategy to secret high level of therapeutic protein even in the brain.

1-35. (canceled)

36. A method of treatment comprising expressing a transgene into the brain of a patient in need thereof comprising the steps:
i) obtaining genetically modified hematopoietic stem cells (HSCs), wherein wherein the genetically modified HSC have been engineered to comprise a transgene encoding a therapeutic protein integrated at an endogenous locus expressed in microglial cells selected from: tmem119, s100a9, cd11b, b2m, cx3cr1, mertk, cd164, tlr4, tlr7, cd14, fcgr1a, fcgr3a, tbxas1, dok3, abca1, tmem195, mr1, csf3r, fgd4, tspan14, tgfbri, ccr5, gpr34, serpine2, slco2b1, p2ryl2, olfml3, p2ryl3, hexb, rhob, jun, rab3il1, ccl2, fcrls, scoc, siglech, slc2a5, lrrc3, plxdc2, usp2, ctsf, cttnbp2nl, atp8a2, lgmn, mafb, egr1, bhlhe41, hpgds, ctsd, hspa1a, lag3, csf1r, adamts1, f11r, golm1, nuak1, crybb1, ltc4s, sgce, pla2g15, ccl311, abhd12, ang, ophn1, sparc, pros1, p2ry6, lair1, il1a, epb4112, adora3, rilpl1, pmepa1, ccl13, pde3b, scamp5, ppp1r9a, tjp1, ak1, b4galt4, gtf2 h2, trem2, ckb, acp2, pon3, agmo, tnfrsf17, fscn1, st3gal6, adap2, ccl4, entpd1, tmem86a, kctd12, dst, ctsl2, abcc3, pdgfb, pald1, tubgcp5, rapgef5, stab1, lacc1, tmc7, nrip1, kcnd1, tmem206, hps4, dagla, extl3, mlph, arhgap22, cxxc5, p4ha1, cysltr1, fgd2, kcnk13, gbgt1, c18orf1, cadm1, bco2, adrb1, c3ar1, large, leprel1, liph, upk1b, p2rx7, slc46a1, ebf3, ppp1r15a, il10ra, rasgrp3, fos, tppp, slc24a3, havcr2, nav2, apbb2, clstn1, bink, gnaq, ptprm, frmd4a, cd86, tnfrsf11a, spint1, ppm11, tgfbr2, cmklr1, tlr6, gas6, hist1h2ab, atf3, acvr1, abi3, Irp12, ttc28, plxna4, adamts16, rgs1, icam1, snx24, ly96, dnajb4, and ppfia4.
ii) engrafting the genetically modified HSCs of step i) into said patient in order to have them differentiate into microglial cells expressing the transgene into the patient's brain.

37. A method of treatment according to claim 36, wherein said transgene has been integrated into an endogenous intronic genomic region at an insertion site comprising
a first strong splice site sequence comprising a branch point and an acceptor site;
a first sequence encoding 2A self-cleaving peptide;
said transgene coding for a therapeutic protein;
a second sequence encoding 2A self-cleaving peptide;
a copy of the coding sequence of the preceding exons endogenous to said locus;
a second strong splice site sequence comprising a splice donor site.

38. A method of treatment according to claim 36, wherein said therapeutic protein is selected from IDUA, IDS, ARSA, ARSB, GUSB, ABCD1, GALC, ARSA, PSAP, GBA, FUCA1, MAN2B1, AGA, ASAH1, HEXA, GAA, SMPD1, LIPA, CDKL5, FANCA, FANCC, FANCG, HBB, PKLR, RPS19, HAX1, CYBA, CYBB, NCF1, NCF2, NCF4, Factor 8, Factor 9, Factor 11, WAS, IL2RG or BTK.

39. A method of treatment according to claim 36, wherein said transgene is integrated at an endogenous locus selected from s100a9 and cd11b.

40. A method of treatment according to claim 36, wherein said therapeutic protein is IDUA.

41. A method of treatment according to claim 36, wherein the HSCs are isolated from the patient.

42. A method of treatment according to claim 36, wherein the HSCs are obtained from induced pluripotent stem (iPS) cells derived from the patient and differentiated into HSCs,

43. The method according to claim 37, wherein said copy of the coding sequence of the preceding exons endogenous to said locus has been rewritten.
